(19) United States
(12) Patent Application Publication
(10) Pub. No.: US 2026/0282647 A1
(43) Pub. Date: Sep. 17, 2026

(54) ORGANIC COMPOUND AND ORGANIC LIGHT-EMITTING DEVICE COMPRISING SAME

(71) Applicant: LG CHEM, LTD., Seoul (KR)

(72) Inventors: Jae Seung HA, Daejeon (KR); Sujeong GEUM, Daejeon (KR); Da Jung LEE, Daejeon (KR); Jiye JEON, Daejeon (KR); Woo Jin CHO, Daejeon (KR); Ji Young CHOI, Daejeon (KR); Woochul LEE

(21) Appl. No.: 19/167,228

(22) PCT Filed: Apr. 9, 2024
(86) PCT No.: PCT/KR2024/004694

§ 371 Date: Sep. 19, 2025

(30) Foreign Application Priority Data
Apr. 12, 2023 (KR) 10-2023-0048395

(51) Int. Cl. H10K 50/12; C07B 59/00; C07D 307/87; H10K 85/60

(52) U.S. Cl. CPC H10K 50/12; C07B 59/002; C07D 307/87; H10K 85/626; H10K 85/633; H10K 85/636; H10K 85/654; H10K 85/6572; H10K 85/6574; H10K 85/658; C07B 2200/05

(57) ABSTRACT

A compound represented by Chemical Formula 1-1 or Chemical Formula 1-2:

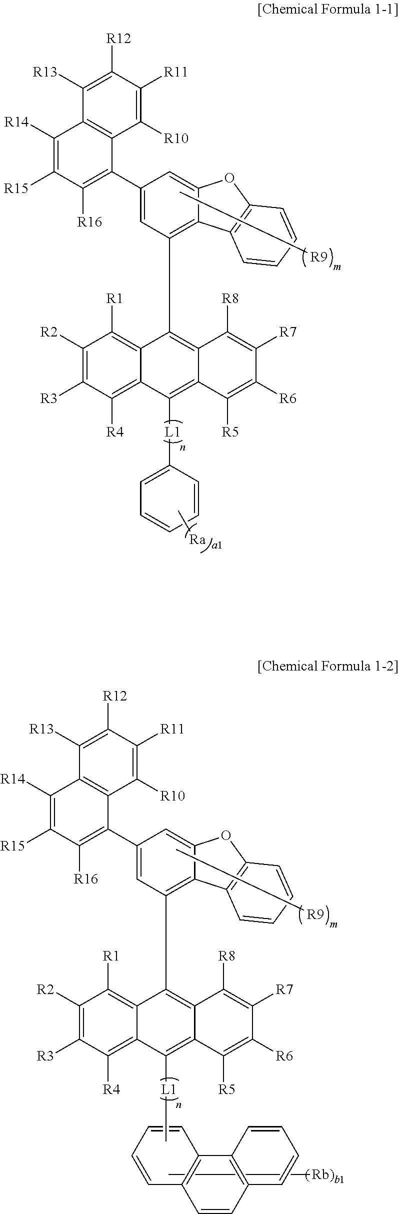

where the substituents are as defined in the specification, and an organic light-emitting device including the same, wherein the compound represented by Chemical Formula 1-1 or Chemical Formula 1-2 is a compound in which anthracene, serving which serves as a key core when functioning as a blue fluorescent host compound, is substituted with deuterium. The compound represented by Chemical Formula 1-1 or Chemical Formula 1-2 can be used in an organic light-emitting device to improve device characteristics, e.g., lowering driving voltages, improving efficiency, or improving lifespan characteristics.

### CROSS-REFERENCE TO RELATED APPLICATION(S)

This application is a National Stage Application of International Application No. PCT/KR2024/004694 filed on Apr. 9, 2024, which claims the benefit of and priority to Korean Patent Application No. 10-2023-0048395, filed in the Korean Intellectual Property Office on Apr. 12, 2023, the entire content of which is incorporated herein by reference.

### TECHNICAL FIELD

The present specification relates to an organic compound and an organic light-emitting device containing the same.

### BACKGROUND ART

In general, the phenomenon of organic light emission refers to a phenomenon wherein electrical energy is converted to light energy using an organic material. An organic light-emitting device, which utilizes the phenomenon of organic light emission, typically has a structure that includes an anode, a cathode, and an organic layer situated between them. Here, the organic layer often has a multilayer structure consisting of different materials to enhance the efficiency and stability of the organic light-emitting device. For example, it can consist of a hole injection layer, a hole transport layer, a light-emitting layer, an electron transport layer, an electron injection layer, etc. In the structure of the organic light-emitting device, when a voltage is applied between the two electrodes, holes are injected from the anode and electrons are injected from the cathode into the organic layer. Excitons are formed when the injected holes and electrons recombine, and light is emitted as the excitons return to the ground state.

It is required to develop new materials for the organic light-emitting device.

### References of Related Art

### Patent Documents

(Patent document 1) KR 10-2022-0081059 A.

### BRIEF DESCRIPTION

The present specification provides an organic compound and an organic light-emitting device containing the same.

An exemplary embodiment of the present specification provides a compound represented by Chemical Formula 1-1 or Chemical Formula 1-2.

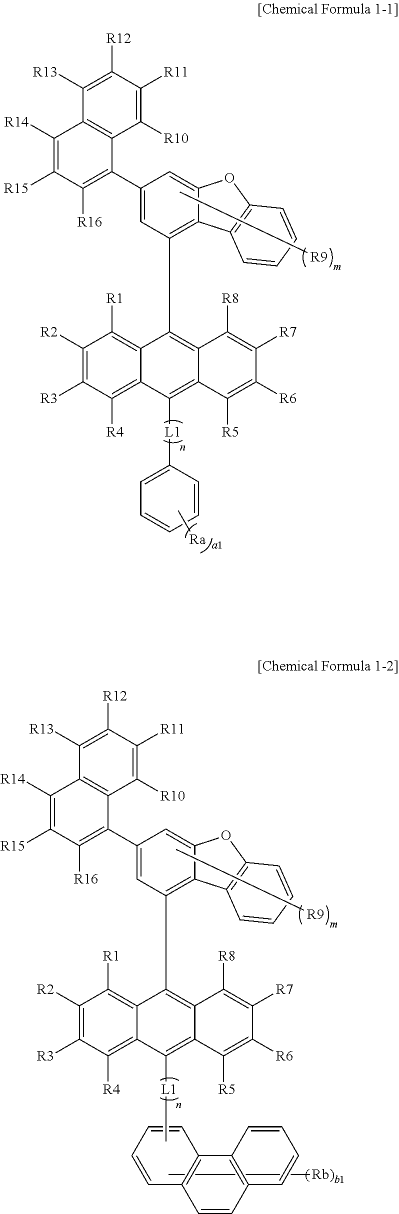

In Chemical Formula 1-1 or Chemical Formula 1-2,
R1 to R8, which are identical or different, are independently deuterium; a substituted or unsubstituted aryl group; or a substituted or unsubstituted heteroaryl group,
R9, which is identical or different, is independently hydrogen; deuterium; or a substituted or unsubstituted alkyl group,
R10 to R16, which are identical or different, are independently hydrogen; deuterium; a substituted or unsubstituted alkyl group; a substituted or unsubstituted cycloalkyl group; or a substituted or unsubstituted aryl group,
L1, which is identical or different, is independently a direct bond; or a substituted or unsubstituted heteroarylene group,
Ra, which is identical or different, is independently hydrogen; deuterium; a substituted or unsubstituted alkyl group; a substituted or unsubstituted aliphatic hydrocarbon ring group; a substituted or unsubstituted aryl group wherein three or more ring are condensed; or a substituted or unsubstituted heteroaryl group, or is bonded to an adjacent substituent to form an aliphatic hydrocarbon ring,
Rb, which is identical or different, is independently hydrogen; deuterium; a substituted or unsubstituted alkyl group; a substituted or unsubstituted cycloalkyl group; a substituted or unsubstituted aryl group; or a substituted or unsubstituted heteroaryl group,
m is an integer from 1 to 6,
n is an integer 1 or 2,
a1 is an integer from 1 to 5,
b1 is an integer from 1 to 9, and
if each of m, n, a1 or b1 is 2 or greater, the R9s, L1s, Ras or Rbs are identical or different.

In addition, an exemplary embodiment of the present specification provides an organic light-emitting device including a first electrode; a second electrode provided opposite to the first electrode; and one or more organic layers provided between the first electrode and the second electrode, wherein at least one of the one or more organic layer contains the compound of Chemical Formula 1-1 or Chemical Formula 1-2.

### Advantageous Effects

A compound described in the present specification can be used as a material for an organic layer of an organic light-emitting device. The compound according to at least one exemplary embodiment of the present specification can improve the efficiency, lower operation voltage and/or improve lifetime characteristics of an organic light-emitting device.

### BRIEF DESCRIPTION OF THE DRAWINGS

FIG. 1 shows an example of an organic light-emitting device in which a substrate 1, a first electrode 2, a light-emitting layer 6, and a second electrode 10 are stacked sequentially.

FIG. 2 shows an example of an organic light-emitting device in which a substrate 1, a first electrode 2, a hole injection layer 3, a hole transport layer 4, an electron-blocking (hole control) layer 5, a light-emitting layer 6, a hole-blocking (electron control) layer 7, an electron transport layer 8, an electron injection layer 9, and a second electrode 10 are stacked sequentially.

FIG. 3 shows an example of an organic light-emitting device in which a first electrode 2, a hole injection layer 3, a hole transport layer 4, an electron-blocking (hole control) layer 5, a light-emitting layer 6, a hole-blocking (electron control) layer 7, an electron transport layer 8, an electron injection layer 9, a second electrode 10, and a capping layer 11 are stacked sequentially on a substrate 1.

### DETAILED DESCRIPTION

Hereinafter, the present specification is described in more detail.

In the present specification, when a certain part is said to “include” a certain component, it means that, unless stated otherwise, additional components may also be included.

In the present specification, when a member is said to be located “on” another member, it includes not only the case where the member is in contact with another member but also the case where a still another member exists between the two members.

In the present disclosure,

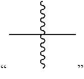

indicates a position that is bonded to a chemical formula or a compound.

In the present specification, at least one deuterium is directly substituted in the anthracene of Chemical Formula 1-1 or 1-2. A light-emitting layer of an organic light-emitting device is the region that emits light and where energy loss occurs. A carbon-deuterium bond is stronger than a carbon-hydrogen bond. And, since deuterium has a larger mass than hydrogen, it has a high binding energy due to decreased zero-point energy with carbon. Therefore, by replacing the carbon-hydrogen bond contained in the compound of Chemical Formula 1-1 or 1-2 with a carbon-deuterium bond, the binding energy of the molecule can be increased to provide a device having excellent lifetime.

In the present specification, the deuterium substitution rate means the substitution rate of deuterium within a molecule, and the distribution of deuterium substitution within a molecule is determined by the following two methods. Among the two methods described below, TLC-MS is a method for controlling the substitution rate of deuterium during the synthetic process of a compound.

### 1. TLC-MS (Thin-Layer Chromatography/Mass Spectrometry) (Method for Confirming the Synthesis Process)

The substitution rate can be calculated based on the maximum value (max. value) of the distribution of molecular weights at the end of the reaction.

### 2. Quantitative Analysis Using NMR

DMF (dimethylformamide) is added as an internal standard, and the D substitution rate is calculated from the total peak integration amount using the integration ratio on the ¹H NMR spectrum.

In the present specification, the examples of substituents are described below, although not being limited thereto.

The term “substitution” refers to replacement of a hydrogen atom bonded to the carbon atom of a compound by another substituent. The position of substitution is not limited as long as it is the position where the hydrogen atom can be replaced. When there are two or more substitutions, the two or more substituents can be identical or different.

In the present specification, the term “substituted or unsubstituted” means substituted with one or more substituent selected from a group consisting of deuterium; an alkyl group; a cycloalkyl group; an aryl group; or a heterocyclic group, substituted with a substituent in which two or more of the above-mentioned substituents are linked together, or having no substituent at all. For example, “a substituent in which two or more of substituents are linked together” may be a biphenyl group. That is to say, a biphenyl group can be seen as an aryl group, or a substituent in which two phenyl groups are linked together.

The examples of the above substituents are described below, although not being limited thereto.

In the present specification, the alkyl group may be linear or branched, and the number of carbon atoms may be specifically 1 to 60, although not being specially limited thereto. According to an exemplary embodiment, the number of carbon atoms in the alkyl group is 1 to 30. According to another exemplary embodiment, the number of carbon atoms in the alkyl group is 1 to 20. According to another exemplary embodiment, the number of carbon atoms in the alkyl group is 1 to 10. Specific examples of the alkyl group include a methyl group, an ethyl group, a propyl group, a n-propyl group, an isopropyl group, a butyl group, a n-butyl group, an isobutyl group, a tert-butyl group, a pentyl group, a n-pentyl group, a hexyl group, a n-hexyl group, a heptyl group, a n-heptyl group, an octyl group, a n-octyl group, etc., although not being limited thereto.

In the present specification, the foregoing description of the alkyl group is applicable to an arylalkyl group, except that it is substituted with an aryl group.

The alkyl groups and other substituents containing alkyl moieties described in the present specification encompass both linear and branched forms.

In the present specification, the number of carbon atoms in the cycloalkyl group is specifically 3 to 60, although not being specially limited thereto. In an exemplary embodiment, the number of carbon atoms in the cycloalkyl group is 3 to 30. According to another exemplary embodiment, the number of carbon atoms in the cycloalkyl group is 3 to 20. According to another exemplary embodiment, the number of carbon atoms in the cycloalkyl group is 3 to 6. Specific examples include a cyclopropyl group, a cyclobutyl group, a cyclopentyl group, a cyclohexyl group, a cycloheptyl group, a cyclooctyl group, etc., although not being limited thereto.

In the present specification, the number of carbon atoms in the aryl group is specifically 6 to 60, although not being specially limited thereto, and the aryl group may be a monocyclic aryl group or a polycyclic aryl group. According to another exemplary embodiment, the number of carbon atoms in the aryl group is 6 to 30. According to an exemplary embodiment, the number of carbon atoms in the aryl group is 6 to 20. The aryl group may be a monocyclic aryl group such as a phenyl group, a biphenyl group, a terphenyl group, a quaterphenyl group, etc., although not being limited thereto. The polycyclic aryl group may be a naphthyl group, an anthracenyl group, a phenanthrenyl group, a pyrenyl group, a perylenyl group, a triphenyl group, a chrysenyl group, a fluorenyl group, a triphenylenyl group, etc., although not being limited thereto.

In the present specification, the fluorenyl group may be substituted, and two substituents may be bonded to each other to form a spiro structure. The spiro structure may be an aromatic hydrocarbon ring or an aliphatic hydrocarbon ring.

When the above fluorenyl group is substituted, it may be a spirofluorenyl group such as

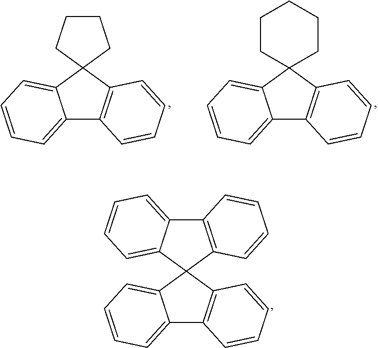

etc., or a substituted fluorenyl group such as,

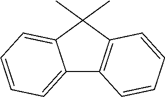

(9,9-dimethylfluorenyl group),

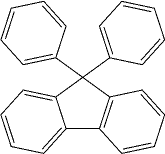 [SMILES: c1ccc(C2(c3ccccc3)c3ccccc3-c3ccccc32)cc1]

(9,9-diphenylfluorenyl group), etc., although not being limited thereto.

In addition, in the present specification, the aryl group may consist only of an aromatic ring, or the aromatic ring may be condensed with an aliphatic ring or an aliphatic group or may be substituted. The aryl group in which the aromatic ring is condensed with an aliphatic ring or an aliphatic group or is substituted may be a tetrahydronaphthyl group, etc., although not being limited thereto.

In the present specification, the heterocyclic group refers to a cyclic group that contains one or more heteroatom selected from N, O, P, S, Si and Se. The number of carbon atoms in the heterocyclic group is specifically 2 to 60, although not being specially limited thereto. According to an exemplary embodiment, the number of carbon atoms in the heterocyclic group is 2 to 30. According to an exemplary embodiment, the number of carbon atoms in the heterocyclic group is 2 to 20. Examples of the heterocyclic group include a pyridine group, a pyrrole group, a pyrimidine group, a quinoline group, a pyridazinyl group, a furan group, a thiophene group, an imidazole group, a pyrazole group, a dibenzofuran group, a dibenzothiophene group, a carbazole group, a benzocarbazole group, a naphthobenzofuran group, a benzonaphthothiophene group, an indenocarbazole group, a triazinyl group, etc., although not being limited thereto.

In the present specification, the foregoing description of the heterocyclic group is applicable to a heteroaryl group, except that it is aromatic.

In the present specification, the foregoing description of the aryl group is applicable to an arylene group, except that it is divalent.

In the present specification, the foregoing description of the heteroaryl group is applicable to a heteroarylene group, except that it is divalent.

In the present specification, in a substituted or unsubstituted ring formed by bonding of adjacent groups, the “ring” means a hydrocarbon ring; or a heterocycle.

The hydrocarbon ring may be an aromatic ring, an aliphatic ring, or a condensed ring of aromatic and aliphatic rings, and may be selected, for example, from the cycloalkyl group or aryl group described above.

In the present disclosure, the formation of a ring by bonding of adjacent groups means that a substituted or unsubstituted aliphatic hydrocarbon ring, a substituted or unsubstituted aromatic hydrocarbon ring, a substituted or unsubstituted aliphatic heterocycle, a substituted or unsubstituted aromatic heterocycle, or a condensed ring thereof is formed by bonding of adjacent groups. The hydrocarbon ring refers to a ring consisting only of carbon and hydrogen atoms. The heterocycle refers to a ring that contains one or more element selected from N, O, P, S, Si, Se, etc. In the present specification, the aliphatic hydrocarbon ring, aromatic hydrocarbon ring, aliphatic heterocyclic ring, and aromatic heterocyclic ring may be monocyclic or polycyclic.

In the present specification, the aliphatic hydrocarbon ring refers to a non-aromatic ring consisting only of carbon and hydrogen atoms. Examples of the aliphatic hydrocarbon ring include cyclopropane, cyclobutane, cyclobutene, cyclopentane, cyclopentene, cyclohexane, cyclohexene, 1,4-cyclohexadiene, cycloheptane, cycloheptene, cyclooctane, cyclooctene, etc., although not being limited thereto.

In the present specification, the aromatic hydrocarbon ring refers to an aromatic ring consisting only of carbon and hydrogen atoms. Examples of the aromatic hydrocarbon ring include benzene, naphthalene, anthracene, phenanthrene, perylene, fluoranthene, triphenylene, phenalene, pyrene, tetracene, chrysene, pentacene, fluorene, indene, acenaphthylene, benzofluorene, spirofluorene, etc., although not being limited thereto. In the present specification, the aromatic hydrocarbon ring may be interpreted as having the same meaning as the aryl group.

In the present specification, the aliphatic heterocycle refers to an aliphatic ring containing one or more heteroatom. Examples of the aliphatic heterocycle include oxirane, tetrahydrofuran, 1,4-dioxane, pyrrolidine, piperidine, morpholine, oxepane, azocane, thiocane, etc., although not being limited thereto.

In the present specification, the aromatic heterocycle refers to an aromatic ring containing one or more heteroatoms. Examples of the aromatic heterocycle include pyridine, pyrrole, pyrimidine, pyridazine, furan, thiophene, imidazole, pyrazole, oxazole, isoxazole, thiazole, isothiazole, triazole, oxadiazole, thiadiazole, dithiazole, tetrazole, pyran, thiopyran, diazine, oxazine, thiazine, dioxin, triazine, tetrazine, isoquinoline, quinoline, quinone, quinazoline, quinoxaline, naphthyridine, acridine, phenanthridine, diazanaphthalene, triazaindene, indole, indolizine, benzothiazole, benzoxazole, benzimidazole, benzothiophene, benzofuran, dibenzothiophene, dibenzofuran, carbazole, benzocarbazole, dibenzocarbazole, phenazine, imidazopyridine, phenoxazine, indolocarbazole, indenocarbazole, etc., although not being limited thereto.

Hereinafter, specific exemplary embodiments of the present disclosure will be described in detail. However, the exemplary embodiments of the present disclosure can be modified in various forms, and the scope of the present disclosure is not limited to the embodiments described below.

Therefore, when the compound represented by Chemical Formula 1-1 or Chemical Formula 1-2 described above is applied to an organic light-emitting device, an organic light-emitting device with high efficiency, low voltage and/or long lifetime characteristics can be obtained.

Hereinafter, Chemical Formula 1-1 or Chemical Formula 1-2 will be described in detail.

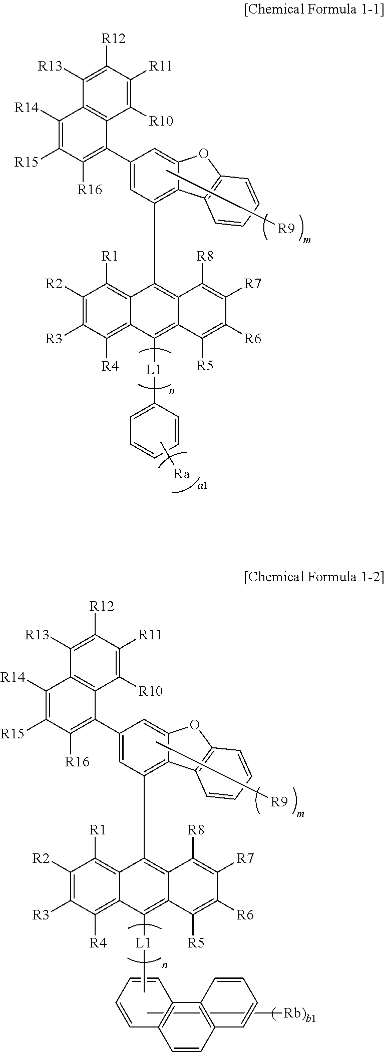

In Chemical Formula 1-1 or Chemical Formula 1-2,
R1 to R8, which are identical or different, are independently deuterium; a substituted or unsubstituted aryl group; or a substituted or unsubstituted heteroaryl group,
R9, which is identical or different, is independently hydrogen; deuterium; or a substituted or unsubstituted alkyl group,
R10 to R16, which are identical or different, are independently hydrogen; deuterium; a substituted or unsubstituted alkyl group; a substituted or unsubstituted cycloalkyl group; or a substituted or unsubstituted aryl group,
L1, which is identical or different, is independently a direct bond; or a substituted or unsubstituted heteroarylene group,
Ra, which is identical or different, is independently hydrogen; deuterium; a substituted or unsubstituted alkyl group; a substituted or unsubstituted aliphatic hydrocarbon ring group; a substituted or unsubstituted aryl group wherein three or more ring are condensed; or a substituted or unsubstituted heteroaryl group, or is bonded to an adjacent substituent to form an aliphatic hydrocarbon ring,
Rb, which is identical or different, is independently hydrogen; deuterium; a substituted or unsubstituted alkyl group; a substituted or unsubstituted cycloalkyl group; a substituted or unsubstituted aryl group; or a substituted or unsubstituted heteroaryl group,
m is an integer from 1 to 6,
n is an integer 1 or 2,
a1 is an integer from 1 to 5,
b1 is an integer from 1 to 9, and
if each of m, n, a1 or b1 is 2 or greater, R9's, L1's, Ra's or Rb's are identical or different.

In an exemplary embodiment of the present specification, Chemical Formula 1-1 is represented by Chemical Formula 1-11.

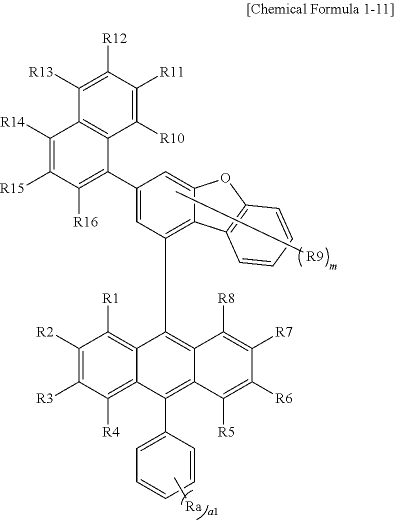

In Chemical Formula 1-11, R1 to R16, Ra, m and a1 are the same as defined in Chemical Formula 1-1.

In an exemplary embodiment of the present specification, Chemical Formula 1-2 is represented by Chemical Formula 1-21.

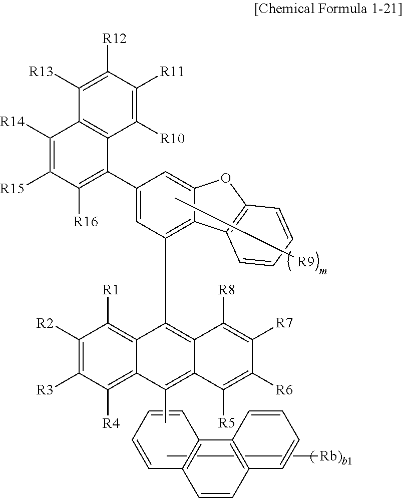

In Chemical Formula 1-21, R1 to R16, Rb, m and b1 are the same as defined in Chemical Formula 1-2.

In an exemplary embodiment of the present specification, Chemical Formula 1-2 is represented by any one of Chemical Formula 1-2a to Chemical Formula 1-2d.

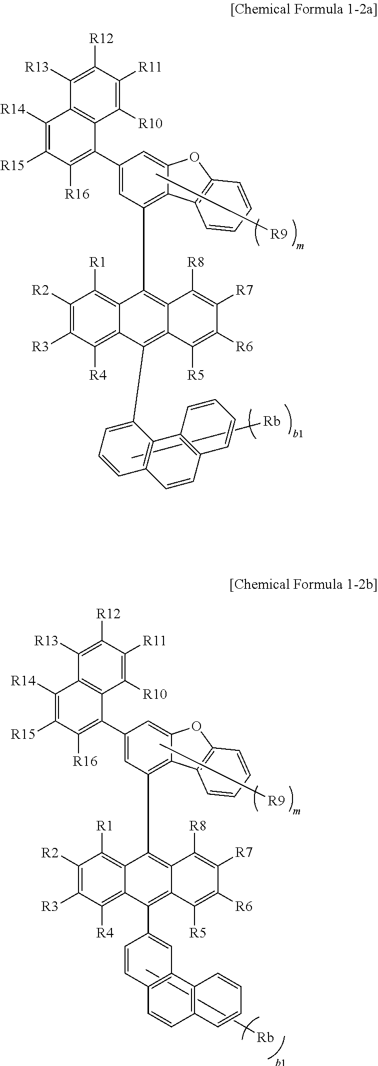

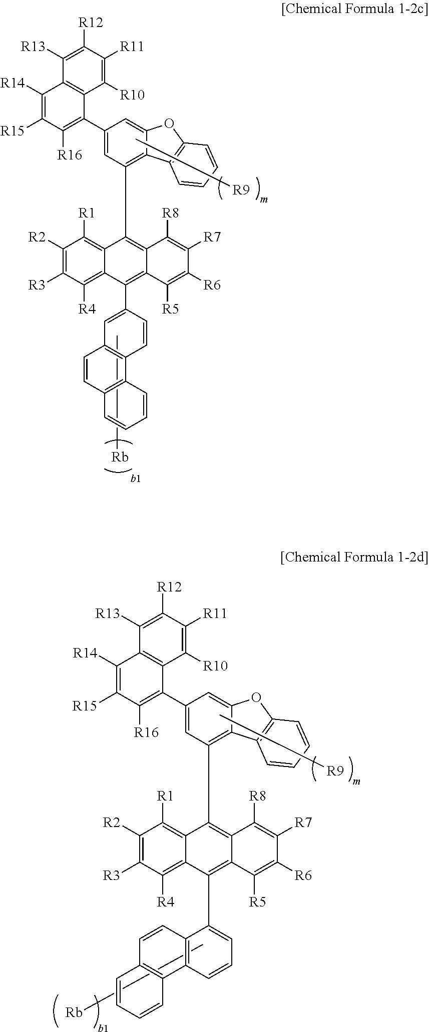

In Chemical Formula 1-2a to Chemical Formula 1-2d, R1 to R16, Rb, m and b1 are the same as defined in Chemical Formula 1-2.

In an exemplary embodiment of the present specification, R1 to R8, which are identical or different, are independently deuterium; a substituted or unsubstituted C₆₋₆₀ aryl group; or a substituted or unsubstituted C₂₋₆₀ heteroaryl group.

In an exemplary embodiment of the present specification, R1 to R8, which are identical or different, are independently deuterium; a substituted or unsubstituted C₆₋₃₀ aryl group; or a substituted or unsubstituted C₂₋₃₀ heteroaryl group.

In an exemplary embodiment of present the specification, R1 to R8, which are identical or different, are independently deuterium; a substituted or unsubstituted C₆₋₂₀ aryl group; or a substituted or unsubstituted C₂₋₂₀ heteroaryl group.

In an exemplary embodiment of the present specification, R1 to R8, which are identical or different, are independently deuterium; a substituted or unsubstituted C₆₋₁₂ aryl group; or a substituted or unsubstituted C₂₋₁₀ heteroaryl group.

In an exemplary embodiment of the present specification, R1 to R8, which are identical or different, are independently deuterium; or a substituted or unsubstituted C₆₋₂₀ aryl group.

In an exemplary embodiment of the present specification, R1 to R8, which are identical or different, are independently deuterium; or a substituted or unsubstituted C₆₋₁₂ aryl group.

In an exemplary embodiment of the present specification, R1 to R8, which are identical or different, are independently deuterium; or a substituted or unsubstituted C₆₋₃₀ aryl group, and at least one of R1 to R8 is a substituted or unsubstituted C₆₋₃₀ aryl group.

In an exemplary embodiment of the present specification, R1 to R8, which are identical or different, are independently deuterium; or a substituted or unsubstituted C₆₋₂₀ aryl group, and at least one of R1 to R8 is a substituted or unsubstituted C₆₋₂₀ aryl group.

In an exemplary embodiment of the present specification, R1 to R8, which are identical or different, are independently deuterium; or a substituted or unsubstituted C₆₋₁₂ aryl group, and at least one of R1 to R8 is a substituted or unsubstituted C₆₋₁₂ aryl group.

In an embodiment of the exemplary present specification, R1 to R8, which are identical or different, are independently deuterium; or a C₆₋₂₀ aryl group that is unsubstituted or substituted with deuterium.

In an exemplary embodiment of the present specification, R1 to R8, which are identical or different, are independently deuterium; or a C₆₋₁₂ aryl group that is unsubstituted or substituted with deuterium.

In an exemplary embodiment of the present specification, R1 to R8, which are identical or different, are independently deuterium; or a C₆₋₂₀ aryl group that is unsubstituted or substituted with deuterium, and at least one of R1 to R8 is a C₆₋₂₀ aryl group that is unsubstituted or substituted with deuterium.

In an exemplary embodiment of the present specification, R1 to R8, which are identical or different, are independently deuterium; or a C₆₋₁₂ aryl group that is unsubstituted or substituted with deuterium, and at least one of R1 to R8 is a C₆₋₁₂ aryl group that is unsubstituted or substituted with deuterium.

In an exemplary embodiment of the present specification, R1 to R8, which are identical or different, are independently deuterium; a substituted or unsubstituted phenyl group; a substituted or unsubstituted biphenyl group; a substituted or unsubstituted terphenyl group; a substituted or unsubstituted naphthyl group; a substituted or unsubstituted phenanthrenyl group; a substituted or unsubstituted triphenyl group; a substituted or unsubstituted fluorenyl group; a substituted or unsubstituted dibenzofuran group; a substituted or unsubstituted dibenzothiophene group; a substituted or unsubstituted benzofuran group; a substituted or unsubstituted benzothiophene group; or a substituted or unsubstituted carbazole group.

In an exemplary embodiment of the present specification, R1 to R8, which are identical or different, are independently deuterium; a substituted or unsubstituted phenyl group; a substituted or unsubstituted biphenyl group; a substituted or unsubstituted terphenyl group; a substituted or unsubstituted naphthyl group; a substituted or unsubstituted dibenzofuran group; a substituted or unsubstituted dibenzothiophene group; or a substituted or unsubstituted carbazole group.

In an exemplary embodiment of the present specification, R1 to R8, which are identical or different, are independently deuterium; a substituted or unsubstituted phenyl group; a substituted or unsubstituted biphenyl group; or a substituted or unsubstituted naphthyl group.

In an exemplary embodiment of the present specification, R1 to R8, which are identical or different, are independently deuterium; a phenyl group that is unsubstituted or substituted with deuterium; a biphenyl group that is unsubstituted or substituted with deuterium; or a naphthyl group that is unsubstituted or substituted with deuterium.

In an exemplary embodiment of the present specification, R1 to R8, which are identical or different, are independently deuterium; a phenyl group; a biphenyl group; or a naphthyl group.

In an exemplary embodiment of the present specification, R9 is hydrogen; deuterium; or a substituted or unsubstituted C₁₋₆₀ alkyl group.

In an exemplary embodiment of the present specification, R9 is hydrogen; deuterium; or a substituted or unsubstituted C₁₋₃₀ alkyl group.

In an exemplary embodiment of the present specification, R9 is hydrogen; deuterium; or a substituted or unsubstituted C₁₋₂₀ alkyl group.

In an exemplary embodiment of the present specification, R9 is hydrogen; deuterium; or a substituted or unsubstituted C₁₋₁₀ alkyl group.

In an exemplary embodiment of the present specification, R9 is hydrogen; deuterium; or a substituted or unsubstituted C₁₋₄ alkyl group.

In an exemplary embodiment of the present specification, R9 is hydrogen; deuterium; or a C₁₋₁₀ alkyl group that is unsubstituted or substituted with deuterium.

In an exemplary embodiment of the present specification, R9 is hydrogen; deuterium; or a C₁₋₄ alkyl group that is unsubstituted or substituted with deuterium.

In an exemplary embodiment of the present specification, R9 is hydrogen; deuterium; or a substituted or unsubstituted tert-butyl group.

In an exemplary embodiment of the present specification, R9 is hydrogen; deuterium; or a tert-butyl group that is unsubstituted or substituted with deuterium.

In an exemplary embodiment of the present specification, R9 is hydrogen; deuterium; or a tert-butyl group.

In an exemplary embodiment of the present specification, R9 is hydrogen; or deuterium.

In an exemplary embodiment of the present specification, R10 to R16, which are identical or different, are independently hydrogen; deuterium; a substituted or unsubstituted C₁₋₆₀ alkyl group; a substituted or unsubstituted C₃₋₆₀ cycloalkyl group; or a substituted or unsubstituted C₆₋₆₀ aryl group.

In an exemplary embodiment of the present specification, R10 to R16, which are identical or different, are independently hydrogen; deuterium; a substituted or unsubstituted C₁₋₃₀ alkyl group; a substituted or unsubstituted C₃₋₃₀ cycloalkyl group; or a substituted or unsubstituted C₆₋₃₀ aryl group.

In an exemplary embodiment of the present specification, R10 to R16, which are identical or different, are independently hydrogen; deuterium; a substituted or unsubstituted C₁₋₂₀ alkyl group; a substituted or unsubstituted C₃₋₂₀ cycloalkyl group; or a substituted or unsubstituted C₆₋₂₀ aryl group.

In an exemplary embodiment of the present specification, R10 to R16, which are identical or different, are independently hydrogen; deuterium; a substituted or unsubstituted C₁₋₁₀ alkyl group; a substituted or unsubstituted C₃₋₁₀ cycloalkyl group; or a substituted or unsubstituted C₆₋₁₂ aryl group.

In an exemplary embodiment of the present specification, R10 to R16, which are identical or different, are independently hydrogen; deuterium; a C₁₋₁₀ alkyl group that is unsubstituted or substituted with deuterium; a C₃₋₁₀ cycloalkyl group that is unsubstituted or substituted with deuterium; or a C₆₋₁₂ aryl group that is unsubstituted or substituted with deuterium.

In an exemplary embodiment of the present specification, R10 to R16, which are identical or different, are independently hydrogen; deuterium; a substituted or unsubstituted methyl group; a substituted or unsubstituted isopropyl group; a substituted or unsubstituted tert-butyl group; a substituted or unsubstituted cyclopentyl group; a substituted or unsubstituted cyclohexyl group; or a substituted or unsubstituted phenyl group.

In an exemplary embodiment of the present specification, R10 to R16, which are identical or different, are independently hydrogen; deuterium; or a substituted or unsubstituted phenyl group.

In an exemplary embodiment of the present specification, R10 to R16, which are identical or different, are independently hydrogen; deuterium; a methyl group that is unsubstituted or substituted with deuterium; an isopropyl group that is unsubstituted or substituted with deuterium; a tert-butyl group that is unsubstituted or substituted with deuterium; a cyclopentyl group that is unsubstituted or substituted with deuterium; a cyclohexyl group that is unsubstituted or substituted with deuterium; or a phenyl group that is unsubstituted or substituted with deuterium.

In an exemplary embodiment of the present specification, R10 to R16, which are identical or different, are independently hydrogen; deuterium; a methyl group; an isopropyl group; a tert-butyl group; a cyclopentyl group; a cyclohexyl group; or a phenyl group.

In an exemplary embodiment of the present specification, R10 to R16, which are identical or different, are independently hydrogen; deuterium; or a phenyl group that is unsubstituted or substituted with deuterium.

In an exemplary embodiment of the present specification, R10 to R16, which are identical or different, are independently hydrogen; deuterium; or a phenyl group.

In an exemplary embodiment of the present specification, L1, which is identical or different, is independently a direct bond; or a substituted or unsubstituted C₂₋₃₀ heteroarylene group.

In an exemplary embodiment of the present specification, L1, which is identical or different, is independently a direct bond; or a substituted or unsubstituted C₂₋₂₀ heteroarylene group.

In an exemplary embodiment of the present specification, L1, which is identical or different, is independently a direct bond; or a substituted or unsubstituted C₂₋₁₀ heteroarylene group.

In an exemplary embodiment of the present specification, L1, which is identical or different, is independently a direct bond; or a substituted or unsubstituted C₂₋₃₀ heteroarylene group containing one or more heteroatoms selected from N, O and S.

In an exemplary embodiment of the present specification, L1, which is identical or different, is independently a direct bond; or a substituted or unsubstituted C₂₋₂₀ heteroarylene group containing one or more heteroatoms selected from N, O and S.

In an exemplary embodiment of the present specification, L1, which is identical or different, is independently a direct substituted or bond; or a unsubstituted C₂₋₁₀ heteroarylene group containing one or more heteroatoms selected from N, O and S.

In an exemplary embodiment of the present specification, L1, which is identical or different, is independently a direct bond; a substituted or unsubstituted divalent dibenzofuran group; a substituted or unsubstituted divalent dibenzothiophene group; or a substituted or unsubstituted divalent carbazole group.

In an exemplary embodiment of the present specification, L1, which is identical or different, is independently a direct bond; a divalent dibenzofuran group that is unsubstituted or substituted with deuterium; or a divalent dibenzothiophene group that is unsubstituted or substituted with deuterium.

In an exemplary embodiment of the present disclosure, L1 is a direct bond.

In an exemplary embodiment of the present specification, Ra, which is identical or different, is independently hydrogen; deuterium; a substituted or unsubstituted C₁₋₆₀ alkyl group; a substituted or unsubstituted C₃₋₆₀ aliphatic hydrocarbon ring group; a substituted or unsubstituted C₁₀₋₆₀ aryl group wherein three or more ring are condensed; or a substituted or unsubstituted C₂₋₆₀ heteroaryl group, or is bonded to an adjacent substituent to form a substituted or unsubstituted aliphatic hydrocarbon ring.

In an exemplary embodiment of the present specification, Ra, which is identical or different, is independently hydrogen; deuterium; a substituted or unsubstituted C₁₋₃₀ alkyl group; a substituted or unsubstituted C₃₋₃₀ aliphatic hydrocarbon ring group; a substituted or unsubstituted C₁₀₋₃₀ aryl group wherein three or more ring are condensed; or a substituted or unsubstituted C₂₋₃₀ heteroaryl group, or is bonded to an adjacent substituent to form a substituted or unsubstituted C₃₋₃₀ aliphatic hydrocarbon ring.

In an exemplary of embodiment the present specification, Ra, which is identical or different, is independently hydrogen; deuterium; a substituted or unsubstituted C₁₋₂₀ alkyl group; a substituted or unsubstituted C₃₋₁₀ aliphatic hydrocarbon ring group; a substituted or unsubstituted C₁₀₋₂₀ aryl group wherein three or more ring are condensed; or a substituted or unsubstituted C₂₋₂₀ heteroaryl group, or is bonded to an adjacent substituent to form a substituted or unsubstituted C₃₋₁₀ aliphatic hydrocarbon ring.

In an exemplary embodiment of the present specification, Ra, which h is identical or different, is independently hydrogen; deuterium; a substituted or unsubstituted C₁₋₂₀ alkyl group; or a substituted or unsubstituted C₃₋₁₀ aliphatic hydrocarbon ring group, or is bonded to an adjacent substituent to form a substituted or unsubstituted cyclohexane ring.

In an exemplary embodiment of the present specification, Ra, which is identical or different, is independently hydrogen; deuterium; a C₁₋₄ alkyl group that is unsubstituted or substituted with deuterium; or a C₃₋₁₀ aliphatic hydrocarbon ring group that is unsubstituted or substituted with deuterium, or is bonded to an adjacent substituent to form a cyclohexane ring substituted or unsubstituted with deuterium or a C₁₋₄ alkyl group.

In an exemplary embodiment of the present specification, Ra, which is identical or different, is independently hydrogen; deuterium; a substituted or unsubstituted methyl group; a substituted or unsubstituted ethyl group; a substituted or unsubstituted isopropyl group; a substituted or unsubstituted tert-butyl group; a substituted or unsubstituted cyclopentyl group; a substituted or unsubstituted cyclohexyl group; a substituted or unsubstituted adamantyl group; a substituted or unsubstituted anthracenyl group; a substituted or unsubstituted phenanthrenyl group; a substituted or unsubstituted triphenylenyl group; a substituted or unsubstituted fluorenyl group; a substituted or unsubstituted dibenzofuran group; a substituted or unsubstituted dibenzothiophene group; or a substituted or unsubstituted carbazole group, or is bonded to an adjacent substituent to form a substituted or unsubstituted cyclohexane ring.

In an exemplary embodiment of the present specification, Ra, which is identical or different, is independently hydrogen; deuterium; a substituted or unsubstituted methyl group; a substituted or unsubstituted isopropyl group; a substituted or unsubstituted tert-butyl group; a substituted or unsubstituted cyclopentyl group; or a substituted or unsubstituted adamantyl group, or is bonded to an adjacent substituent to form a cyclohexane ring that is unsubstituted or substituted with deuterium or a methyl group.

In an exemplary embodiment of the present specification, Ra, which is identical or different, is independently hydrogen; deuterium; a methyl group that is unsubstituted or substituted with deuterium; an ethyl group that is unsubstituted or substituted with deuterium; an isopropyl group that is unsubstituted or substituted with deuterium; a tert-butyl group that is unsubstituted or substituted with deuterium; a cyclopentyl group that is unsubstituted or substituted with deuterium; a cyclohexyl group that is unsubstituted or substituted with deuterium; an adamantyl group that is unsubstituted or substituted with deuterium; an anthracenyl group that is unsubstituted or substituted with deuterium; a phenanthrenyl group that is unsubstituted or substituted with deuterium; a triphenylenyl group that is unsubstituted or substituted with deuterium; a fluorenyl group that is unsubstituted or substituted with deuterium; a dibenzofuran group that is unsubstituted or substituted with deuterium; a dibenzothiophene group that is unsubstituted or substituted with deuterium; or a carbazole group that is unsubstituted or substituted with deuterium, or is bonded to an adjacent substituent to form a cyclohexane ring that is unsubstituted or substituted with deuterium or a methyl group.

In an exemplary embodiment of the present specification, Ra, which is identical or different, is independently hydrogen; deuterium; a methyl group that is unsubstituted or substituted with deuterium; an isopropyl group that is unsubstituted or substituted with deuterium; a tert-butyl group that is unsubstituted or substituted with deuterium; a cyclopentyl group that is unsubstituted or substituted with deuterium; or an adamantyl group that is unsubstituted or substituted with deuterium, or is bonded to an adjacent substituent to form a cyclohexane ring that is unsubstituted or substituted with deuterium or a methyl group.

In an exemplary embodiment of the present specification, Ra, which is identical or different, is independently hydrogen; deuterium; a methyl group; an isopropyl group; a tert-butyl group; a cyclopentyl group; or an adamantyl group, or is bonded to an adjacent substituent to form a cyclohexane ring that is unsubstituted or substituted with a methyl group.

In an exemplary embodiment of the present specification, Ra, which is identical or different, is independently hydrogen or deuterium.

In an exemplary embodiment of the present specification, Rb, which is identical or different, is independently hydrogen; deuterium; a substituted or unsubstituted C₁₋₃₀ alkyl group; a substituted or unsubstituted C₃₋₃₀ cycloalkyl group; a substituted or unsubstituted C₆₋₃₀ aryl group; or a substituted or unsubstituted C₂₋₃₀ heteroaryl group.

In an exemplary embodiment of the present specification, Rb, which is identical or different, is independently hydrogen; deuterium; a substituted or unsubstituted C₁₋₂₀ alkyl group; a substituted or unsubstituted C₃₋₂₀ cycloalkyl group; a substituted or unsubstituted C₆₋₂₀ aryl group; or a substituted or unsubstituted C₂₋₂₀ heteroaryl group.

In an exemplary embodiment of the present specification, Rb, which is identical or different, is independently hydrogen; deuterium; a substituted or unsubstituted C₁₋₁₀ alkyl group; a substituted or unsubstituted C₃₋₁₀ cycloalkyl group; a substituted or unsubstituted C₆₋₁₂ aryl group; or a substituted or unsubstituted C₂₋₁₀ heteroaryl group.

In an exemplary embodiment of the present specification, Rb, which is identical or different, is independently deuterium; or a substituted or unsubstituted C₁₋₁₀ alkyl group.

In an exemplary embodiment of the present specification, Rb, which is identical or different, is independently hydrogen; deuterium; a C₁₋₂₀ alkyl group that is unsubstituted or substituted with deuterium; a C₃₋₂₀ cycloalkyl group that is unsubstituted or substituted with deuterium; a C₆₋₂₀ aryl group that is unsubstituted or substituted with deuterium; or a C₂₋₂₀ heteroaryl group that is unsubstituted or substituted with deuterium.

In an exemplary embodiment of the present specification, Rb, which is identical or different, is independently hydrogen; deuterium; a C₁₋₁₀ alkyl group that is unsubstituted or substituted with deuterium; a C₃₋₁₀ cycloalkyl group that is unsubstituted or substituted with deuterium; a C₆₋₁₂ aryl group that is unsubstituted or substituted with deuterium; or a C₂₋₁₀ heteroaryl group that is unsubstituted or substituted with deuterium.

In an exemplary embodiment of the present specification, Rb, which is identical or different, is independently deuterium; or a C₁₋₁₀ alkyl group that is unsubstituted or substituted with deuterium.

In an exemplary embodiment of the present specification, Rb, which is identical or different, is independently hydrogen or deuterium.

In an exemplary embodiment of the present specification, all of Rb is hydrogen or deuterium.

In an exemplary embodiment of the present specification, m is an integer from 1 to 5.

In an exemplary embodiment of the present specification, m is an integer from 1 to 4.

In an exemplary embodiment of the present specification, m is an integer from 1 to 3.

In an exemplary embodiment of the present specification, m is an integer 1 or 2.

In an exemplary embodiment of the present specification, m is 6.

In an exemplary embodiment of the present specification, m is 5.

In an exemplary embodiment of the present specification, m is 4.

In an exemplary embodiment of the present specification, m is 3.

In an exemplary embodiment of the present specification, m is 2.

In an exemplary embodiment of the present specification, m is 1.

In an exemplary embodiment of the present specification, n is an integer 1 or 2.

In an exemplary embodiment of the present specification, n is 2.

In an exemplary embodiment of the present specification, n is 1.

In an exemplary embodiment of the present specification, a1 is an integer from 1 to 5.

In an exemplary embodiment of the present specification, a1 is an integer from 1 to 4.

In an exemplary embodiment of the present specification, a1 is an integer from 1 to 3.

In an exemplary embodiment of the present specification, a1 is an integer 1 or 2.

In an exemplary embodiment of the present specification, a1 is 5.

In an exemplary embodiment of the present specification, a1 is 4.

In an exemplary embodiment of the present specification, a1 is 3.

In an exemplary embodiment of the present specification, a1 is 2.

In an exemplary embodiment of the present specification, a1 is 1.

In an exemplary embodiment of the present specification, b1 is an integer from 1 to 9.

In an exemplary embodiment of the present specification, b1 is an integer from 1 to 8.

In an exemplary embodiment of the present specification, b1 is an integer from 1 to 7.

In an exemplary embodiment of the present specification, b1 is an integer from 1 to 6.

In an exemplary embodiment of the present specification, b1 is an integer from 1 to 5.

In an exemplary embodiment of the present specification, b1 is an integer from 1 to 4.

In an exemplary embodiment of the present specification, b1 is an integer from 1 to 3.

In an exemplary embodiment of the present specification, b1 is an integer 1 or 2.

In an exemplary embodiment of the present specification, b1 is 9.

In an exemplary embodiment of the present specification, b1 is 8.

In an exemplary embodiment of the present specification, b1 is 7.

In an exemplary embodiment of the present specification, b1 is 6.

In an exemplary embodiment of the present specification, b1 is 5.

In an exemplary embodiment of the present specification, b1 is 4.

In an exemplary embodiment of the present specification, b1 is 3.

In an exemplary embodiment of the present specification, b1 is 2.

In an exemplary embodiment of the present specification, b1 is 1.

In an exemplary embodiment of the present specification, Chemical Formula 1-1 or Chemical Formula 1-2 has a deuterium substitution rate of 1% to 100%.

In an exemplary embodiment of the present specification, Chemical Formula 1-1 or Chemical Formula 1-2 has a deuterium substitution rate of 30% to 100%.

In an exemplary embodiment of the present specification, Chemical Formula 1-1 or Chemical Formula 1-2 has a deuterium substitution rate of 40% to 99%.

In an exemplary embodiment of the present specification, Chemical Formula 1-1 has a deuterium substitution rate of 1% to 100%, specifically 30% to 100%, more specifically 40% to 99%.

In an exemplary embodiment of the present specification, Chemical Formula 1-2 has a deuterium substitution rate of 1% to 100%, specifically 30% to 100%, more specifically 40% to 99%.

In an exemplary embodiment of the present specification, Chemical Formula 1-1 or Chemical Formula 1-2 is any one of the following compounds.

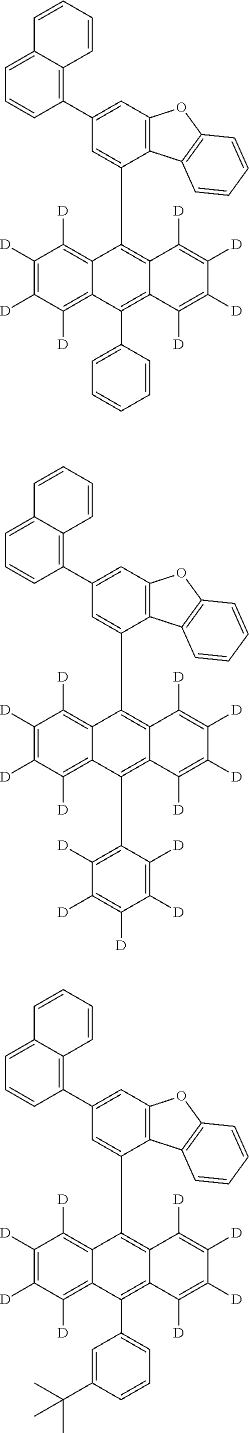 [SMILES: [2H]c1c([2H])c([2H])c2c(-c3cc(-c4cccc5ccccc45)cc4oc5ccccc5c34)c3c([2H])c([2H])c([2H])c([2H])c3c(-c3ccccc3)c2c1[2H] ; [2H]c1c([2H])c([2H])c(-c2c3c([2H])c([2H])c([2H])c([2H])c3c(-c3cc(-c4cccc5ccccc45)cc4oc5ccccc5c34)c3c([2H])c([2H])c([2H])c([2H])c23)c([2H])c1[2H] ; [2H]c1c([2H])c([2H])c2c(-c3cc(-c4cccc5ccccc45)cc4oc5ccccc5c34)c3c([2H])c([2H])c([2H])c([2H])c3c(-c3cccc(C(C)(C)C)c3)c2c1[2H]]

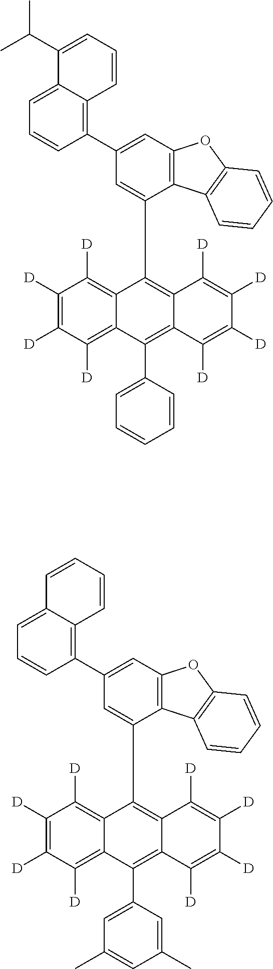 [SMILES: [2H]c1c([2H])c([2H])c2c(-c3cc(-c4cccc5c(C(C)C)cccc45)cc4oc5ccccc5c34)c3c([2H])c([2H])c([2H])c([2H])c3c(-c3ccccc3)c2c1[2H] ; [2H]c1c([2H])c([2H])c2c(-c3cc(-c4cccc5ccccc45)cc4oc5ccccc5c34)c3c([2H])c([2H])c([2H])c([2H])c3c(-c3cc(C)cc(C)c3)c2c1[2H]]

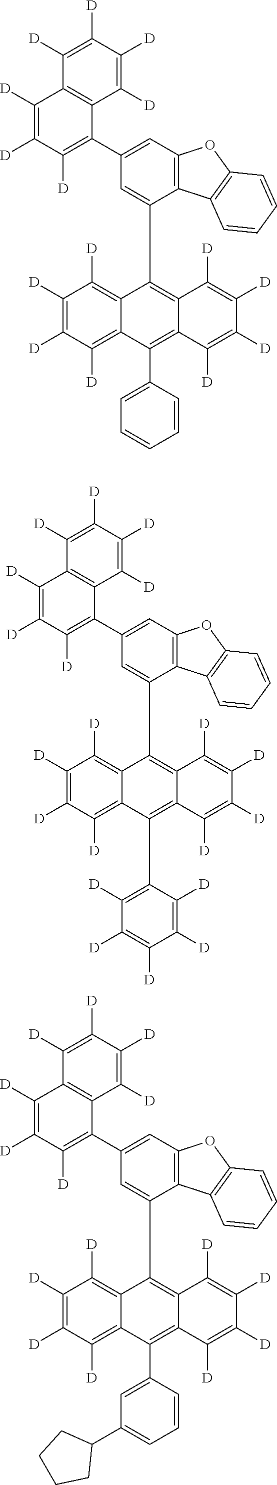

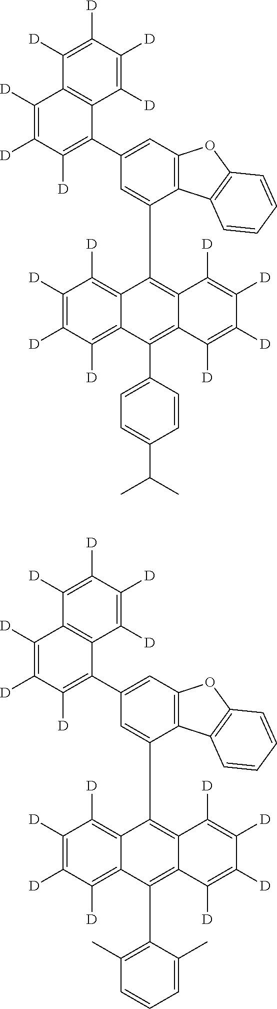 [SMILES: [2H]c1c([2H])c([2H])c2c(-c3cc(-c4c5c([2H])c([2H])c([2H])c([2H])c5c(-c5ccc(C(C)C)cc5)c5c([2H])c([2H])c([2H])c([2H])c45)c4c(c3)oc3ccccc34)c([2H])c([2H])c([2H])c2c1[2H] ; [2H]c1c([2H])c([2H])c2c(-c3cc(-c4c5c([2H])c([2H])c([2H])c([2H])c5c(-c5c(C)cccc5C)c5c([2H])c([2H])c([2H])c([2H])c45)c4c(c3)oc3ccccc34)c([2H])c([2H])c([2H])c2c1[2H]]

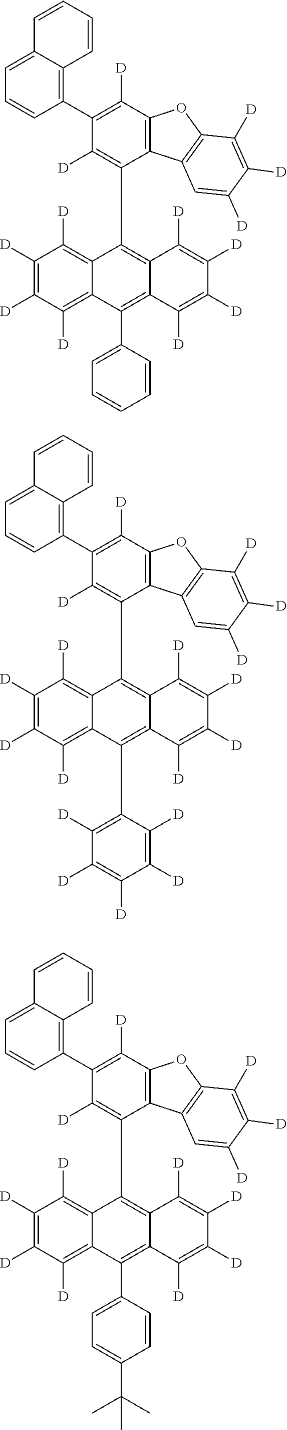 [SMILES: [2H]c1cc2c(oc3c([2H])c(-c4cccc5ccccc45)c([2H])c(-c4c5c([2H])c([2H])c([2H])c([2H])c5c(-c5ccccc5)c5c([2H])c([2H])c([2H])c([2H])c45)c32)c([2H])c1[2H] ; [2H]c1cc2c(oc3c([2H])c(-c4cccc5ccccc45)c([2H])c(-c4c5c([2H])c([2H])c([2H])c([2H])c5c(-c5c([2H])c([2H])c([2H])c([2H])c5[2H])c5c([2H])c([2H])c([2H])c([2H])c45)c32)c([2H])c1[2H] ; [2H]c1cc2c(oc3c([2H])c(-c4cccc5ccccc45)c([2H])c(-c4c5c([2H])c([2H])c([2H])c([2H])c5c(-c5ccc(C(C)(C)C)cc5)c5c([2H])c([2H])c([2H])c([2H])c45)c32)c([2H])c1[2H]]

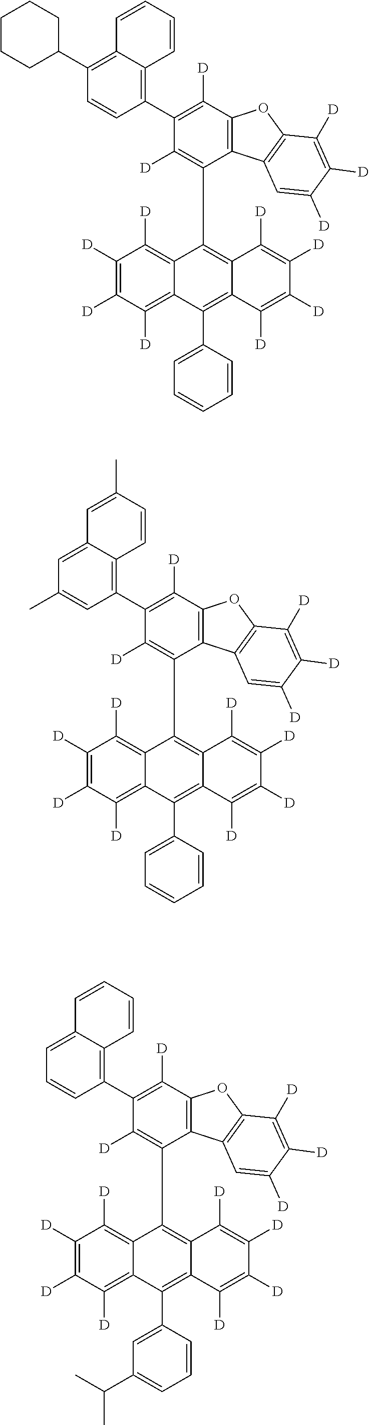 [SMILES: [2H]c1cc2c(oc3c([2H])c(-c4ccc(C5CCCCC5)c5ccccc45)c([2H])c(-c4c5c([2H])c([2H])c([2H])c([2H])c5c(-c5ccccc5)c5c([2H])c([2H])c([2H])c([2H])c45)c32)c([2H])c1[2H] ; [2H]c1cc2c(oc3c([2H])c(-c4cc(C)cc5cc(C)ccc45)c([2H])c(-c4c5c([2H])c([2H])c([2H])c([2H])c5c(-c5ccccc5)c5c([2H])c([2H])c([2H])c([2H])c45)c32)c([2H])c1[2H] ; [2H]c1cc2c(oc3c([2H])c(-c4cccc5ccccc45)c([2H])c(-c4c5c([2H])c([2H])c([2H])c([2H])c5c(-c5cccc(C(C)C)c5)c5c([2H])c([2H])c([2H])c([2H])c45)c32)c([2H])c1[2H]]

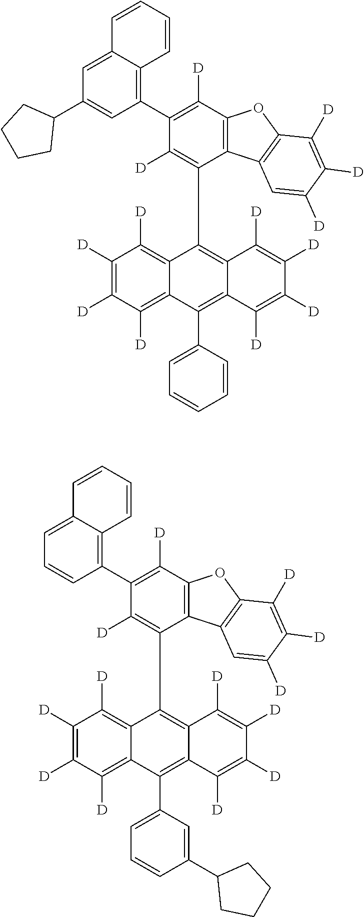 [SMILES: [2H]c1cc2c(oc3c([2H])c(-c4cc(C5CCCC5)cc5ccccc45)c([2H])c(-c4c5c([2H])c([2H])c([2H])c([2H])c5c(-c5ccccc5)c5c([2H])c([2H])c([2H])c([2H])c45)c32)c([2H])c1[2H] ; [2H]c1cc2c(oc3c([2H])c(-c4cccc5ccccc45)c([2H])c(-c4c5c([2H])c([2H])c([2H])c([2H])c5c(-c5cccc(C6CCCC6)c5)c5c([2H])c([2H])c([2H])c([2H])c45)c32)c([2H])c1[2H]]

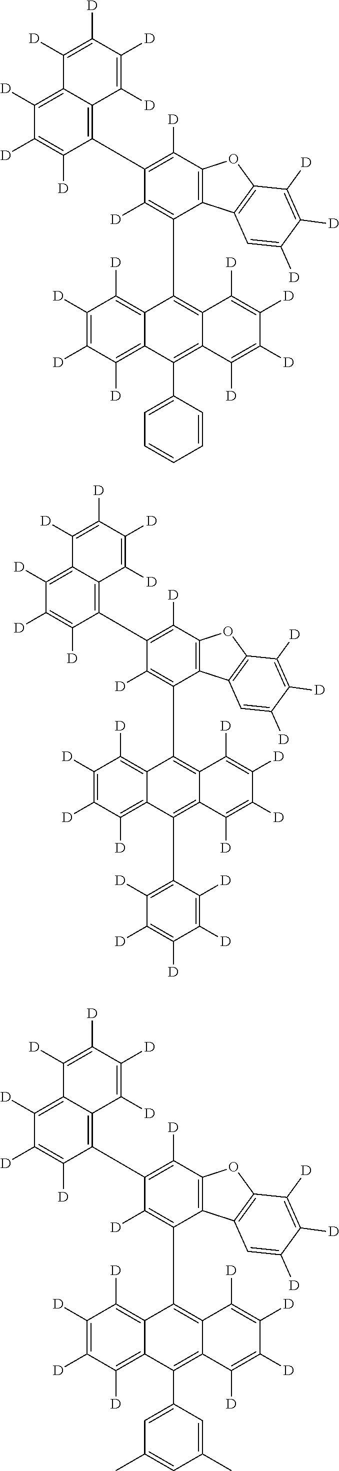

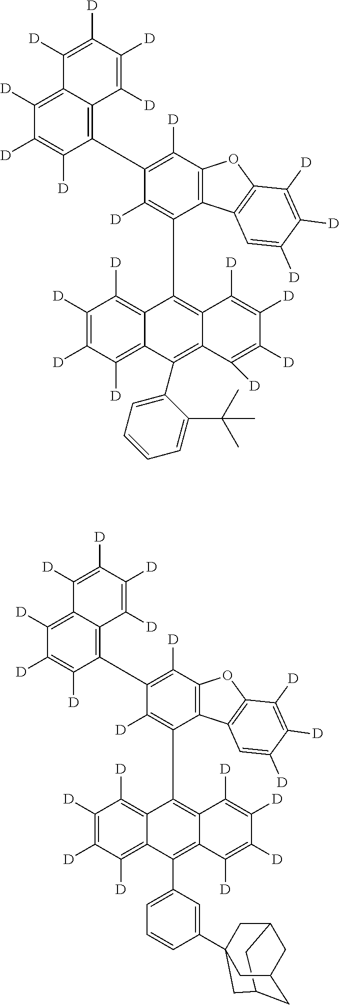 [SMILES: [2H]c1cc2c(oc3c([2H])c(-c4c([2H])c([2H])c([2H])c5c([2H])c([2H])c([2H])c([2H])c45)c([2H])c(-c4c5c([2H])c([2H])c([2H])c([2H])c5c(-c5ccccc5C(C)(C)C)c5c([2H])c([2H])c([2H])c([2H])c45)c32)c([2H])c1[2H] ; [2H]c1cc2c(oc3c([2H])c(-c4c([2H])c([2H])c([2H])c5c([2H])c([2H])c([2H])c([2H])c45)c([2H])c(-c4c5c([2H])c([2H])c([2H])c([2H])c5c(-c5cccc(C67CC8CC(CC(C8)C6)C7)c5)c5c([2H])c([2H])c([2H])c([2H])c45)c32)c([2H])c1[2H]]

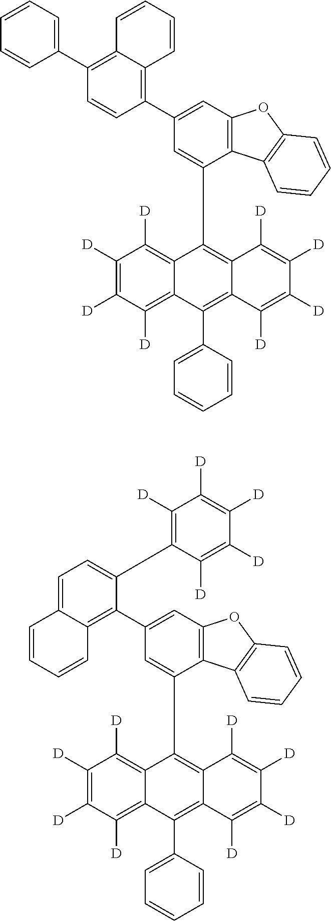 [SMILES: [2H]c1c([2H])c([2H])c2c(-c3cc(-c4ccc(-c5ccccc5)c5ccccc45)cc4oc5ccccc5c34)c3c([2H])c([2H])c([2H])c([2H])c3c(-c3ccccc3)c2c1[2H] ; [2H]c1c([2H])c([2H])c(-c2ccc3ccccc3c2-c2cc(-c3c4c([2H])c([2H])c([2H])c([2H])c4c(-c4ccccc4)c4c([2H])c([2H])c([2H])c([2H])c34)c3c(c2)oc2ccccc23)c([2H])c1[2H]]

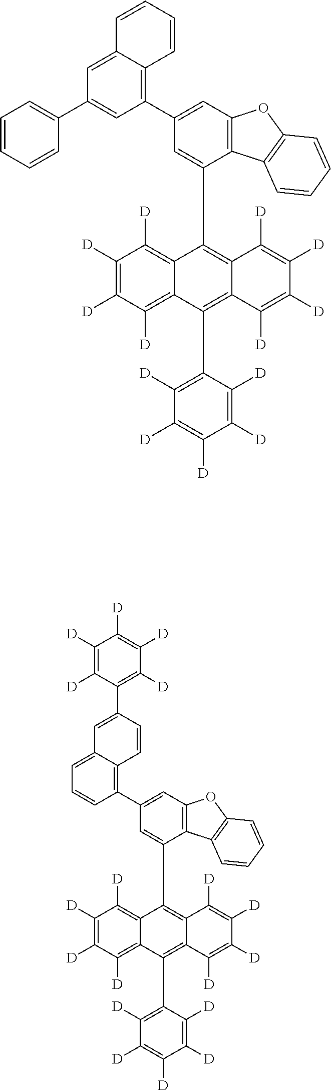 [SMILES: [2H]c1c([2H])c([2H])c(-c2c3c([2H])c([2H])c([2H])c([2H])c3c(-c3cc(-c4cc(-c5ccccc5)cc5ccccc45)cc4oc5ccccc5c34)c3c([2H])c([2H])c([2H])c([2H])c23)c([2H])c1[2H] ; [2H]c1c([2H])c([2H])c(-c2ccc3c(-c4cc(-c5c6c([2H])c([2H])c([2H])c([2H])c6c(-c6c([2H])c([2H])c([2H])c([2H])c6[2H])c6c([2H])c([2H])c([2H])c([2H])c56)c5c(c4)oc4ccccc45)cccc3c2)c([2H])c1[2H]]

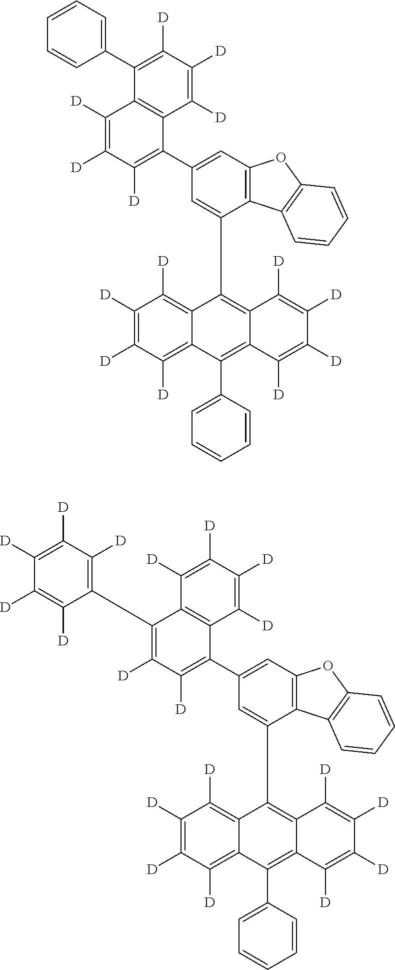 [SMILES: [2H]c1c([2H])c(-c2cc(-c3c4c([2H])c([2H])c([2H])c([2H])c4c(-c4ccccc4)c4c([2H])c([2H])c([2H])c([2H])c34)c3c(c2)oc2ccccc23)c2c([2H])c([2H])c([2H])c(-c3ccccc3)c2c1[2H] ; [2H]c1c([2H])c([2H])c(-c2c([2H])c([2H])c(-c3cc(-c4c5c([2H])c([2H])c([2H])c([2H])c5c(-c5ccccc5)c5c([2H])c([2H])c([2H])c([2H])c45)c4c(c3)oc3ccccc34)c3c([2H])c([2H])c([2H])c([2H])c23)c([2H])c1[2H]]

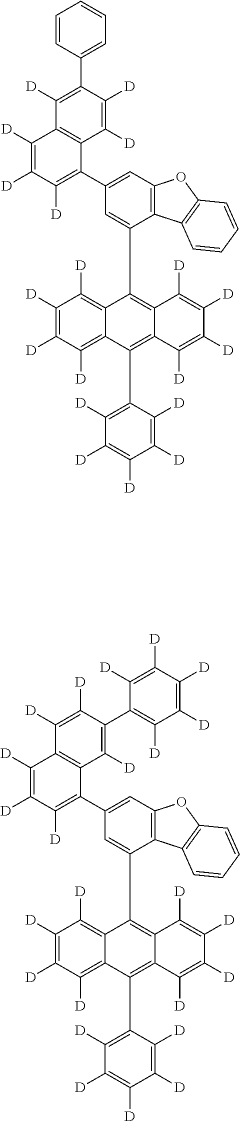 [SMILES: [2H]c1c([2H])c([2H])c(-c2c3c([2H])c([2H])c([2H])c([2H])c3c(-c3cc(-c4c([2H])c([2H])c([2H])c5c([2H])c(-c6ccccc6)c([2H])c([2H])c45)cc4oc5ccccc5c34)c3c([2H])c([2H])c([2H])c([2H])c23)c([2H])c1[2H] ; [2H]c1c([2H])c([2H])c(-c2c([2H])c([2H])c3c([2H])c([2H])c([2H])c(-c4cc(-c5c6c([2H])c([2H])c([2H])c([2H])c6c(-c6c([2H])c([2H])c([2H])c([2H])c6[2H])c6c([2H])c([2H])c([2H])c([2H])c56)c5c(c4)oc4ccccc45)c3c2[2H])c([2H])c1[2H]]

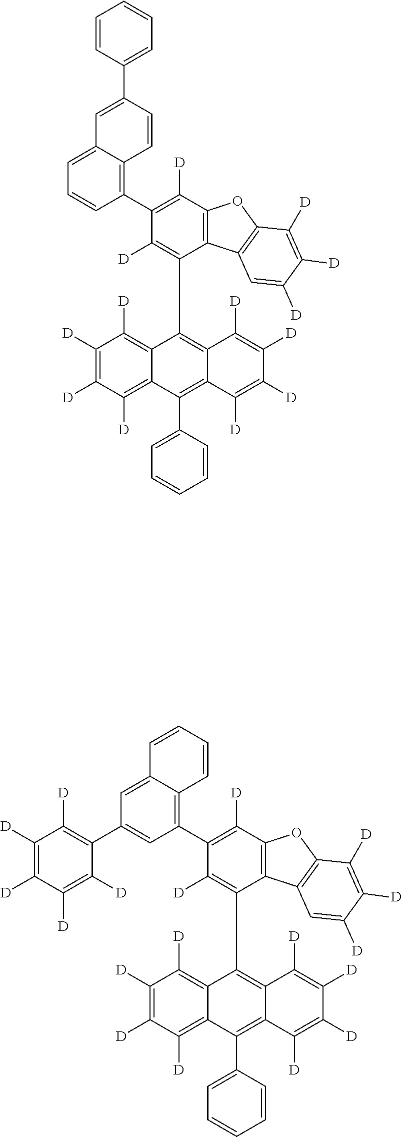 [SMILES: [2H]c1cc2c(oc3c([2H])c(-c4cccc5cc(-c6ccccc6)ccc45)c([2H])c(-c4c5c([2H])c([2H])c([2H])c([2H])c5c(-c5ccccc5)c5c([2H])c([2H])c([2H])c([2H])c45)c32)c([2H])c1[2H] ; [2H]c1cc2c(oc3c([2H])c(-c4cc(-c5c([2H])c([2H])c([2H])c([2H])c5[2H])cc5ccccc45)c([2H])c(-c4c5c([2H])c([2H])c([2H])c([2H])c5c(-c5ccccc5)c5c([2H])c([2H])c([2H])c([2H])c45)c32)c([2H])c1[2H]]

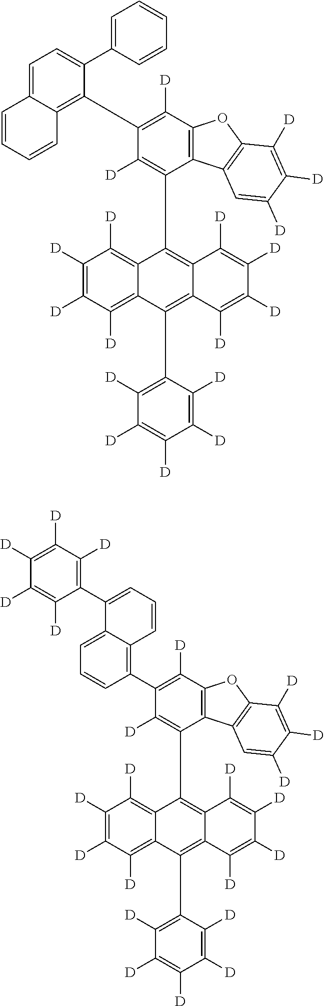 [SMILES: [2H]c1cc2c(oc3c([2H])c(-c4c(-c5ccccc5)ccc5ccccc45)c([2H])c(-c4c5c([2H])c([2H])c([2H])c([2H])c5c(-c5c([2H])c([2H])c([2H])c([2H])c5[2H])c5c([2H])c([2H])c([2H])c([2H])c45)c32)c([2H])c1[2H] ; [2H]c1cc2c(oc3c([2H])c(-c4cccc5c(-c6c([2H])c([2H])c([2H])c([2H])c6[2H])cccc45)c([2H])c(-c4c5c([2H])c([2H])c([2H])c([2H])c5c(-c5c([2H])c([2H])c([2H])c([2H])c5[2H])c5c([2H])c([2H])c([2H])c([2H])c45)c32)c([2H])c1[2H]]

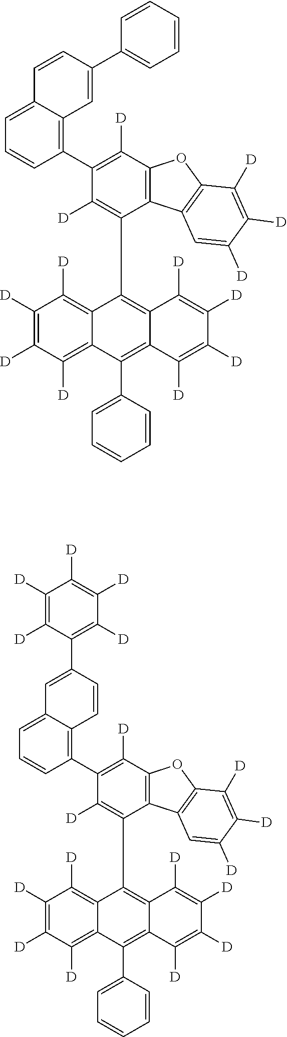 [SMILES: [2H]c1cc2c(oc3c([2H])c(-c4cccc5ccc(-c6ccccc6)cc45)c([2H])c(-c4c5c([2H])c([2H])c([2H])c([2H])c5c(-c5ccccc5)c5c([2H])c([2H])c([2H])c([2H])c45)c32)c([2H])c1[2H] ; [2H]c1cc2c(oc3c([2H])c(-c4cccc5cc(-c6c([2H])c([2H])c([2H])c([2H])c6[2H])ccc45)c([2H])c(-c4c5c([2H])c([2H])c([2H])c([2H])c5c(-c5ccccc5)c5c([2H])c([2H])c([2H])c([2H])c45)c32)c([2H])c1[2H]]

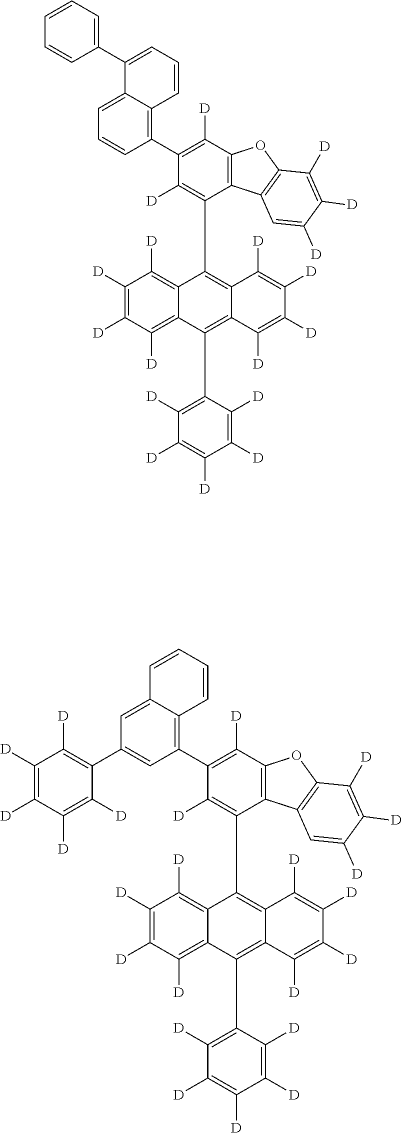 [SMILES: [2H]c1cc2c(oc3c([2H])c(-c4cccc5c(-c6ccccc6)cccc45)c([2H])c(-c4c5c([2H])c([2H])c([2H])c([2H])c5c(-c5c([2H])c([2H])c([2H])c([2H])c5[2H])c5c([2H])c([2H])c([2H])c([2H])c45)c32)c([2H])c1[2H] ; [2H]c1cc2c(oc3c([2H])c(-c4cc(-c5c([2H])c([2H])c([2H])c([2H])c5[2H])cc5ccccc45)c([2H])c(-c4c5c([2H])c([2H])c([2H])c([2H])c5c(-c5c([2H])c([2H])c([2H])c([2H])c5[2H])c5c([2H])c([2H])c([2H])c([2H])c45)c32)c([2H])c1[2H]]

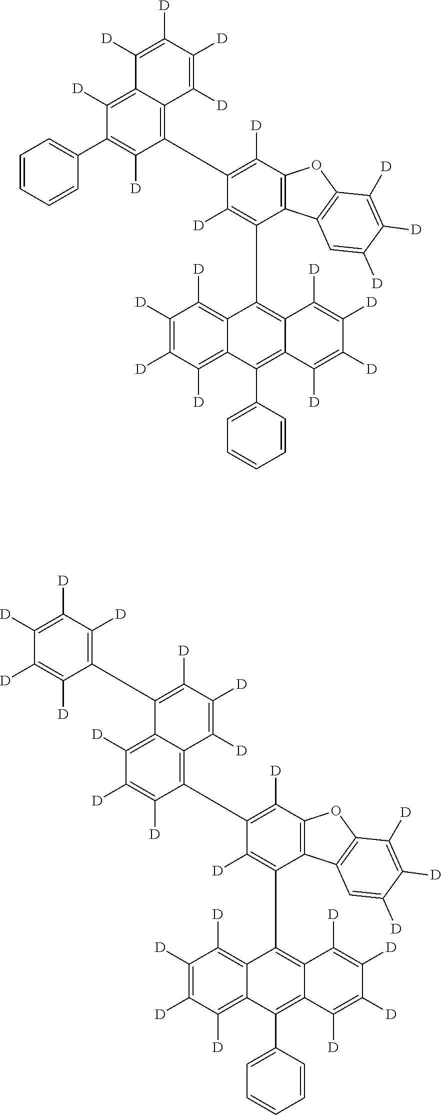 [SMILES: [2H]c1cc2c(oc3c([2H])c(-c4c([2H])c(-c5ccccc5)c([2H])c5c([2H])c([2H])c([2H])c([2H])c45)c([2H])c(-c4c5c([2H])c([2H])c([2H])c([2H])c5c(-c5ccccc5)c5c([2H])c([2H])c([2H])c([2H])c45)c32)c([2H])c1[2H] ; [2H]c1cc2c(oc3c([2H])c(-c4c([2H])c([2H])c([2H])c5c(-c6c([2H])c([2H])c([2H])c([2H])c6[2H])c([2H])c([2H])c([2H])c45)c([2H])c(-c4c5c([2H])c([2H])c([2H])c([2H])c5c(-c5ccccc5)c5c([2H])c([2H])c([2H])c([2H])c45)c32)c([2H])c1[2H]]

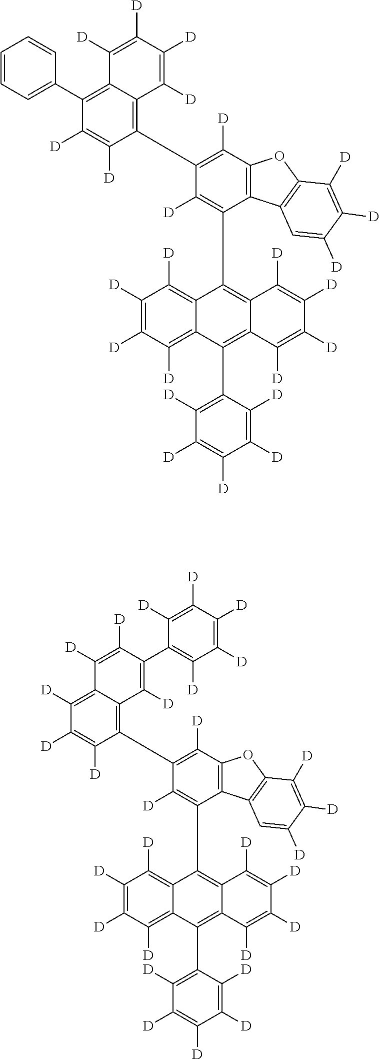 [SMILES: [2H]c1cc2c(oc3c([2H])c(-c4c([2H])c([2H])c(-c5ccccc5)c5c([2H])c([2H])c([2H])c([2H])c45)c([2H])c(-c4c5c([2H])c([2H])c([2H])c([2H])c5c(-c5c([2H])c([2H])c([2H])c([2H])c5[2H])c5c([2H])c([2H])c([2H])c([2H])c45)c32)c([2H])c1[2H] ; [2H]c1cc2c(oc3c([2H])c(-c4c([2H])c([2H])c([2H])c5c([2H])c([2H])c(-c6c([2H])c([2H])c([2H])c([2H])c6[2H])c([2H])c45)c([2H])c(-c4c5c([2H])c([2H])c([2H])c([2H])c5c(-c5c([2H])c([2H])c([2H])c([2H])c5[2H])c5c([2H])c([2H])c([2H])c([2H])c45)c32)c([2H])c1[2H]]

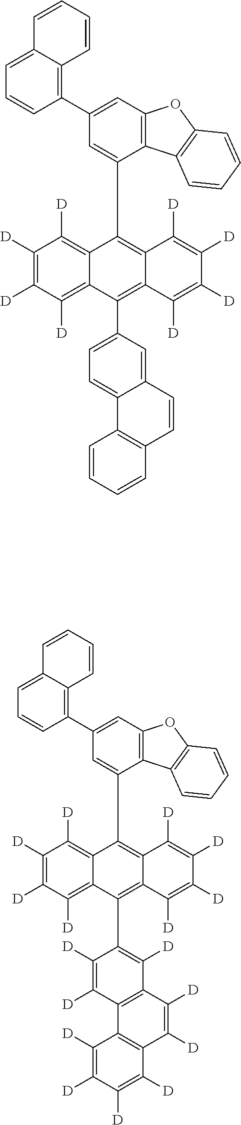 [SMILES: [2H]c1c([2H])c([2H])c2c(-c3cc(-c4cccc5ccccc45)cc4oc5ccccc5c34)c3c([2H])c([2H])c([2H])c([2H])c3c(-c3ccc4c(ccc5ccccc54)c3)c2c1[2H] ; [2H]c1c([2H])c([2H])c2c(-c3cc(-c4cccc5ccccc45)cc4oc5ccccc5c34)c3c([2H])c([2H])c([2H])c([2H])c3c(-c3c([2H])c([2H])c4c(c3[2H])c([2H])c([2H])c3c([2H])c([2H])c([2H])c([2H])c34)c2c1[2H]]

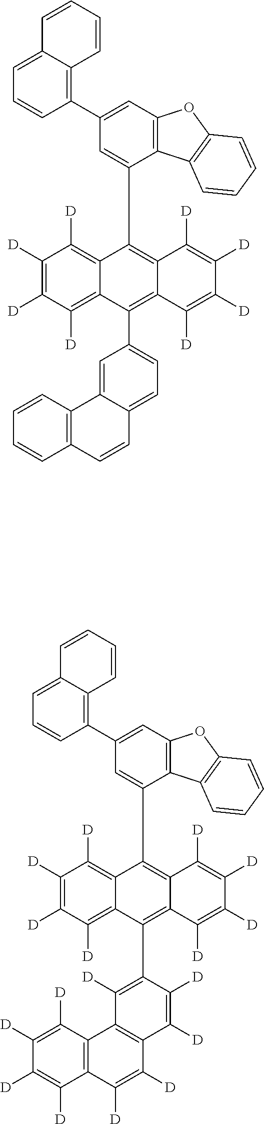 [SMILES: [2H]c1c([2H])c([2H])c2c(-c3cc(-c4cccc5ccccc45)cc4oc5ccccc5c34)c3c([2H])c([2H])c([2H])c([2H])c3c(-c3ccc4ccc5ccccc5c4c3)c2c1[2H] ; [2H]c1c([2H])c([2H])c2c(-c3cc(-c4cccc5ccccc45)cc4oc5ccccc5c34)c3c([2H])c([2H])c([2H])c([2H])c3c(-c3c([2H])c([2H])c4c([2H])c([2H])c5c([2H])c([2H])c([2H])c([2H])c5c4c3[2H])c2c1[2H]]

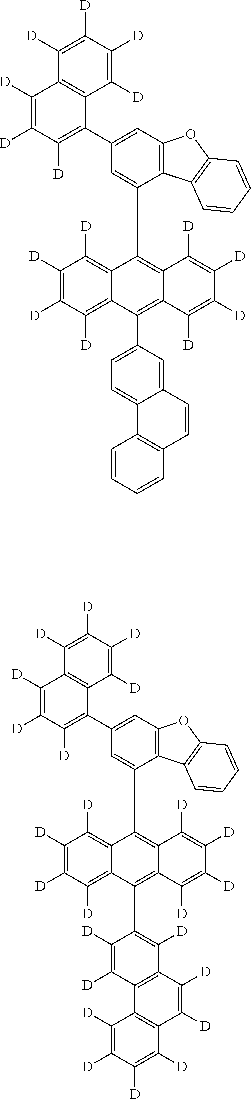 [SMILES: [2H]c1c([2H])c([2H])c2c(-c3cc(-c4c5c([2H])c([2H])c([2H])c([2H])c5c(-c5ccc6c(ccc7ccccc76)c5)c5c([2H])c([2H])c([2H])c([2H])c45)c4c(c3)oc3ccccc34)c([2H])c([2H])c([2H])c2c1[2H] ; [2H]c1c([2H])c([2H])c2c(-c3cc(-c4c5c([2H])c([2H])c([2H])c([2H])c5c(-c5c([2H])c([2H])c6c(c5[2H])c([2H])c([2H])c5c([2H])c([2H])c([2H])c([2H])c56)c5c([2H])c([2H])c([2H])c([2H])c45)c4c(c3)oc3ccccc34)c([2H])c([2H])c([2H])c2c1[2H]]

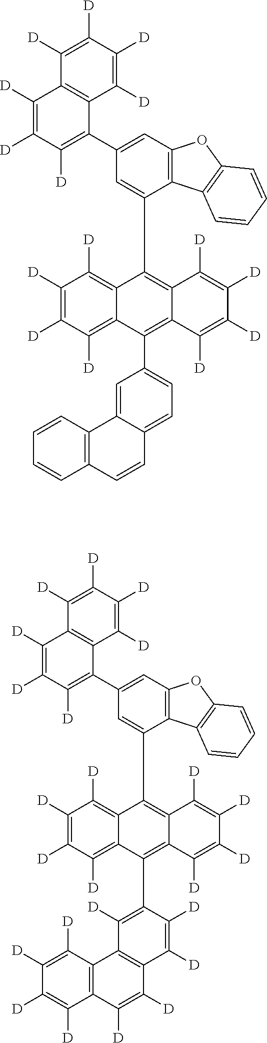 [SMILES: [2H]c1c([2H])c([2H])c2c(-c3cc(-c4c5c([2H])c([2H])c([2H])c([2H])c5c(-c5ccc6ccc7ccccc7c6c5)c5c([2H])c([2H])c([2H])c([2H])c45)c4c(c3)oc3ccccc34)c([2H])c([2H])c([2H])c2c1[2H] ; [2H]c1c([2H])c([2H])c2c(-c3cc(-c4c5c([2H])c([2H])c([2H])c([2H])c5c(-c5c([2H])c([2H])c6c([2H])c([2H])c7c([2H])c([2H])c([2H])c([2H])c7c6c5[2H])c5c([2H])c([2H])c([2H])c([2H])c45)c4c(c3)oc3ccccc34)c([2H])c([2H])c([2H])c2c1[2H]]

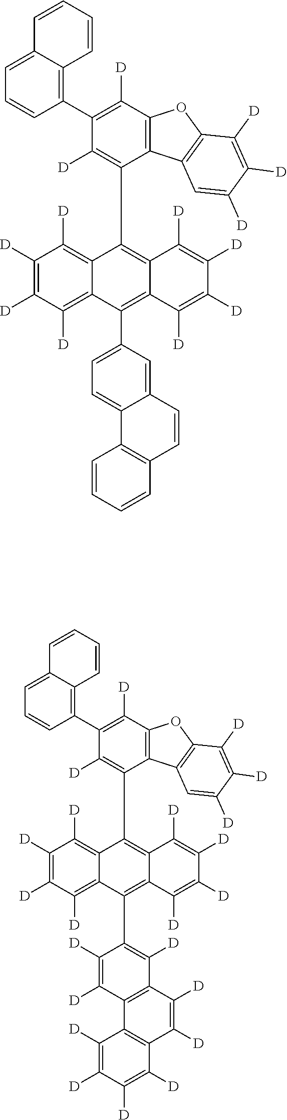 [SMILES: [2H]c1cc2c(oc3c([2H])c(-c4cccc5ccccc45)c([2H])c(-c4c5c([2H])c([2H])c([2H])c([2H])c5c(-c5ccc6c(ccc7ccccc76)c5)c5c([2H])c([2H])c([2H])c([2H])c45)c32)c([2H])c1[2H] ; [2H]c1cc2c(oc3c([2H])c(-c4cccc5ccccc45)c([2H])c(-c4c5c([2H])c([2H])c([2H])c([2H])c5c(-c5c([2H])c([2H])c6c(c5[2H])c([2H])c([2H])c5c([2H])c([2H])c([2H])c([2H])c56)c5c([2H])c([2H])c([2H])c([2H])c45)c32)c([2H])c1[2H]]

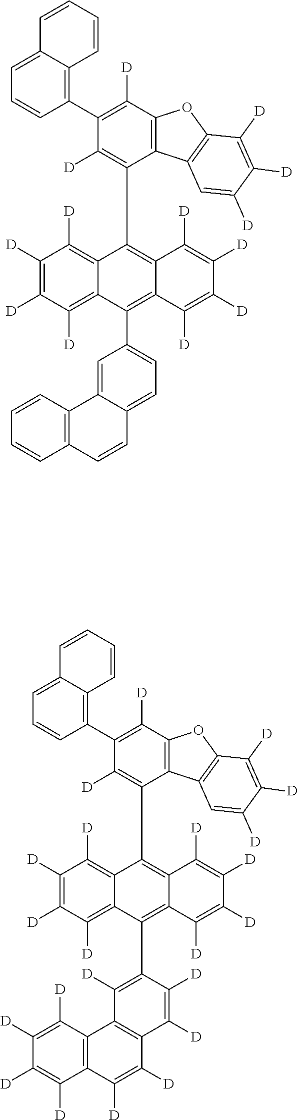 [SMILES: [2H]c1cc2c(oc3c([2H])c(-c4cccc5ccccc45)c([2H])c(-c4c5c([2H])c([2H])c([2H])c([2H])c5c(-c5ccc6ccc7ccccc7c6c5)c5c([2H])c([2H])c([2H])c([2H])c45)c32)c([2H])c1[2H] ; [2H]c1cc2c(oc3c([2H])c(-c4cccc5ccccc45)c([2H])c(-c4c5c([2H])c([2H])c([2H])c([2H])c5c(-c5c([2H])c([2H])c6c([2H])c([2H])c7c([2H])c([2H])c([2H])c([2H])c7c6c5[2H])c5c([2H])c([2H])c([2H])c([2H])c45)c32)c([2H])c1[2H]]

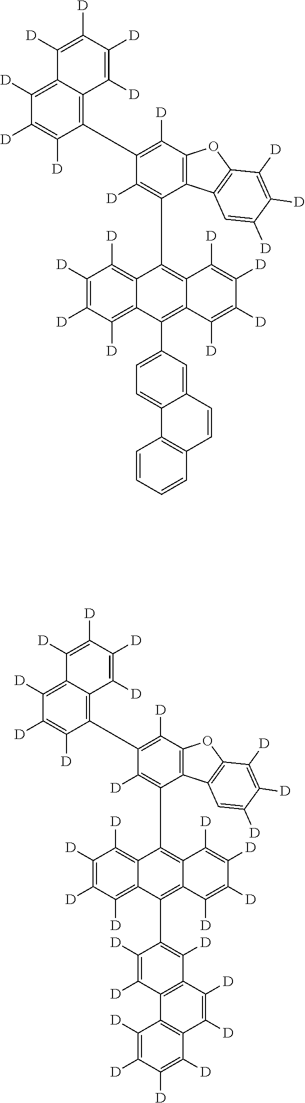 [SMILES: [2H]c1cc2c(oc3c([2H])c(-c4c([2H])c([2H])c([2H])c5c([2H])c([2H])c([2H])c([2H])c45)c([2H])c(-c4c5c([2H])c([2H])c([2H])c([2H])c5c(-c5ccc6c(ccc7ccccc76)c5)c5c([2H])c([2H])c([2H])c([2H])c45)c32)c([2H])c1[2H] ; [2H]c1cc2c(oc3c([2H])c(-c4c([2H])c([2H])c([2H])c5c([2H])c([2H])c([2H])c([2H])c45)c([2H])c(-c4c5c([2H])c([2H])c([2H])c([2H])c5c(-c5c([2H])c([2H])c6c(c5[2H])c([2H])c([2H])c5c([2H])c([2H])c([2H])c([2H])c56)c5c([2H])c([2H])c([2H])c([2H])c45)c32)c([2H])c1[2H]]

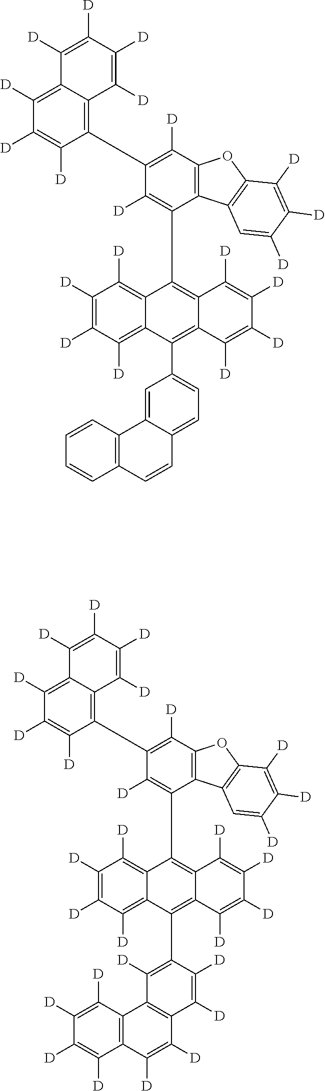 [SMILES: [2H]c1cc2c(oc3c([2H])c(-c4c([2H])c([2H])c([2H])c5c([2H])c([2H])c([2H])c([2H])c45)c([2H])c(-c4c5c([2H])c([2H])c([2H])c([2H])c5c(-c5ccc6ccc7ccccc7c6c5)c5c([2H])c([2H])c([2H])c([2H])c45)c32)c([2H])c1[2H] ; [2H]c1cc2c(oc3c([2H])c(-c4c([2H])c([2H])c([2H])c5c([2H])c([2H])c([2H])c([2H])c45)c([2H])c(-c4c5c([2H])c([2H])c([2H])c([2H])c5c(-c5c([2H])c([2H])c6c([2H])c([2H])c7c([2H])c([2H])c([2H])c([2H])c7c6c5[2H])c5c([2H])c([2H])c([2H])c([2H])c45)c32)c([2H])c1[2H]]

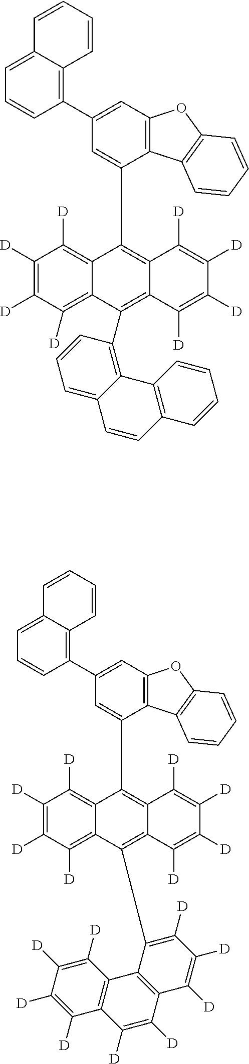 [SMILES: [2H]c1c([2H])c([2H])c2c(-c3cc(-c4cccc5ccccc45)cc4oc5ccccc5c34)c3c([2H])c([2H])c([2H])c([2H])c3c(-c3cccc4ccc5ccccc5c34)c2c1[2H] ; [2H]c1c([2H])c([2H])c2c(-c3c([2H])c([2H])c([2H])c4c([2H])c([2H])c5c([2H])c([2H])c([2H])c([2H])c5c34)c3c([2H])c([2H])c([2H])c([2H])c3c(-c3cc(-c4cccc5ccccc45)cc4oc5ccccc5c34)c2c1[2H]]

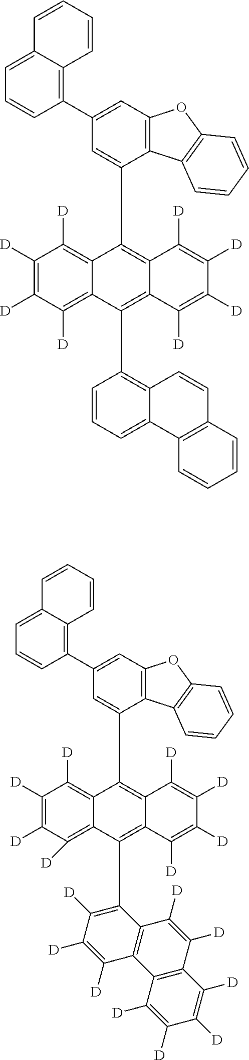 [SMILES: [2H]c1c([2H])c([2H])c2c(-c3cc(-c4cccc5ccccc45)cc4oc5ccccc5c34)c3c([2H])c([2H])c([2H])c([2H])c3c(-c3cccc4c3ccc3ccccc34)c2c1[2H] ; [2H]c1c([2H])c([2H])c2c(-c3cc(-c4cccc5ccccc45)cc4oc5ccccc5c34)c3c([2H])c([2H])c([2H])c([2H])c3c(-c3c([2H])c([2H])c([2H])c4c3c([2H])c([2H])c3c([2H])c([2H])c([2H])c([2H])c34)c2c1[2H]]

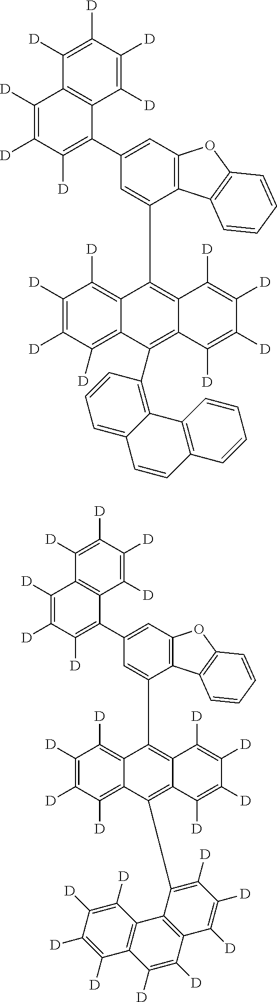 [SMILES: [2H]c1c([2H])c([2H])c2c(-c3cc(-c4c5c([2H])c([2H])c([2H])c([2H])c5c(-c5cccc6ccc7ccccc7c56)c5c([2H])c([2H])c([2H])c([2H])c45)c4c(c3)oc3ccccc34)c([2H])c([2H])c([2H])c2c1[2H] ; [2H]c1c([2H])c([2H])c2c(-c3cc(-c4c5c([2H])c([2H])c([2H])c([2H])c5c(-c5c([2H])c([2H])c([2H])c6c([2H])c([2H])c7c([2H])c([2H])c([2H])c([2H])c7c56)c5c([2H])c([2H])c([2H])c([2H])c45)c4c(c3)oc3ccccc34)c([2H])c([2H])c([2H])c2c1[2H]]

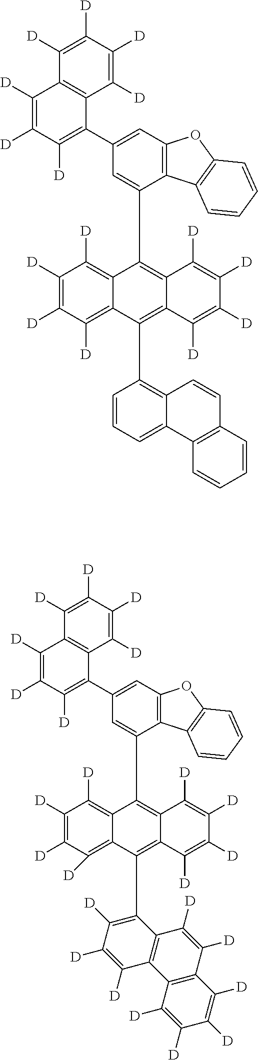 [SMILES: [2H]c1c([2H])c([2H])c2c(-c3cc(-c4c5c([2H])c([2H])c([2H])c([2H])c5c(-c5cccc6c5ccc5ccccc56)c5c([2H])c([2H])c([2H])c([2H])c45)c4c(c3)oc3ccccc34)c([2H])c([2H])c([2H])c2c1[2H] ; [2H]c1c([2H])c([2H])c2c(-c3cc(-c4c5c([2H])c([2H])c([2H])c([2H])c5c(-c5c([2H])c([2H])c([2H])c6c5c([2H])c([2H])c5c([2H])c([2H])c([2H])c([2H])c56)c5c([2H])c([2H])c([2H])c([2H])c45)c4c(c3)oc3ccccc34)c([2H])c([2H])c([2H])c2c1[2H]]

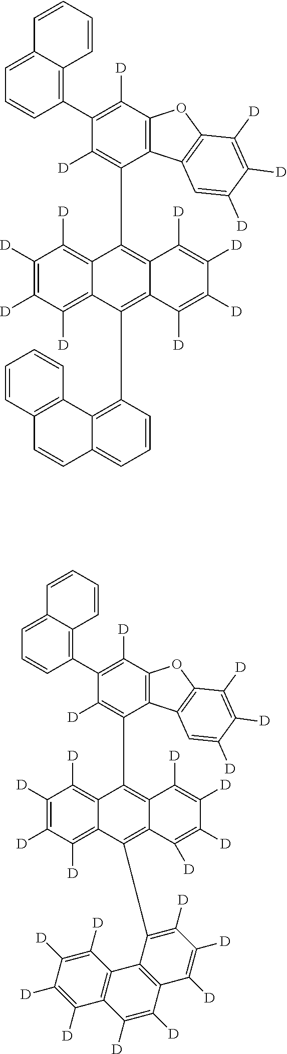 [SMILES: [2H]c1cc2c(oc3c([2H])c(-c4cccc5ccccc45)c([2H])c(-c4c5c([2H])c([2H])c([2H])c([2H])c5c(-c5cccc6ccc7ccccc7c56)c5c([2H])c([2H])c([2H])c([2H])c45)c32)c([2H])c1[2H] ; [2H]c1cc2c(oc3c([2H])c(-c4cccc5ccccc45)c([2H])c(-c4c5c([2H])c([2H])c([2H])c([2H])c5c(-c5c([2H])c([2H])c([2H])c6c([2H])c([2H])c7c([2H])c([2H])c([2H])c([2H])c7c56)c5c([2H])c([2H])c([2H])c([2H])c45)c32)c([2H])c1[2H]]

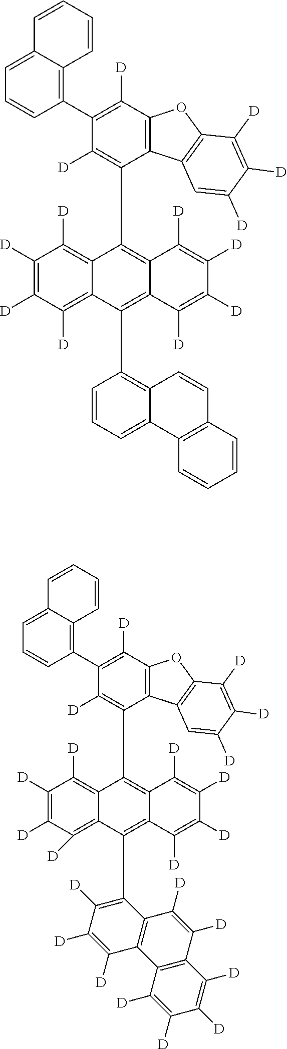 [SMILES: [2H]c1cc2c(oc3c([2H])c(-c4cccc5ccccc45)c([2H])c(-c4c5c([2H])c([2H])c([2H])c([2H])c5c(-c5cccc6c5ccc5ccccc56)c5c([2H])c([2H])c([2H])c([2H])c45)c32)c([2H])c1[2H] ; [2H]c1cc2c(oc3c([2H])c(-c4cccc5ccccc45)c([2H])c(-c4c5c([2H])c([2H])c([2H])c([2H])c5c(-c5c([2H])c([2H])c([2H])c6c5c([2H])c([2H])c5c([2H])c([2H])c([2H])c([2H])c56)c5c([2H])c([2H])c([2H])c([2H])c45)c32)c([2H])c1[2H]]

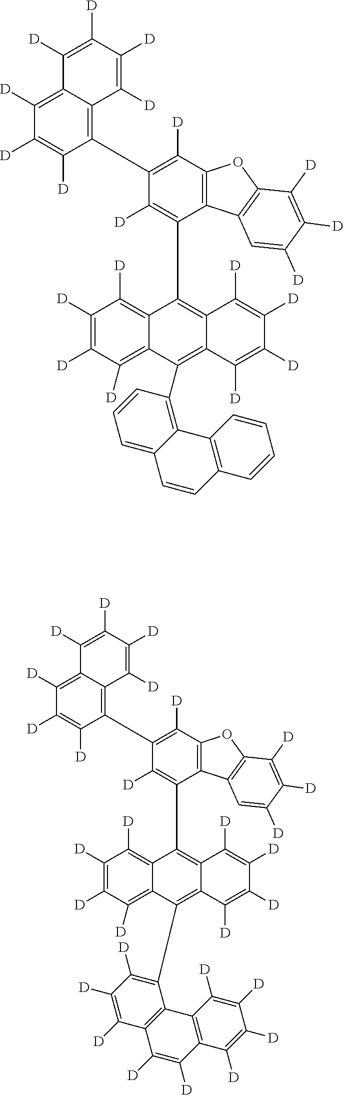 [SMILES: [2H]c1cc2c(oc3c([2H])c(-c4c([2H])c([2H])c([2H])c5c([2H])c([2H])c([2H])c([2H])c45)c([2H])c(-c4c5c([2H])c([2H])c([2H])c([2H])c5c(-c5cccc6ccc7ccccc7c56)c5c([2H])c([2H])c([2H])c([2H])c45)c32)c([2H])c1[2H] ; [2H]c1cc2c(oc3c([2H])c(-c4c([2H])c([2H])c([2H])c5c([2H])c([2H])c([2H])c([2H])c45)c([2H])c(-c4c5c([2H])c([2H])c([2H])c([2H])c5c(-c5c([2H])c([2H])c([2H])c6c([2H])c([2H])c7c([2H])c([2H])c([2H])c([2H])c7c56)c5c([2H])c([2H])c([2H])c([2H])c45)c32)c([2H])c1[2H]]

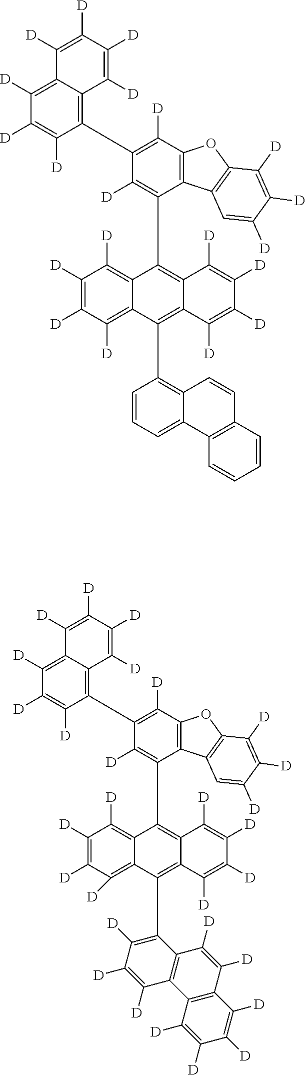 [SMILES: [2H]c1cc2c(oc3c([2H])c(-c4c([2H])c([2H])c([2H])c5c([2H])c([2H])c([2H])c([2H])c45)c([2H])c(-c4c5c([2H])c([2H])c([2H])c([2H])c5c(-c5cccc6c5ccc5ccccc56)c5c([2H])c([2H])c([2H])c([2H])c45)c32)c([2H])c1[2H] ; [2H]c1cc2c(oc3c([2H])c(-c4c([2H])c([2H])c([2H])c5c([2H])c([2H])c([2H])c([2H])c45)c([2H])c(-c4c5c([2H])c([2H])c([2H])c([2H])c5c(-c5c([2H])c([2H])c([2H])c6c5c([2H])c([2H])c5c([2H])c([2H])c([2H])c([2H])c56)c5c([2H])c([2H])c([2H])c([2H])c45)c32)c([2H])c1[2H]]

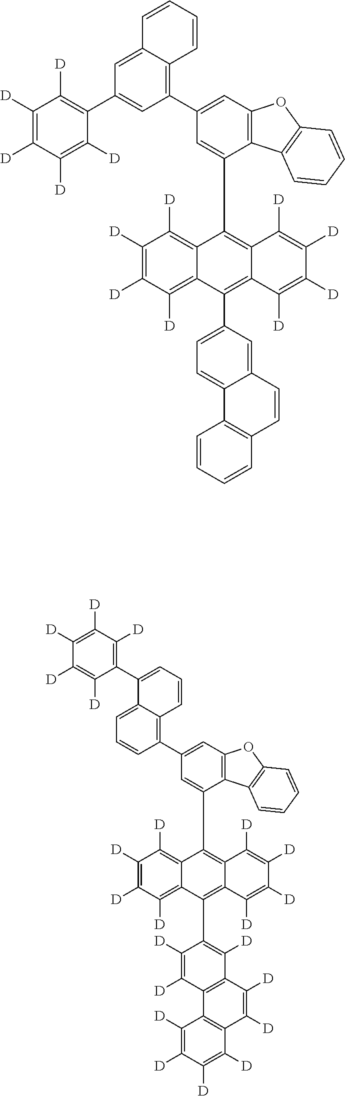 [SMILES: [2H]c1c([2H])c([2H])c(-c2cc(-c3cc(-c4c5c([2H])c([2H])c([2H])c([2H])c5c(-c5ccc6c(ccc7ccccc76)c5)c5c([2H])c([2H])c([2H])c([2H])c45)c4c(c3)oc3ccccc34)c3ccccc3c2)c([2H])c1[2H] ; [2H]c1c([2H])c([2H])c(-c2cccc3c(-c4cc(-c5c6c([2H])c([2H])c([2H])c([2H])c6c(-c6c([2H])c([2H])c7c(c6[2H])c([2H])c([2H])c6c([2H])c([2H])c([2H])c([2H])c67)c6c([2H])c([2H])c([2H])c([2H])c56)c5c(c4)oc4ccccc45)cccc23)c([2H])c1[2H]]

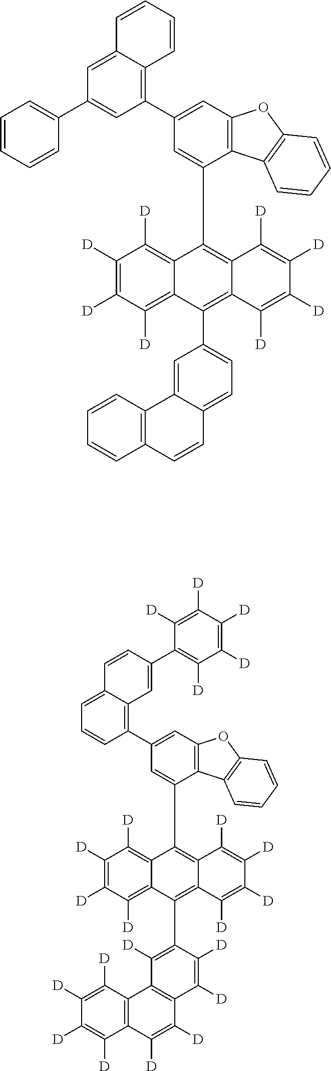 [SMILES: [2H]c1c([2H])c([2H])c2c(-c3cc(-c4cc(-c5ccccc5)cc5ccccc45)cc4oc5ccccc5c34)c3c([2H])c([2H])c([2H])c([2H])c3c(-c3ccc4ccc5ccccc5c4c3)c2c1[2H] ; [2H]c1c([2H])c([2H])c(-c2ccc3cccc(-c4cc(-c5c6c([2H])c([2H])c([2H])c([2H])c6c(-c6c([2H])c([2H])c7c([2H])c([2H])c8c([2H])c([2H])c([2H])c([2H])c8c7c6[2H])c6c([2H])c([2H])c([2H])c([2H])c56)c5c(c4)oc4ccccc45)c3c2)c([2H])c1[2H]]

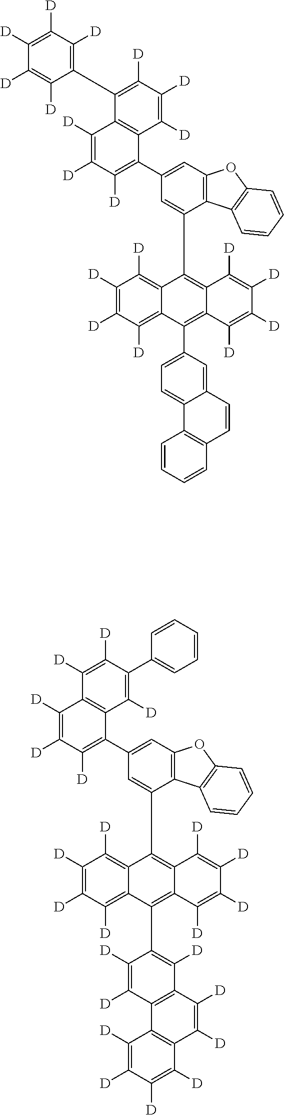 [SMILES: [2H]c1c([2H])c([2H])c(-c2c([2H])c([2H])c([2H])c3c(-c4cc(-c5c6c([2H])c([2H])c([2H])c([2H])c6c(-c6ccc7c(ccc8ccccc87)c6)c6c([2H])c([2H])c([2H])c([2H])c56)c5c(c4)oc4ccccc45)c([2H])c([2H])c([2H])c23)c([2H])c1[2H] ; [2H]c1c([2H])c(-c2cc(-c3c4c([2H])c([2H])c([2H])c([2H])c4c(-c4c([2H])c([2H])c5c(c4[2H])c([2H])c([2H])c4c([2H])c([2H])c([2H])c([2H])c45)c4c([2H])c([2H])c([2H])c([2H])c34)c3c(c2)oc2ccccc23)c2c([2H])c(-c3ccccc3)c([2H])c([2H])c2c1[2H]]

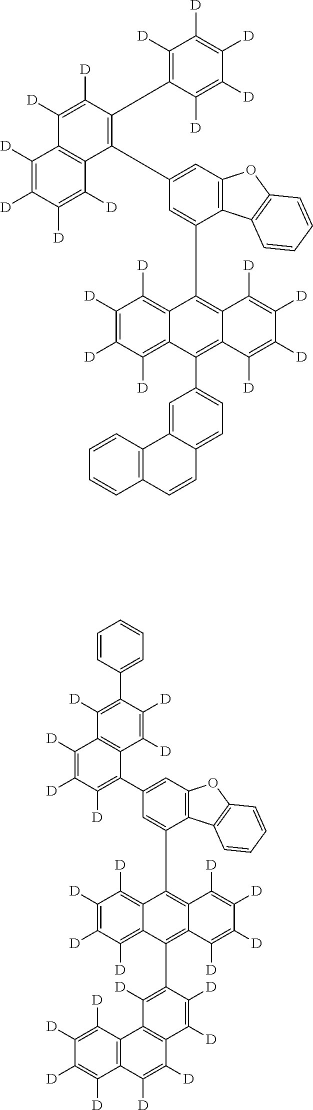 [SMILES: [2H]c1c([2H])c([2H])c(-c2c([2H])c([2H])c3c([2H])c([2H])c([2H])c([2H])c3c2-c2cc(-c3c4c([2H])c([2H])c([2H])c([2H])c4c(-c4ccc5ccc6ccccc6c5c4)c4c([2H])c([2H])c([2H])c([2H])c34)c3c(c2)oc2ccccc23)c([2H])c1[2H] ; [2H]c1c([2H])c(-c2cc(-c3c4c([2H])c([2H])c([2H])c([2H])c4c(-c4c([2H])c([2H])c5c([2H])c([2H])c6c([2H])c([2H])c([2H])c([2H])c6c5c4[2H])c4c([2H])c([2H])c([2H])c([2H])c34)c3c(c2)oc2ccccc23)c2c([2H])c([2H])c(-c3ccccc3)c([2H])c2c1[2H]]

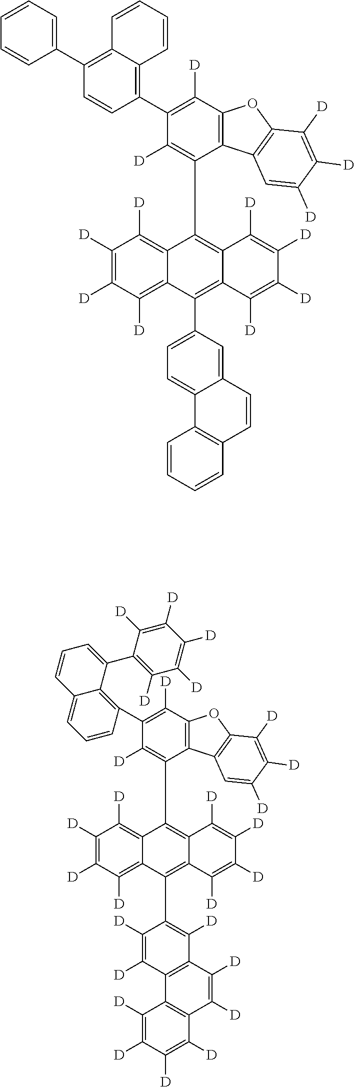 [SMILES: [2H]c1cc2c(oc3c([2H])c(-c4ccc(-c5ccccc5)c5ccccc45)c([2H])c(-c4c5c([2H])c([2H])c([2H])c([2H])c5c(-c5ccc6c(ccc7ccccc76)c5)c5c([2H])c([2H])c([2H])c([2H])c45)c32)c([2H])c1[2H] ; [2H]c1cc2c(oc3c([2H])c(-c4cccc5cccc(-c6c([2H])c([2H])c([2H])c([2H])c6[2H])c45)c([2H])c(-c4c5c([2H])c([2H])c([2H])c([2H])c5c(-c5c([2H])c([2H])c6c(c5[2H])c([2H])c([2H])c5c([2H])c([2H])c([2H])c([2H])c56)c5c([2H])c([2H])c([2H])c([2H])c45)c32)c([2H])c1[2H]]

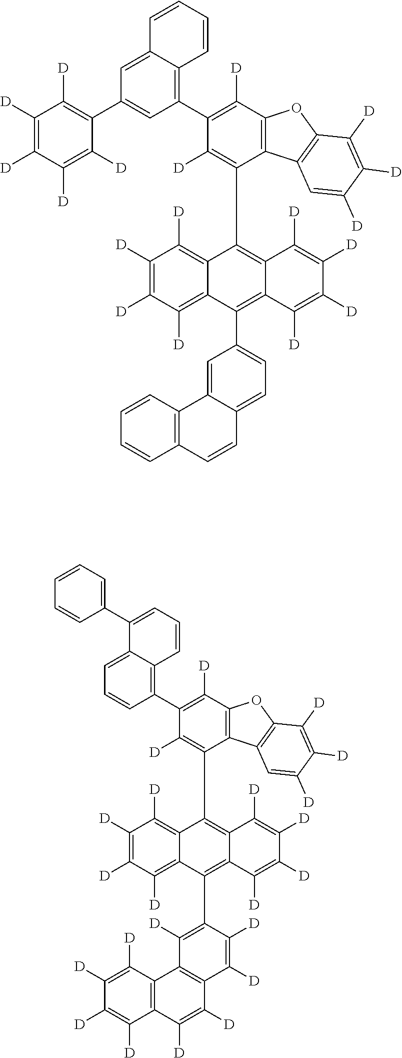 [SMILES: [2H]c1cc2c(oc3c([2H])c(-c4cc(-c5c([2H])c([2H])c([2H])c([2H])c5[2H])cc5ccccc45)c([2H])c(-c4c5c([2H])c([2H])c([2H])c([2H])c5c(-c5ccc6ccc7ccccc7c6c5)c5c([2H])c([2H])c([2H])c([2H])c45)c32)c([2H])c1[2H] ; [2H]c1cc2c(oc3c([2H])c(-c4cccc5c(-c6ccccc6)cccc45)c([2H])c(-c4c5c([2H])c([2H])c([2H])c([2H])c5c(-c5c([2H])c([2H])c6c([2H])c([2H])c7c([2H])c([2H])c([2H])c([2H])c7c6c5[2H])c5c([2H])c([2H])c([2H])c([2H])c45)c32)c([2H])c1[2H]]

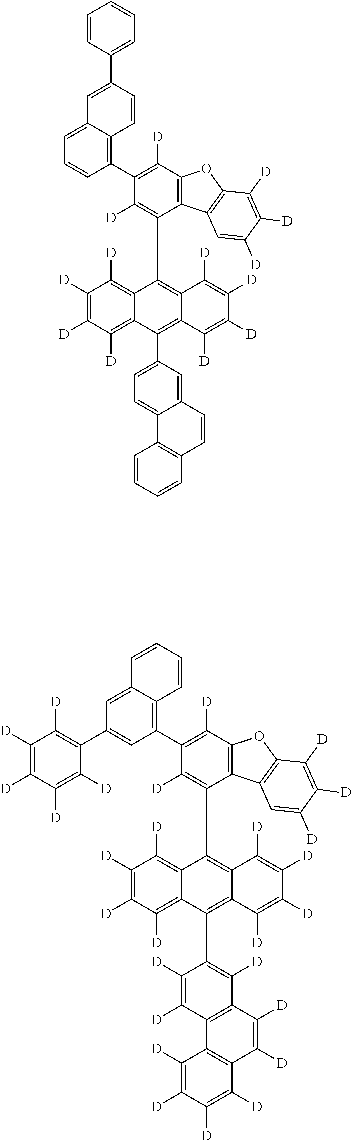 [SMILES: [2H]c1cc2c(oc3c([2H])c(-c4cccc5cc(-c6ccccc6)ccc45)c([2H])c(-c4c5c([2H])c([2H])c([2H])c([2H])c5c(-c5ccc6c(ccc7ccccc76)c5)c5c([2H])c([2H])c([2H])c([2H])c45)c32)c([2H])c1[2H] ; [2H]c1cc2c(oc3c([2H])c(-c4cc(-c5c([2H])c([2H])c([2H])c([2H])c5[2H])cc5ccccc45)c([2H])c(-c4c5c([2H])c([2H])c([2H])c([2H])c5c(-c5c([2H])c([2H])c6c(c5[2H])c([2H])c([2H])c5c([2H])c([2H])c([2H])c([2H])c56)c5c([2H])c([2H])c([2H])c([2H])c45)c32)c([2H])c1[2H]]

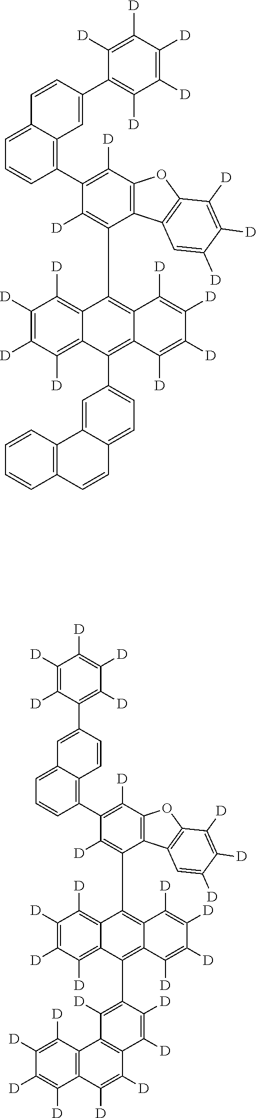 [SMILES: [2H]c1cc2c(oc3c([2H])c(-c4cccc5ccc(-c6c([2H])c([2H])c([2H])c([2H])c6[2H])cc45)c([2H])c(-c4c5c([2H])c([2H])c([2H])c([2H])c5c(-c5ccc6ccc7ccccc7c6c5)c5c([2H])c([2H])c([2H])c([2H])c45)c32)c([2H])c1[2H] ; [2H]c1cc2c(oc3c([2H])c(-c4cccc5cc(-c6c([2H])c([2H])c([2H])c([2H])c6[2H])ccc45)c([2H])c(-c4c5c([2H])c([2H])c([2H])c([2H])c5c(-c5c([2H])c([2H])c6c([2H])c([2H])c7c([2H])c([2H])c([2H])c([2H])c7c6c5[2H])c5c([2H])c([2H])c([2H])c([2H])c45)c32)c([2H])c1[2H]]

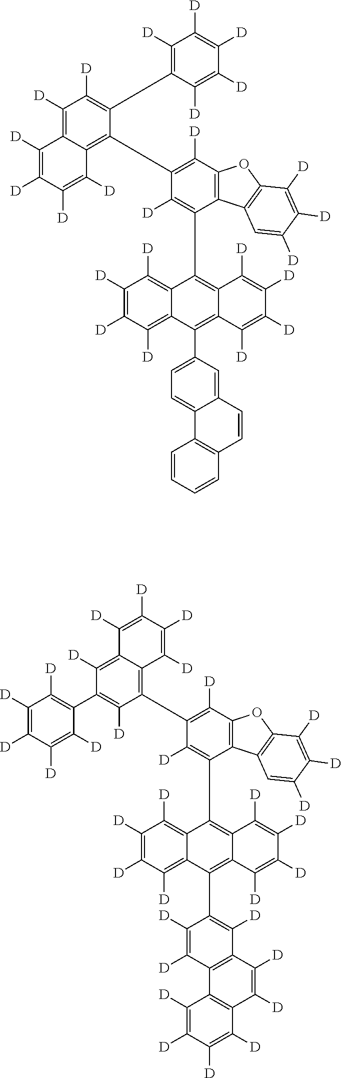

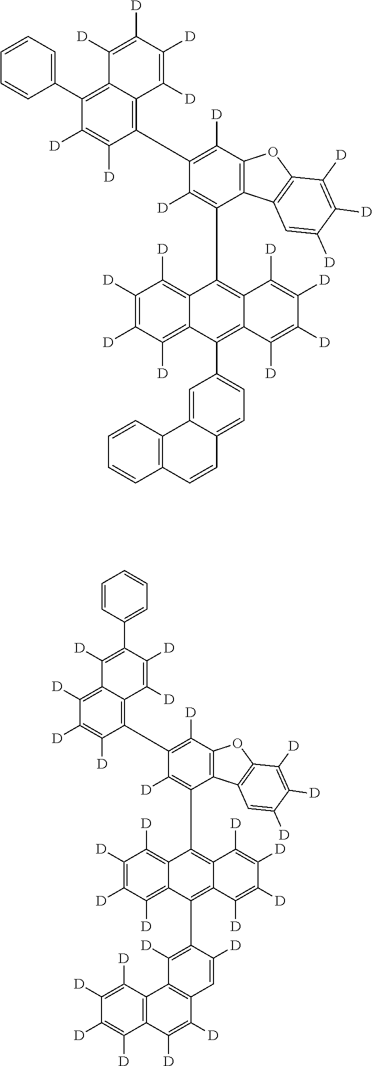 [SMILES: [2H]c1cc2c(oc3c([2H])c(-c4c([2H])c([2H])c(-c5ccccc5)c5c([2H])c([2H])c([2H])c([2H])c45)c([2H])c(-c4c5c([2H])c([2H])c([2H])c([2H])c5c(-c5ccc6ccc7ccccc7c6c5)c5c([2H])c([2H])c([2H])c([2H])c45)c32)c([2H])c1[2H] ; [2H]c1cc2c(oc3c([2H])c(-c4c([2H])c([2H])c([2H])c5c([2H])c(-c6ccccc6)c([2H])c([2H])c45)c([2H])c(-c4c5c([2H])c([2H])c([2H])c([2H])c5c(-c5c([2H])cc6c([2H])c([2H])c7c([2H])c([2H])c([2H])c([2H])c7c6c5[2H])c5c([2H])c([2H])c([2H])c([2H])c45)c32)c([2H])c1[2H]]

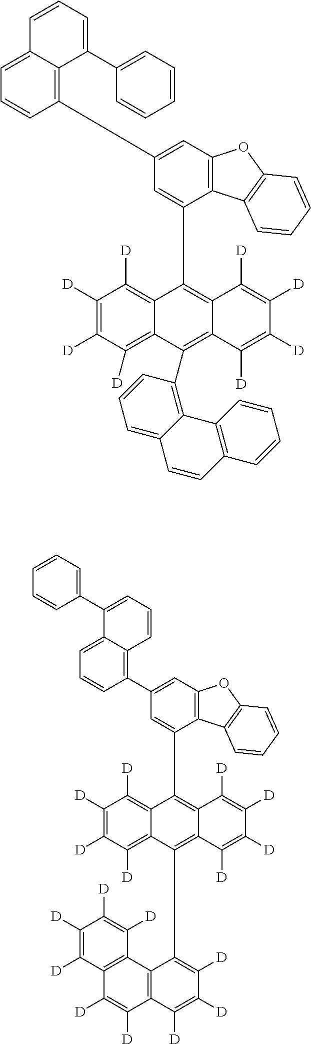 [SMILES: [2H]c1c([2H])c([2H])c2c(-c3cc(-c4cccc5cccc(-c6ccccc6)c45)cc4oc5ccccc5c34)c3c([2H])c([2H])c([2H])c([2H])c3c(-c3cccc4ccc5ccccc5c34)c2c1[2H] ; [2H]c1c([2H])c([2H])c2c(-c3c([2H])c([2H])c([2H])c4c([2H])c([2H])c5c([2H])c([2H])c([2H])c([2H])c5c34)c3c([2H])c([2H])c([2H])c([2H])c3c(-c3cc(-c4cccc5c(-c6ccccc6)cccc45)cc4oc5ccccc5c34)c2c1[2H]]

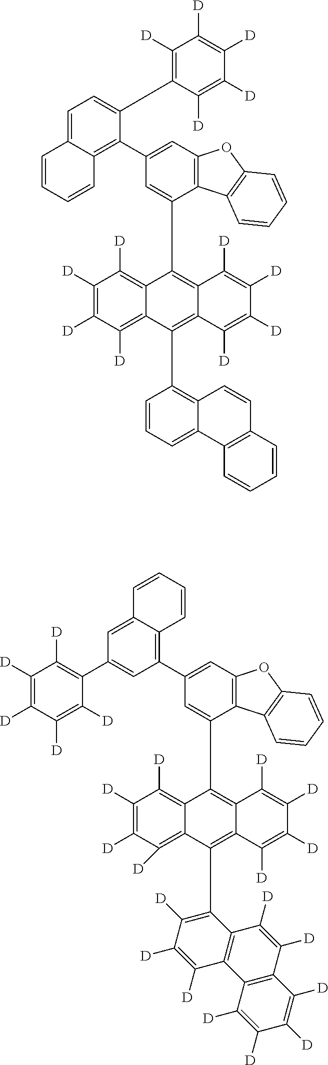 [SMILES: [2H]c1c([2H])c([2H])c(-c2ccc3ccccc3c2-c2cc(-c3c4c([2H])c([2H])c([2H])c([2H])c4c(-c4cccc5c4ccc4ccccc45)c4c([2H])c([2H])c([2H])c([2H])c34)c3c(c2)oc2ccccc23)c([2H])c1[2H] ; [2H]c1c([2H])c([2H])c(-c2cc(-c3cc(-c4c5c([2H])c([2H])c([2H])c([2H])c5c(-c5c([2H])c([2H])c([2H])c6c5c([2H])c([2H])c5c([2H])c([2H])c([2H])c([2H])c56)c5c([2H])c([2H])c([2H])c([2H])c45)c4c(c3)oc3ccccc34)c3ccccc3c2)c([2H])c1[2H]]

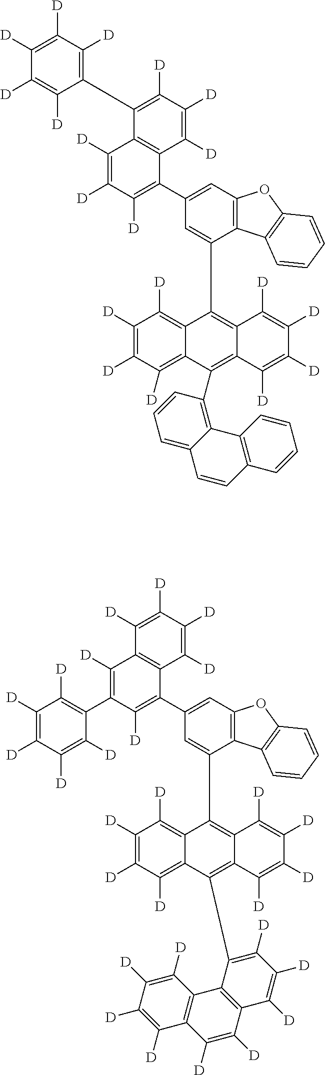

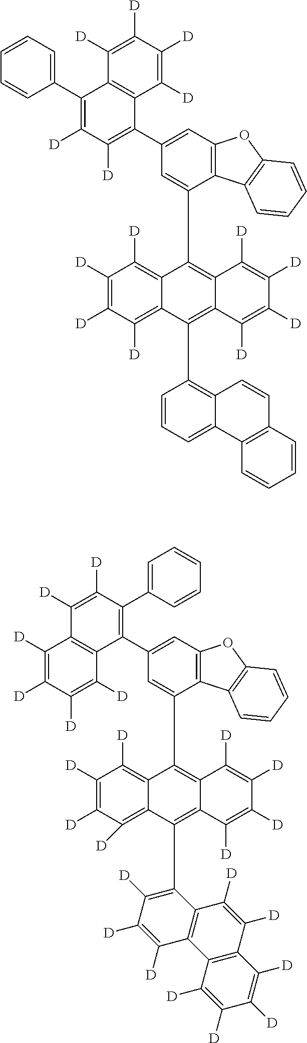 [SMILES: [2H]c1c([2H])c([2H])c2c(-c3cc(-c4c5c([2H])c([2H])c([2H])c([2H])c5c(-c5cccc6c5ccc5ccccc56)c5c([2H])c([2H])c([2H])c([2H])c45)c4c(c3)oc3ccccc34)c([2H])c([2H])c(-c3ccccc3)c2c1[2H] ; [2H]c1c([2H])c([2H])c2c(-c3cc(-c4c5c([2H])c([2H])c([2H])c([2H])c5c(-c5c([2H])c([2H])c([2H])c6c5c([2H])c([2H])c5c([2H])c([2H])c([2H])c([2H])c56)c5c([2H])c([2H])c([2H])c([2H])c45)c4c(c3)oc3ccccc34)c(-c3ccccc3)c([2H])c([2H])c2c1[2H]]

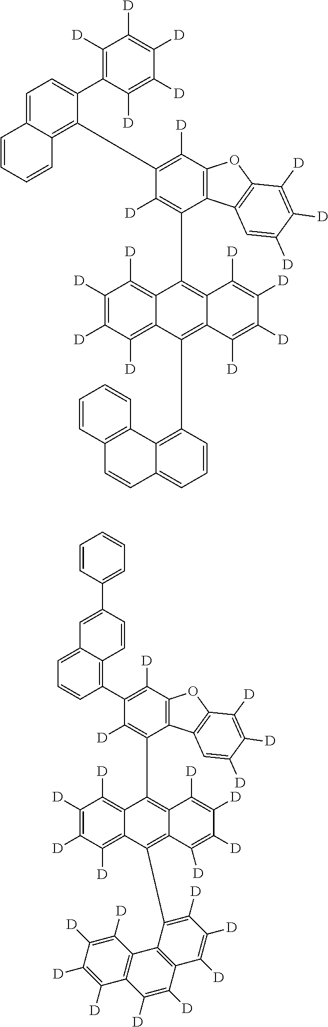 [SMILES: [2H]c1cc2c(oc3c([2H])c(-c4c(-c5c([2H])c([2H])c([2H])c([2H])c5[2H])ccc5ccccc45)c([2H])c(-c4c5c([2H])c([2H])c([2H])c([2H])c5c(-c5cccc6ccc7ccccc7c56)c5c([2H])c([2H])c([2H])c([2H])c45)c32)c([2H])c1[2H] ; [2H]c1cc2c(oc3c([2H])c(-c4cccc5cc(-c6ccccc6)ccc45)c([2H])c(-c4c5c([2H])c([2H])c([2H])c([2H])c5c(-c5c([2H])c([2H])c([2H])c6c([2H])c([2H])c7c([2H])c([2H])c([2H])c([2H])c7c56)c5c([2H])c([2H])c([2H])c([2H])c45)c32)c([2H])c1[2H]]

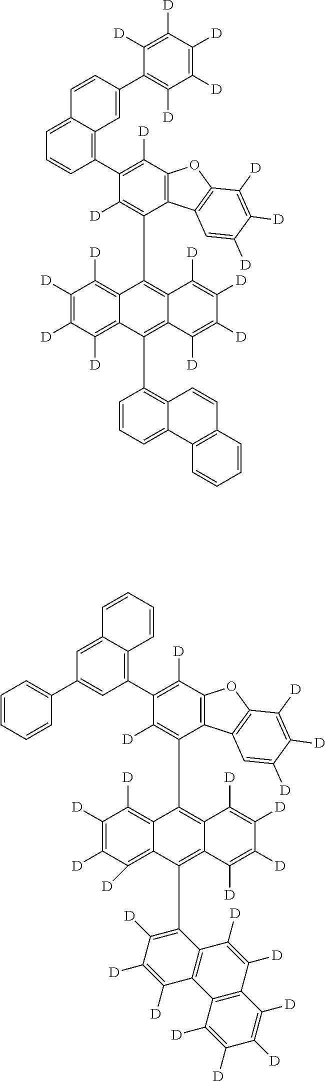 [SMILES: [2H]c1cc2c(oc3c([2H])c(-c4cccc5ccc(-c6c([2H])c([2H])c([2H])c([2H])c6[2H])cc45)c([2H])c(-c4c5c([2H])c([2H])c([2H])c([2H])c5c(-c5cccc6c5ccc5ccccc56)c5c([2H])c([2H])c([2H])c([2H])c45)c32)c([2H])c1[2H] ; [2H]c1cc2c(oc3c([2H])c(-c4cc(-c5ccccc5)cc5ccccc45)c([2H])c(-c4c5c([2H])c([2H])c([2H])c([2H])c5c(-c5c([2H])c([2H])c([2H])c6c5c([2H])c([2H])c5c([2H])c([2H])c([2H])c([2H])c56)c5c([2H])c([2H])c([2H])c([2H])c45)c32)c([2H])c1[2H]]

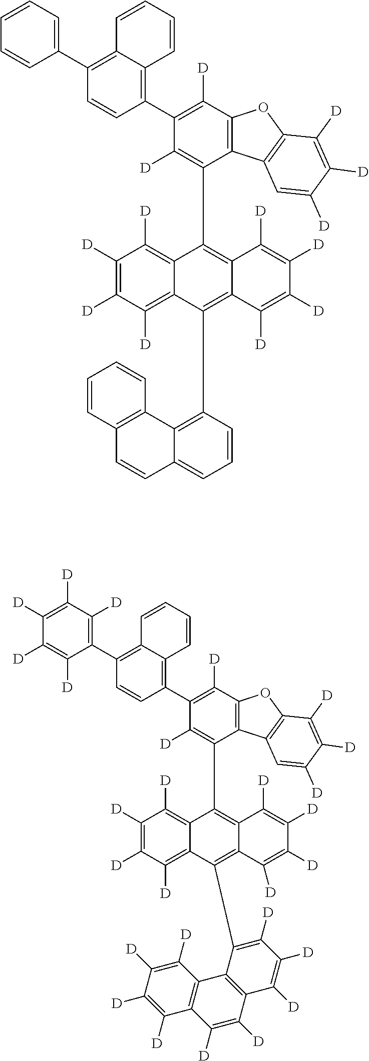 [SMILES: [2H]c1cc2c(oc3c([2H])c(-c4ccc(-c5ccccc5)c5ccccc45)c([2H])c(-c4c5c([2H])c([2H])c([2H])c([2H])c5c(-c5cccc6ccc7ccccc7c56)c5c([2H])c([2H])c([2H])c([2H])c45)c32)c([2H])c1[2H] ; [2H]c1cc2c(oc3c([2H])c(-c4ccc(-c5c([2H])c([2H])c([2H])c([2H])c5[2H])c5ccccc45)c([2H])c(-c4c5c([2H])c([2H])c([2H])c([2H])c5c(-c5c([2H])c([2H])c([2H])c6c([2H])c([2H])c7c([2H])c([2H])c([2H])c([2H])c7c56)c5c([2H])c([2H])c([2H])c([2H])c45)c32)c([2H])c1[2H]]

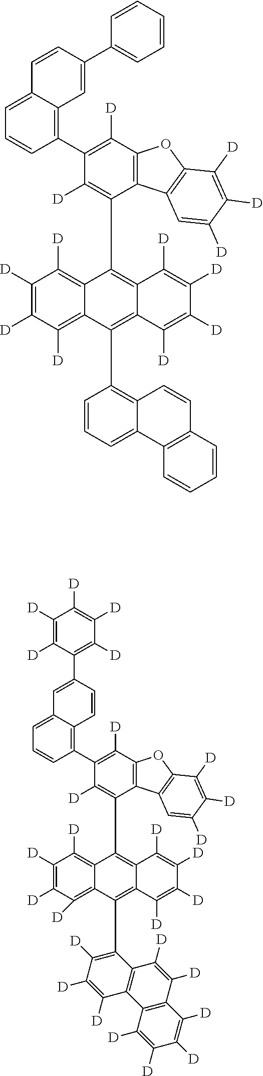 [SMILES: [2H]c1cc2c(oc3c([2H])c(-c4cccc5ccc(-c6ccccc6)cc45)c([2H])c(-c4c5c([2H])c([2H])c([2H])c([2H])c5c(-c5cccc6c5ccc5ccccc56)c5c([2H])c([2H])c([2H])c([2H])c45)c32)c([2H])c1[2H] ; [2H]c1cc2c(oc3c([2H])c(-c4cccc5cc(-c6c([2H])c([2H])c([2H])c([2H])c6[2H])ccc45)c([2H])c(-c4c5c([2H])c([2H])c([2H])c([2H])c5c(-c5c([2H])c([2H])c([2H])c6c5c([2H])c([2H])c5c([2H])c([2H])c([2H])c([2H])c56)c5c([2H])c([2H])c([2H])c([2H])c45)c32)c([2H])c1[2H]]

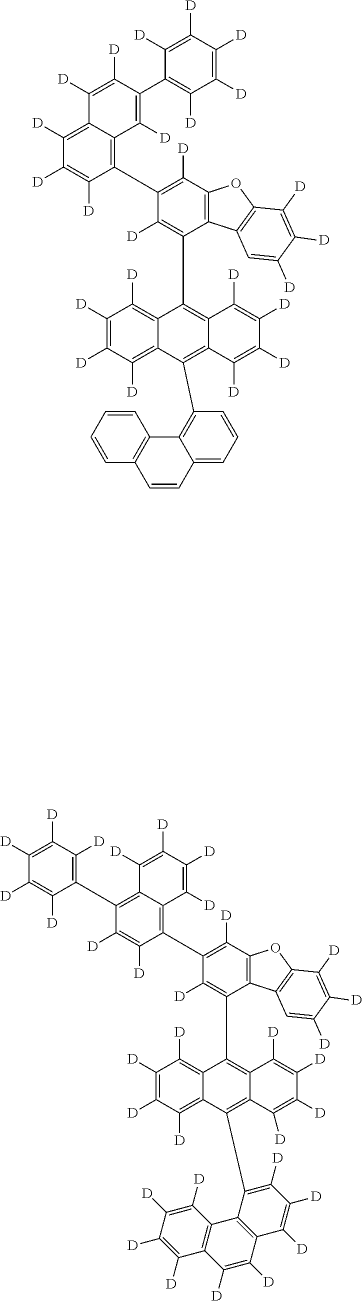

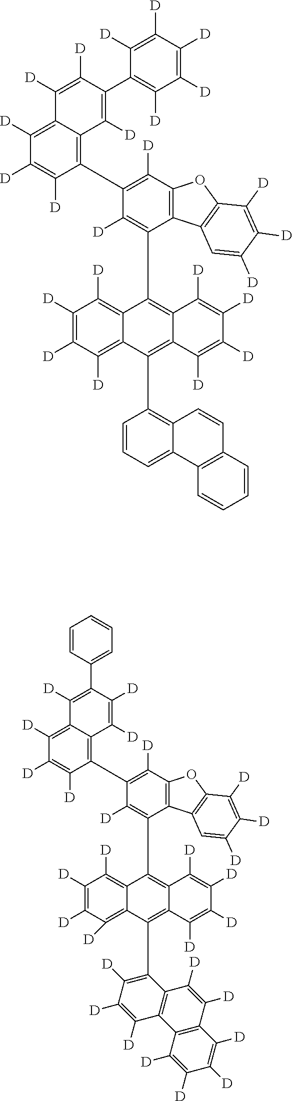 [SMILES: [2H]c1cc2c(oc3c([2H])c(-c4c([2H])c([2H])c([2H])c5c([2H])c([2H])c(-c6c([2H])c([2H])c([2H])c([2H])c6[2H])c([2H])c45)c([2H])c(-c4c5c([2H])c([2H])c([2H])c([2H])c5c(-c5cccc6c5ccc5ccccc56)c5c([2H])c([2H])c([2H])c([2H])c45)c32)c([2H])c1[2H] ; [2H]c1cc2c(oc3c([2H])c(-c4c([2H])c([2H])c([2H])c5c([2H])c(-c6ccccc6)c([2H])c([2H])c45)c([2H])c(-c4c5c([2H])c([2H])c([2H])c([2H])c5c(-c5c([2H])c([2H])c([2H])c6c5c([2H])c([2H])c5c([2H])c([2H])c([2H])c([2H])c56)c5c([2H])c([2H])c([2H])c([2H])c45)c32)c([2H])c1[2H]]

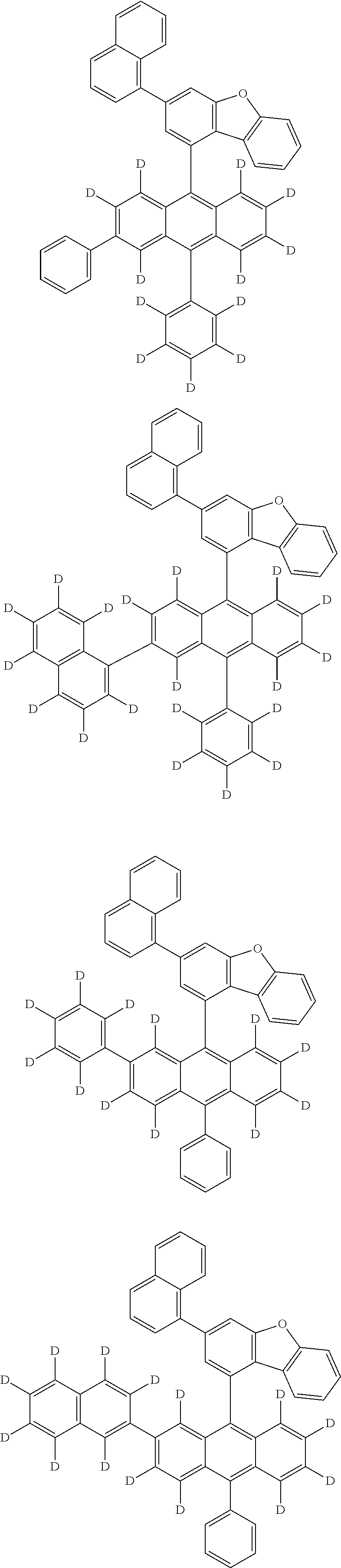

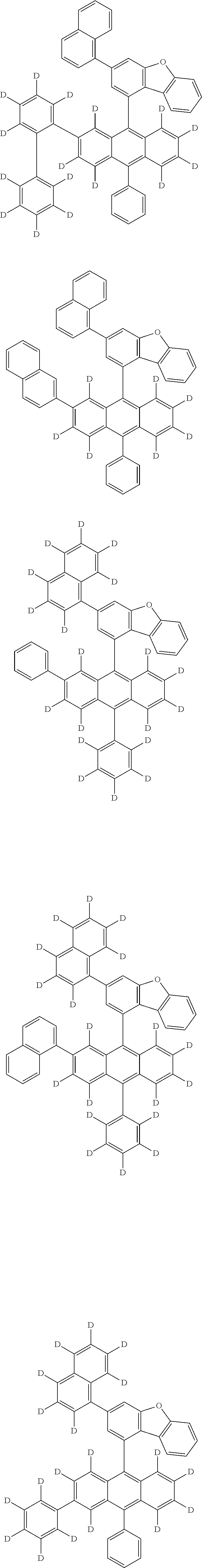

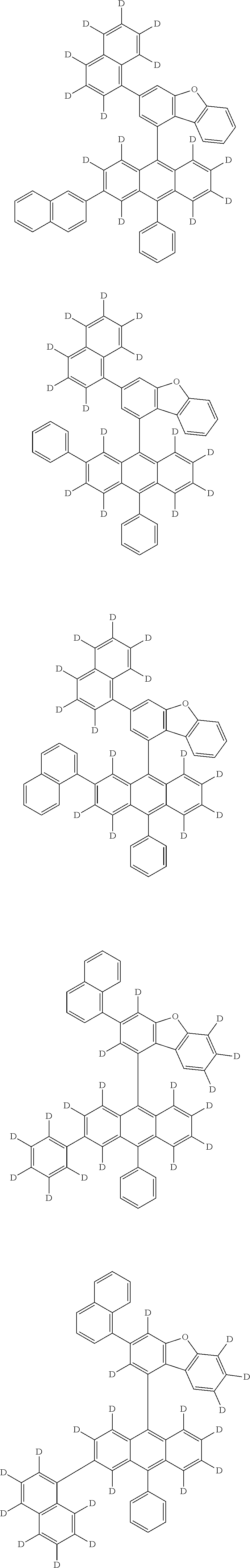

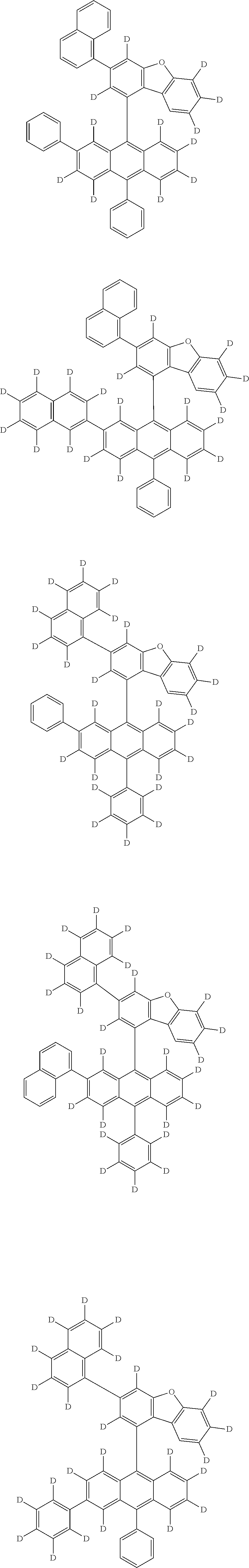

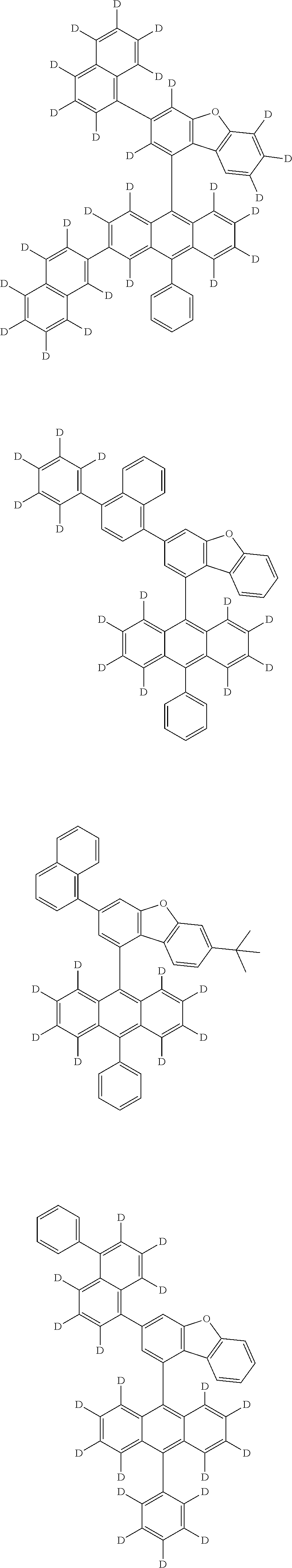

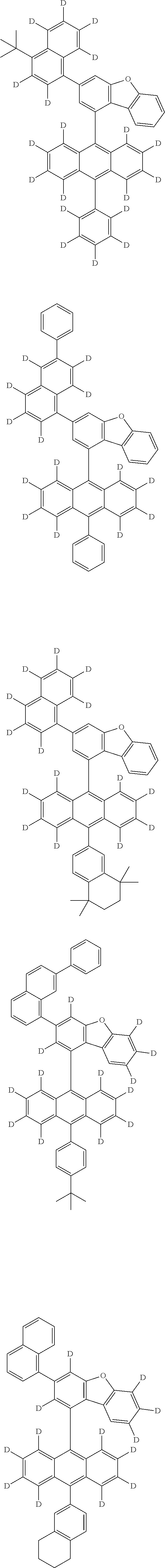

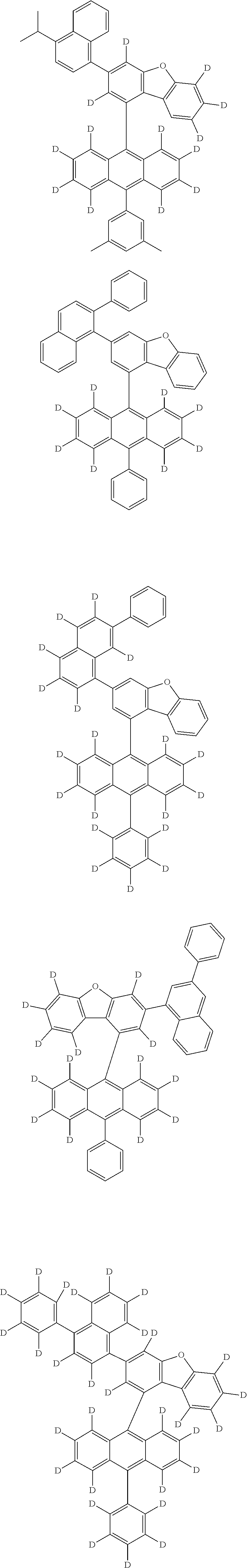

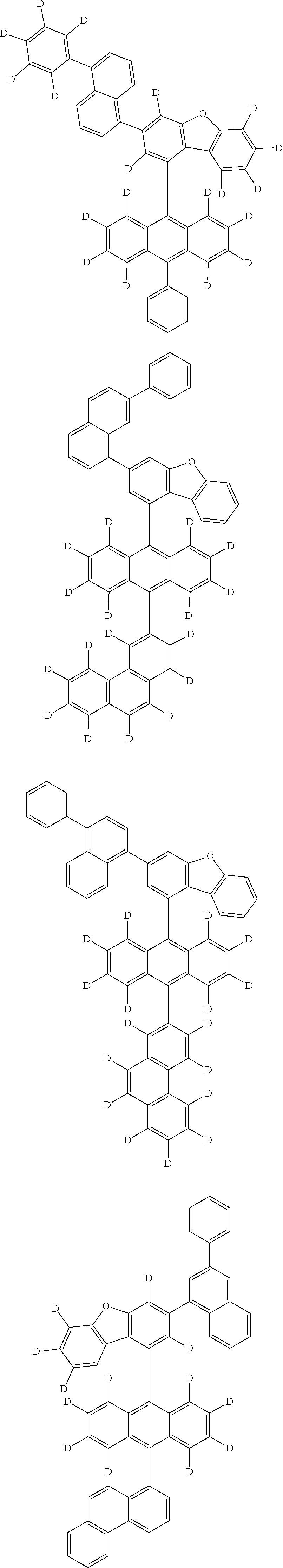

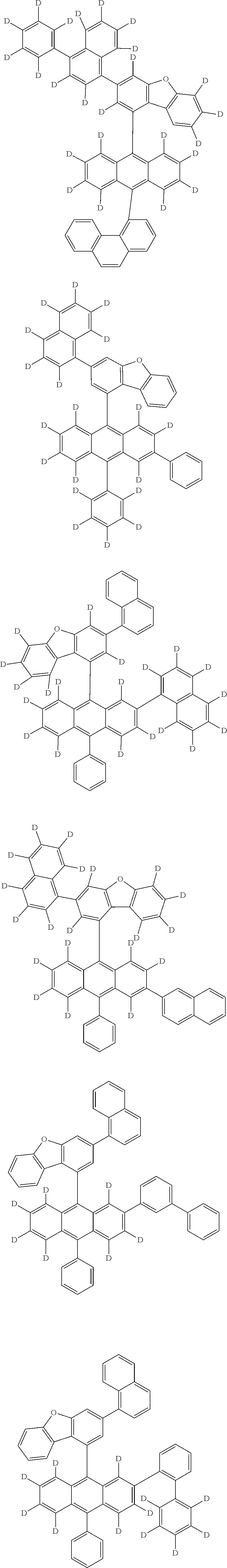

The present specification provides an organic light-emitting device containing the compound described above.

In the present specification, when a member is said to be located “on” another member, it includes not only the case where the member is in contact with another member but also the case where a still another member exists between the two members.

In the present specification, when a certain part is said to “include” a certain component, it means that, unless stated otherwise, additional components may also be included.

In the present specification, the term “layer” is interchangeable with the term “film”, which is mainly used in the related art, and means a coating covering a desired area. The size of the ‘layer’ is not limited, and each ‘layer’ may be the same or different in size. In an exemplary embodiment, the size of the ‘layer’ can be the same as that of the entire device, can correspond to the size of a particular functional area, or can be as small as a single sub-pixel.

In the present specification, the meaning of a specific material A being included in a layer B includes both i) one or more A material being included in one B layer, and ii) the B layer consisting of one or more layers, and the A material being included in one or more of the multiple B layers.

In the present specification, the meaning of a specific material A being included in a layer C or a layer D includes all of i) the material A being included in at least one of one or more of the C layer, ii) being included in at least one of one or more of the D layer, and iii) being included in one or more of the C layer and one or more of the D layer, respectively.

The present specification provides an organic light-emitting device including a: first electrode; a second electrode provided opposite to the first electrode; and one or more organic layers provided between the first electrode and the second electrode, wherein at least one of the one or more organic layers contains the compound of Chemical Formula 1-1 or Chemical Formula 1-2.

The organic layer of the organic light-emitting device of the present specification may be formed as a single-layer structure, but may also be formed as a multi-layer structure in which two or more organic layers are stacked. For example, it may have a structure including a hole injection layer, a hole transport layer, a light-emitting layer, an electron transport layer, an electron injection layer, an electron-blocking layer, a hole-blocking layer, etc. However, the structure of the organic light-emitting device is not limited thereto and may include a smaller number of organic layers.

In an exemplary embodiment of the present specification, the organic layer includes a light-emitting layer, and the light-emitting layer contains the compound represented by Chemical Formula 1-1 or Chemical Formula 1-2.

In an exemplary embodiment of the present specification, the organic layer includes a light-emitting layer, and the light-emitting layer contains the compound represented by Chemical Formula 1-1 or Chemical Formula 1-2 as a host of the light-emitting layer.

In an exemplary embodiment of the present specification, the light-emitting layer further contains a dopant, and the dopant includes a fluorescent dopant.

In an exemplary embodiment of the present specification, the fluorescent dopant is a pyrene-based compound or a non-pyrene-based compound.

In an exemplary embodiment of the present specification, the non-pyrene-based compound includes a boron-based compound.

In an exemplary embodiment of the present specification, the light-emitting layer further contains one or more host different from the compound of Chemical Formula 1-1 or Chemical Formula 1-2.

The host different from the compound of Chemical Formula 1-1 or Chemical Formula 1-2 is not limited as long as it is an anthracene-based host used in the art, which is different from the compound of Chemical Formula 1-1 or Chemical Formula 1-2, although not being limited thereto.

In an exemplary embodiment of the present specification, the light-emitting layer contains a host and a dopant.

In an exemplary embodiment of the present specification, the light-emitting layer contains a host and a dopant, and the host includes the compound represented by Chemical Formula 1-1 or Chemical Formula 1-2.

In an exemplary embodiment of the present specification, the dopant is a blue dopant.

In an exemplary embodiment of the present specification, the organic light-emitting device is a blue organic light-emitting device.

In an exemplary embodiment of the present specification, the light-emitting layer contains two or more mixed hosts, and one or more of the two or more mixed hosts includes the compound represented by Chemical Formula 1-1 or Chemical Formula 1-2.

In an exemplary embodiment of the present specification, the light-emitting layer contains two or more mixed hosts, at least one of the two or more mixed hosts includes the compound represented by Chemical Formula 1-1 or Chemical Formula 1-2, and the remainder includes an anthracene-based compound different from the compound represented by Chemical Formula 1-1 or Chemical Formula 1-2.

At least one of the two or more mixed hosts includes the compound represented by Chemical Formula 1-1 or Chemical Formula 1-2, and the remainder is not limited as long as it is an anthracene-based host used in the art, which is different from the compound represented by Chemical Formula 1-1 or Chemical Formula 1-2, although not being limited thereto.

In an exemplary embodiment of the present specification, at least one of the mixed hosts necessarily includes an anthracene compound substituted with deuterium, and the remainder includes an anthracene compound not substituted with deuterium, one of them including a compound represented by Chemical Formula 1-1 or Chemical Formula 1-2.

In an exemplary embodiment of the present specification, at least two of the mixed hosts necessarily includes an anthracene compound substituted with deuterium, and one of them includes a compound represented by Chemical Formula 1-1 or Chemical Formula 1-2.

The organic light-emitting device using two or more mixed hosts in an exemplary embodiment of the present specification is intended to improve the performance of the device by combining the advantages of each host. For example, when two hosts are mixed, an organic light-emitting device having the effects of high efficiency, low voltage and long lifetime can be prepared by mixing one host having the effects of high efficiency and low voltage and one host having the effect of long lifetime.

In an exemplary embodiment of the present specification, the organic light-emitting device has a maximum emission wavelength (λₘₐₓ) of 400 nm to 470 nm.

In an exemplary embodiment of the present specification, the light-emitting layer contains a host and a dopant, and the dopant is a fluorescent dopant.

In an exemplary embodiment of the present specification, the light-emitting layer contains a host and a dopant, and the dopant includes at least one selected from a pyrene-based compound and a non-pyrene-based compound.

The pyrene-based compound and the non-pyrene-based compound are not limited as long as they are compounds used in the art, although not being limited thereto.

In an exemplary embodiment of the present specification, the non-pyrene-based compound includes a boron-based compound.

In an exemplary embodiment of the present specification, the light-emitting layer contains a host and a dopant, the host includes a compound represented by Chemical Formula 1-1 or Chemical Formula 1-2, and the dopant includes at least one selected from a pyrene-based compound and a non-pyrene-based compound.

In an exemplary embodiment of the present specification, the light-emitting layer contains a host and a dopant, and the light-emitting layer contains the host and the dopant at a weight ratio of 0.1:99.9 to 20:80.

In an exemplary embodiment of the present specification, the light-emitting layer contains a host and a dopant, and the light-emitting layer contains the host and the dopant at a weight ratio of 95:5 to 99:1.

In an exemplary embodiment of the present specification, the organic layer contains the compound described above, and the band gap energy of the compound is 2.9 eV or higher.

In an exemplary embodiment of the present specification, the organic light-emitting device further includes one or two or more layers selected from a group consisting of a hole injection layer, a hole transport layer, a light-emitting layer, an electron transport layer, an electron injection layer, a hole-blocking layer, and an electron-blocking layer.

In an exemplary embodiment of the present specification, the organic light-emitting device includes a first electrode; a second electrode provided opposite the first electrode; a light-emitting layer provided between the first electrode and the second electrode; and two or more organic layers provided between the light-emitting layer and the first electrode, or between the light-emitting layer and the second electrode.

In an exemplary embodiment of the present specification, the two or more organic layers between the light-emitting layer and the first electrode, or between the light-emitting layer and the second electrode may include at least two selected from a group consisting of a light-emitting layer, a hole transport layer, a hole injection layer, a hole injection and transport layer, an electron-blocking layer, a hole-blocking layer, an electron injection layer, an electron transport layer, and an electron injection and transport layer.

In an exemplary embodiment of the present specification, two or more hole transport layers are included between the light-emitting layer and the first electrode. The two or more hole transport layers may contain the same or different materials.

In an exemplary embodiment of the present specification, the first electrode is an anode or a cathode.

In an exemplary embodiment of the present specification, the second electrode is a cathode or an anode.

In an exemplary embodiment of the present specification, the organic light-emitting device may be an organic light-emitting device with a structure in which an anode, one or more organic layers, and a cathode are stacked sequentially on a substrate (normal type).

In an exemplary embodiment of the present specification, the organic light-emitting device may be an organic light-emitting device with a structure in which a cathode, one or more organic layers, and an anode are stacked sequentially on a substrate (inverted type).

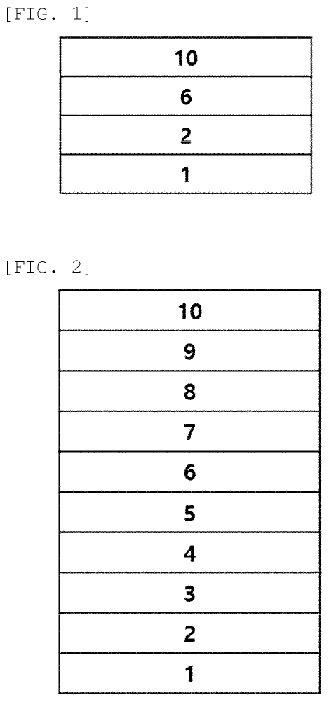

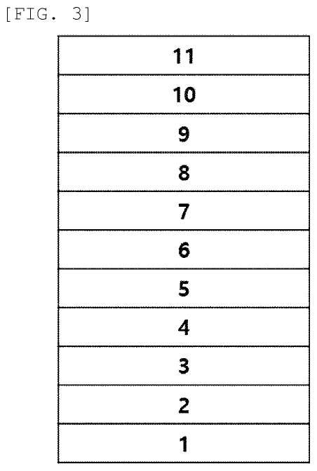

For example, the structures of the organic light-emitting device in an exemplary embodiment of the present specification are illustrated in FIGS. 1 and 2. FIGS. 1 and 2 show non-limiting exemplary organic light-emitting devices.

FIG. 1 shows an example of an organic light-emitting device in which a first electrode 2, a light-emitting layer 6, and a second electrode 10 are stacked sequentially on a substrate 1. The compound is contained in the light-emitting layer 6.

FIG. 2 shows an example of an organic light-emitting device in which a first electrode 2, a hole injection layer 3, a hole transport layer 4, an electron-blocking (hole control) layer 5, a light-emitting layer 6, a hole-blocking (electron control) layer 7, an electron transport layer 8, an electron injection layer 9, and a second electrode 10 are stacked sequentially on a substrate 1. The compound described above is contained in the light-emitting layer 6.

FIG. 3 shows an example of an organic light-emitting device in which a first electrode 2, a hole injection layer 3, a hole transport layer 4, an electron-blocking (hole control) layer 5, a light-emitting layer 6, a hole-blocking (electron control) layer 7, an electron transport layer 8, an electron injection layer 9, a second electrode 10, and a capping layer 11 are stacked sequentially on a substrate 1. The compound described above is contained in the light-emitting layer 6.

The organic light-emitting device of the present specification may be prepared using materials and methods known in the art, except that the light-emitting layer contains the compound described above, i.e., the compound represented by Chemical Formula 1-1 or 1-2.

When the organic light-emitting device includes a plurality of organic layers, the organic layers may be formed of identical or different materials.

For example, the organic light-emitting device of the present specification may be prepared by stacking a first electrode, an organic layer, and a second electrode sequentially on a substrate. A PVD (physical vapor deposition) method such as sputtering or e-beam evaporation may be used to deposit a metal, a conductive metal oxide or an alloy thereof on a substrate to form a first electrode, an organic layer including a hole injection layer, a hole transport layer, a light-emitting layer, and an electron transport layer may be formed thereon, and then a material that can be used as a second electrode may be deposited thereon to prepare the device. In addition to this method, the organic light-emitting device may be prepared by sequentially depositing a second electrode material, an organic layer, and a first electrode material on a substrate.

In addition, the compound represented by Chemical Formula 1-1 or Chemical Formula 1-2 may be formed into an organic layer by a solution coating method as well as the vacuum deposition method when preparing the organic light-emitting device. The solution coating method refers to spin coating, dip coating, doctor blading, inkjet printing, screen printing, spraying, roll coating, etc., although not being limited thereto.

In addition to these methods, the organic light-emitting device may also be prepared by sequentially depositing a second electrode material, an organic layer, and a first electrode material on a substrate. However, the preparation method is not limited thereto.

As the first electrode material, a material having a high work function is preferred so that hole injection into the organic layer can be facilitated. Examples include metals such as vanadium, chromium, copper, zinc and gold, or alloys thereof; metal oxides such as zinc oxide, indium oxide, indium tin oxide (ITO) and indium zinc oxide (IZO); combinations of metals and oxides such as Zno:Al or SnO₂:Sb; conductive polymers such as poly(3-methylthiophene), poly[3,4-(ethylene-1,2-dioxy)thiophene] (PEDOT), polypyrrole and polyaniline, etc., although not being limited thereto.

It is preferred that the second electrode material be a material having a low work function so that electron injection into the organic layer can be facilitated. Examples include metals such as magnesium, calcium, sodium, potassium, titanium, indium, yttrium, lithium, gadolinium, aluminum, silver, tin and lead, or alloys thereof; multilayer-structured materials such as LiF/Al or LiO₂/Al, etc., although not being limited thereto.

The light-emitting layer may contain a host material and a dopant material. When the organic light-emitting device includes an additional light-emitting layer in addition to the light-emitting layer containing the compound of Chemical Formula 1-1 or Chemical Formula 1-2 according to an exemplary embodiment of the present specification, the host material may be a condensed and/or non-condensed aromatic ring derivative, a heterocycle-containing compound, etc. Specifically, the condensed aromatic ring derivative includes an anthracene derivative, a pyrene derivative, a naphthalene derivative, a pentacene derivative, a phenanthrene compound, a fluoranthene compound, etc., and the heterocycle-containing compound includes a dibenzofuran derivative, a ladder-type furan compound, a pyrimidine derivative, etc., although not being limited thereto.

The dopant material may be an aromatic amine derivative, a styrylamine compound, a boron complex, a fluoranthene compound, a metal complex, etc. Specifically, the aromatic amine derivative is a condensed aromatic ring derivative having a substituted or unsubstituted arylamine group, and includes pyrene, anthracene, chrysene, periflanthene, etc. having an arylamine group. And, the styrylamine compound is a compound in which at least one arylvinyl group is substituted in a substituted or unsubstituted arylamine. One or more substituent selected from a group consisting of an aryl group, a silyl group, an alkyl group, a cycloalkyl group and an arylamine group is substituted or unsubstituted. Specifically, the styrylamine compound may be styrylamine, styryldiamine, styryltriamine, styryltetramine, etc., although not being limited thereto. And, the metal complex may include an iridium complex, a platinum complex, etc., although not being limited thereto.

In an exemplary embodiment of the present specification, the dopant material includes a compound of Chemical Formula D-1 or D-2, although not being limited thereto.

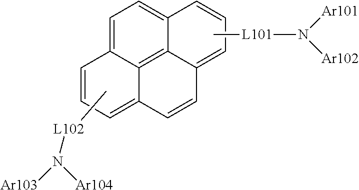

In Chemical Formula D-1,
L101 and L102, which are identical or different, are independently a direct bond or a substituted or unsubstituted arylene group, and
Ar101 to Ar104, which are identical or different, are independently a substituted or unsubstituted aryl group; or a substituted or unsubstituted heteroaryl group.

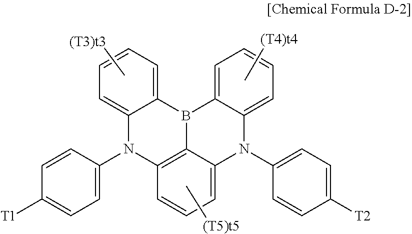

In Chemical Formula D-2,
T1 to T5, which are identical or different, are independently hydrogen; a substituted or unsubstituted alkyl group; a substituted or unsubstituted amine group; or a substituted or unsubstituted aryl group,
each of t3 and t4 is an integer from 1 to 4,
t5 is an integer from 1 to 3,
if t3 is 2 or greater, two or more T3s are identical or different,
if t4 is 2 or greater, two or more T4s are identical or different, and
if t5 is 2 or greater, two or more T5s are identical or different.

In an exemplary embodiment of the present specification, L101 and L102 are direct bonds.

In an exemplary embodiment of the present specification, Ar101 to Ar104, which are identical or different, are independently a substituted or unsubstituted C₆₋₃₀ monocyclic or polycyclic aryl group; or a substituted or unsubstituted C₂₋₃₀ monocyclic or polycyclic heteroaryl group.

According to an exemplary embodiment of the present specification, Ar101 to Ar104, which are identical or different, are independently a C₆₋₃₀ monocyclic or polycyclic aryl group substituted or unsubstituted with a C₁₋₃₀ linear or branched alkyl group; or a C₂₋₃₀ monocyclic or polycyclic heteroaryl group.

In an exemplary embodiment of the present specification, Ar101 to Ar104, which are identical or different, are independently a phenyl group substituted with a methyl group; or a dibenzofuran group.

In an exemplary embodiment of the present disclosure, Chemical Formula D-1 is represented by the following compound.

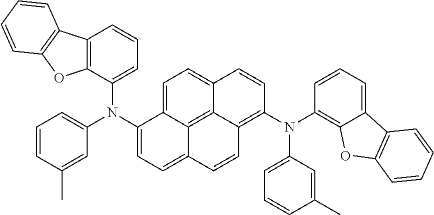 [SMILES: Cc1cccc(N(c2ccc3ccc4c(N(c5cccc(C)c5)c5cccc6c5oc5ccccc56)ccc5ccc2c3c54)c2cccc3c2oc2ccccc23)c1]

According to an exemplary embodiment of the present specification, T1 to T5, which are identical or different, are independently hydrogen; a substituted or unsubstituted C₁₋₃₀ linear or branched alkyl group; a substituted or unsubstituted C₆₋₃₀ monocyclic or polycyclic arylamine group; or a substituted or unsubstituted C₆₋₃₀ monocyclic or polycyclic aryl group.

According to an exemplary embodiment of the present specification, T1 to T5, which are identical or different, are independently hydrogen; a C₁₋₃₀ linear or branched alkyl group; a C₆₋₃₀ monocyclic or polycyclic arylamine group; or a C₆₋₃₀ monocyclic or polycyclic aryl group substituted or unsubstituted with a C₁₋₃₀ linear or branched alkyl group.

According to embodiment of the present an specification, T1 to T5, which are identical or different, are independently hydrogen; a methyl group; a tert-butyl group; a diphenylamine group; or a phenyl group that is unsubstituted or substituted with a methyl group or a tert-butyl group.

In an exemplary embodiment of the present disclosure, Chemical Formula D-2 is represented by the following compound.

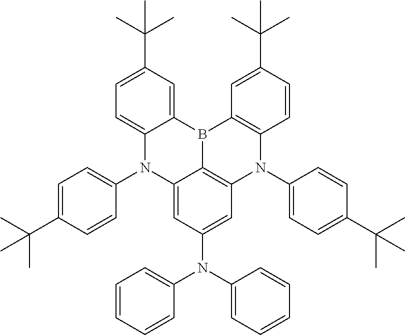 [SMILES: CC(C)(C)c1ccc(N2c3ccc(C(C)(C)C)cc3B3c4cc(C(C)(C)C)ccc4N(c4ccc(C(C)(C)C)cc4)c4cc(N(c5ccccc5)c5ccccc5)cc2c43)cc1]

The hole injection layer is a layer that receives holes from the electrode. Specifically, a material having the ability of transporting holes and exhibiting excellent hole receiving effect from the anode and hole injection effect for the light-emitting layer or the light-emitting material may be used as a hole injection material. Also, a material with excellent ability to prevent the movement of excitons generated in the light-emitting layer to the electron injection layer or an electron injection material is desirable. Also, a material with excellent thin film-forming ability is desirable. Additionally, it is desirable that the HOMO (highest occupied molecular orbital) of the hole injection material is between the work function of the anode material and the HOMO of the nearby organic layer. Specific examples of the hole injection material include a metal porphyrin, an oligothiophene, an arylamine-based organic material, a hexanitrilehexaazatriphenylene-based organic material, a quinacridone-based organic material, a perylene-based organic material, a polythiophene-based conductive polymer such as anthraquinone and polyaniline, etc., although not being limited thereto.

In an exemplary embodiment of the present specification, the hole injection layer contains a compound represented by Chemical Formula HI-1, although not being limited thereto.

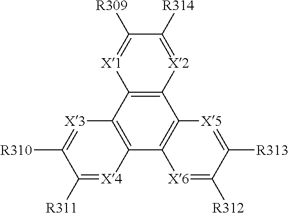

In Chemical Formula HI-1,
at least one of X′1 to X′6 is N, the remainder being CH, and
R309 to R314, which are identical or different, are independently hydrogen; deuterium; a cyano group; a substituted or unsubstituted alkyl group; a substituted or unsubstituted amine group; a substituted or unsubstituted aryl group; or a substituted or unsubstituted heteroaryl group, or is bonded to an adjacent group to form a substituted or unsubstituted ring.

In an exemplary embodiment of the present disclosure, X′1 to X′6 are N.

In an embodiment of the present specification, R309 to R314 are cyano groups.

In an exemplary embodiment of the present disclosure, Chemical Formula HI-1 is represented by the following compound.

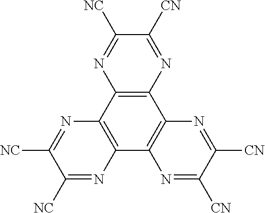 [SMILES: N#Cc1nc2c3nc(C#N)c(C#N)nc3c3nc(C#N)c(C#N)nc3c2nc1C#N]

The hole transport layer is a layer that receives holes from the hole injection layer and transports them to the light-emitting layer. As a hole transport material, a material with high mobility for holes is desirable, which can receive holes from the anode or the hole injection layer and transfer them to the light-emitting layer. Specific examples include an arylamine-based organic material, a conductive polymer, a block copolymer with both conjugated and non-conjugated segments, etc., although not being limited thereto.

The electron-blocking (hole control) layer is a layer that can improve the lifetime and efficiency of a device by preventing electrons injected from the electron injection layer from entering the hole injection layer through the light-emitting layer. Any known material may be used without limitation, and the electron-blocking (hole control) layer may be formed between the light-emitting layer and the hole injection layer, between the light-emitting layer and the hole transport layer, or between the light-emitting layer and a layer that simultaneously injects and transports holes.

In an exemplary embodiment of the present specification, the hole transport layer or the electron-blocking layer contains a compound of Chemical Formula HT-1, although not being limited thereto.

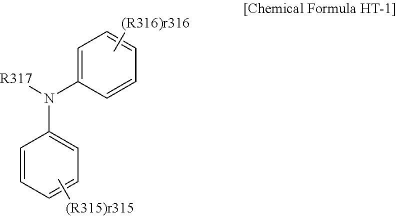

In Chemical Formula HT-1,
R315 to R317, which are identical or different, are independently any one selected from a group consisting of hydrogen; deuterium; a substituted or unsubstituted alkyl group; a substituted or unsubstituted aryl group; a substituted or unsubstituted heteroaryl group; and a combination thereof, or is bonded to an adjacent group to form a substituted or unsubstituted ring,
r315 is an integer from 1 to 5, and if r315 is 2 or greater, two or more R315's are identical or different, and
r316 is an integer from 1 to 5, and if r316 is 2 or greater, two or more R316's are identical or different.

In an embodiment of exemplary the present specification, R317 is a substituted or unsubstituted aryl group; or a substituted or unsubstituted heteroaryl group, or is bonded to an adjacent group to form an aromatic hydrocarbon ring substituted with an aryl group or an alkyl group.

In an exemplary embodiment of the present specification, R317 is any one selected from a group consisting of a substituted or unsubstituted carbazole group; a substituted or unsubstituted phenyl group; a substituted or unsubstituted biphenyl group; a substituted or unsubstituted fluorene group; and a combination thereof.

In an exemplary embodiment of the present specification, R315 and R316, which are identical or different, are independently a substituted or unsubstituted aryl group, or is bonded to an adjacent group to form an aromatic hydrocarbon ring substituted with an aryl group or an alkyl group.

In an exemplary embodiment of the present specification, R315 and R316, which are identical or different, are independently a substituted or unsubstituted phenyl group; a substituted or unsubstituted naphthyl group; or a substituted or unsubstituted carbazole group.

In an exemplary embodiment of the present specification, Chemical Formula HT-1 is represented by any one of the following compounds.

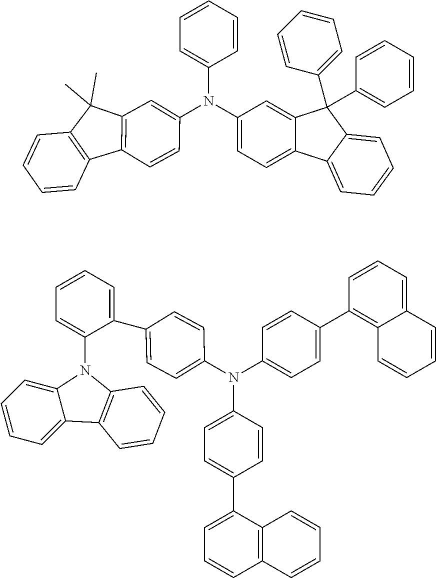 [SMILES: CC1(C)c2ccccc2-c2ccc(N(c3ccccc3)c3ccc4c(c3)C(c3ccccc3)(c3ccccc3)c3ccccc3-4)cc21 ; c1ccc(-n2c3ccccc3c3ccccc32)c(-c2ccc(N(c3ccc(-c4cccc5ccccc45)cc3)c3ccc(-c4cccc5ccccc45)cc3)cc2)c1]

The electron transport layer is a layer that receives electrons from the electron injection layer and transports them to the light-emitting layer. As an electron transport material, a material with high mobility for electrons, which can efficiently receive electrons from the cathode and transport them to the light-emitting layer, is desirable. Specific examples include an Al complex of 8-hydroxyquinoline; a complex including Alq₃; an organic radical compound; a hydroxyflavone-metal complex, etc., although not being limited thereto. The electron transport layer can be used together with any desired cathode material, as used in the prior art. In particular, a suitable cathode material is a conventional material having a low work function and followed by an aluminum layer or a silver layer. Specifically, it may be cesium, barium, calcium, ytterbium, samarium, etc., and is followed by an aluminum layer or a silver layer for each case.

In an exemplary embodiment of the present disclosure, the electron transport layer contains a compound of Chemical Formula ET-1 or ET-2.

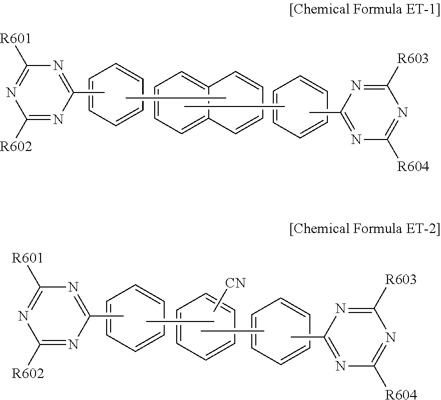

In Chemical Formula ET-1 or Chemical Formula ET-2,
R601 to R604, which are identical or different, are independently hydrogen; deuterium; a substituted or unsubstituted alkyl group; a substituted or unsubstituted aryl group; or a substituted or unsubstituted heterocyclic group.

In an exemplary embodiment of the present specification, R601 to R604, which are identical or different, are independently a substituted or unsubstituted aryl group; or a substituted or unsubstituted heterocyclic group.

In an exemplary embodiment of the present disclosure, R601 to R604, which are identical or different, are independently a substituted or unsubstituted aryl group.

In an exemplary embodiment of the present specification, R601 to R604, which are identical or different, are independently a substituted or unsubstituted C₆₋₃₀ aryl group.

In an exemplary embodiment of the present specification, R601 to R604, which are identical or different, are independently a substituted or unsubstituted C₆₋₂₀ aryl group.

In an exemplary embodiment of the present specification, R601 to R604, which are identical or different, are independently a substituted or unsubstituted C₆₋₁₀ aryl group.

In an exemplary embodiment of the present specification, R601 to R604 are phenyl groups.

In an exemplary embodiment of the present specification, the compound of Chemical Formula ET-1 is represented by the following compound.

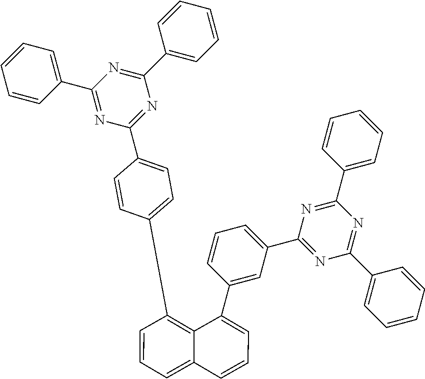 [SMILES: c1ccc(-c2nc(-c3ccccc3)nc(-c3ccc(-c4cccc5cccc(-c6cccc(-c7nc(-c8ccccc8)nc(-c8ccccc8)n7)c6)c45)cc3)n2)cc1]

In an exemplary embodiment of the present specification, the compound of Chemical Formula ET-2 is represented by the following compound.

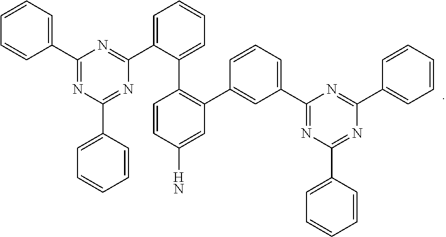 [SMILES: Nc1ccc(-c2ccccc2-c2nc(-c3ccccc3)nc(-c3ccccc3)n2)c(-c2cccc(-c3nc(-c4ccccc4)nc(-c4ccccc4)n3)c2)c1]

In an exemplary embodiment of the present disclosure, the electron transport layer may further contain lithium quinolate (Liq).

The electron injection layer is a layer that receives electrons from the electrode. As an electron injection material, one having excellent electron transport ability, superior effect of receiving electrons from the second electrode, and superior electron injection effect for the light-emitting layer or the light-emitting material is desirable. Additionally, it is desirable that it is a material that prevents excitons generated in the light-emitting layer from moving to the hole injection layer and has excellent thin-film forming ability. Specifically, it may be fluorenone, anthraquinodimethane, diphenoquinone, thiopyran dioxide, oxazole, oxadiazole, triazole, imidazole, perylenetetracarboxylic acid, fluorenylidenemethane, anthrone, etc., derivatives thereof, a metal complex compound, a nitrogen-containing five-membered ring derivative, etc., although not being limited thereto.

The metal complex compound includes 8-hydroxyquinolinatolithium, bis(8-hydroxyquinolinato) zinc, bis(8-hydroxyquinolinato) copper, bis(8-hydroxyquinolinato) manganese, tris(8-hydroxyquinolinato)aluminum, tris(2-methyl-8-hydroxyquinolinato)aluminum, tris(8-hydroxyquinolinato) gallium, bis(10-hydroxybenzo[h]quinolinato) beryllium, bis(10-hydroxybenzo[h]quinolinato) zinc, bis(2-methyl-8-quinolinato) chlorogallium, bis(2-methyl-8-quinolinato) (o-cresolato) gallium, bis(2-methyl-8-quinolinato)(1-naphtholato)aluminum, bis(2-methyl-8-quinolinato)(2-naphtholato) gallium, etc., although not being limited thereto.

In an exemplary embodiment of the present disclosure, the electron injection layer is a layer that transports electrons to the light-emitting layer. The materials exemplified regarding the electron transport layer may be used in the electron injection layer, although not being limited thereto.

In an exemplary embodiment of the present disclosure, the electron injection material may include at least one of magnesium and lithium fluoride (LiF). Specifically, it may include both magnesium and lithium fluoride (LiF).

In an exemplary embodiment of the present specification, the electron injection layer may further contain a metal complex compound. The metal complex compound is the same as described above.

The hole-blocking (electron control) layer is a layer that prevents holes from reaching the cathode, and may generally be formed under the same condition as the electron injection layer. Specifically, an oxadiazole derivative, a triazole derivative, a phenanthroline derivative, an aluminum complex, etc. may be used, although not being limited thereto.

In an exemplary embodiment of the present specification, the hole-blocking layer contains a compound of Chemical Formula EG-1, although not being limited thereto.

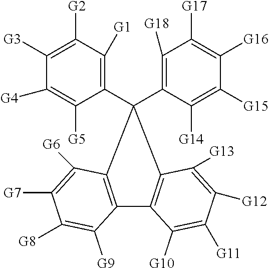

In Chemical Formula EG-1,
at least one of G1 to G18 is -L5-Ar5, the remainder being hydrogen, or G1 and G18 are linked by -L51- to form a substituted or unsubstituted ring,
L5 is a direct bond or a substituted or unsubstituted arylene group,
Ar5 is a substituted or unsubstituted aryl group or a substituted or unsubstituted heteroaryl group, and
L51 is O or S.

In an exemplary embodiment of the present specification, L51 is O.

In an exemplary embodiment of the present specification, L51 is S.

In an exemplary embodiment of the present specification, G1 and G18 are linked by -L51- to form a substituted or unsubstituted heterocycle.

In an exemplary embodiment of the present specification, G1 and G18 are linked by -L51- to form a substituted or unsubstituted xanthene ring, or a substituted or unsubstituted thioxanthene ring.

In an exemplary embodiment of the present specification, G1 and G18 are linked by —O— to form a substituted or unsubstituted xanthene ring.

In an exemplary embodiment of the present specification, G1 and G18 are linked by —S— to form a substituted or unsubstituted thioxanthene ring.

In an exemplary embodiment of the present specification, G1 and G18 are linked by —O— to form a xanthene ring.

In an exemplary embodiment of the present specification, G1 and G18 are linked by —S— to form a thioxanthene ring.

In an exemplary embodiment of the present specification, L5 is a direct bond, or a substituted or unsubstituted C₆₋₃₀ monocyclic or polycyclic arylene group.

In an exemplary embodiment of the present specification, L5 is a direct bond, or a substituted or unsubstituted C₆₋₂₀ monocyclic or polycyclic arylene group.

In an exemplary embodiment of the present specification, L5 is a direct bond, or a C₆₋₃₀ monocyclic or polycyclic arylene group.

In an exemplary embodiment of the present specification, L5 is a direct bond, or a C₆₋₂₀ monocyclic or polycyclic arylene group.

In an exemplary embodiment of the present specification, L5 is a direct bond or a phenylene group.

In an exemplary embodiment of the present specification, Ar5 is a substituted or unsubstituted triazine group.

In an exemplary embodiment of the present specification, Ar5 is a triazine group that is unsubstituted or substituted with a C₆₋₃₀ monocyclic or polycyclic aryl group.

In an exemplary embodiment of the present specification, Ar5 is a triazine group substituted with a phenyl group.

In an exemplary embodiment of the present specification, Chemical Formula EG-1 is represented by the following compound.

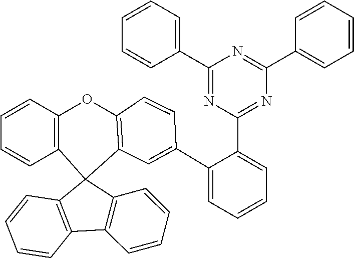 [SMILES: c1ccc(-c2nc(-c3ccccc3)nc(-c3ccccc3-c3ccc4c(c3)C3(c5ccccc5O4)c4ccccc4-c4ccccc43)n2)cc1]

In an exemplary embodiment of the present specification, a capping layer (CPL) may be deposited outside the cathode. The capping layer may serve to maximize the light extraction effect or prevent deterioration of the organic light-emitting device, and may contain a capping layer material known in the art. Specifically, the capping layer material includes a compound of Chemical Formula CP-1, although not being limited thereto.

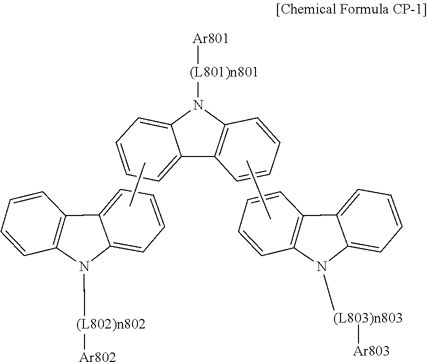

In Chemical Formula CP-1,
L801 to L803, which are identical or different, are independently a direct bond, or a substituted or unsubstituted arylene group,
Ar801 to Ar803, which are identical or different, are independently hydrogen; deuterium; or a substituted or unsubstituted aryl group, and
n801 to n803 are integers 1 or 2, and if n801 to n803 are 2 or greater, two or more L801s to L803s are identical or different.

In an exemplary embodiment of the present specification, L801 to L803, which are identical or different, are independently a direct bond, or a substituted or unsubstituted C₆₋₂₀ arylene group.

In an exemplary embodiment of the present specification, L801 to L803, which are identical or different, are independently a direct bond, a substituted or unsubstituted phenylene group, or a substituted or unsubstituted naphthylene group.

In an exemplary embodiment of the present specification, L801 to L803 are direct bonds.

In an exemplary embodiment of the present specification, Ar801 to Ar803, which are identical or different, are independently hydrogen, deuterium, or a substituted or unsubstituted C₆₋₂₀ aryl group.

In an exemplary embodiment of the present specification, Ar801 to Ar803, which are identical or different, are independently hydrogen, deuterium, a substituted or unsubstituted phenyl group, a substituted or unsubstituted biphenyl group, or a substituted or unsubstituted naphthyl group.

In an exemplary embodiment of the present specification, Ar801 to Ar803, which are identical or different, are independently hydrogen, deuterium, or a substituted or unsubstituted phenyl group.

In an exemplary embodiment of the present specification, Ar801 to Ar803, which are identical or different, are independently hydrogen, deuterium, or a phenyl group.

In an exemplary embodiment of the present specification, the compound of Chemical Formula CP-1 is represented by the following compound.

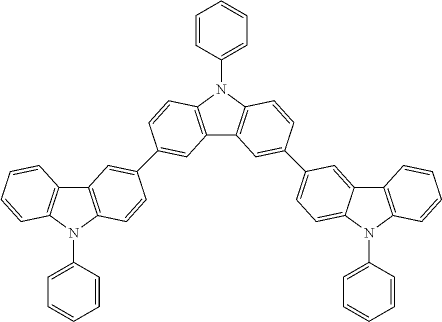 [SMILES: c1ccc(-n2c3ccccc3c3cc(-c4ccc5c(c4)c4cc(-c6ccc7c(c6)c6ccccc6n7-c6ccccc6)ccc4n5-c4ccccc4)ccc32)cc1]

The organic light-emitting device according to the present specification may be a front-emitting, back-emitting or double-sided emitting type depending on the materials used.

The organic light-emitting device according to the present specification may be included and used in various electronic devices. For example, the electronic device may be a display panel, a touch panel, a solar module, a lighting device, etc., although not being limited thereto.

### EXAMPLES

Hereinafter, the present specification will be explained specifically through examples, comparative examples, etc. However, the examples and comparative examples described in the present specification may be modified in various different forms, and the scope of the present specification is not construed as being limited to the examples and comparative examples. The examples and comparative examples in the present specification are provided to more completely explain the present specification to a person having average knowledge in the art.

### <Preparation Example 1> Synthesis of Chemical Formula W

### 1) Synthesis of Chemical Formula M

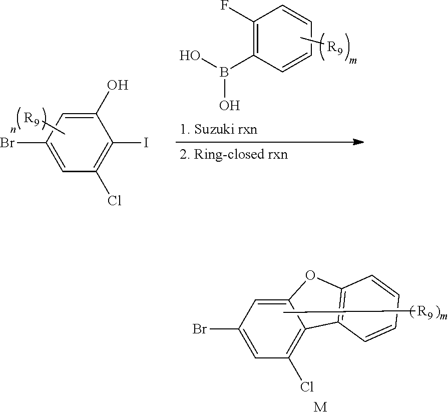

SM1 (1 eq.) and SM2 (1.05 eq.) in Table 1 below were added to tetrahydrofuran (excess). Then, after adding a 2 M potassium carbonate aqueous solution (30 vol. eq. of THE) and tetrakistriphenyl-phosphinopalladium (2 mol %), the mixture was heated for 10 hours under stirring. After cooling to room temperature and completing the reaction, the potassium carbonate aqueous solution was removed through layer separation. After confirming the completion of the reaction, the temperature was lowered to room temperature and the solvent was removed. Then, after adding potassium carbonate (1.5 eq.) and dimethylformamide (excess), the mixture was heated and stirred for 5 hours under reflux. After confirming the completion of the reaction, water (1.5 vol. eq. of dimethylformamide) was added and the precipitated solid was filtered. The solid was extracted with chloroform and water, and then purified by recrystallization using chloroform and ethanol to prepare Chemical Formula M (M1).

In Chemical Formula M, R9, which is identical or different, is independently hydrogen; deuterium; or a substituted or unsubstituted alkyl group, and m is an integer from 1 to 6.

TABLE 1

| | | | | | MS |
|---|---|---|---|---|---|
| | SM1 | SM2 | Product | Yield | [M + H]⁺ |
| M1 | 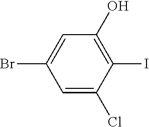 | 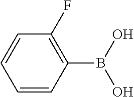 | 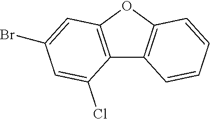 [SMILES: Clc1cc(Br)cc2oc3ccccc3c12] | 53% | 282.53 |

### 2) Synthesis of Chemical Formula T

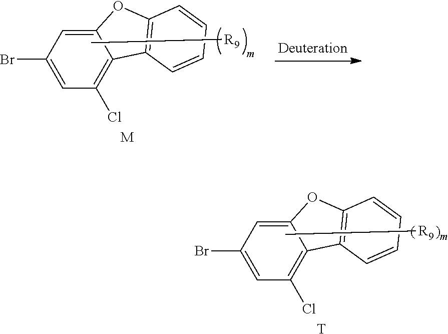

SM1 (1 eq., Chemical Formula M) was added to tetrachloroethane (excess). After adding SM2 (10 eq., in pre-mixed solution (D₂O:Tf₂O=4:1)) dropwise, the mixture was stirred at 140° C. for 1 hour. After cooling to room temperature, the mixture was extracted with a saturated sodium bicarbonate aqueous solution to separate layers, and then the solidified compound was filtered by adding ethanol. The above process was repeated one more time, and Chemical Formula T (T1) was prepared by recrystallizing with ethyl acetate and ethanol.

In Chemical Formula T, R9, which is identical or different, is independently hydrogen; deuterium; or a substituted or unsubstituted alkyl group, and m is an integer from 1 to 6.

TABLE 2

| | | | | | MS |
|---|---|---|---|---|---|
| | SM1 | SM2 | Product | Yield | [M + H]⁺ |
| T1 | M1 | D₂O:Tf₂O = 4:1 | 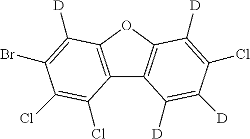 [SMILES: [2H]c1c(Cl)c([2H])c2oc3c([2H])c(Br)c(Cl)c(Cl)c3c2c1[2H]] | 49% | 322.01 |

### 3) Synthesis of Chemical Formula N

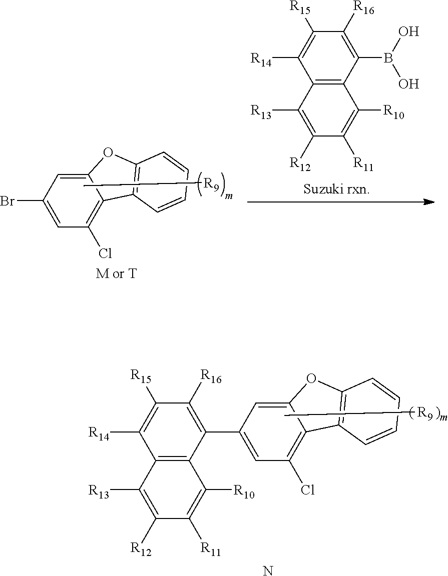

SM1 (1 eq., Chemical Formula M or T) and SM2 (1.1 eq.) were added to tetrahydrofuran (excess). Then, after adding a 2 M potassium carbonate aqueous solution (30 vol. eq. of THF) and tetrakistriphenyl-phosphinopalladium (2 mol %), the mixture was heated at 85° C. for 10 hours under stirring. After lowering the temperature to room temperature and completing the reaction, the potassium carbonate aqueous solution was removed to separate layers. Then, Chemical Formula N (N1 to N13) was prepared by recrystallizing with ethyl acetate and ethanol.

In Chemical Formula N, R9, which is identical or different, is independently hydrogen; deuterium; or a substituted or unsubstituted alkyl group, and m is an integer from 1 to 6.

N1 to N13 described in Table 3 were synthesized in the same manner as in the synthesis of Chemical Formula N, except that SM1 and SM2 were changed.

TABLE 3

| | | | | | MS |
|---|---|---|---|---|---|
| | SM1 | SM2 | Product | Yield | [M + H]⁺ |
| N1 | M1 | 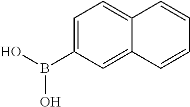 | 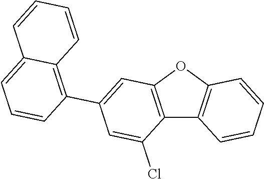 [SMILES: Clc1cc(-c2cccc3ccccc23)cc2oc3ccccc3c12] | 79% | 329.80 |
| N2 | M1 | 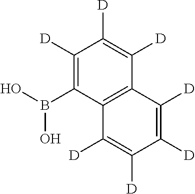 | 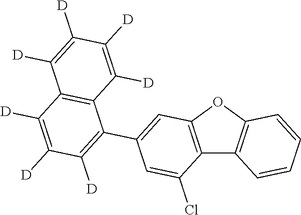 [SMILES: [2H]c1c([2H])c([2H])c2c(-c3cc(Cl)c4c(c3)oc3ccccc34)c([2H])c([2H])c([2H])c2c1[2H]] | 80% | 336.84 |
| N3 | T1 | 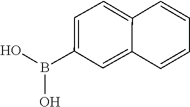 | 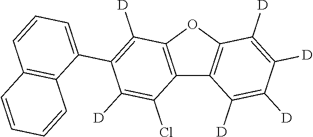 [SMILES: [2H]c1c([2H])c([2H])c2c(oc3c([2H])c(-c4cccc5ccccc45)c([2H])c(Cl)c32)c1[2H]] | 78% | 335.83 |
| N4 | T1 | 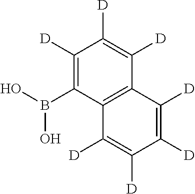 | 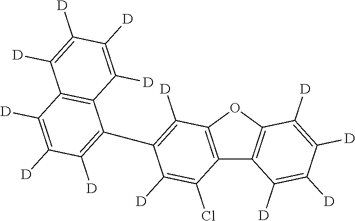 [SMILES: [2H]c1c([2H])c([2H])c2c(oc3c([2H])c(-c4c([2H])c([2H])c([2H])c5c([2H])c([2H])c([2H])c([2H])c45)c([2H])c(Cl)c32)c1[2H]] | 80% | 342.87 |
| N5 | M1 | 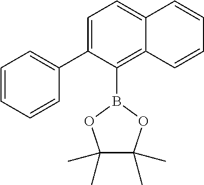 | 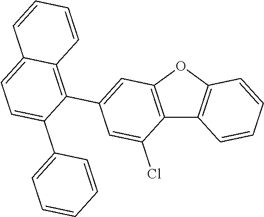 [SMILES: Clc1cc(-c2c(-c3ccccc3)ccc3ccccc23)cc2oc3ccccc3c12] | 75% | 405.89 |
| N6 | M1 | 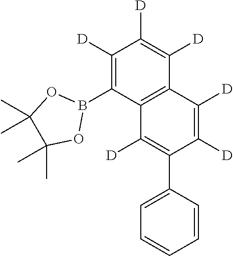 | 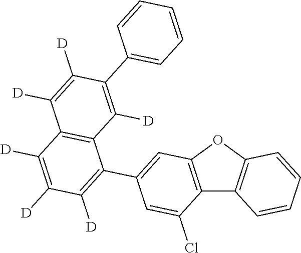 [SMILES: [2H]c1c([2H])c(-c2cc(Cl)c3c(c2)oc2ccccc23)c2c([2H])c(-c3ccccc3)c([2H])c([2H])c2c1[2H]] | 74% | 411.93 |
| N7 | T1 | 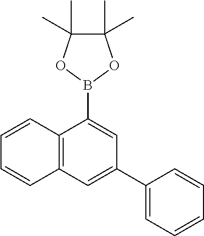 | 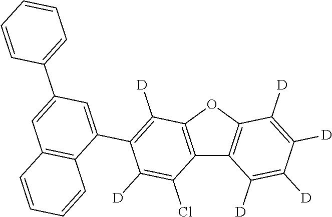 [SMILES: [2H]c1c([2H])c([2H])c2c(oc3c([2H])c(-c4cc(-c5ccccc5)cc5ccccc45)c([2H])c(Cl)c32)c1[2H]] | 70% | 411.93 |
| N8 | T1 | 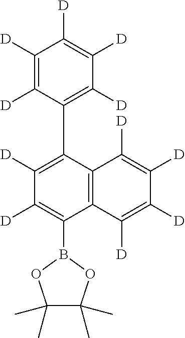 | 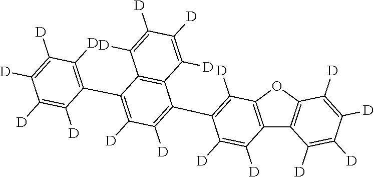 [SMILES: [2H]c1c([2H])c([2H])c(-c2c([2H])c([2H])c(-c3c([2H])c([2H])c4c(oc5c([2H])c([2H])c([2H])c([2H])c54)c3[2H])c3c([2H])c([2H])c([2H])c([2H])c23)c([2H])c1[2H]] | 68% | 423.00 |
| N9 | T1 | 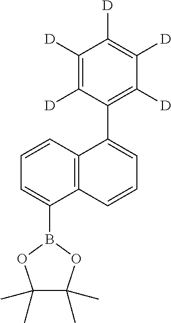 | 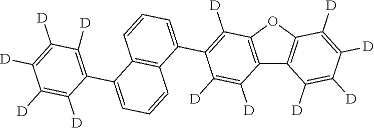 [SMILES: [2H]c1c([2H])c([2H])c(-c2cccc3c(-c4c([2H])c([2H])c5c(oc6c([2H])c([2H])c([2H])c([2H])c65)c4[2H])cccc23)c([2H])c1[2H]] | 72% | 416.96 |
| N10 | M1 | 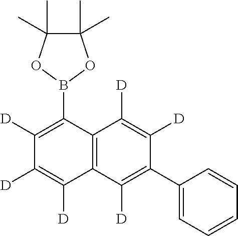 | 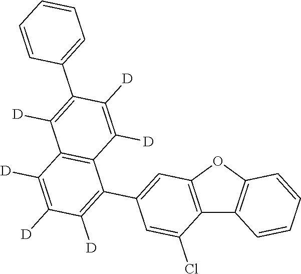 [SMILES: [2H]c1c([2H])c(-c2cc(Cl)c3c(c2)oc2ccccc23)c2c([2H])c([2H])c(-c3ccccc3)c([2H])c2c1[2H]] | 73% | 411.93 |
| N11 | M1 | 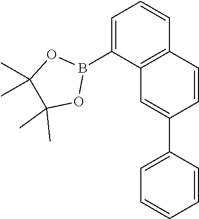 | 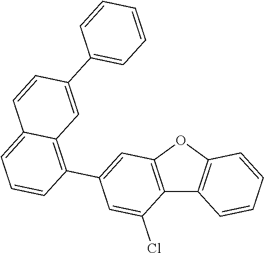 [SMILES: Clc1cc(-c2cccc3ccc(-c4ccccc4)cc23)cc2oc3ccccc3c12] | 70% | 405.89 |
| N12 | M1 | 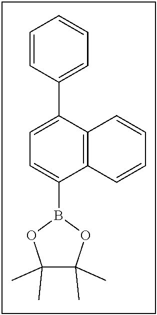 | 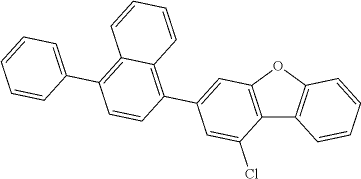 [SMILES: Clc1cc(-c2ccc(-c3ccccc3)c3ccccc23)cc2oc3ccccc3c12] | 76% | 405.89 |
| N13 | M1 | 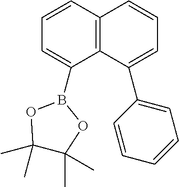 | 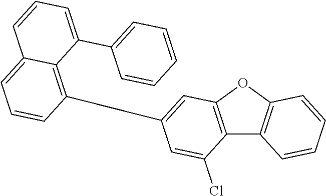 [SMILES: Clc1cc(-c2cccc3cccc(-c4ccccc4)c23)cc2oc3ccccc3c12] | 62% | 405.89 |

### 4) Synthesis of Chemical Formula W

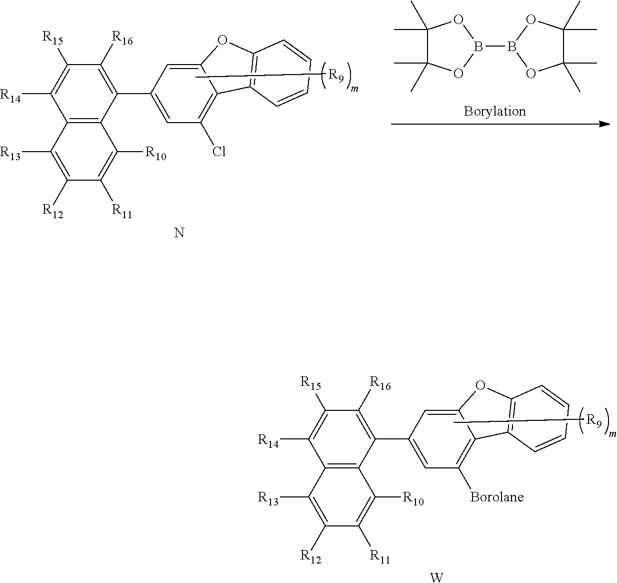

SM1 (1 eq., Chemical Formula N) and SM2 (1.3 eq.) were added to 1,4-dioxane (12 mass eq. of SM1). After adding potassium acetate (3 eq.), the mixture was stirred under reflux. Palladium acetate (0.02 eq.) and tricyclohexylphosphine (0.04 eq.) in 1,4-dioxane were added for 5 minutes under stirring. Two hours later, the completion of the reaction was confirmed and the reaction mixture was cool to room temperature. After adding ethanol and water, the mixture was filtered and purified by recrystallizing with ethyl acetate and ethanol to prepare Chemical Formula W (W1 to W13).

In Chemical Formula W, R9, which is identical or different, is independently hydrogen; deuterium; or a substituted or unsubstituted alkyl group, and m is an integer from 1 to 6.

W1 to W13 described in Table 4 were synthesized in the same manner as in the synthesis of Chemical Formula W, except that SM1 and SM2 were changed.

TABLE 4

| | | | | | MS |
|---|---|---|---|---|---|
| | | | | | [M + |
| | SM1 | SM2 | Product | Yield | H]⁺ |
| W1 | N1 | 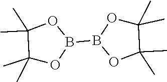 | 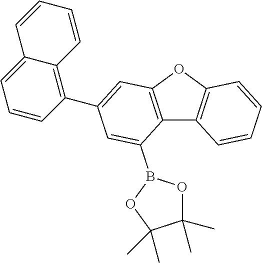 [SMILES: CC1(C)OB(c2cc(-c3cccc4ccccc34)cc3oc4ccccc4c23)OC1(C)C] | 81% | 421.32 |
| W2 | N2 | 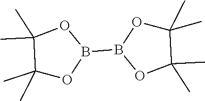 | 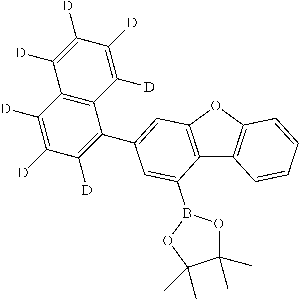 [SMILES: [2H]c1c([2H])c([2H])c2c(-c3cc(B4OC(C)(C)C(C)(C)O4)c4c(c3)oc3ccccc34)c([2H])c([2H])c([2H])c2c1[2H]] | 80% | 428.36 |
| W3 | N3 | 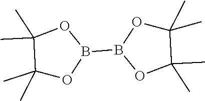 | 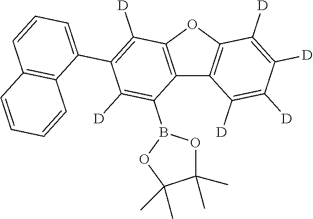 [SMILES: [2H]c1c([2H])c([2H])c2c(oc3c([2H])c(-c4cccc5ccccc45)c([2H])c(B4OC(C)(C)C(C)(C)O4)c32)c1[2H]] | 83% | 427.35 |
| W4 | N4 | 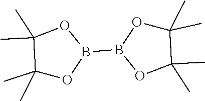 | 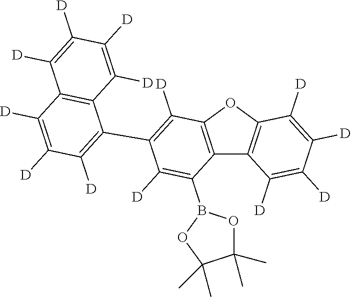 [SMILES: [2H]c1c([2H])c([2H])c2c(oc3c([2H])c(-c4c([2H])c([2H])c([2H])c5c([2H])c([2H])c([2H])c([2H])c45)c([2H])c(B4OC(C)(C)C(C)(C)O4)c32)c1[2H]] | 81% | 434.39 |
| W5 | N5 | 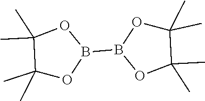 | 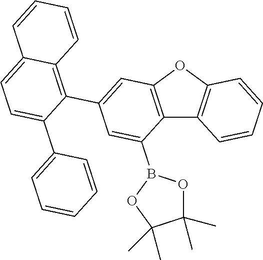 [SMILES: CC1(C)OB(c2cc(-c3c(-c4ccccc4)ccc4ccccc34)cc3oc4ccccc4c23)OC1(C)C] | 79% | 497.41 |
| W6 | N6 | 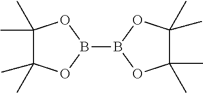 | 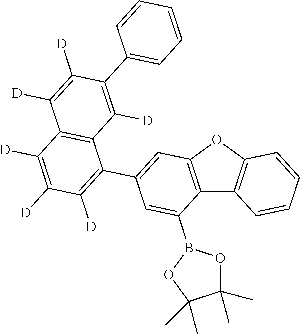 [SMILES: [2H]c1c([2H])c(-c2cc(B3OC(C)(C)C(C)(C)O3)c3c(c2)oc2ccccc23)c2c([2H])c(-c3ccccc3)c([2H])c([2H])c2c1[2H]] | 77% | 503.45 |
| W7 | N7 | 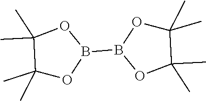 | 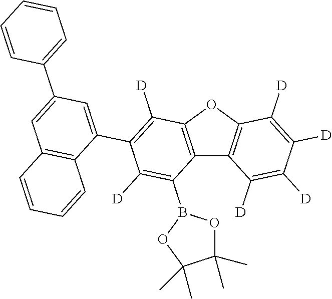 [SMILES: [2H]c1c([2H])c([2H])c2c(oc3c([2H])c(-c4cc(-c5ccccc5)cc5ccccc45)c([2H])c(B4OC(C)(C)C(C)(C)O4)c32)c1[2H]] | 80% | 504.45 |
| W8 | N8 | 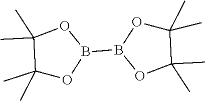 | 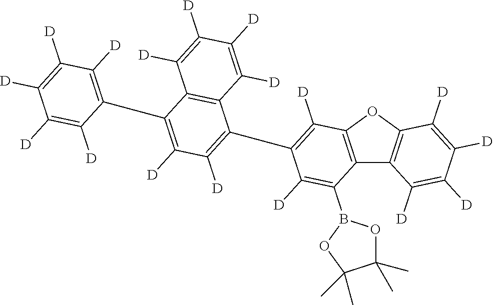 [SMILES: [2H]c1c([2H])c([2H])c(-c2c([2H])c([2H])c(-c3c([2H])c(B4OC(C)(C)C(C)(C)O4)c4c(oc5c([2H])c([2H])c([2H])c([2H])c54)c3[2H])c3c([2H])c([2H])c([2H])c([2H])c23)c([2H])c1[2H]] | 71% | 514.52 |
| W9 | N9 | 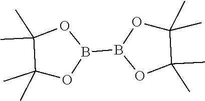 | 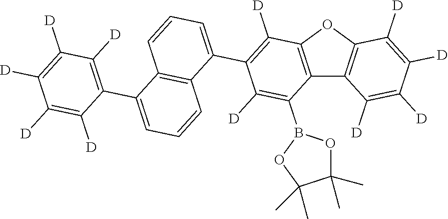 [SMILES: [2H]c1c([2H])c([2H])c(-c2cccc3c(-c4c([2H])c(B5OC(C)(C)C(C)(C)O5)c5c(oc6c([2H])c([2H])c([2H])c([2H])c65)c4[2H])cccc23)c([2H])c1[2H]] | 74% | 508.48 |
| W10 | N10 | 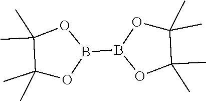 | 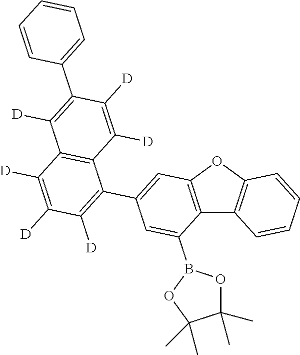 [SMILES: [2H]c1c([2H])c(-c2cc(B3OC(C)(C)C(C)(C)O3)c3c(c2)oc2ccccc23)c2c([2H])c([2H])c(-c3ccccc3)c([2H])c2c1[2H]] | 75% | 503.45 |
| W11 | N11 | 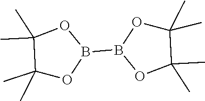 | 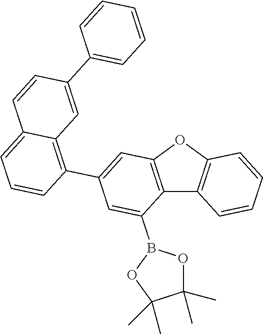 [SMILES: CC1(C)OB(c2cc(-c3cccc4ccc(-c5ccccc5)cc34)cc3oc4ccccc4c23)OC1(C)C] | 73% | 497.41 |
| W12 | N12 | 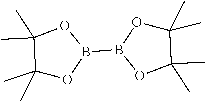 | 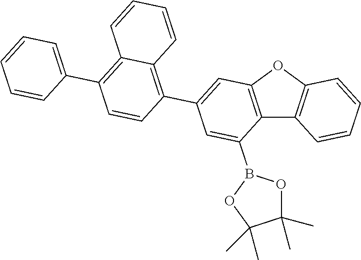 [SMILES: CC1(C)OB(c2cc(-c3ccc(-c4ccccc4)c4ccccc34)cc3oc4ccccc4c23)OC1(C)C] | 76% | 497.41 |
| W13 | N13 | 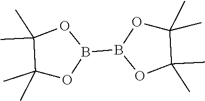 | 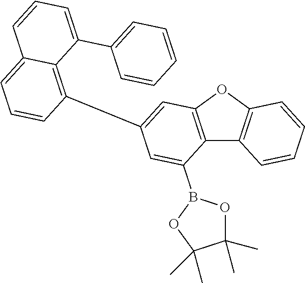 [SMILES: CC1(C)OB(c2cc(-c3cccc4cccc(-c5ccccc5)c34)cc3oc4ccccc4c23)OC1(C)C] | 63% | 497.41 |

### <Preparation Example 2> Synthesis of Chemical Formula P1

### 1) Synthesis of Chemical Formula A

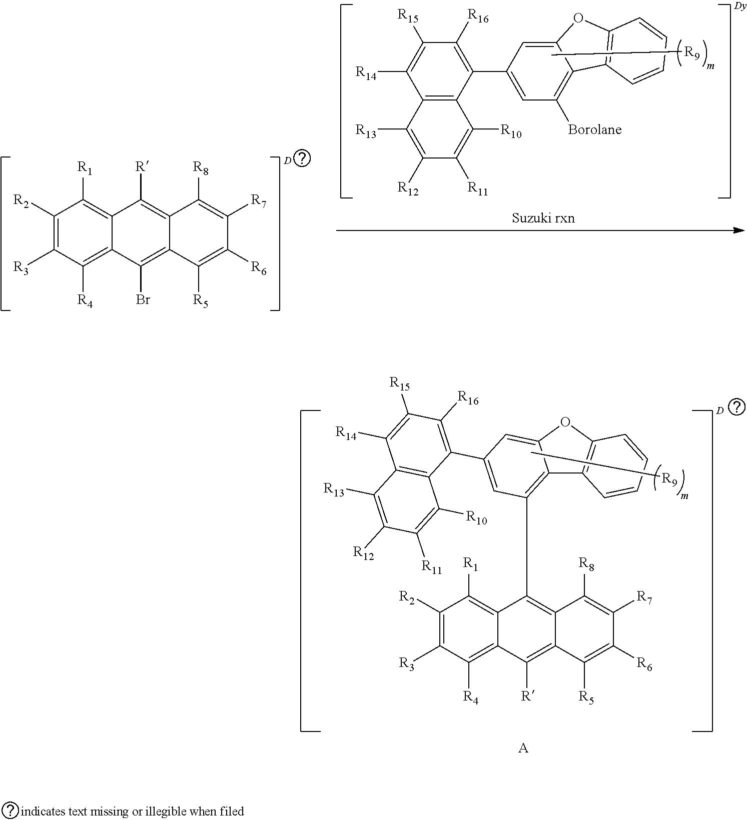

SM1 (1 eq.) and SM2 (1.1 eq.) were added to tetrahydrofuran (excess). Then, after adding a 2 M potassium carbonate aqueous solution (30 vol. eq. of THE) and tetrakistriphenyl-phosphinopalladium (2 mol %), the mixture was heated at 85° C. for 10 hours under stirring. After lowering the temperature to room temperature and completing the reaction, the potassium carbonate aqueous solution was removed. After separation of layers, Chemical Formula A (A1 to A19) was prepared by recrystallizing with chloroform and ethyl acetate.

In Chemical Formula A, R9, which is identical or different, is independently hydrogen; deuterium; or a substituted or unsubstituted alkyl group, m is an integer from 1 to 6, and R′ is deuterium; a substituted or unsubstituted aryl group; or a substituted or unsubstituted heteroaryl group.

A1 to A19 described in Table 5 were synthesized in the same manner as in the synthesis of Chemical Formula A, except that SM1 and SM2 were changed.

TABLE 5

| | | | | | MS |
|---|---|---|---|---|---|
| | | | | | [M + |
| | SM1 | SM2 | Product | Yield | H]+ |
| A1 | W1 | 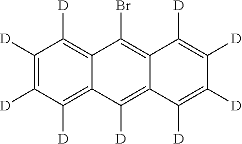 [SMILES: [2H]c1c([2H])c([2H])c2c(Br)c3c([2H])c([2H])c([2H])c([2H])c3c([2H])c2c1[2H]] | 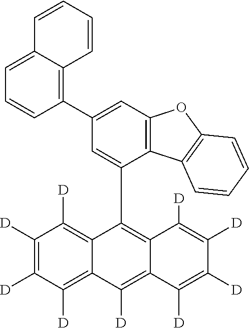 [SMILES: [2H]c1c([2H])c([2H])c2c(-c3cc(-c4cccc5ccccc45)cc4oc5ccccc5c34)c3c([2H])c([2H])c([2H])c([2H])c3c([2H])c2c1[2H]] | 80% | 480.63 |
| A2 | W2 | 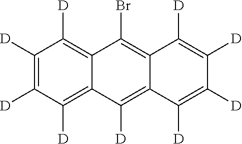 [SMILES: [2H]c1c([2H])c([2H])c2c(Br)c3c([2H])c([2H])c([2H])c([2H])c3c([2H])c2c1[2H]] | 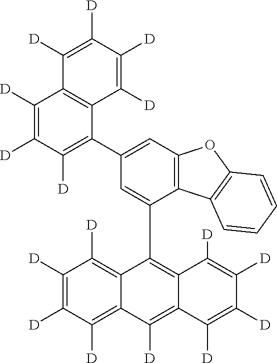 [SMILES: [2H]c1c([2H])c([2H])c2c(-c3cc(-c4c5c([2H])c([2H])c([2H])c([2H])c5c([2H])c5c([2H])c([2H])c([2H])c([2H])c45)c4c(c3)oc3ccccc34)c([2H])c([2H])c([2H])c2c1[2H]] | 81% | 487.67 |
| A3 | W3 | 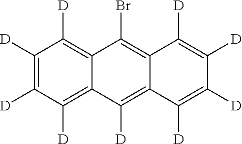 [SMILES: [2H]c1c([2H])c([2H])c2c(Br)c3c([2H])c([2H])c([2H])c([2H])c3c([2H])c2c1[2H]] | 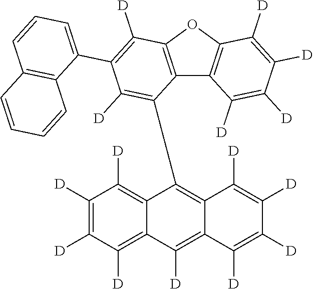 [SMILES: [2H]c1c([2H])c([2H])c2c(oc3c([2H])c(-c4cccc5ccccc45)c([2H])c(-c4c5c([2H])c([2H])c([2H])c([2H])c5c([2H])c5c([2H])c([2H])c([2H])c([2H])c45)c32)c1[2H]] | 83% | 486.66 |
| A4 | W4 | 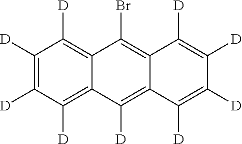 [SMILES: [2H]c1c([2H])c([2H])c2c(Br)c3c([2H])c([2H])c([2H])c([2H])c3c([2H])c2c1[2H]] | 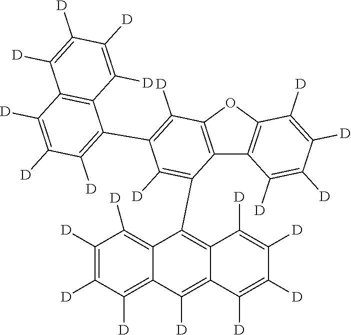 [SMILES: [2H]c1c([2H])c([2H])c2c(oc3c([2H])c(-c4c([2H])c([2H])c([2H])c5c([2H])c([2H])c([2H])c([2H])c45)c([2H])c(-c4c5c([2H])c([2H])c([2H])c([2H])c5c([2H])c5c([2H])c([2H])c([2H])c([2H])c45)c32)c1[2H]] | 80% | 493.71 |
| A5 | W5 | 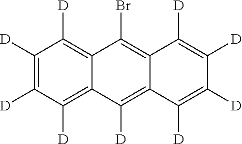 [SMILES: [2H]c1c([2H])c([2H])c2c(Br)c3c([2H])c([2H])c([2H])c([2H])c3c([2H])c2c1[2H]] | 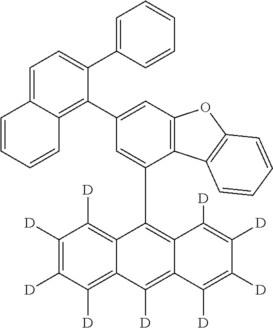 [SMILES: [2H]c1c([2H])c([2H])c2c(-c3cc(-c4c(-c5ccccc5)ccc5ccccc45)cc4oc5ccccc5c34)c3c([2H])c([2H])c([2H])c([2H])c3c([2H])c2c1[2H]] | 79% | 556.72 |
| A6 | W6 | 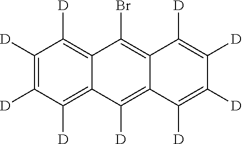 [SMILES: [2H]c1c([2H])c([2H])c2c(Br)c3c([2H])c([2H])c([2H])c([2H])c3c([2H])c2c1[2H]] | 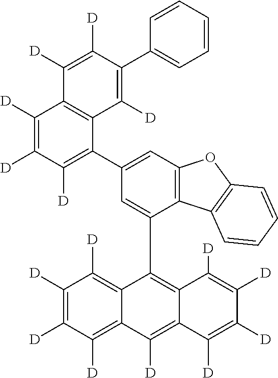 [SMILES: [2H]c1c([2H])c(-c2cc(-c3c4c([2H])c([2H])c([2H])c([2H])c4c([2H])c4c([2H])c([2H])c([2H])c([2H])c34)c3c(c2)oc2ccccc23)c2c([2H])c(-c3ccccc3)c([2H])c([2H])c2c1[2H]] | 77% | 562.76 |
| A7 | W7 | 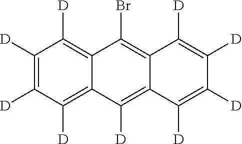 [SMILES: [2H]c1c([2H])c([2H])c2c(Br)c3c([2H])c([2H])c([2H])c([2H])c3c([2H])c2c1[2H]] | 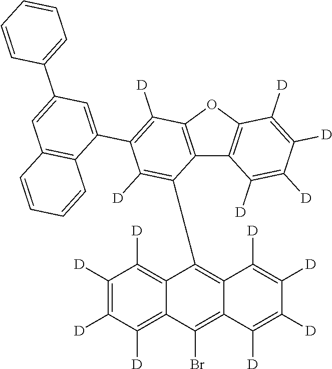 [SMILES: [2H]c1c([2H])c([2H])c2c(oc3c([2H])c(-c4cc(-c5ccccc5)cc5ccccc45)c([2H])c(-c4c5c([2H])c([2H])c([2H])c([2H])c5c(Br)c5c([2H])c([2H])c([2H])c([2H])c45)c32)c1[2H]] | 74% | 562.76 |
| A8 | W8 | 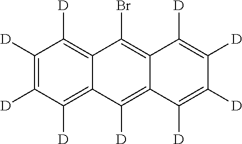 [SMILES: [2H]c1c([2H])c([2H])c2c(Br)c3c([2H])c([2H])c([2H])c([2H])c3c([2H])c2c1[2H]] | 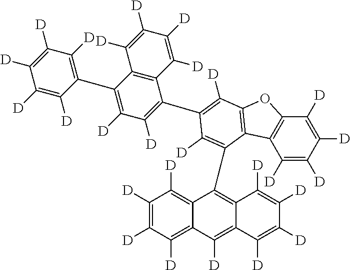 [SMILES: [2H]c1c([2H])c([2H])c(-c2c([2H])c([2H])c(-c3c([2H])c(-c4c5c([2H])c([2H])c([2H])c([2H])c5c([2H])c5c([2H])c([2H])c([2H])c([2H])c45)c4c(oc5c([2H])c([2H])c([2H])c([2H])c54)c3[2H])c3c([2H])c([2H])c([2H])c([2H])c23)c([2H])c1[2H]] | 79% | 573.83 |
| A9 | W9 | 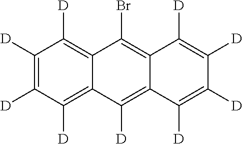 [SMILES: [2H]c1c([2H])c([2H])c2c(Br)c3c([2H])c([2H])c([2H])c([2H])c3c([2H])c2c1[2H]] | 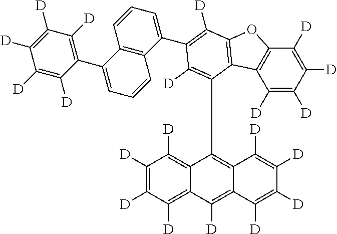 [SMILES: [2H]c1c([2H])c([2H])c(-c2cccc3c(-c4c([2H])c(-c5c6c([2H])c([2H])c([2H])c([2H])c6c([2H])c6c([2H])c([2H])c([2H])c([2H])c56)c5c(oc6c([2H])c([2H])c([2H])c([2H])c65)c4[2H])cccc23)c([2H])c1[2H]] | 80% | 567.79 |
| A10 | W10 | 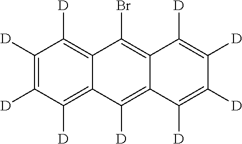 [SMILES: [2H]c1c([2H])c([2H])c2c(Br)c3c([2H])c([2H])c([2H])c([2H])c3c([2H])c2c1[2H]] | 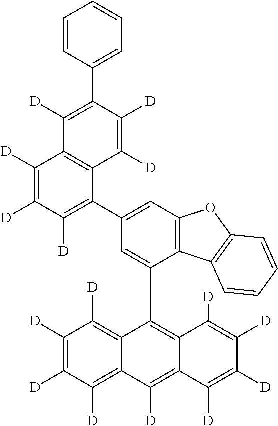 [SMILES: [2H]c1c([2H])c(-c2cc(-c3c4c([2H])c([2H])c([2H])c([2H])c4c([2H])c4c([2H])c([2H])c([2H])c([2H])c34)c3c(c2)oc2ccccc23)c2c([2H])c([2H])c(-c3ccccc3)c([2H])c2c1[2H]] | 74% | 562.76 |
| A11 | W11 | 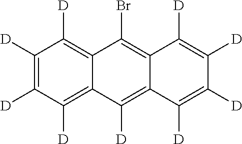 [SMILES: [2H]c1c([2H])c([2H])c2c(Br)c3c([2H])c([2H])c([2H])c([2H])c3c([2H])c2c1[2H]] | 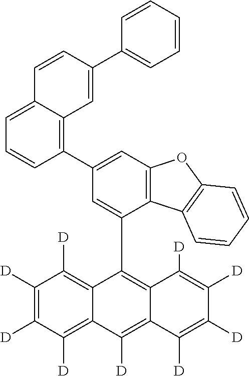 [SMILES: [2H]c1c([2H])c([2H])c2c(-c3cc(-c4cccc5ccc(-c6ccccc6)cc45)cc4oc5ccccc5c34)c3c([2H])c([2H])c([2H])c([2H])c3c([2H])c2c1[2H]] | 73% | 556.72 |
| A12 | W12 | 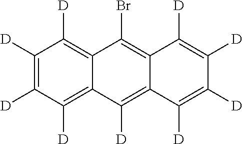 [SMILES: [2H]c1c([2H])c([2H])c2c(Br)c3c([2H])c([2H])c([2H])c([2H])c3c([2H])c2c1[2H]] | 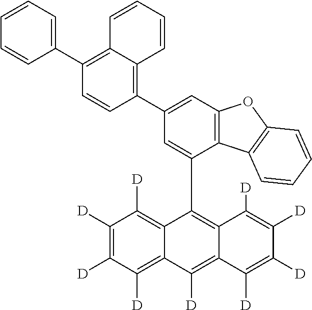 [SMILES: [2H]c1c([2H])c([2H])c2c(-c3cc(-c4ccc(-c5ccccc5)c5ccccc45)cc4oc5ccccc5c34)c3c([2H])c([2H])c([2H])c([2H])c3c([2H])c2c1[2H]] | 70% | 556.72 |
| A13 | W13 | 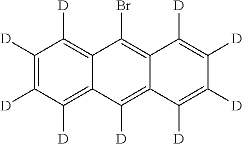 [SMILES: [2H]c1c([2H])c([2H])c2c(Br)c3c([2H])c([2H])c([2H])c([2H])c3c([2H])c2c1[2H]] | 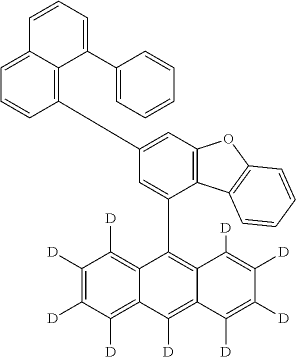 [SMILES: [2H]c1c([2H])c([2H])c2c(-c3cc(-c4cccc5cccc(-c6ccccc6)c45)cc4oc5ccccc5c34)c3c([2H])c([2H])c([2H])c([2H])c3c([2H])c2c1[2H]] | 61% | 556.72 |
| A14 | W1 | 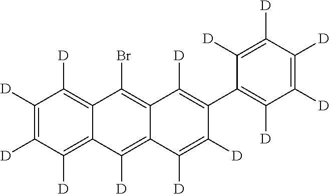 [SMILES: [2H]c1c([2H])c([2H])c(-c2c([2H])c([2H])c3c([2H])c4c([2H])c([2H])c([2H])c([2H])c4c(Br)c3c2[2H])c([2H])c1[2H]] | 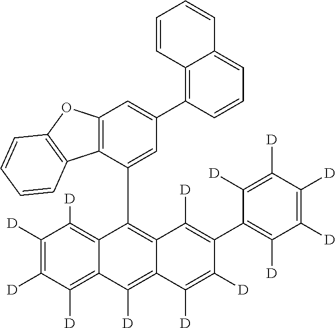 [SMILES: [2H]c1c([2H])c([2H])c(-c2c([2H])c([2H])c3c([2H])c4c([2H])c([2H])c([2H])c([2H])c4c(-c4cc(-c5cccc6ccccc56)cc5oc6ccccc6c45)c3c2[2H])c([2H])c1[2H]] | 75% | 560.75 |
| A15 | W2 | 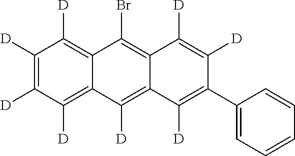 [SMILES: [2H]c1c([2H])c([2H])c2c(Br)c3c([2H])c([2H])c(-c4ccccc4)c([2H])c3c([2H])c2c1[2H]] | 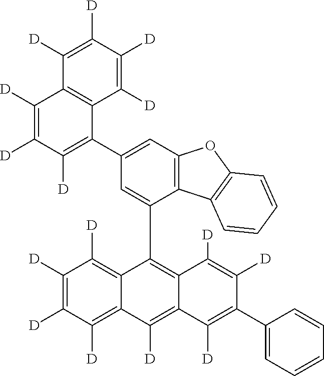 [SMILES: [2H]c1c([2H])c([2H])c2c(-c3cc(-c4c5c([2H])c([2H])c([2H])c([2H])c5c([2H])c5c([2H])c(-c6ccccc6)c([2H])c([2H])c45)c4c(c3)oc3ccccc34)c([2H])c([2H])c([2H])c2c1[2H]] | 70% | 562.76 |
| A16 | W3 | 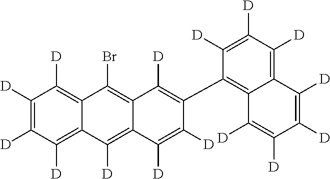 [SMILES: [2H]c1c([2H])c([2H])c2c(-c3c([2H])c([2H])c4c([2H])c5c([2H])c([2H])c([2H])c([2H])c5c(Br)c4c3[2H])c([2H])c([2H])c([2H])c2c1[2H]] | 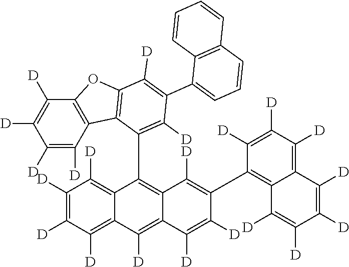 [SMILES: [2H]c1c([2H])c([2H])c2c(oc3c([2H])c(-c4cccc5ccccc45)c([2H])c(-c4c5c([2H])c([2H])c([2H])c([2H])c5c([2H])c5c([2H])c([2H])c(-c6c([2H])c([2H])c([2H])c7c([2H])c([2H])c([2H])c([2H])c67)c([2H])c45)c32)c1[2H]] | 74% | 618.86 |
| A17 | W4 | 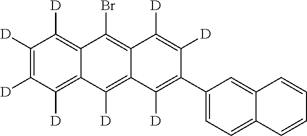 [SMILES: [2H]c1c([2H])c([2H])c2c(Br)c3c([2H])c([2H])c(-c4ccc5ccccc5c4)c([2H])c3c([2H])c2c1[2H]] | 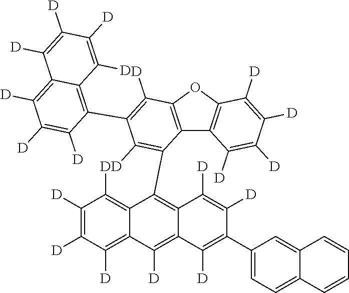 [SMILES: [2H]c1c([2H])c([2H])c2c(oc3c([2H])c(-c4c([2H])c([2H])c([2H])c5c([2H])c([2H])c([2H])c([2H])c45)c([2H])c(-c4c5c([2H])c([2H])c([2H])c([2H])c5c([2H])c5c([2H])c(-c6ccc7ccccc7c6)c([2H])c([2H])c45)c32)c1[2H]] | 73% | 618.86 |
| A18 | W1 | 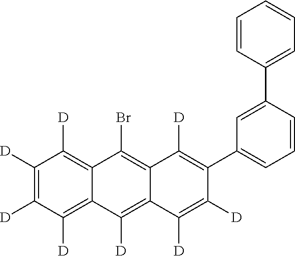 [SMILES: [2H]c1c([2H])c([2H])c2c(Br)c3c([2H])c(-c4cccc(-c5ccccc5)c4)c([2H])c([2H])c3c([2H])c2c1[2H]] | 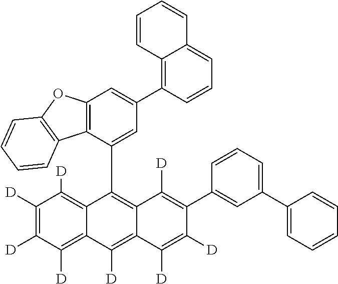 [SMILES: [2H]c1c([2H])c([2H])c2c(-c3cc(-c4cccc5ccccc45)cc4oc5ccccc5c34)c3c([2H])c(-c4cccc(-c5ccccc5)c4)c([2H])c([2H])c3c([2H])c2c1[2H]] | 70% | 631.82 |
| A19 | W1 | 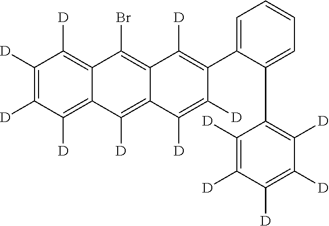 [SMILES: [2H]c1c([2H])c([2H])c(-c2ccccc2-c2c([2H])c([2H])c3c([2H])c4c([2H])c([2H])c([2H])c([2H])c4c(Br)c3c2[2H])c([2H])c1[2H]] | 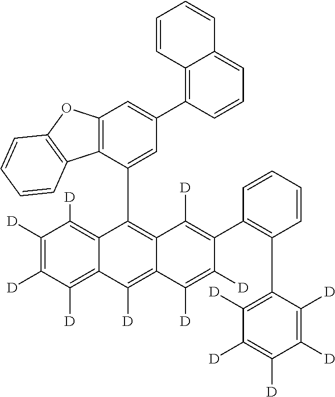 [SMILES: [2H]c1c([2H])c([2H])c(-c2ccccc2-c2c([2H])c([2H])c3c([2H])c4c([2H])c([2H])c([2H])c([2H])c4c(-c4cc(-c5cccc6ccccc56)cc5oc6ccccc6c45)c3c2[2H])c([2H])c1[2H]] | 69% | 636.85 |

### 2) Synthesis of Chemical Formula B

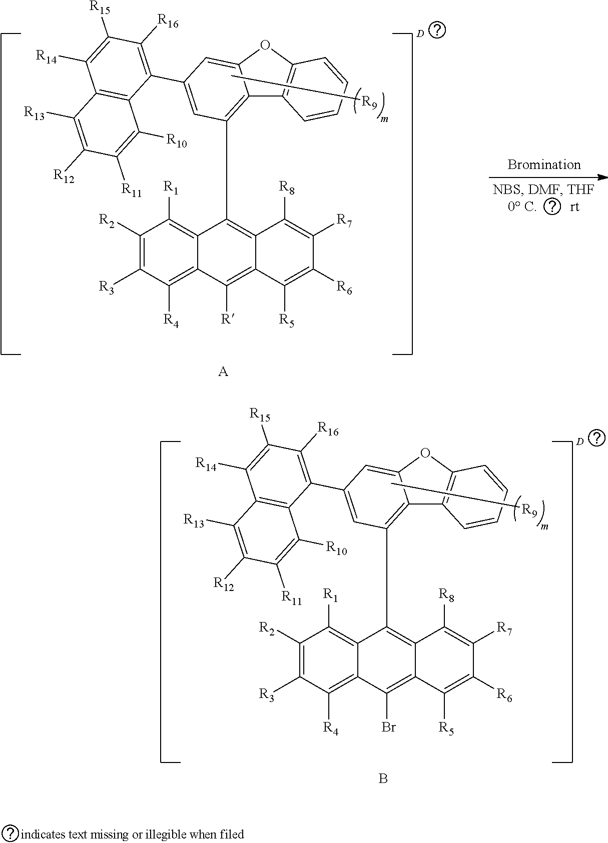

SM1 (Chemical Formula A, 1 eq.) was dissolved in tetrahydrofuran (excess). After lowering the temperature to 0° C. and stabilizing the temperature, N-bromosuccinimide (1 eq.) dissolved in dimethylformamide (3.5 eq. of NBS) was added dropwise. Afterwards, the reaction mixture was heated to room temperature, and stirred for 1 hour. Then, the reaction was completed by adding 1 N HCl (excess). After the reaction was completed, followed by the removal of the solvent and layer separation, Chemical Formula B (B1 to B19) was prepared by recrystallizing with chloroform and ethyl acetate.

In Chemical Formula B, R9, which is identical or different, is independently hydrogen; deuterium; or a substituted or unsubstituted alkyl group, and m is an integer from 1 to 6.

B1 to B19 described in Table 6 were synthesized in the same manner as in the synthesis of Chemical Formula B, except that SM1 and SM2 were changed.

TABLE 6

| | | | | | MS |
|---|---|---|---|---|---|
| | SM1 | SM2 | Product | Yield | [M + H]+ |
| B1 | A1 | NBS, DMF, THF | 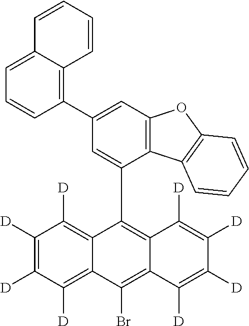 [SMILES: [2H]c1c([2H])c([2H])c2c(-c3cc(-c4cccc5ccccc45)cc4oc5ccccc5c34)c3c([2H])c([2H])c([2H])c([2H])c3c(Br)c2c1[2H]] | 79% | 558.52 |
| B2 | A2 | NBS, DMF, THF | 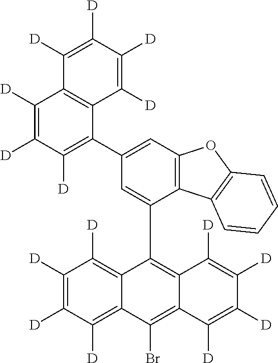 [SMILES: [2H]c1c([2H])c([2H])c2c(-c3cc(-c4c5c([2H])c([2H])c([2H])c([2H])c5c(Br)c5c([2H])c([2H])c([2H])c([2H])c45)c4c(c3)oc3ccccc34)c([2H])c([2H])c([2H])c2c1[2H]] | 80% | 565.56 |
| B3 | A3 | NBS, DMF, THF | 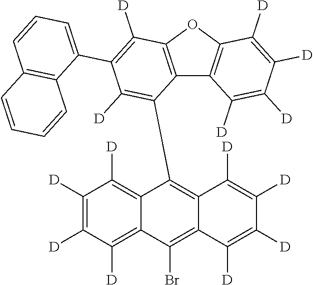 [SMILES: [2H]c1c([2H])c([2H])c2c(oc3c([2H])c(-c4cccc5ccccc45)c([2H])c(-c4c5c([2H])c([2H])c([2H])c([2H])c5c(Br)c5c([2H])c([2H])c([2H])c([2H])c45)c32)c1[2H]] | 79% | 564.55 |
| B4 | A4 | NBS, DMF, THF | 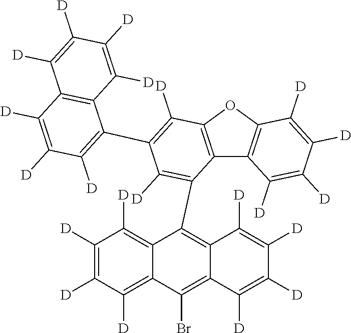 [SMILES: [2H]c1c([2H])c([2H])c2c(oc3c([2H])c(-c4c([2H])c([2H])c([2H])c5c([2H])c([2H])c([2H])c([2H])c45)c([2H])c(-c4c5c([2H])c([2H])c([2H])c([2H])c5c(Br)c5c([2H])c([2H])c([2H])c([2H])c45)c32)c1[2H]] | 81% | 571.60 |
| B5 | A5 | NBS, DMF, THF | 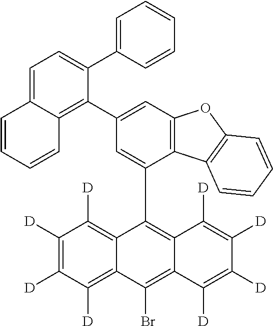 [SMILES: [2H]c1c([2H])c([2H])c2c(-c3cc(-c4c(-c5ccccc5)ccc5ccccc45)cc4oc5ccccc5c34)c3c([2H])c([2H])c([2H])c([2H])c3c(Br)c2c1[2H]] | 77% | 634.61 |
| B6 | A6 | NBS, DMF, THF | 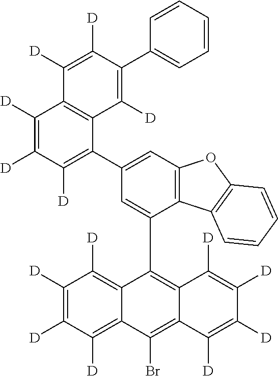 [SMILES: [2H]c1c([2H])c(-c2cc(-c3c4c([2H])c([2H])c([2H])c([2H])c4c(Br)c4c([2H])c([2H])c([2H])c([2H])c34)c3c(c2)oc2ccccc23)c2c([2H])c(-c3ccccc3)c([2H])c([2H])c2c1[2H]] | 78% | 640.65 |
| B7 | A7 | NBS, DMF, THF | 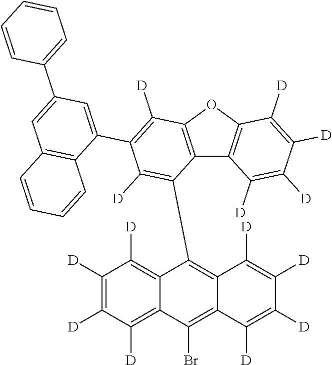 [SMILES: [2H]c1c([2H])c([2H])c2c(oc3c([2H])c(-c4cc(-c5ccccc5)cc5ccccc45)c([2H])c(-c4c5c([2H])c([2H])c([2H])c([2H])c5c(Br)c5c([2H])c([2H])c([2H])c([2H])c45)c32)c1[2H]] | 80% | 640.65 |
| B8 | A8 | NBS, DMF, THF | 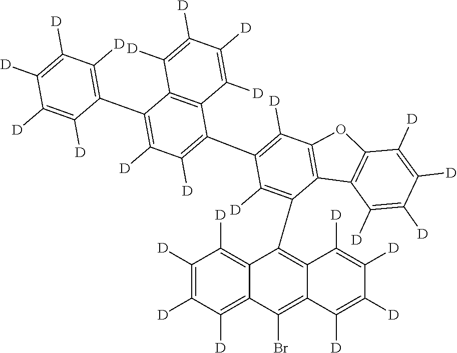 [SMILES: [2H]c1c([2H])c([2H])c(-c2c([2H])c([2H])c(-c3c([2H])c(-c4c5c([2H])c([2H])c([2H])c([2H])c5c(Br)c5c([2H])c([2H])c([2H])c([2H])c45)c4c(oc5c([2H])c([2H])c([2H])c([2H])c54)c3[2H])c3c([2H])c([2H])c([2H])c([2H])c23)c([2H])c1[2H]] | 77% | 651.72 |
| B9 | A9 | NBS, DMF, THF | 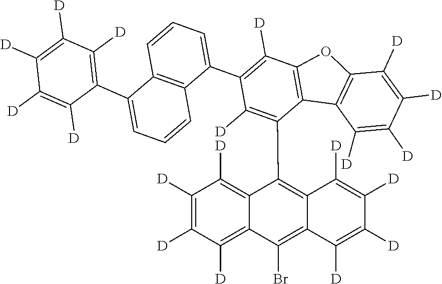 [SMILES: [2H]c1c([2H])c([2H])c(-c2cccc3c(-c4c([2H])c(-c5c6c([2H])c([2H])c([2H])c([2H])c6c(Br)c6c([2H])c([2H])c([2H])c([2H])c56)c5c(oc6c([2H])c([2H])c([2H])c([2H])c65)c4[2H])cccc23)c([2H])c1[2H]] | 79% | 645.68 |
| B10 | A10 | NBS, DMF, THF | 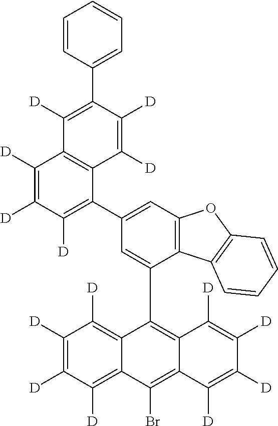 [SMILES: [2H]c1c([2H])c(-c2cc(-c3c4c([2H])c([2H])c([2H])c([2H])c4c(Br)c4c([2H])c([2H])c([2H])c([2H])c34)c3c(c2)oc2ccccc23)c2c([2H])c([2H])c(-c3ccccc3)c([2H])c2c1[2H]] | 81% | 640.65 |
| B11 | A11 | NBS, DMF, THF | 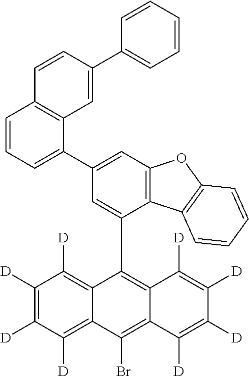 [SMILES: [2H]c1c([2H])c([2H])c2c(-c3cc(-c4cccc5ccc(-c6ccccc6)cc45)cc4oc5ccccc5c34)c3c([2H])c([2H])c([2H])c([2H])c3c(Br)c2c1[2H]] | 74% | 634.61 |
| B12 | A12 | NBS, DMF, THF | 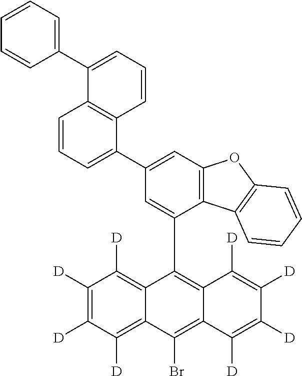 [SMILES: [2H]c1c([2H])c([2H])c2c(-c3cc(-c4cccc5c(-c6ccccc6)cccc45)cc4oc5ccccc5c34)c3c([2H])c([2H])c([2H])c([2H])c3c(Br)c2c1[2H]] | 75% | 634.61 |
| B13 | A13 | NBS, DMF, THF | 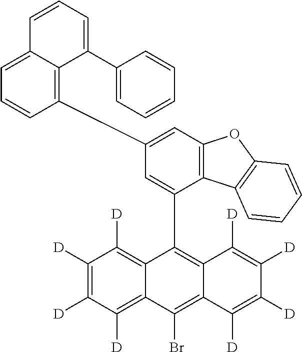 [SMILES: [2H]c1c([2H])c([2H])c2c(-c3cc(-c4cccc5cccc(-c6ccccc6)c45)cc4oc5ccccc5c34)c3c([2H])c([2H])c([2H])c([2H])c3c(Br)c2c1[2H]] | 64% | 634.61 |
| B14 | A14 | NBS, DMF, THF | 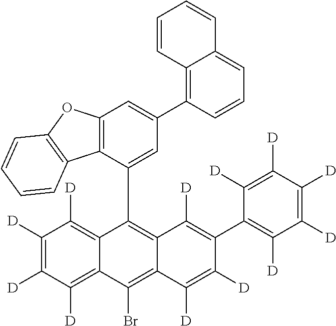 [SMILES: [2H]c1c([2H])c([2H])c(-c2c([2H])c([2H])c3c(Br)c4c([2H])c([2H])c([2H])c([2H])c4c(-c4cc(-c5cccc6ccccc56)cc5oc6ccccc6c45)c3c2[2H])c([2H])c1[2H]] | 70% | 635.84 |
| B15 | A15 | NBS, DMF, THF | 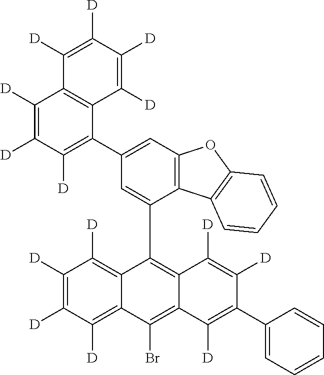 [SMILES: [2H]c1c([2H])c([2H])c2c(-c3cc(-c4c5c([2H])c([2H])c([2H])c([2H])c5c(Br)c5c([2H])c(-c6ccccc6)c([2H])c([2H])c45)c4c(c3)oc3ccccc34)c([2H])c([2H])c([2H])c2c1[2H]] | 71% | 642.88 |
| B16 | A16 | NBS, DMF, THF | 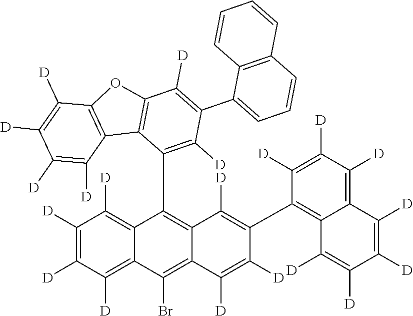 [SMILES: [2H]c1c([2H])c([2H])c2c(oc3c([2H])c(-c4cccc5ccccc45)c([2H])c(-c4c5c([2H])c([2H])c([2H])c([2H])c5c(Br)c5c([2H])c([2H])c(-c6c([2H])c([2H])c([2H])c7c([2H])c([2H])c([2H])c([2H])c67)c([2H])c45)c32)c1[2H]] | 70% | 693.95 |
| B17 | A17 | NBS, DMF, THF | 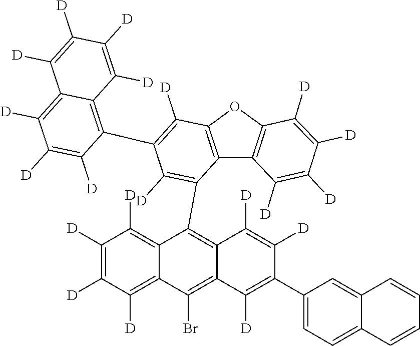 [SMILES: [2H]c1c([2H])c([2H])c2c(oc3c([2H])c(-c4c([2H])c([2H])c([2H])c5c([2H])c([2H])c([2H])c([2H])c45)c([2H])c(-c4c5c([2H])c([2H])c([2H])c([2H])c5c(Br)c5c([2H])c(-c6ccc7ccccc7c6)c([2H])c([2H])c45)c32)c1[2H]] | 74% | 693.95 |
| B18 | A18 | NBS, DMF, THF | 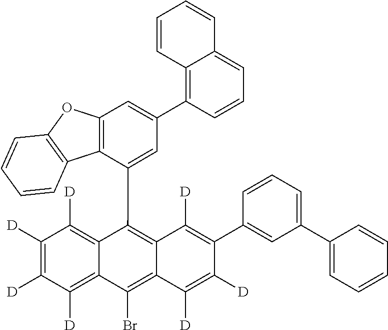 [SMILES: [2H]c1c([2H])c([2H])c2c(-c3cc(-c4cccc5ccccc45)cc4oc5ccccc5c34)c3c([2H])c(-c4cccc(-c5ccccc5)c4)c([2H])c([2H])c3c(Br)c2c1[2H]] | 70% | 709.71 |
| B19 | A19 | NBS, DMF, THF | 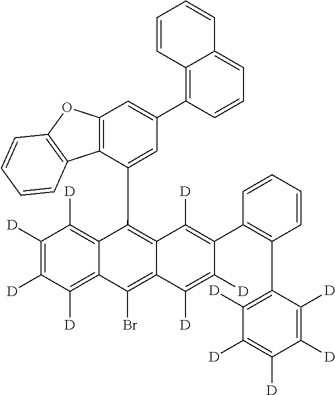 [SMILES: [2H]c1c([2H])c([2H])c(-c2ccccc2-c2c([2H])c([2H])c3c(Br)c4c([2H])c([2H])c([2H])c([2H])c4c(-c4cc(-c5cccc6ccccc56)cc5oc6ccccc6c45)c3c2[2H])c([2H])c1[2H]] | 71% | 714.74 |

### 3) Synthesis of Chemical Formula P1

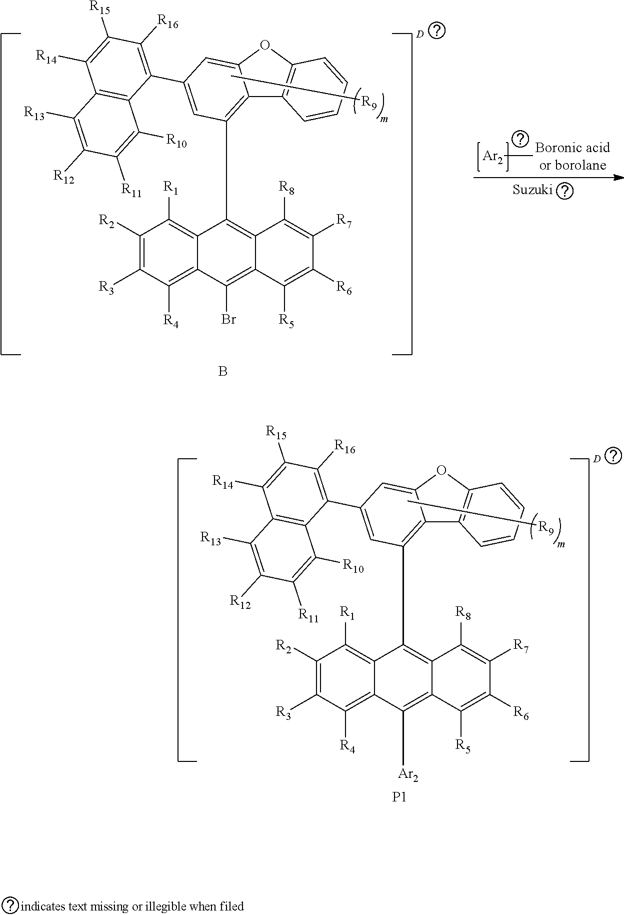

SM1 (Chemical Formula B, 1 eq.) and SM2 (1.1 eq.) were added to tetrahydrofuran (excess). After adding a 2 M potassium carbonate aqueous solution (30 vol. eq. of THE) and tetrakistriphenyl-phosphinopalladium (2 mol %), the mixture was heated at 85° C. for 10 hours under stirring. After lowering the temperature to room temperature and completing the reaction, the potassium carbonate aqueous solution was removed. After separation of layers, Chemical Formula P1 (compounds 1 to 31) was prepared by recrystallizing with chloroform and ethyl acetate.

In Chemical Formula P1, R9, which is identical or different, is independently hydrogen; deuterium; or a substituted or unsubstituted alkyl group, and m is an integer from 0 to 6.

Compounds 1 to 31 described in Table 7 were synthesized in the same manner as in the synthesis of Chemical Formula P1, except that SM1 and SM2 were changed.

TABLE 7

| | | | | Total | | |
|---|---|---|---|---|---|---|
| | | | | D | | |
| | | | | substi- | | |
| | | | | tution | | MS |
| Com- | | | | rate | | [M + |
| pound | SM1 | SM2 | Product | (%) | Yield | H]+ |
| 1 | B1 | 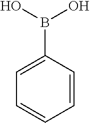 | 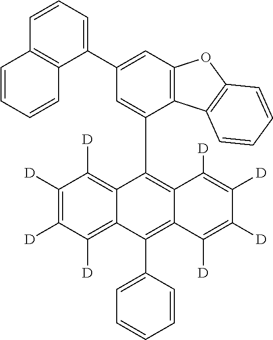 [SMILES: [2H]c1c([2H])c([2H])c2c(-c3cc(-c4cccc5ccccc45)cc4oc5ccccc5c34)c3c([2H])c([2H])c([2H])c([2H])c3c(-c3ccccc3)c2c1[2H]] | 31% | 68% | 555.72 |
| 2 | B1 | 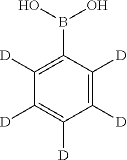 [SMILES: [2H]c1c([2H])c([2H])c(B(O)O)c([2H])c1[2H]] | 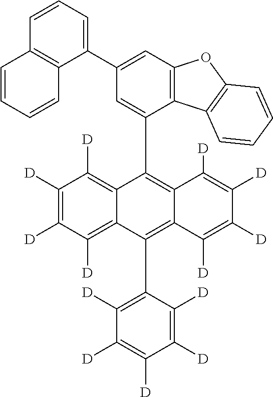 [SMILES: [2H]c1c([2H])c([2H])c(-c2c3c([2H])c([2H])c([2H])c([2H])c3c(-c3cc(-c4cccc5ccccc45)cc4oc5ccccc5c34)c3c([2H])c([2H])c([2H])c([2H])c23)c([2H])c1[2H]] | 50% | 66% | 560.76 |
| 3 | B2 | 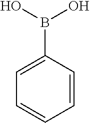 | 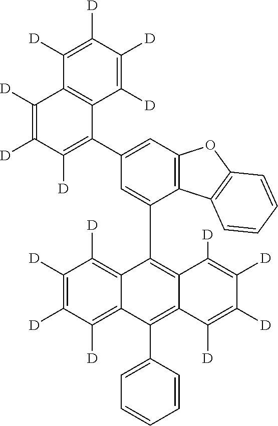 [SMILES: [2H]c1c([2H])c([2H])c2c(-c3cc(-c4c5c([2H])c([2H])c([2H])c([2H])c5c(-c5ccccc5)c5c([2H])c([2H])c([2H])c([2H])c45)c4c(c3)oc3ccccc34)c([2H])c([2H])c([2H])c2c1[2H]] | 58% | 70% | 562.76 |
| 4 | B2 | 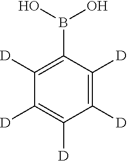 [SMILES: [2H]c1c([2H])c([2H])c(B(O)O)c([2H])c1[2H]] | 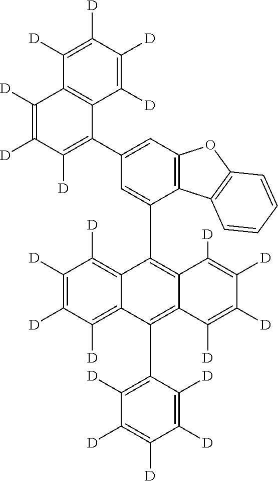 [SMILES: [2H]c1c([2H])c([2H])c(-c2c3c([2H])c([2H])c([2H])c([2H])c3c(-c3cc(-c4c([2H])c([2H])c([2H])c5c([2H])c([2H])c([2H])c([2H])c45)cc4oc5ccccc5c34)c3c([2H])c([2H])c([2H])c([2H])c23)c([2H])c1[2H]] | 77% | 64% | 567.79 |
| 5 | B3 | 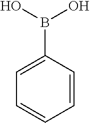 | 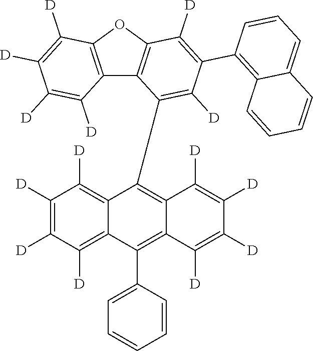 [SMILES: [2H]c1c([2H])c([2H])c2c(oc3c([2H])c(-c4cccc5ccccc45)c([2H])c(-c4c5c([2H])c([2H])c([2H])c([2H])c5c(-c5ccccc5)c5c([2H])c([2H])c([2H])c([2H])c45)c32)c1[2H]] | 54% | 68% | 561.75 |
| 6 | B3 | 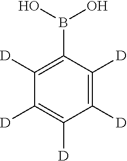 [SMILES: [2H]c1c([2H])c([2H])c(B(O)O)c([2H])c1[2H]] | 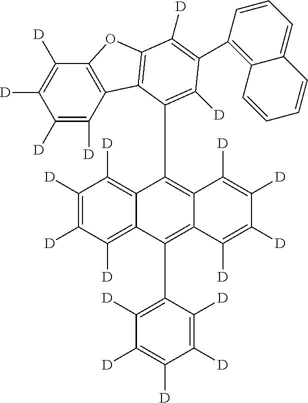 [SMILES: [2H]c1c([2H])c([2H])c(-c2c3c([2H])c([2H])c([2H])c([2H])c3c(-c3c([2H])c(-c4cccc5ccccc45)c([2H])c4oc5c([2H])c([2H])c([2H])c([2H])c5c34)c3c([2H])c([2H])c([2H])c([2H])c23)c([2H])c1[2H]] | 73% | 67% | 566.78 |
| 7 | B4 | 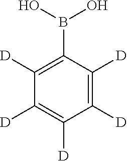 [SMILES: [2H]c1c([2H])c([2H])c(B(O)O)c([2H])c1[2H]] | 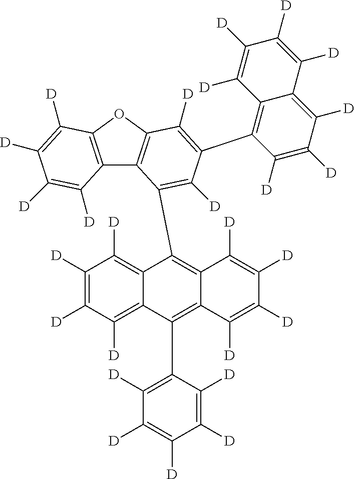 [SMILES: [2H]c1c([2H])c([2H])c(-c2c3c([2H])c([2H])c([2H])c([2H])c3c(-c3c([2H])c(-c4c([2H])c([2H])c([2H])c5c([2H])c([2H])c([2H])c([2H])c45)c([2H])c4oc5c([2H])c([2H])c([2H])c([2H])c5c34)c3c([2H])c([2H])c([2H])c([2H])c23)c([2H])c1[2H]] | 100% | 66% | 573.83 |
| 8 | B1 | 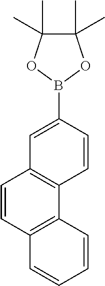 [SMILES: CC1(C)OB(c2ccc3c(ccc4ccccc43)c2)OC1(C)C] | 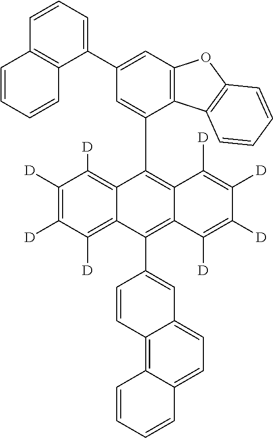 [SMILES: [2H]c1c([2H])c([2H])c2c(-c3cc(-c4cccc5ccccc45)cc4oc5ccccc5c34)c3c([2H])c([2H])c([2H])c([2H])c3c(-c3ccc4c(ccc5ccccc54)c3)c2c1[2H]] | 27% | 63% | 655.84 |
| 9 | B1 | 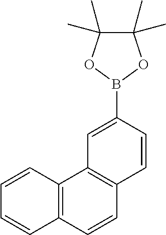 [SMILES: CC1(C)OB(c2ccc3ccc4ccccc4c3c2)OC1(C)C] | 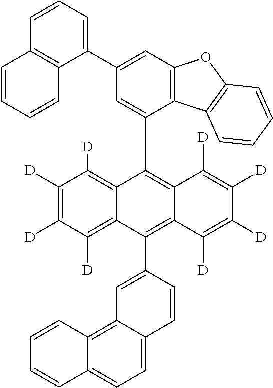 [SMILES: [2H]c1c([2H])c([2H])c2c(-c3cc(-c4cccc5ccccc45)cc4oc5ccccc5c34)c3c([2H])c([2H])c([2H])c([2H])c3c(-c3ccc4ccc5ccccc5c4c3)c2c1[2H]] | 57% | 62% | 664.89 |
| 10 | B2 | 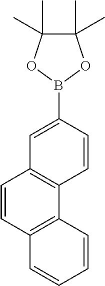 [SMILES: CC1(C)OB(c2ccc3c(ccc4ccccc43)c2)OC1(C)C] | 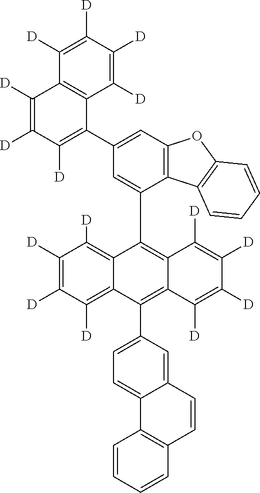 [SMILES: [2H]c1c([2H])c([2H])c2c(-c3cc(-c4c5c([2H])c([2H])c([2H])c([2H])c5c(-c5ccc6c(ccc7ccccc76)c5)c5c([2H])c([2H])c([2H])c([2H])c45)c4c(c3)oc3ccccc34)c([2H])c([2H])c([2H])c2c1[2H]] | 50% | 70% | 662.88 |
| 11 | B2 | 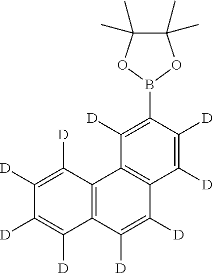 [SMILES: [2H]c1c([2H])c([2H])c2c(c1[2H])c([2H])c([2H])c1c([2H])c([2H])c(B3OC(C)(C)C(C)(C)O3)c([2H])c12] | 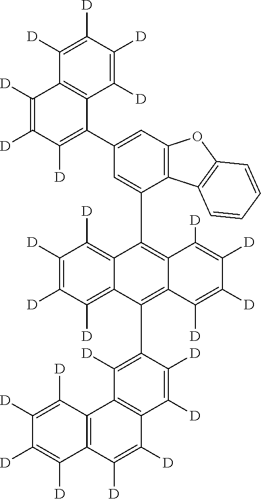 [SMILES: [2H]c1c([2H])c([2H])c2c(-c3cc(-c4c5c([2H])c([2H])c([2H])c([2H])c5c(-c5c([2H])c([2H])c6c([2H])c([2H])c7c([2H])c([2H])c([2H])c([2H])c7c6c5[2H])c5c([2H])c([2H])c([2H])c([2H])c45)c4c(c3)oc3ccccc34)c([2H])c([2H])c([2H])c2c1[2H]] | 80% | 71% | 671.94 |
| 12 | B3 | 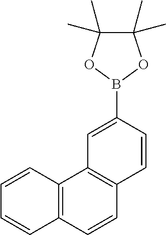 [SMILES: CC1(C)OB(c2ccc3ccc4ccccc4c3c2)OC1(C)C] | 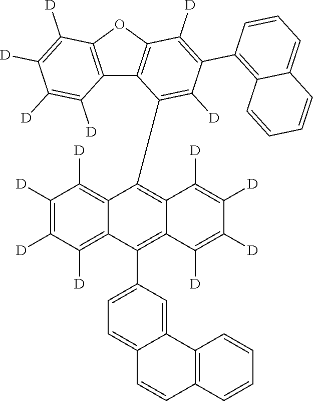 [SMILES: [2H]c1c([2H])c([2H])c2c(oc3c([2H])c(-c4cccc5ccccc45)c([2H])c(-c4c5c([2H])c([2H])c([2H])c([2H])c5c(-c5ccc6ccc7ccccc7c6c5)c5c([2H])c([2H])c([2H])c([2H])c45)c32)c1[2H]] | 47% | 63% | 660.87 |
| 13 | B3 | 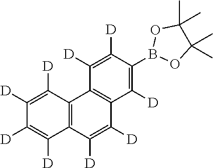 [SMILES: [2H]c1c([2H])c([2H])c2c(c1[2H])c([2H])c([2H])c1c([2H])c(B3OC(C)(C)C(C)(C)O3)c([2H])c([2H])c12] | 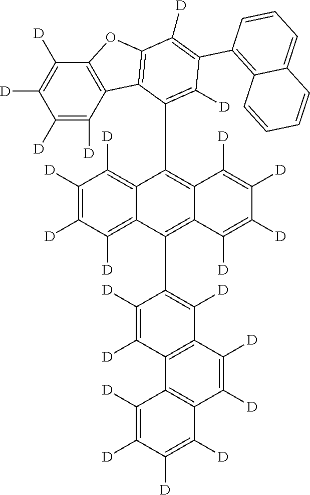 [SMILES: [2H]c1c([2H])c([2H])c2c(oc3c([2H])c(-c4cccc5ccccc45)c([2H])c(-c4c5c([2H])c([2H])c([2H])c([2H])c5c(-c5c([2H])c([2H])c6c(c5[2H])c([2H])c([2H])c5c([2H])c([2H])c([2H])c([2H])c56)c5c([2H])c([2H])c([2H])c([2H])c45)c32)c1[2H]] | 77% | 66% | 670.93 |
| 14 | B4 | 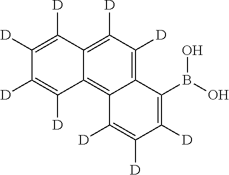 [SMILES: [2H]c1c([2H])c([2H])c2c(c1[2H])c([2H])c([2H])c1c(B(O)O)c([2H])c([2H])c([2H])c12] | 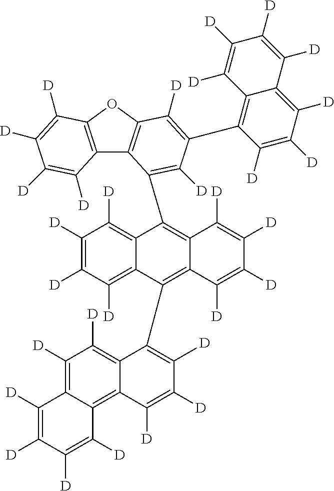 [SMILES: [2H]c1c([2H])c([2H])c2c(oc3c([2H])c(-c4c([2H])c([2H])c([2H])c5c([2H])c([2H])c([2H])c([2H])c45)c([2H])c(-c4c5c([2H])c([2H])c([2H])c([2H])c5c(-c5c([2H])c([2H])c([2H])c6c5c([2H])c([2H])c5c([2H])c([2H])c([2H])c([2H])c56)c5c([2H])c([2H])c([2H])c([2H])c45)c32)c1[2H]] | 100% | 65% | 677.97 |
| 15 | B5 | 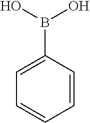 | 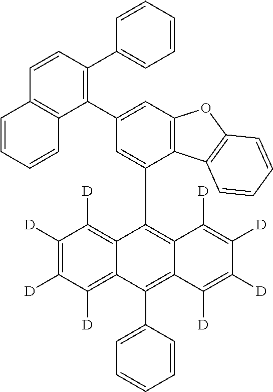 [SMILES: [2H]c1c([2H])c([2H])c2c(-c3cc(-c4c(-c5ccccc5)ccc5ccccc45)cc4oc5ccccc5c34)c3c([2H])c([2H])c([2H])c([2H])c3c(-c3ccccc3)c2c1[2H]] | 27% | 61% | 631.82 |
| 16 | B6 | 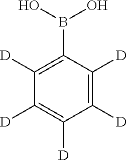 [SMILES: [2H]c1c([2H])c([2H])c(B(O)O)c([2H])c1[2H]] | 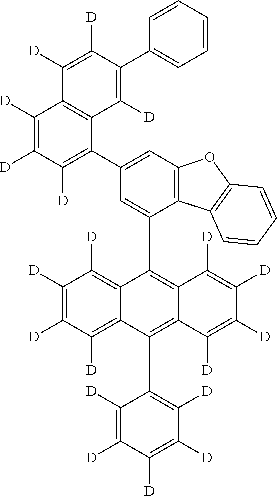 [SMILES: [2H]c1c([2H])c([2H])c(-c2c3c([2H])c([2H])c([2H])c([2H])c3c(-c3cc(-c4c([2H])c([2H])c([2H])c5c([2H])c([2H])c(-c6ccccc6)c([2H])c45)cc4oc5ccccc5c34)c3c([2H])c([2H])c([2H])c([2H])c23)c([2H])c1[2H]] | 63% | 60% | 642.88 |
| 17 | B7 | 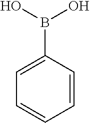 | 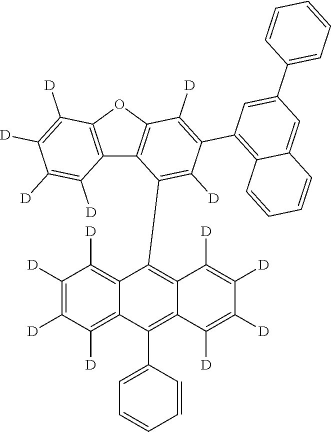 [SMILES: [2H]c1c([2H])c([2H])c2c(oc3c([2H])c(-c4cc(-c5ccccc5)cc5ccccc45)c([2H])c(-c4c5c([2H])c([2H])c([2H])c([2H])c5c(-c5ccccc5)c5c([2H])c([2H])c([2H])c([2H])c45)c32)c1[2H]] | 47% | 67% | 637.85 |
| 18 | B8 | 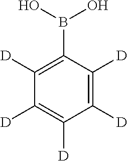 [SMILES: [2H]c1c([2H])c([2H])c(B(O)O)c([2H])c1[2H]] | 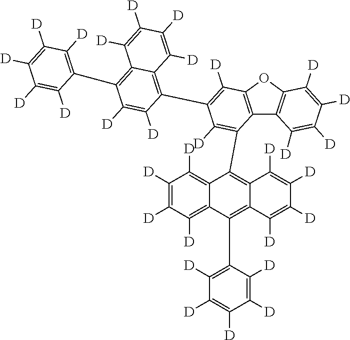 [SMILES: [2H]c1c([2H])c([2H])c(-c2c([2H])c([2H])c(-c3c([2H])c(-c4c5c([2H])c([2H])c([2H])c([2H])c5c(-c5c([2H])c([2H])c([2H])c([2H])c5[2H])c5c([2H])c([2H])c([2H])c([2H])c45)c4c(oc5c([2H])c([2H])c([2H])c([2H])c54)c3[2H])c3c([2H])c([2H])c([2H])c([2H])c23)c([2H])c1[2H]] | 100% | 66% | 653.95 |
| 19 | B9 | 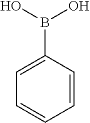 | 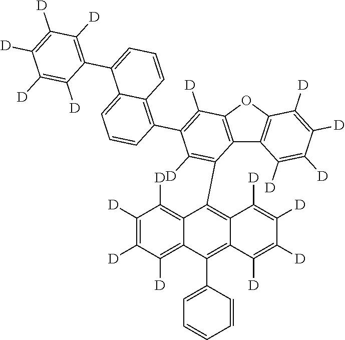 [SMILES: [2H]c1c([2H])c([2H])c(-c2cccc3c(-c4c([2H])c(-c5c6c([2H])c([2H])c([2H])c([2H])c6c(-c6ccccc6)c6c([2H])c([2H])c([2H])c([2H])c56)c5c(oc6c([2H])c([2H])c([2H])c([2H])c65)c4[2H])cccc23)c([2H])c1[2H]] | 63% | 69% | 642.88 |
| 20 | B10 | 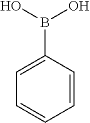 | 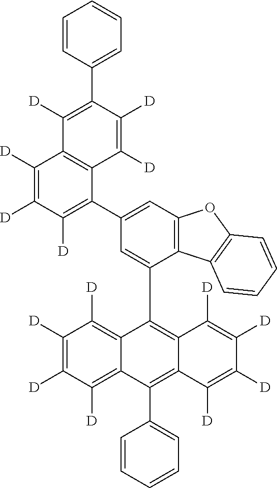 [SMILES: [2H]c1c([2H])c(-c2cc(-c3c4c([2H])c([2H])c([2H])c([2H])c4c(-c4ccccc4)c4c([2H])c([2H])c([2H])c([2H])c34)c3c(c2)oc2ccccc23)c2c([2H])c([2H])c(-c3ccccc3)c([2H])c2c1[2H]] | 47% | 61% | 637.85 |
| 21 | B11 | 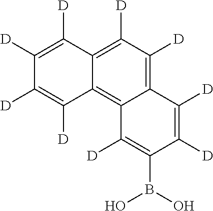 [SMILES: [2H]c1c([2H])c([2H])c2c(c1[2H])c([2H])c([2H])c1c([2H])c([2H])c(B(O)O)c([2H])c12] | 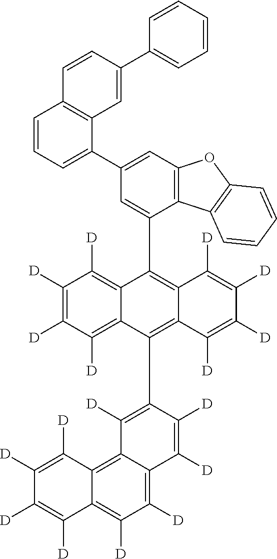 [SMILES: [2H]c1c([2H])c([2H])c2c(-c3cc(-c4cccc5ccc(-c6ccccc6)cc45)cc4oc5ccccc5c34)c3c([2H])c([2H])c([2H])c([2H])c3c(-c3c([2H])c([2H])c4c([2H])c([2H])c5c([2H])c([2H])c([2H])c([2H])c5c4c3[2H])c2c1[2H]] | 50% | 67% | 740.99 |
| 22 | B12 | 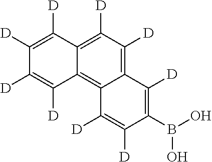 [SMILES: [2H]c1c([2H])c([2H])c2c(c1[2H])c([2H])c([2H])c1c([2H])c(B(O)O)c([2H])c([2H])c12] | 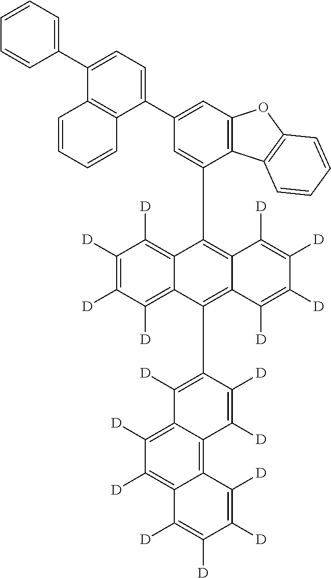 [SMILES: [2H]c1c([2H])c([2H])c2c(-c3cc(-c4ccc(-c5ccccc5)c5ccccc45)cc4oc5ccccc5c34)c3c([2H])c([2H])c([2H])c([2H])c3c(-c3c([2H])c([2H])c4c(c3[2H])c([2H])c([2H])c3c([2H])c([2H])c([2H])c([2H])c34)c2c1[2H]] | 50% | 64% | 740.99 |
| 23 | B13 | 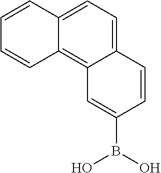 [SMILES: OB(O)c1ccc2ccc3ccccc3c2c1] | 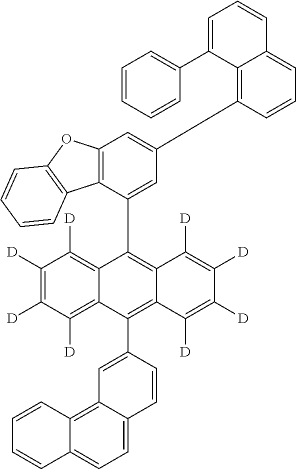 [SMILES: [2H]c1c([2H])c([2H])c2c(-c3cc(-c4cccc5cccc(-c6ccccc6)c45)cc4oc5ccccc5c34)c3c([2H])c([2H])c([2H])c([2H])c3c(-c3ccc4ccc5ccccc5c4c3)c2c1[2H]] | 24% | 66% | 731.94 |
| 24 | B7 | 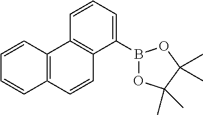 [SMILES: CC1(C)OB(c2cccc3c2ccc2ccccc23)OC1(C)C] | 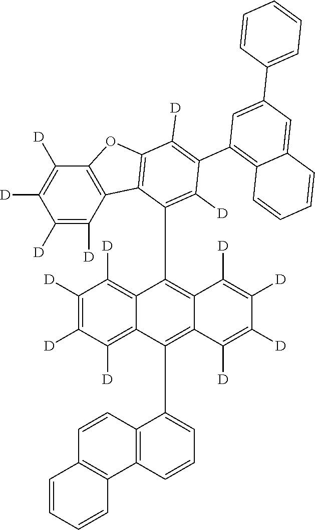 [SMILES: [2H]c1c([2H])c([2H])c2c(oc3c([2H])c(-c4cc(-c5ccccc5)cc5ccccc45)c([2H])c(-c4c5c([2H])c([2H])c([2H])c([2H])c5c(-c5cccc6c5ccc5ccccc56)c5c([2H])c([2H])c([2H])c([2H])c45)c32)c1[2H]] | 41% | 65% | 737.97 |
| 25 | B8 | 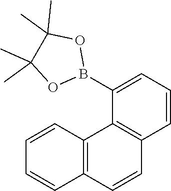 [SMILES: CC1(C)OB(c2cccc3ccc4ccccc4c23)OC1(C)C] | 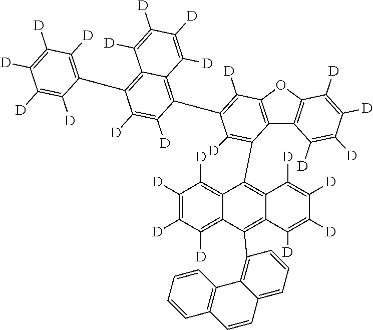 [SMILES: [2H]c1c([2H])c([2H])c(-c2c([2H])c([2H])c(-c3c([2H])c(-c4c5c([2H])c([2H])c([2H])c([2H])c5c(-c5cccc6ccc7ccccc7c56)c5c([2H])c([2H])c([2H])c([2H])c45)c4c(oc5c([2H])c([2H])c([2H])c([2H])c54)c3[2H])c3c([2H])c([2H])c([2H])c([2H])c23)c([2H])c1[2H]] | 74% | 63% | 749.04 |
| 26 | B14 | 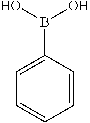 | 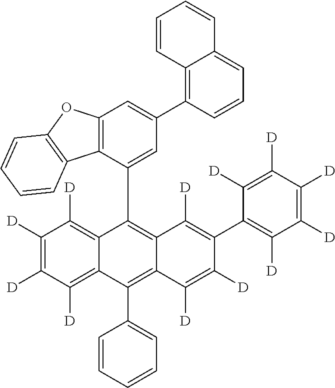 [SMILES: [2H]c1c([2H])c([2H])c(-c2c([2H])c([2H])c3c(-c4ccccc4)c4c([2H])c([2H])c([2H])c([2H])c4c(-c4cc(-c5cccc6ccccc56)cc5oc6ccccc6c45)c3c2[2H])c([2H])c1[2H]] | 40% | 69% | 635.84 |
| 27 | B15 | 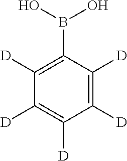 [SMILES: [2H]c1c([2H])c([2H])c(B(O)O)c([2H])c1[2H]] | 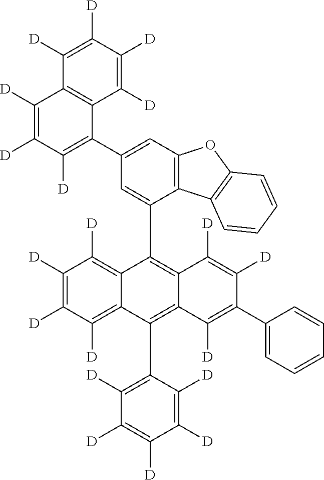 [SMILES: [2H]c1c([2H])c([2H])c(-c2c3c([2H])c([2H])c([2H])c([2H])c3c(-c3cc(-c4c([2H])c([2H])c([2H])c5c([2H])c([2H])c([2H])c([2H])c45)cc4oc5ccccc5c34)c3c([2H])c([2H])c(-c4ccccc4)c([2H])c23)c([2H])c1[2H]] | 63% | 68% | 642.88 |
| 28 | B16 | 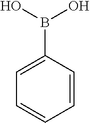 | 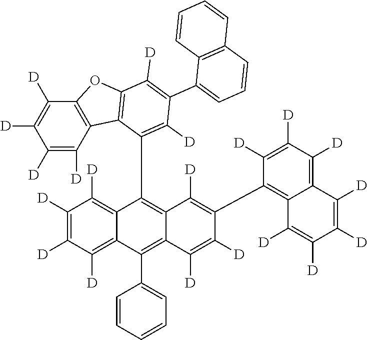 [SMILES: [2H]c1c([2H])c([2H])c2c(oc3c([2H])c(-c4cccc5ccccc45)c([2H])c(-c4c5c([2H])c([2H])c([2H])c([2H])c5c(-c5ccccc5)c5c([2H])c([2H])c(-c6c([2H])c([2H])c([2H])c7c([2H])c([2H])c([2H])c([2H])c67)c([2H])c45)c32)c1[2H]] | 63% | 70% | 693.95 |
| 29 | B17 | 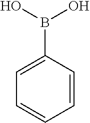 | 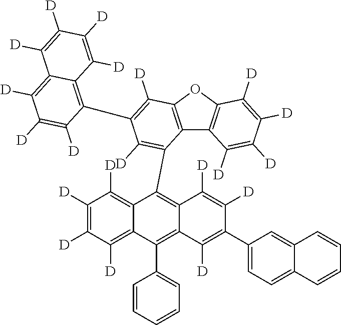 [SMILES: [2H]c1c([2H])c([2H])c2c(oc3c([2H])c(-c4c([2H])c([2H])c([2H])c5c([2H])c([2H])c([2H])c([2H])c45)c([2H])c(-c4c5c([2H])c([2H])c([2H])c([2H])c5c(-c5ccccc5)c5c([2H])c(-c6ccc7ccccc7c6)c([2H])c([2H])c45)c32)c1[2H]] | 63% | 71% | 693.95 |
| 30 | B18 | 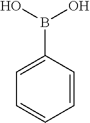 | 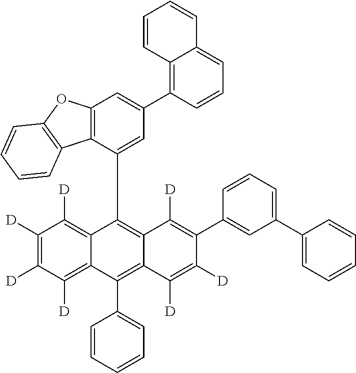 [SMILES: [2H]c1c([2H])c([2H])c2c(-c3cc(-c4cccc5ccccc45)cc4oc5ccccc5c34)c3c([2H])c(-c4cccc(-c5ccccc5)c4)c([2H])c([2H])c3c(-c3ccccc3)c2c1[2H]] | 21% | 69% | 706.91 |
| 31 | B19 | 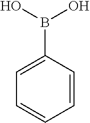 | 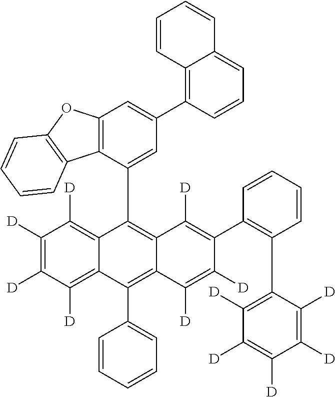 [SMILES: [2H]c1c([2H])c([2H])c(-c2ccccc2-c2c([2H])c([2H])c3c(-c4ccccc4)c4c([2H])c([2H])c([2H])c([2H])c4c(-c4cc(-c5cccc6ccccc56)cc5oc6ccccc6c45)c3c2[2H])c([2H])c1[2H]] | 35% | 68% | 711.94 |

### <Preparation Example 3> Synthesis of Chemical Formula P2

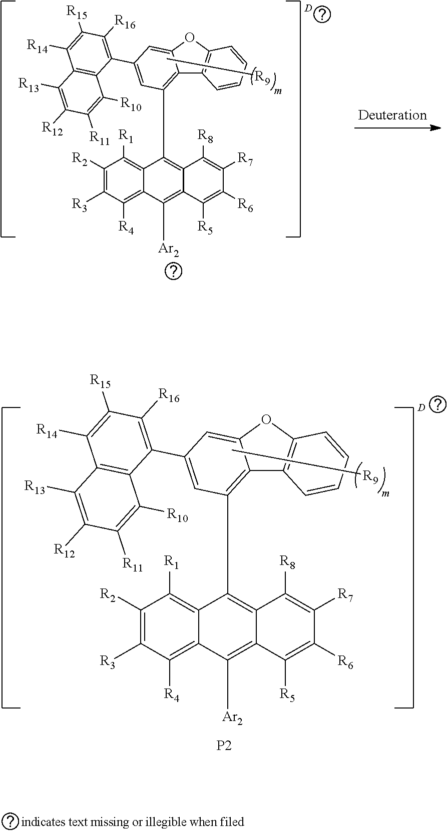

After adding a reactant (Chemical Formula C, 1 eq.) and trifluoromethanesulfonic acid (cat.) to C₆D₆ (10 to 50 mass eq. of reactant), the mixture was stirred at 70° C. for 10 to 100 minutes. After the reaction was completed, D₂O (excess) was added. After stirring for 30 minutes, trimethylamine (excess) was added dropwise. The reaction solution was transferred to a separatory funnel and extracted with water and chloroform. After drying the extract with MgSO₄, compounds 32 to 35 described in Table 8 were obtained by heating and recrystallizing with toluene.

In Chemical Formula P2, R9, which is identical or different, is independently hydrogen; deuterium; or a substituted or unsubstituted alkyl group, and m is an integer from 1 to 6.

Compounds 32 to 35 described in Table 8 were synthesized in the same manner as in the synthesis of Chemical Formula P2, except that the reactants (Chemical Formula C) were changed.

The reactants (Chemical Formula C) described in Table 8 were compounds 1, 8, 15 and 23 (compounds in which only the hydrogen of anthracene is deuterated, AN-D8 compounds) synthesized in Preparation Example 2. The compounds 32 to 35 (AN-D8+Z compounds) described in Table 8 were synthesized by the deuterium substitution method using benzene-D6 and TFA (trifluoromethanesulfonic acid) as described above. They were prepared by deuterium substitution of the compounds in which only the hydrogen of anthracene is substituted with deuterium among the products described in Table 7 (AN-D8 compounds), and they have D (8+z) deuteriums. z represents the number of deuteriums additionally substituted randomly (depending on reactivity) at the aryl unit substituted at positions 9 (dibenzofuran) and 10 (phenyl, phenanthrenyl, etc.) of anthracene. They were synthesized to compare electrical performance with compound in which only eight hydrogens of anthracene are substituted with deuterium (AN-D8 compound) and determine their performance in blue organic light-emitting devices.

TABLE 8

| | | | | | | | | Number | D | | | |
|---|---|---|---|---|---|---|---|---|---|---|---|---|
| | | | | | | | | of D | substi- | | Total | |
| | | | | Product, | | | Number | subst- | tution | Total | D | |
| | | | | theo- | Reac- | | of | tutions | rate | number | substi- | |
| | | | | retical | tion | Product, | hydrogens | after | via | of D | tution | |
| Com- | React- | | Reactant | max | time | actual | before | reaction | reaction | substi- | rate | |
| pound | ant | Product | m/z | m/z | (min) | max m/z | reaction | (Z) | (%) | tutions | (%) | Yield |
| 32 | Com- pound 1 | 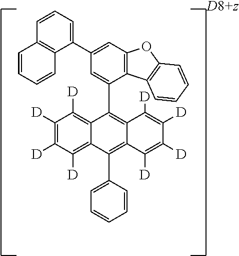 | 554 | 572 | 50 | 568 | 18 | 14 | 78% | 22 | 85% | 64% |
| 33 | Com- pound 8 | 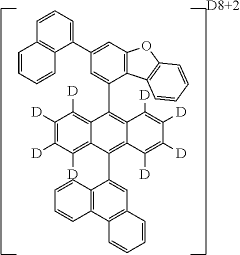 | 654 | 676 | 50 | 672 | 22 | 18 | 82% | 26 | 87% | 65% |
| 34 | Com- pound 15 | 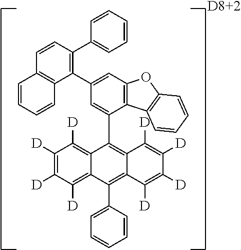 | 630 | 652 | 50 | 646 | 22 | 16 | 73% | 24 | 80% | 66% |
| 35 | Com- pound 23 | 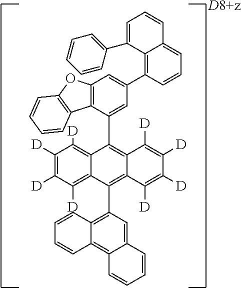 | 730 | 756 | 50 | 746 | 26 | 16 | 62% | 24 | 71% | 64% |

### Example 1. Preparation of OLED

As a first electrode, a substrate on which ITO/Ag/ITO were deposited to 70/1000/70 Å was cut into a size of 50 mm×50 mm×0.5 mm, placed in distilled water in which a dispersant was dissolved, and washed ultrasonically. As the detergent, the product of Fischer Co. was used. And, as the distilled water, distilled water filtered twice with a filter available from Millipore Co. was used. After washing the ITO for 30 minutes, ultrasonic cleaning was performed twice with distilled water for 10 minutes. After the washing with distilled water was completed, the ITO was ultrasonically cleansed using isopropyl alcohol, acetone and methanol sequentially, and then dried.

A hole injection layer was formed by thermally vacuum-depositing the following compound HI-1 on the prepared first electrode to a thickness of 50 Å, and on top of that, the following compound HT1, which is a material for transporting holes, was vacuum-deposited to a thickness of 1150 Å to form a hole transport layer. Then, after forming a hole control layer using the following compound EB1 (150 Å), a light-emitting layer was formed by vacuum-depositing the host compound 1 synthesized in Preparation Example 2 and the following dopant BD1 (2 wt %) to a thickness of 360 Å. Next, an electron control layer was formed by depositing the following compound ET1 to a thickness of 50 Å, and an electron transport layer was formed by depositing a 7:3 mixture of the following compounds ET2 and Liq to a thickness of 250 Å. A device was completed by sequentially depositing magnesium and lithium fluoride (LiF) to a thickness of 50 Å, as an electron injection layer, forming a second electrode to 200 Å using magnesium and silver (1:4), and then depositing the following compound CP1 (as a capping layer) to a thickness of 600 Å. The deposition rate of organic materials in the above procedure was maintained at 1 Å/sec.

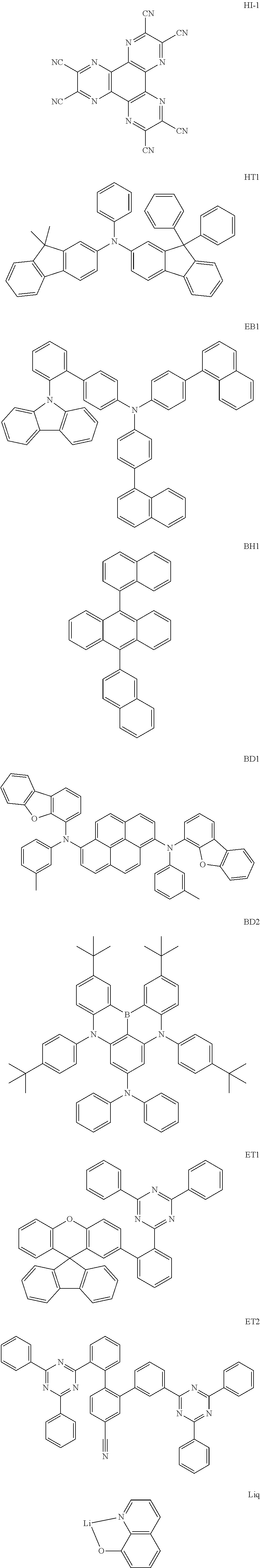

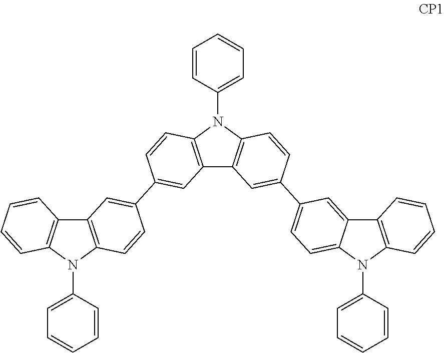 [SMILES: c1ccc(-n2c3ccccc3c3cc(-c4ccc5c(c4)c4cc(-c6ccc7c(c6)c6ccccc6n7-c6ccccc6)ccc4n5-c4ccccc4)ccc32)cc1]

### Examples 2 to 35

Organic light-emitting devices of Examples 2 to 35 were prepared in the same manner as in Example 1, except that compounds 2 to 31 were used as host materials of the light-emitting layer, and compounds BD1 or BD2 was used as dopant materials of the light-emitting layer, as shown in Table 10.

### Comparative Example 1-1

An organic light-emitting device of Comparative Example 1-1 was prepared in the same manner as in Example 1, except that the compound BH1 was used as a host material of the light-emitting layer and compound BD1 was used as a dopant material of the light-emitting layer.

### Comparative Examples 1-2 to 1-13

Organic light-emitting devices of Comparative Examples 1-2 to 1-13 were prepared in the same manner as in Example 1, except that compounds BH2 to BH10 described in Table 10 were used as host materials of the light-emitting layer, and compound BD1 or BD2 was used as dopant materials of the light-emitting layer, respectively.

The compounds BH2 to BH10 are as follows.

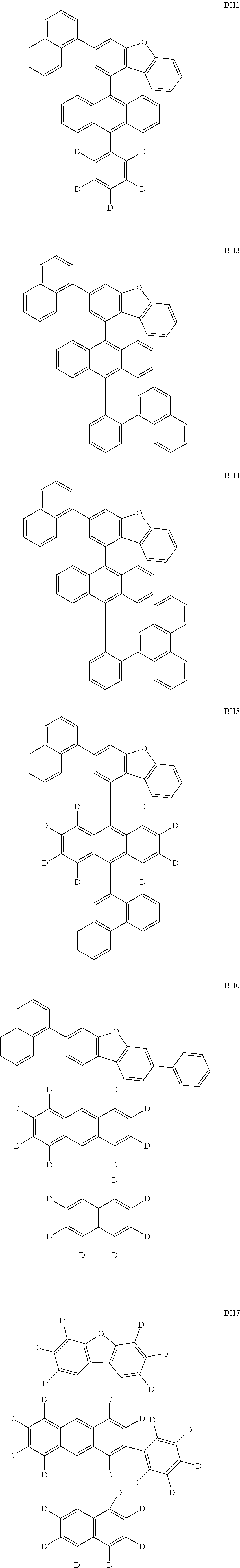

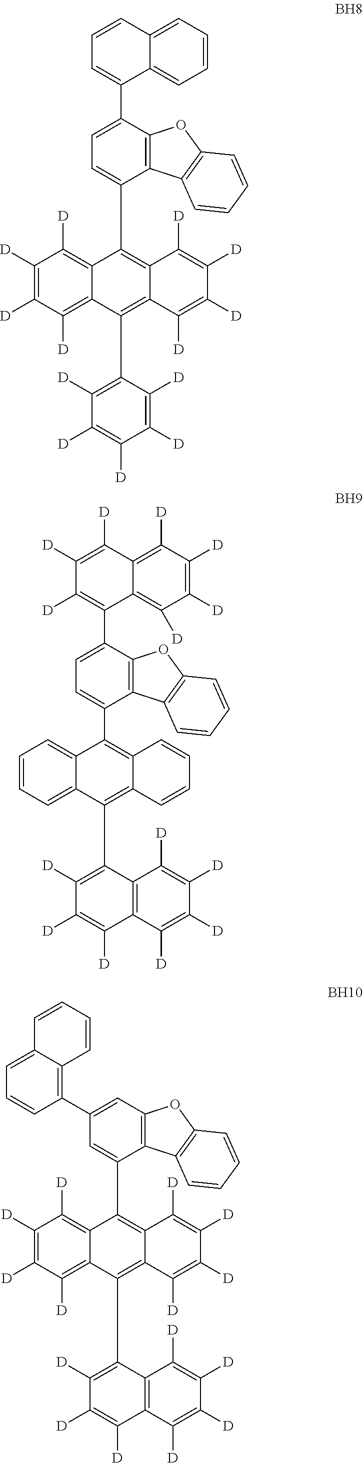

### Comparative Examples 3-1 to 3-25

Organic light-emitting devices of Comparative Examples 3-1 to 3-25 were prepared in the same manner as in Example 1, except that compounds in which all hydrogen-substitutable sites of the backbones of the compounds 1 to 31 described in Table 9 were replaced with hydrogen were used as host materials of the light-emitting layer, and compound BD1 or BD2 was used as the dopant material of the light-emitting layer, respectively.

### Test Example 1

While applying a current of 20 mA/cm² to the organic light-emitting devices prepared in Examples 1 to 35 and Comparative Examples 1-1 to 1-10, voltage, efficiency, color coordinates, lifetime, deuterium (D) substitution rate, and lifetime increase rate A (%) were measured, and the results are shown in Table 10. T95 refers to the time required for the brightness to decrease from the initial brightness (1600 nit) to 95%.

The lifetime increase rate A (%) is the lifetime increase rate of Examples 1 to 35 as compared to H compounds (compounds containing hydrogen only). The results for the compounds of Comparative Examples 3-1 to 3-25 described in Table 9 as compared to their H compounds (synthesized through the same process as the synthesis of Chemical Formula P1) are described in Table 10.

$${}^{*} Lifetime increase rate A ( \% ) = [ ( T 95 lifetime of final compound ( h ) - T 95 lifetime of H compound ( h ) ) / T 95 lifetime of final compound ( h ) ] \times 100$$

TABLE 9

| | | | | | | Lifetime |
|---|---|---|---|---|---|---|
| Test | | | Voltage (V) | Cd/A | Color | (T95, h) |
| Example | | | (@ 20 | (@ 20 | coordinates | (@ 20 |
| 20 mA/cm² | Host | Dopant | mA/cm²) | mA/cm²) | (x, y) | mA/cm²) |
| Comparative | H compound of | BD1 | 3.45 | 6.94 | (0.134, | 38.9 |
| Example | compounds 1 | | | | 0.094) | |
| 3-1 | to 7 | | | | | |
| Comparative | H compound of | BD1 | 3.36 | 7.10 | (0.134, | 33.5 |
| Example | compounds 8, | | | | 0.102) | |
| 3-2 | 10 and 13 | | | | | |
| Comparative | H compound of | BD1 | 3.39 | 7.03 | (0.134, | 31.8 |
| Example | compounds 9, | | | | 0.099) | |
| 3-3 | 11 and 12 | | | | | |
| Comparative | H compound of | BD1 | 3.40 | 7.02 | (0.135, | 32.6 |
| Example | compound 14 | | | | 0.101) | |
| 3-4 | | | | | | |
| Comparative | H compound of | BD1 | 3.43 | 6.89 | (0.134, | 37.4 |
| Example | compound 15 | | | | 0.096) | |
| 3-5 | | | | | | |
| Comparative | H compound of | BD1 | 3.41 | 6.91 | (0.135, | 35.8 |
| Example | compound 16 | | | | 0.096) | |
| 3-6 | | | | | | |
| Comparative | H compound of | BD1 | 3.42 | 6.90 | (0.135, | 36.1 |
| Example | compound 17 | | | | 0.096) | |
| 3-7 | | | | | | |
| Comparative | H compound of | BD1 | 3.39 | 6.85 | (0.134, | 36.4 |
| Example | compound 18 | | | | 0.097) | |
| 3-8 | | | | | | |
| Comparative | H compound of | BD1 | 3.40 | 6.90 | (0.134, | 37.0 |
| Example | compound 19 | | | | 0.097) | |
| 3-9 | | | | | | |
| Comparative | H compound of | BD1 | 3.43 | 6.88 | (0.135, | 34.1 |
| Example | compound 20 | | | | 0.096) | |
| 3-10 | | | | | | |
| Comparative | H compound of | BD1 | 3.38 | 6.93 | (0.134, | 35.0 |
| Example | compound 21 | | | | 0.103) | |
| 3-11 | | | | | | |
| Comparative | H compound of | BD1 | 3.35 | 7.00 | (0.135, | 32.8 |
| Example | compound 22 | | | | 0.105) | |
| 3-12 | | | | | | |
| Comparative | H compound of | BD1 | 3.39 | 6.99 | (0.134, | 33.6 |
| Example | compound 23 | | | | 0.103) | |
| 3-13 | | | | | | |
| Comparative | H compound of | BD1 | 3.37 | 7.01 | (0.134, | 32.7 |
| Example | compound 24 | | | | 0.101) | |
| 3-14 | | | | | | |
| Comparative | H compound of | BD1 | 3.36 | 6.92 | (0.134, | 33.9 |
| Example | compound 25 | | | | 0.101) | |
| 3-15 | | | | | | |
| Comparative | H compound of | BD1 | 3.32 | 7.03 | (0.136, | 38.1 |
| Example | compound 26 | | | | 0.112) | |
| 3-16 | | | | | | |
| Comparative | H compound of | BD1 | 3.35 | 7.05 | (0.135, | 37.9 |
| Example | compound 27 | | | | 0.112) | |
| 3-17 | | | | | | |
| Comparative | H compound of | BD1 | 3.30 | 7.11 | (0.136, | 39.1 |
| Example | compound 28 | | | | 0.107) | |
| 3-18 | | | | | | |
| Comparative | H compound of | BD1 | 3.31 | 7.08 | (0.136, | 39.4 |
| Example | compound 29 | | | | 0.106) | |
| 3-19 | | | | | | |
| Comparative | H compound of | BD1 | 3.32 | 7.03 | (0.135, | 37.1 |
| Example | compound 30 | | | | 0.112) | |
| 3-20 | | | | | | |
| Comparative | H compound of | BD1 | 3.31 | 7.01 | (0.135, | 36.8 |
| Example | compound 31 | | | | 0.113) | |
| 3-21 | | | | | | |
| Comparative | H compound of | BD2 | 3.53 | 7.08 | (0.134, | 30.4 |
| Example | compounds 1 | | | | 0.094) | |
| 3-22 | to 7 | | | | | |
| Comparative | H compound of | BD2 | 3.46 | 7.23 | (0.134, | 25.7 |
| Example | compounds 8, | | | | 0.102) | |
| 3-23 | 10 and 13 | | | | | |
| Comparative | H compound of | BD2 | 3.52 | 7.03 | (0.134, | 29.1 |
| Example | compound 15 | | | | 0.096) | |
| 3-24 | | | | | | |
| Comparative | H compound of | BD2 | 3.50 | 7.10 | (0.134, | 26.4 |
| Example | compound 23 | | | | 0.103) | |
| 3-25 | | | | | | |

TABLE 10

| | | | | | | | Total | |
|---|---|---|---|---|---|---|---|---|
| | | | Voltage | | | Lifetime | deuterium | Lifetime |
| Test | | | (V) | Cd/A | Color | (T95, h) | (D) | increase |
| Example | | | (@ 20 | (@ 20 | coordinates | (@ 20 | substitution | rate |
| 20 mA/cm² | Host | Dopant | mA/cm²) | mA/cm²) | (x, y) | mA/cm²) | rate (%) | A (%) |
| Example | Compound | BD1 | 3.45 | 6.94 | (0.134, | 61.3 | 31% | 37% |
| 1 | 1 | | | | 0.094) | | | |
| Example | Compound | BD1 | 3.46 | 6.94 | (0.134, | 63.8 | 50% | 39% |
| 2 | 2 | | | | 0.094) | | | |
| Example | Compound | BD1 | 3.45 | 6.94 | (0.134, | 65.1 | 58% | 40% |
| 3 | 3 | | | | 0.094) | | | |
| Example | Compound | BD1 | 3.45 | 6.95 | (0.134, | 66.8 | 77% | 42% |
| 4 | 4 | | | | 0.094) | | | |
| Example | Compound | BD1 | 3.45 | 6.94 | (0.134, | 65 | 54% | 40% |
| 5 | 5 | | | | 0.094) | | | |
| Example | Compound | BD1 | 3.44 | 6.94 | (0.134, | 68.4 | 73% | 43% |
| 6 | 6 | | | | 0.094) | | | |
| Example | Compound | BD1 | 3.45 | 6.94 | (0.134, | 76.4 | 100% | 49% |
| 7 | 7 | | | | 0.094) | | | |
| Example | Compound | BD1 | 3.36 | 7.10 | (0.134, | 50.8 | 27% | 34% |
| 8 | 8 | | | | 0.102) | | | |
| Example | Compound | BD1 | 3.39 | 7.03 | (0.134, | 50.2 | 57% | 37% |
| 9 | 9 | | | | 0.099) | | | |
| Example | Compound | BD1 | 3.37 | 7.09 | (0.134, | 55.2 | 50% | 39% |
| 10 | 10 | | | | 0.102) | | | |
| Example | Compound | BD1 | 3.39 | 7.03 | (0.134, | 55.1 | 80% | 42% |
| 11 | 11 | | | | 0.099) | | | |
| Example | Compound | BD1 | 3.39 | 7.03 | (0.134, | 51 | 47% | 38% |
| 12 | 12 | | | | 0.099) | | | |
| Example | Compound | BD1 | 3.36 | 7.11 | (0.134, | 58.4 | 77% | 43% |
| 13 | 13 | | | | 0.102) | | | |
| Example | Compound | BD1 | 3.40 | 7.02 | (0.135, | 60.1 | 100% | 46% |
| 14 | 14 | | | | 0.101) | | | |
| Example | Compound | BD1 | 3.44 | 6.89 | (0.134, | 56.8 | 27% | 34% |
| 15 | 15 | | | | 0.096) | | | |
| Example | Compound | BD1 | 3.41 | 6.91 | (0.135, | 58.1 | 63% | 38% |
| 16 | 16 | | | | 0.096) | | | |
| Example | Compound | BD1 | 3.42 | 6.90 | (0.135, | 55.3 | 47% | 35% |
| 17 | 17 | | | | 0.096) | | | |
| Example | Compound | BD1 | 3.39 | 6.86 | (0.134, | 66.2 | 100% | 45% |
| 18 | 18 | | | | 0.097) | | | |
| Example | Compound | BD1 | 3.40 | 6.90 | (0.134, | 60.2 | 63% | 39% |
| 19 | 19 | | | | 0.097) | | | |
| Example | Compound | BD1 | 3.43 | 6.88 | (0.135, | 55.4 | 47% | 38% |
| 20 | 20 | | | | 0.096) | | | |
| Example | Compound | BD1 | 3.38 | 6.93 | (0.134, | 57.8 | 50% | 39% |
| 21 | 21 | | | | 0.103) | | | |
| Example | Compound | BD1 | 3.35 | 6.99 | (0.135, | 59.5 | 50% | 45% |
| 22 | 22 | | | | 0.105) | | | |
| Example | Compound | BD1 | 3.38 | 6.99 | (0.134, | 51.8 | 24% | 35% |
| 23 | 23 | | | | 0.103) | | | |
| Example | Compound | BD1 | 3.37 | 7.01 | (0.134, | 53.1 | 41% | 38% |
| 24 | 24 | | | | 0.101) | | | |
| Example | Compound | BD1 | 3.37 | 6.92 | (0.134, | 62.1 | 74% | 45% |
| 25 | 25 | | | | 0.101) | | | |
| Example | Compound | BD1 | 3.32 | 7.03 | (0.136, | 63.1 | 40% | 40% |
| 26 | 26 | | | | 0.112) | | | |
| Example | Compound | BD1 | 3.35 | 7.05 | (0.135, | 66.2 | 63% | 43% |
| 27 | 27 | | | | 0.112) | | | |
| Example | Compound | BD1 | 3.30 | 7.10 | (0.136, | 66.3 | 63% | 41% |
| 28 | 28 | | | | 0.107) | | | |
| Example | Compound | BD1 | 3.30 | 7.08 | (0.136, | 63.1 | 63% | 38% |
| 29 | 29 | | | | 0.106) | | | |
| Example | Compound | BD1 | 3.33 | 7.03 | (0.135, | 62.4 | 21% | 41% |
| 30 | 30 | | | | 0.112) | | | |
| Example | Compound | BD1 | 3.31 | 7.01 | (0.135, | 63.4 | 35% | 42% |
| 31 | 31 | | | | 0.113) | | | |
| Example | Compound | BD2 | 3.53 | 7.08 | (0.134, | 50.4 | 31% | 40% |
| 32 | 1 | | | | 0.094) | | | |
| Example | Compound | BD2 | 3.46 | 7.23 | (0.134, | 45.3 | 27% | 43% |
| 33 | 8 | | | | 0.102) | | | |
| Example | Compound | BD2 | 3.52 | 7.03 | (0.134, | 43.8 | 27% | 34% |
| 34 | 15 | | | | 0.096) | | | |
| Example | Compound | BD2 | 3.50 | 7.10 | (0.134, | 44.2 | 24% | 40% |
| 35 | 23 | | | | 0.103) | | | |
| Comparative | BH1 | BD1 | 3.72 | 6.49 | (0.134, | 25.8 | 0% | — |
| Example | | | | | 0.090) | | | |
| 1-1 | | | | | | | | |
| Comparative | BH2 | BD1 | 3.45 | 6.94 | (0.134, | 42.3 | 19% | 8% |
| Example | | | | | 0.094) | | | |
| 1-2 | | | | | | | | |
| Comparative | BH3 | BD1 | 3.43 | 6.83 | (0.135, | 21.8 | 0% | — |
| Example | | | | | 0.109) | | | |
| 1-3 | | | | | | | | |
| Comparative | BH4 | BD1 | 3.44 | 6.79 | (0.135, | 23.5 | 0% | — |
| Example | | | | | 0.108) | | | |
| 1-4 | | | | | | | | |
| Comparative | BH5 | BD1 | 3.48 | 6.74 | (0.134, | 45.9 | 27% | — |
| Example | | | | | 0.099) | | | |
| 1-5 | | | | | | | | |
| Comparative | BH6 | BD1 | 3.46 | 6.77 | (0.134, | 30.9 | 47% | — |
| Example | | | | | 0.102) | | | |
| 1-6 | | | | | | | | |
| Comparative | BH7 | BD1 | 3.60 | 6.70 | (0.135, | 50.3 | 100% | — |
| Example | | | | | 0.116) | | | |
| 1-7 | | | | | | | | |
| Comparative | BH2 | BD2 | 3.54 | 7.07 | (0.134, | 32.8 | 19% | 7% |
| Example | | | | | 0.094) | | | |
| 1-8 | | | | | | | | |
| Comparative | BH5 | BD2 | 3.6 | 6.82 | (0.134, | 34.2 | 27% | — |
| Example | | | | | 0.099) | | | |
| 1-9 | | | | | | | | |
| Comparative | BH7 | BD2 | 3.73 | 6.85 | (0.135, | 42.9 | 100% | — |
| Example | | | | | 0.116) | | | |
| 1-10 | | | | | | | | |
| Comparative | BH8 | BD1 | 3.51 | 6.75 | (0.134, | 34.6 | 50% | — |
| Example | | | | | 0.098) | | | |
| 1-11 | | | | | | | | |
| Comparative | BH9 | BD1 | 3.43 | 6.23 | (0.134, | 30.2 | 50% | — |
| Example | | | | | 0.101) | | | |
| 1-12 | | | | | | | | |
| Comparative | BH10 | BD1 | 3.44 | 6.61 | (0.134, | 40.6 | 50% | — |
| Example | | | | | 0.098) | | | |
| 1-13 | | | | | | | | |

As shown in Table 10, the organic light-emitting devices of Examples 1 to 35, wherein the compound represented by Chemical Formula 1-1 or Chemical Formula 1-2 of the present disclosure, i.e., the compound obtained by deuteration of an anthracene derivative containing dibenzofuran, was used, exhibited superior characteristics in terms of operation voltage, efficiency and lifetime as compared to Comparative Examples 1-1 to 1-10. In particular, it was confirmed that the lifetime characteristics, which are a chronic difficulty in the performance of blue organic light-emitting devices, were improved.

Specifically, although the lifetime increase rate varies depending on the characteristics of each compound and the suitability of the device, Examples 1 to 31 showed increase rate of about 30 to 50%, and it was confirmed that the stability of the blue organic light-emitting device can be improved through the enhancement of lifetime resulting from deuterium substitution.

Examples 32 to 35, wherein only the blue dopant in Examples 1 to 31 was changed (from BD1 to BD2), showed a lifetime increase rate of about 30 to 45%. The overall increase in voltage and efficiency can be seen as the effect of the blue fluorescent dopant. But, it was confirmed that increase of lifetime achieved through deuteration of the anthracene blue host was maintained despite the change of the dopant.

Comparative Example 1-1, wherein the deuterium-unsubstituted compound of an aryl-based anthracene blue host was used, was confirmed to have a significantly higher voltage than the device having an anthracene blue host into which dibenzofuran was introduced.

Comparative Example 1-2, wherein deuterium was substituted only at the phenyl-D₅ at position 10 in the backbones of Examples 1 to 7, showed lifetime improvement of about 8% as compared to the H compounds with the same backbones. This differs more than 30% as compared to the 37% lifetime improvement of Example 1 (compound in which all the hydrogens of the anthracene backbone are replaced with deuteriums), revealing that the lifetime improvement effect was reduced significantly. In addition, Examples 1 to 7, wherein all the hydrogens of the anthracene backbone in the H compound of Comparative Example 1-2 were substituted with deuterium and deuterium was additionally substituted at substituents 9 and 10, show that deuterium substitution of the anthracene backbone plays a key role in improving lifetime. For example, the difference in the lifetime increase rate A between Example 1 (deuterium substitution rate 31%) and Example 7 (deuterium substitution rate 100%) was 12%, which is in contrast to the lifetime improvement rate A of 37% for Example 1 as compared to the H compound.

For Comparative Examples 1-3 and 1-4, wherein the substituent at position 10 of anthracene was phenyl-o-naphthyl and phenyl-o-phenanthrene, respectively, not substituted with deuterium, it was confirmed that the overall efficiency characteristics were similar to or slightly decreased as compared to the examples of the present disclosure due to the influence of carrier balance, but the difference in lifetime was very large.

For Comparative Examples 1-5, wherein the substituent at position 10 of anthracene was 9-phenanthrene and all hydrogens in the anthracene backbone were substituted with deuterium, it was confirmed that the overall efficiency characteristics were different as compared to Examples 8 to 14 with structural difference and similarity in deuterium substitution. This was also confirmed to be due to the effect of the carrier balance of the device.

For Comparative Example 1-6, wherein a substituent (phenyl) was introduced additionally in the 3′ direction to the dibenzofuran (substitution with 1-naphthyl at position 3) backbone at position 9 of anthracene, it was confirmed that the deuterium substitution rate was quite high at 47%, but the lifetime was very low due to the characteristics of the compound.

For Comparative Examples 1-7, wherein a phenyl group was introduced at position 2 of anthracene, but with no additional substituent at the dibenzofuran unit at position 9, it was confirmed that the operation voltage was considerably higher than that of Examples 26 to 31 according to the present disclosure, although all hydrogen were replaced with deuterium.

In Comparative Examples 1-8 to 1-10, only the blue dopant was changed (from BD1 to BD2) from Comparative Examples 1-2, 1-5 and 1-7, respectively. The overall increase in voltage and efficiency can be seen as the influence of the blue fluorescent dopant. The devices also showed a lifetime increase rate of about 30 to 45%, confirming that the lifetime increase through deuteration of the anthracene blue host was maintained despite the change in the dopant. In particular, Comparative Example 1-8 showed the same tendency as Comparative Example 1-2, with the lifetime increase rate A being low at about 7%, despite the introduction of a substituent (phenyl-D₅) at position 10 in addition to the hydrogens of the anthracene backbone.

Comparative Example 1-11, wherein 1-naphthyl was substituted in a direction different from that of the chemical formula according to the present disclosure, exhibited lower device performance as compared to the examples of the present disclosure. This shows that the direction of introduction of 1-naphthyl also has a significant effect on electrical properties.

Comparative Example 1-12 showed lower efficiency as compared to the examples of the present disclosure because the naphthyl group at position 10 of anthracene affected the initial injection of holes and showed gradation characteristics at low current.

For Comparative Example 1-13, wherein 1-naphthyl was substituted at the same position, it was confirmed that the device performance was deteriorated because the naphthyl group at position 10 of anthracene showed gradation characteristics in the device, as in Comparative Example 1-12.

### Test Example 2

Examples 36 to 43 in Table 11 below show the results for the organic light-emitting devices prepared using compounds 32 to 35, which were synthesized by additionally introducing deuterium to the substituents of compounds in which only anthracene was substituted with deuterium in compounds 1 to 31 (compounds 1, 8, 15 and 23), as host materials for the light-emitting layer, and BD1 or BD2 as a dopant material for the light-emitting layer. The total deuterium substitution rate (%) of compounds 32 to 35 as compared to the H compounds, the lifetime increase rate B (%) as compared to the H compounds, and the lifetime increase rate C (%) as compared to the compounds in which only anthracene was substituted with deuterium (compounds 1, 8, 15 and 23) are shown in Table 11.

In particular, it was confirmed that the same tendency was maintained for Examples 40 to 43, wherein only the blue dopant was changed from BD1 to BD2 in Examples 36 to 39.

While applying a current of 20 mA/cm² to the organic light-emitting devices prepared in Examples 36 to 43, voltage, efficiency, color coordinates, lifetime, deuterium (D) substitution rate, and lifetime increase rate B and C (%) were measured, and the results are shown in Table 11. T95 refers to the time required for the brightness to decrease from the initial brightness (1600 nit) to 95%.

TABLE 11

| | | | | | | | Total | | |
|---|---|---|---|---|---|---|---|---|---|
| | | | Voltage | | | Lifetime | deuterium | Lifetime | Lifetime |
| Test | | | (V) | Cd/A | Color | (T95, h) | (D) | increase | increase |
| Example | | | (@ 20 | (@ 20 | coordinates | (@ 20 | substitution | rate | rate |
| 20 mA/cm² | Host | Dopant | mA/cm²) | mA/cm²) | (x, y) | mA/cm²) | rate (%) | B (%) | C (%) |
| Example | Compound | BD1 | 3.45 | 6.94 | (0.134, | 69.2 | 85% | 44% | 11.42% |
| 36 | 32 | | | | 0.094) | | | | |
| Example | Compound | BD1 | 3.36 | 7.1 | (0.134, | 55.8 | 87% | 40% | 8.96% |
| 37 | 33 | | | | 0.102) | | | | |
| Example | Compound | BD1 | 3.44 | 6.89 | (0.134, | 60.9 | 80% | 39% | 6.73% |
| 38 | 34 | | | | 0.096) | | | | |
| Example | Compound | BD1 | 3.38 | 6.99 | (0.134, | 58.1 | 71% | 42% | 10.84% |
| 39 | 35 | | | | 0.103) | | | | |
| Example | Compound | BD2 | 3.53 | 7.08 | (0.134, | 57.3 | 85% | 47% | 12.04% |
| 40 | 32 | | | | 0.094) | | | | |
| Example | Compound | BD2 | 3.46 | 7.23 | (0.134, | 50.1 | 87% | 49% | 9.58% |
| 41 | 33 | | | | 0.102) | | | | |
| Example | Compound | BD2 | 3.52 | 7.03 | (0.134, | 47.5 | 80% | 39% | 7.79% |
| 42 | 34 | | | | 0.096) | | | | |
| Example | Compound | BD2 | 3.5 | 7.1 | (0.134, | 50.1 | 71% | 47% | 11.78% |
| 43 | 35 | | | | 0.103) | | | | |

For detailed analysis, Table 12 below was created.

TABLE 12

| | | | | | | | | Difference in | | |
|---|---|---|---|---|---|---|---|---|---|---|
| | | | | | | | | deuterium | | |
| | | | | | | | | substitution | | |
| | | | | | | | | rate | | |
| | | | | | | | | <substitution | Lifetime | Lifetime |
| | | | Deuterium | Lifetime | | | Deuterium | rate B (%) − | increase | increase |
| | | | substitution | increase | | | substitution | substitution | rate | rate |
| | Test | | rate A | rate A | Test | | rate | rate | B | C |
| Device | Example | Compound | (%) | (%) | Example | Compound | B (%) | A (%)> | (%) | (%) |
| BD1 | Example | Compound | 31% | 37% | Example | Compound | 85% | 54% | 44% | 11.42% |
| | 1 | 1 | | | 36 | 32 | | | | |
| | Example | Compound | 27% | 34% | Example | Compound | 87% | 60% | 40% | 8.96% |
| | 8 | 8 | | | 37 | 33 | | | | |
| | Example | Compound | 27% | 34% | Example | Compound | 80% | 53% | 39% | 6.73% |
| | 15 | 15 | | | 38 | 34 | | | | |
| | Example | Compound | 24% | 35% | Example | Compound | 71% | 47% | 42% | 10.84% |
| | 23 | 23 | | | 39 | 35 | | | | |
| BD2 | Example | Compound | 31% | 40% | Example | Compound | 85% | 54% | 47% | 12.04% |
| | 32 | 1 | | | 40 | 32 | | | | |
| | Example | Compound | 27% | 43% | Example | Compound | 87% | 60% | 49% | 9.58% |
| | 33 | 8 | | | 41 | 33 | | | | |

As shown in Table 12, compounds 32 to 35 exhibited different additional deuterium substitution rates although they were formed under the same reaction conditions (same temperature and reagent), which was due to the difference in chemical structure. Because compound 1 according to the present disclosure is composed of delocalized electron-rich groups and has no steric influence by additional substituents, it was synthesized into compound 32 having a high deuterium substitution rate. On the other hand, for compound 23 according to the present disclosure, because the substitution of hydrogen with deuterium is hindered due to the steric hindrance of the phenyl substituent of the naphthyl group and anthracene, it was synthesized into compound 35 with a lower deuterium substitution rate than compound 1 under the same reaction condition.

In addition, as shown in Table 12, it was confirmed that the increase in deuterium substitution rate through deuterium substitution of compounds 1, 8, 15 and 23 led to a slight increase in the device lifetime (increase of about 6 to 12%). Although the introduction of deuterium to additional substituents other than the anthracene backbone may also have affected the increase in lifetime, it was confirmed in Examples 1 to 31 that the introduction of deuterium to the anthracene backbone led to a major lifetime enhancement effect (35 to 50%).

In conclusion, it was confirmed that the organic light-emitting device using the compound according to the present disclosure exhibited excellent characteristics in terms of efficiency, operation voltage and lifetime.

### DETAILED DESCRIPTION OF MAIN ELEMENTS

1: substrate
2: first electrode
3: hole injection layer
4: hole transport layer
5: electron-blocking (hole control) layer
6: light-emitting layer
7: hole-blocking (electron control) layer
8: electron transport layer
9: electron injection layer
10: second electrode
11: capping layer

1. A compound represented by Chemical Formula 1-1 or Chemical Formula 1-2: 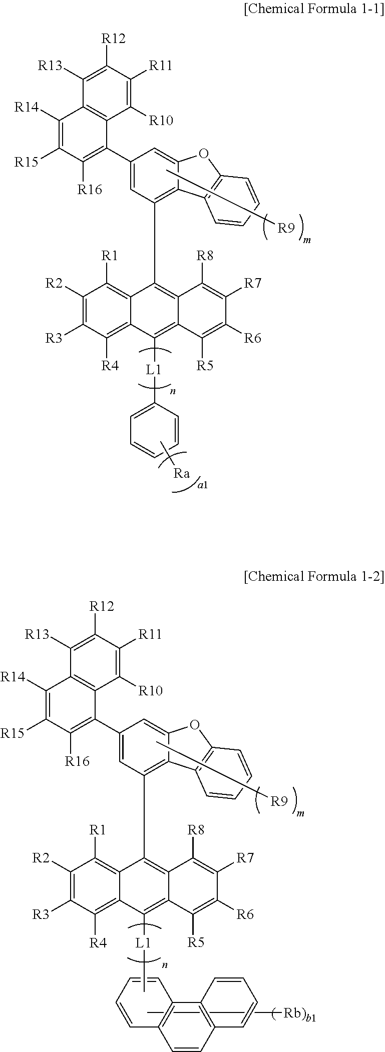
wherein:
R1 to R8, which are identical or different, are independently deuterium, a substituted or unsubstituted aryl group, or a substituted or unsubstituted heteroaryl group;
R9, which is identical or different, is independently hydrogen, deuterium, or a substituted or unsubstituted alkyl group;
R10 to R16, which are identical or different, are independently hydrogen, deuterium, a substituted or unsubstituted alkyl group, a substituted or unsubstituted cycloalkyl group, or a substituted or unsubstituted aryl group;
L1, which is identical or different, is independently a direct bond or a substituted or unsubstituted heteroarylene group;
Ra, which is identical or different, is independently hydrogen, deuterium, a substituted or unsubstituted alkyl group, a substituted or unsubstituted aliphatic hydrocarbon ring group, a substituted or unsubstituted aryl group wherein three or more ring are condensed, or a substituted or unsubstituted heteroaryl group, or is bonded to an adjacent substituent to form an aliphatic hydrocarbon ring;
Rb, which is identical or different, is independently hydrogen, deuterium, a substituted or unsubstituted alkyl group, a substituted or unsubstituted cycloalkyl group, a substituted or unsubstituted aryl group, or a substituted or unsubstituted heteroaryl group;
m is an integer from 1 to 6, 6;
n is an integer 1 or 2;
a1 is an integer from 1 to 5;
b1 is an integer from 1 to 9; and
if each of m, n, a1 or b1 is 2 or greater, R9's, L1s, Ras, or Rbs are identical or different.

2. The compound according to claim 1, wherein Chemical Formula 1-1 is Chemical Formula 1-11: 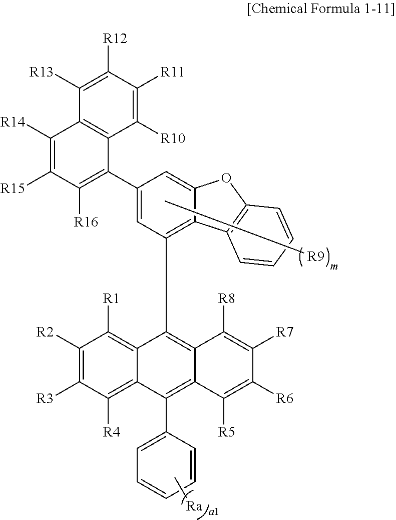
wherein R1 to R16, Ra, m and a1 are the same as defined in Chemical Formula 1-1.

3. The compound according to claim 1, wherein Chemical Formula 1-2 is Chemical Formula 1-21: 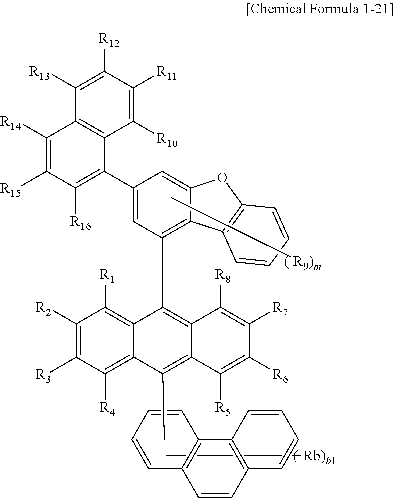
wherein R1 to R16, Rb, m and b1 are the same as defined in Chemical Formula 1-2.

4. The compound according to claim 1, wherein Chemical Formula 1-2 is any one of Chemical Formula 1-2a to Chemical Formula 1-2d: 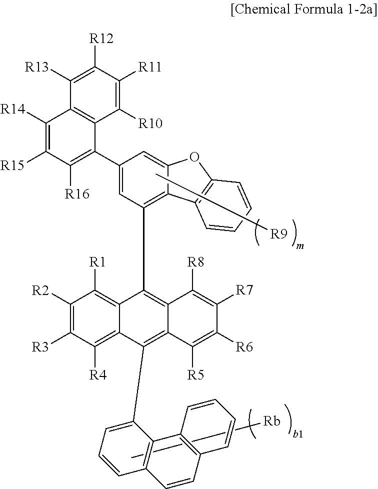 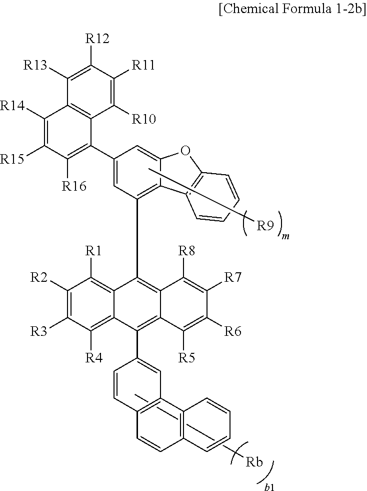 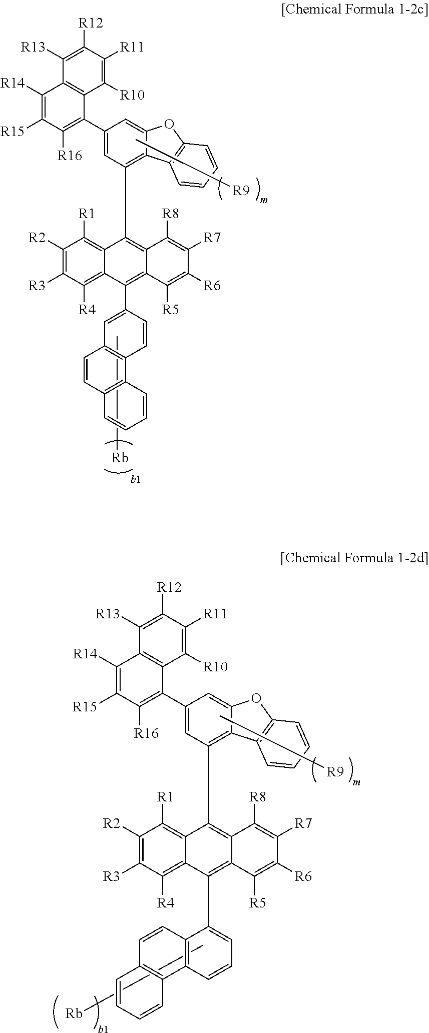
wherein R1 to R16, Rb, m and b1 are the same as defined in Chemical Formula 1-2.

5. The compound according to claim 1, wherein R1 to R8, which are identical or different, are independently deuterium or a substituted or unsubstituted C₆₋₂₀ aryl group.

6. The compound according to claim 1, wherein R1 to R8, which are identical or different, are independently deuterium; or a C₆₋₁₂ aryl group that is unsubstituted or substituted with deuterium, and at least one of R1 to R8 is a C₆₋₁₂ aryl group substituted or unsubstituted with deuterium.

7. The compound according to claim 1, wherein R9 is hydrogen, deuterium, or a substituted or unsubstituted C₁₋₁₀ alkyl group.

8. The compound according to claim 1, wherein R10 to R16, which are identical or different, are independently hydrogen, deuterium, a substituted or unsubstituted C₁₋₂₀ alkyl group, a substituted or unsubstituted C₃₋₂₀ cycloalkyl group, or a substituted or unsubstituted C₆₋₂₀ aryl group.

9. The compound according to claim 1, wherein Ra, which is identical or different, is independently hydrogen, deuterium, a substituted or unsubstituted C₁₋₂₀ alkyl group, or a substituted or unsubstituted C₃₋₁₀ aliphatic hydrocarbon ring group, or is bonded to an adjacent substituent to form a substituted or unsubstituted cyclohexane ring.

10. The compound according to claim 1, wherein Rb, which is identical or different, is independently hydrogen or deuterium.

11. The compound according to claim 1, wherein Chemical Formula 1-1 or Chemical Formula 1-2 has a deuterium substitution rate of 1% to 100%.

12. The compound according to claim 1, wherein Chemical Formula 1-1 or Chemical Formula 1-2 has a deuterium substitution rate of 40% to 99%.

13. The compound according to claim 1, wherein Chemical Formula 1-1 or Chemical Formula 1-2 is represented by any one of the following compounds:

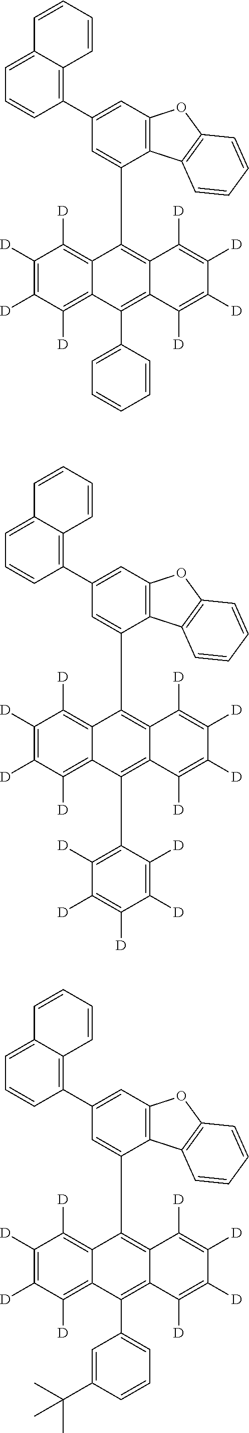 [SMILES: [2H]c1c([2H])c([2H])c2c(-c3cc(-c4cccc5ccccc45)cc4oc5ccccc5c34)c3c([2H])c([2H])c([2H])c([2H])c3c(-c3ccccc3)c2c1[2H] ; [2H]c1c([2H])c([2H])c(-c2c3c([2H])c([2H])c([2H])c([2H])c3c(-c3cc(-c4cccc5ccccc45)cc4oc5ccccc5c34)c3c([2H])c([2H])c([2H])c([2H])c23)c([2H])c1[2H] ; [2H]c1c([2H])c([2H])c2c(-c3cc(-c4cccc5ccccc45)cc4oc5ccccc5c34)c3c([2H])c([2H])c([2H])c([2H])c3c(-c3cccc(C(C)(C)C)c3)c2c1[2H]]

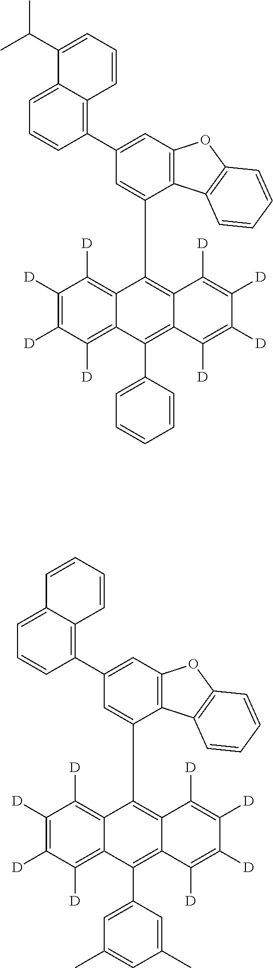 [SMILES: [2H]c1c([2H])c([2H])c2c(-c3cc(-c4cccc5c(C(C)C)cccc45)cc4oc5ccccc5c34)c3c([2H])c([2H])c([2H])c([2H])c3c(-c3ccccc3)c2c1[2H] ; [2H]c1c([2H])c([2H])c2c(-c3cc(-c4cccc5ccccc45)cc4oc5ccccc5c34)c3c([2H])c([2H])c([2H])c([2H])c3c(-c3cc(C)cc(C)c3)c2c1[2H]]

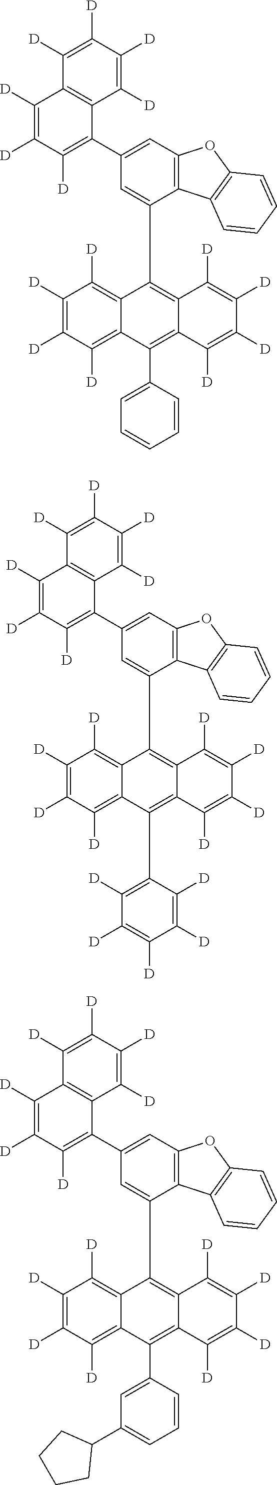

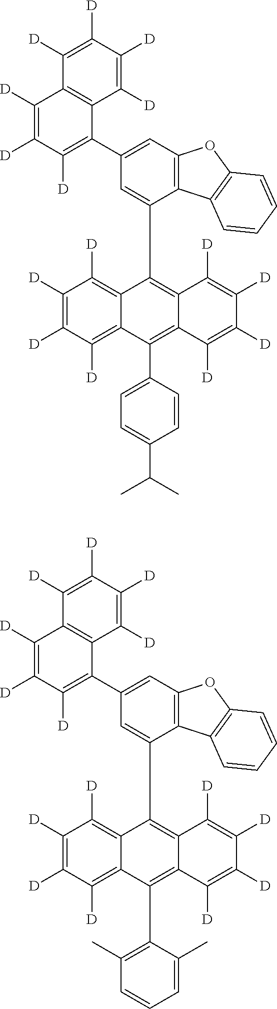 [SMILES: [2H]c1c([2H])c([2H])c2c(-c3cc(-c4c5c([2H])c([2H])c([2H])c([2H])c5c(-c5ccc(C(C)C)cc5)c5c([2H])c([2H])c([2H])c([2H])c45)c4c(c3)oc3ccccc34)c([2H])c([2H])c([2H])c2c1[2H] ; [2H]c1c([2H])c([2H])c2c(-c3cc(-c4c5c([2H])c([2H])c([2H])c([2H])c5c(-c5c(C)cccc5C)c5c([2H])c([2H])c([2H])c([2H])c45)c4c(c3)oc3ccccc34)c([2H])c([2H])c([2H])c2c1[2H]]

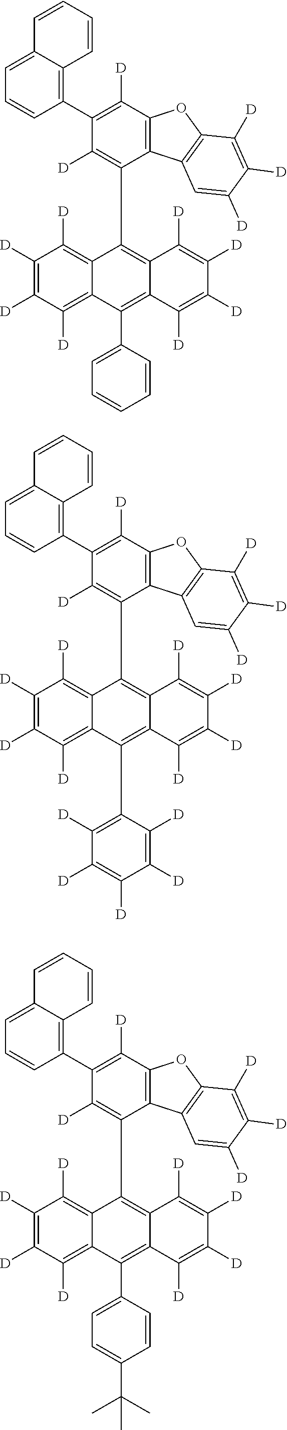 [SMILES: [2H]c1cc2c(oc3c([2H])c(-c4cccc5ccccc45)c([2H])c(-c4c5c([2H])c([2H])c([2H])c([2H])c5c(-c5ccccc5)c5c([2H])c([2H])c([2H])c([2H])c45)c32)c([2H])c1[2H] ; [2H]c1cc2c(oc3c([2H])c(-c4cccc5ccccc45)c([2H])c(-c4c5c([2H])c([2H])c([2H])c([2H])c5c(-c5c([2H])c([2H])c([2H])c([2H])c5[2H])c5c([2H])c([2H])c([2H])c([2H])c45)c32)c([2H])c1[2H] ; [2H]c1cc2c(oc3c([2H])c(-c4cccc5ccccc45)c([2H])c(-c4c5c([2H])c([2H])c([2H])c([2H])c5c(-c5ccc(C(C)(C)C)cc5)c5c([2H])c([2H])c([2H])c([2H])c45)c32)c([2H])c1[2H]]

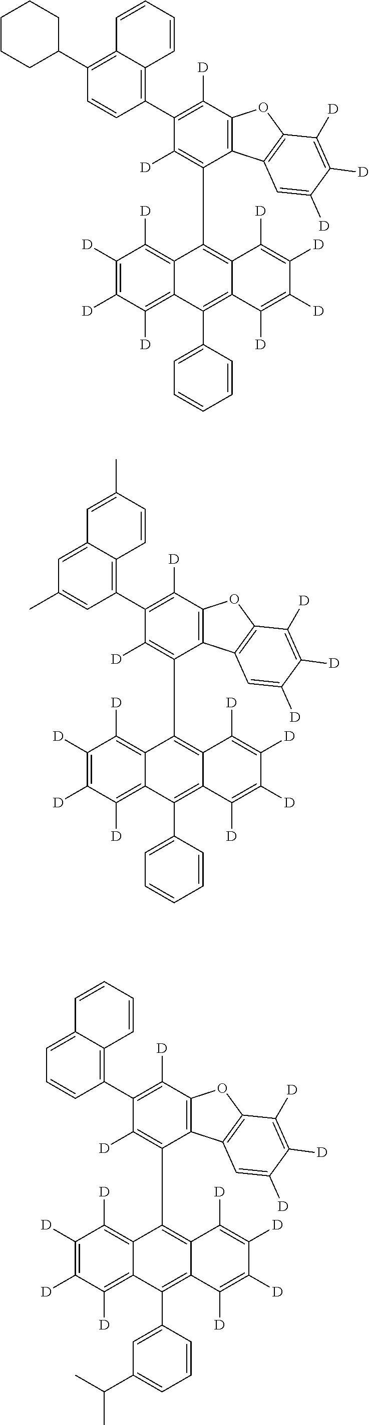 [SMILES: [2H]c1cc2c(oc3c([2H])c(-c4ccc(C5CCCCC5)c5ccccc45)c([2H])c(-c4c5c([2H])c([2H])c([2H])c([2H])c5c(-c5ccccc5)c5c([2H])c([2H])c([2H])c([2H])c45)c32)c([2H])c1[2H] ; [2H]c1cc2c(oc3c([2H])c(-c4cc(C)cc5cc(C)ccc45)c([2H])c(-c4c5c([2H])c([2H])c([2H])c([2H])c5c(-c5ccccc5)c5c([2H])c([2H])c([2H])c([2H])c45)c32)c([2H])c1[2H] ; [2H]c1cc2c(oc3c([2H])c(-c4cccc5ccccc45)c([2H])c(-c4c5c([2H])c([2H])c([2H])c([2H])c5c(-c5cccc(C(C)C)c5)c5c([2H])c([2H])c([2H])c([2H])c45)c32)c([2H])c1[2H]]

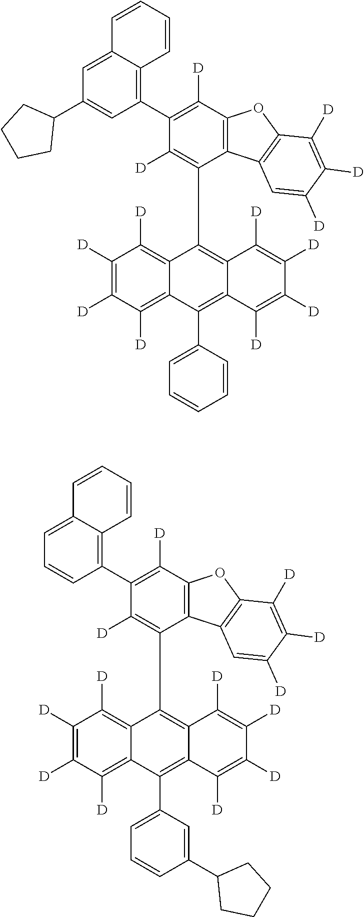 [SMILES: [2H]c1cc2c(oc3c([2H])c(-c4cc(C5CCCC5)cc5ccccc45)c([2H])c(-c4c5c([2H])c([2H])c([2H])c([2H])c5c(-c5ccccc5)c5c([2H])c([2H])c([2H])c([2H])c45)c32)c([2H])c1[2H] ; [2H]c1cc2c(oc3c([2H])c(-c4cccc5ccccc45)c([2H])c(-c4c5c([2H])c([2H])c([2H])c([2H])c5c(-c5cccc(C6CCCC6)c5)c5c([2H])c([2H])c([2H])c([2H])c45)c32)c([2H])c1[2H]]

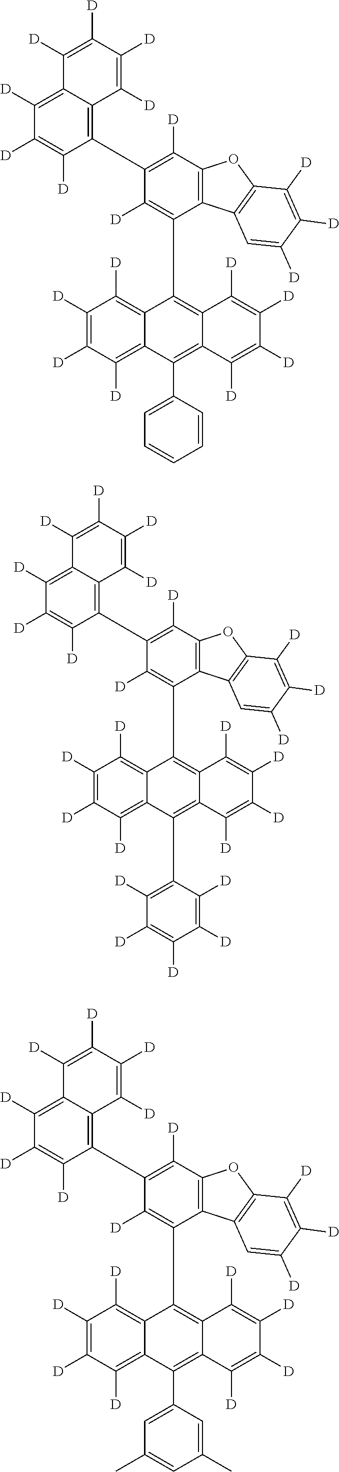

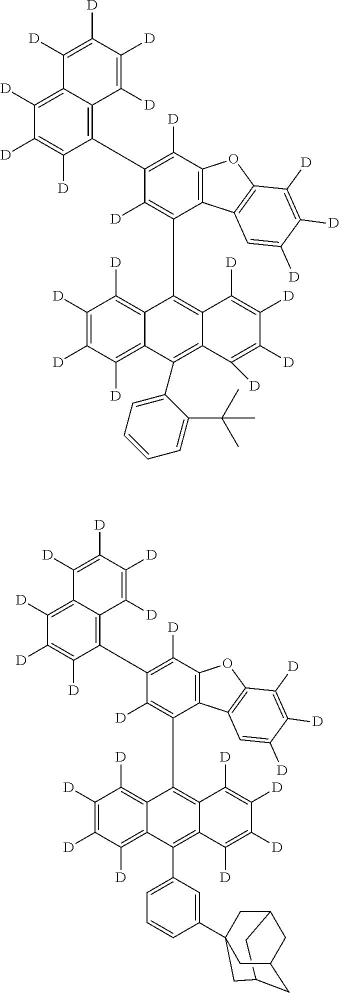 [SMILES: [2H]c1cc2c(oc3c([2H])c(-c4c([2H])c([2H])c([2H])c5c([2H])c([2H])c([2H])c([2H])c45)c([2H])c(-c4c5c([2H])c([2H])c([2H])c([2H])c5c(-c5ccccc5C(C)(C)C)c5c([2H])c([2H])c([2H])c([2H])c45)c32)c([2H])c1[2H] ; [2H]c1cc2c(oc3c([2H])c(-c4c([2H])c([2H])c([2H])c5c([2H])c([2H])c([2H])c([2H])c45)c([2H])c(-c4c5c([2H])c([2H])c([2H])c([2H])c5c(-c5cccc(C67CC8CC(CC(C8)C6)C7)c5)c5c([2H])c([2H])c([2H])c([2H])c45)c32)c([2H])c1[2H]]

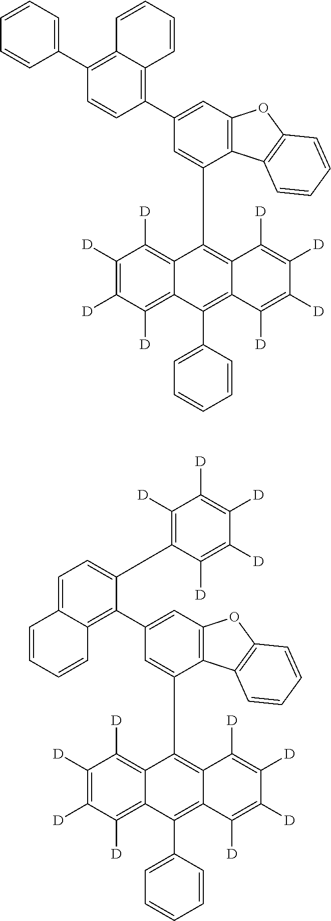 [SMILES: [2H]c1c([2H])c([2H])c2c(-c3cc(-c4ccc(-c5ccccc5)c5ccccc45)cc4oc5ccccc5c34)c3c([2H])c([2H])c([2H])c([2H])c3c(-c3ccccc3)c2c1[2H] ; [2H]c1c([2H])c([2H])c(-c2ccc3ccccc3c2-c2cc(-c3c4c([2H])c([2H])c([2H])c([2H])c4c(-c4ccccc4)c4c([2H])c([2H])c([2H])c([2H])c34)c3c(c2)oc2ccccc23)c([2H])c1[2H]]

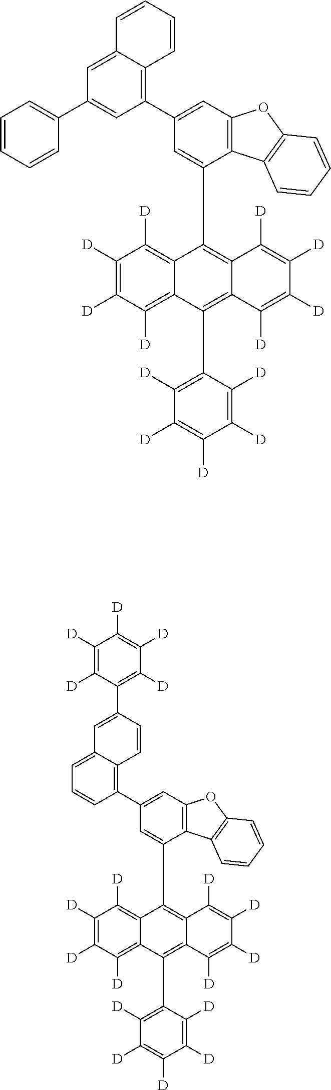 [SMILES: [2H]c1c([2H])c([2H])c(-c2c3c([2H])c([2H])c([2H])c([2H])c3c(-c3cc(-c4cc(-c5ccccc5)cc5ccccc45)cc4oc5ccccc5c34)c3c([2H])c([2H])c([2H])c([2H])c23)c([2H])c1[2H] ; [2H]c1c([2H])c([2H])c(-c2ccc3c(-c4cc(-c5c6c([2H])c([2H])c([2H])c([2H])c6c(-c6c([2H])c([2H])c([2H])c([2H])c6[2H])c6c([2H])c([2H])c([2H])c([2H])c56)c5c(c4)oc4ccccc45)cccc3c2)c([2H])c1[2H]]

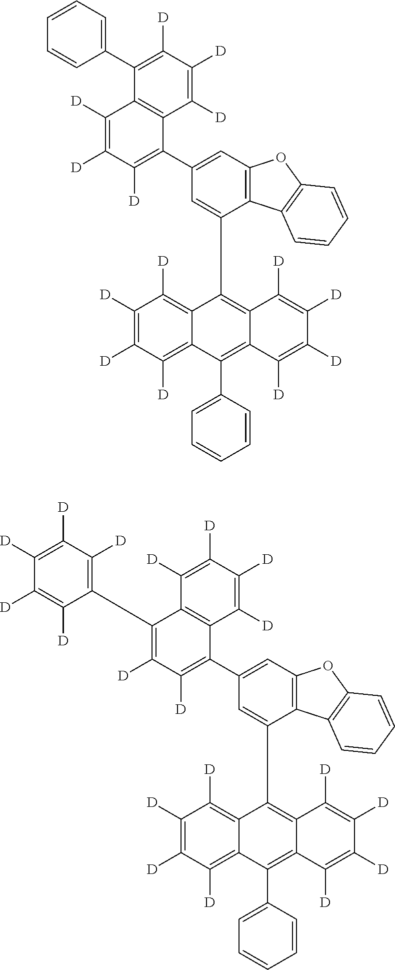 [SMILES: [2H]c1c([2H])c(-c2cc(-c3c4c([2H])c([2H])c([2H])c([2H])c4c(-c4ccccc4)c4c([2H])c([2H])c([2H])c([2H])c34)c3c(c2)oc2ccccc23)c2c([2H])c([2H])c([2H])c(-c3ccccc3)c2c1[2H] ; [2H]c1c([2H])c([2H])c(-c2c([2H])c([2H])c(-c3cc(-c4c5c([2H])c([2H])c([2H])c([2H])c5c(-c5ccccc5)c5c([2H])c([2H])c([2H])c([2H])c45)c4c(c3)oc3ccccc34)c3c([2H])c([2H])c([2H])c([2H])c23)c([2H])c1[2H]]

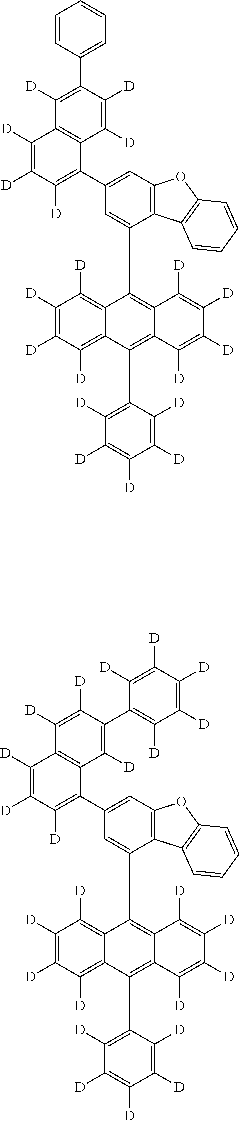 [SMILES: [2H]c1c([2H])c([2H])c(-c2c3c([2H])c([2H])c([2H])c([2H])c3c(-c3cc(-c4c([2H])c([2H])c([2H])c5c([2H])c(-c6ccccc6)c([2H])c([2H])c45)cc4oc5ccccc5c34)c3c([2H])c([2H])c([2H])c([2H])c23)c([2H])c1[2H] ; [2H]c1c([2H])c([2H])c(-c2c([2H])c([2H])c3c([2H])c([2H])c([2H])c(-c4cc(-c5c6c([2H])c([2H])c([2H])c([2H])c6c(-c6c([2H])c([2H])c([2H])c([2H])c6[2H])c6c([2H])c([2H])c([2H])c([2H])c56)c5c(c4)oc4ccccc45)c3c2[2H])c([2H])c1[2H]]

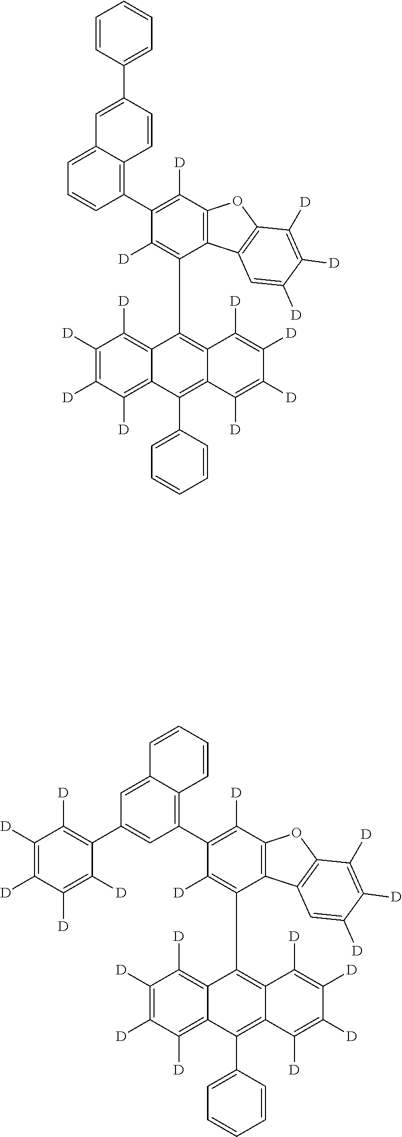 [SMILES: [2H]c1cc2c(oc3c([2H])c(-c4cccc5cc(-c6ccccc6)ccc45)c([2H])c(-c4c5c([2H])c([2H])c([2H])c([2H])c5c(-c5ccccc5)c5c([2H])c([2H])c([2H])c([2H])c45)c32)c([2H])c1[2H] ; [2H]c1cc2c(oc3c([2H])c(-c4cc(-c5c([2H])c([2H])c([2H])c([2H])c5[2H])cc5ccccc45)c([2H])c(-c4c5c([2H])c([2H])c([2H])c([2H])c5c(-c5ccccc5)c5c([2H])c([2H])c([2H])c([2H])c45)c32)c([2H])c1[2H]]

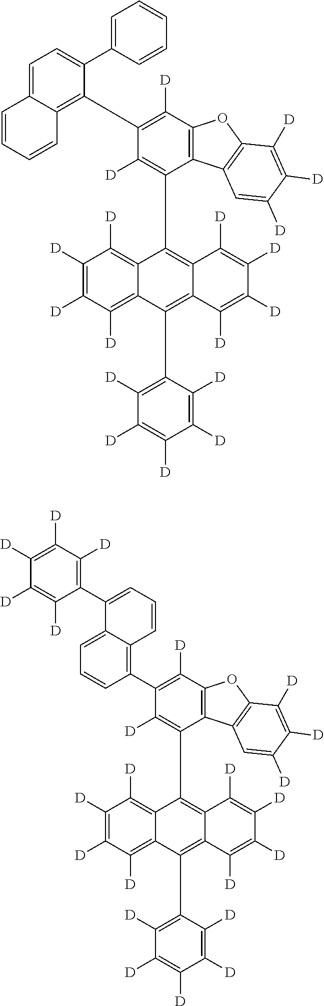 [SMILES: [2H]c1cc2c(oc3c([2H])c(-c4c(-c5ccccc5)ccc5ccccc45)c([2H])c(-c4c5c([2H])c([2H])c([2H])c([2H])c5c(-c5c([2H])c([2H])c([2H])c([2H])c5[2H])c5c([2H])c([2H])c([2H])c([2H])c45)c32)c([2H])c1[2H] ; [2H]c1cc2c(oc3c([2H])c(-c4cccc5c(-c6c([2H])c([2H])c([2H])c([2H])c6[2H])cccc45)c([2H])c(-c4c5c([2H])c([2H])c([2H])c([2H])c5c(-c5c([2H])c([2H])c([2H])c([2H])c5[2H])c5c([2H])c([2H])c([2H])c([2H])c45)c32)c([2H])c1[2H]]

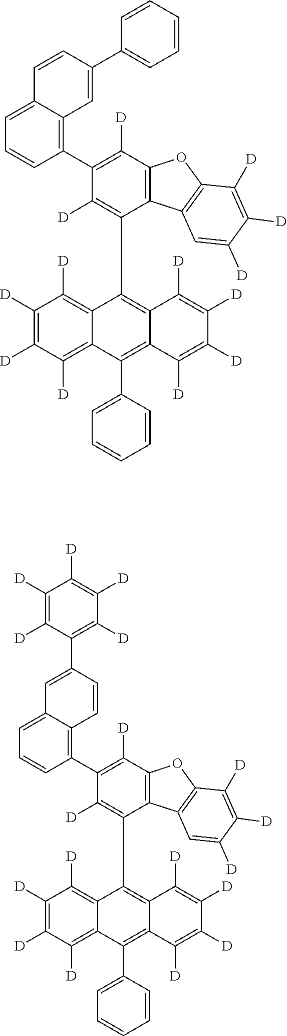 [SMILES: [2H]c1cc2c(oc3c([2H])c(-c4cccc5ccc(-c6ccccc6)cc45)c([2H])c(-c4c5c([2H])c([2H])c([2H])c([2H])c5c(-c5ccccc5)c5c([2H])c([2H])c([2H])c([2H])c45)c32)c([2H])c1[2H] ; [2H]c1cc2c(oc3c([2H])c(-c4cccc5cc(-c6c([2H])c([2H])c([2H])c([2H])c6[2H])ccc45)c([2H])c(-c4c5c([2H])c([2H])c([2H])c([2H])c5c(-c5ccccc5)c5c([2H])c([2H])c([2H])c([2H])c45)c32)c([2H])c1[2H]]

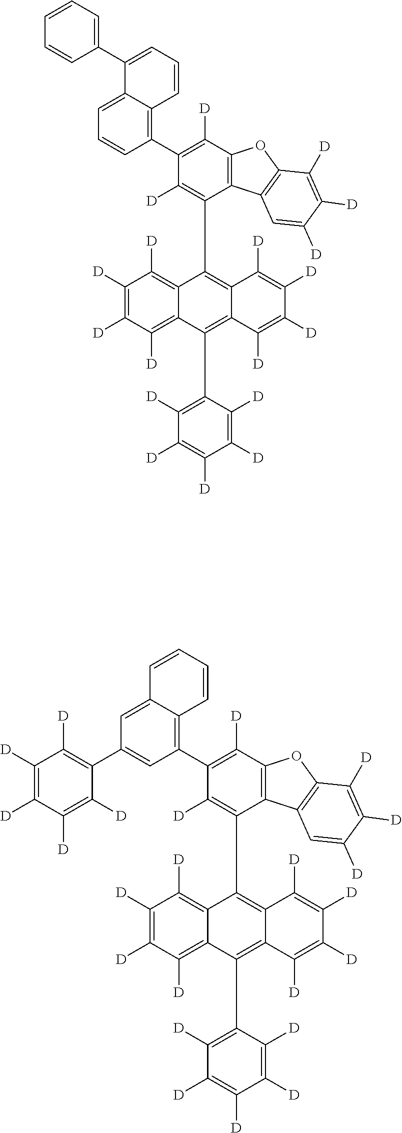 [SMILES: [2H]c1cc2c(oc3c([2H])c(-c4cccc5c(-c6ccccc6)cccc45)c([2H])c(-c4c5c([2H])c([2H])c([2H])c([2H])c5c(-c5c([2H])c([2H])c([2H])c([2H])c5[2H])c5c([2H])c([2H])c([2H])c([2H])c45)c32)c([2H])c1[2H] ; [2H]c1cc2c(oc3c([2H])c(-c4cc(-c5c([2H])c([2H])c([2H])c([2H])c5[2H])cc5ccccc45)c([2H])c(-c4c5c([2H])c([2H])c([2H])c([2H])c5c(-c5c([2H])c([2H])c([2H])c([2H])c5[2H])c5c([2H])c([2H])c([2H])c([2H])c45)c32)c([2H])c1[2H]]

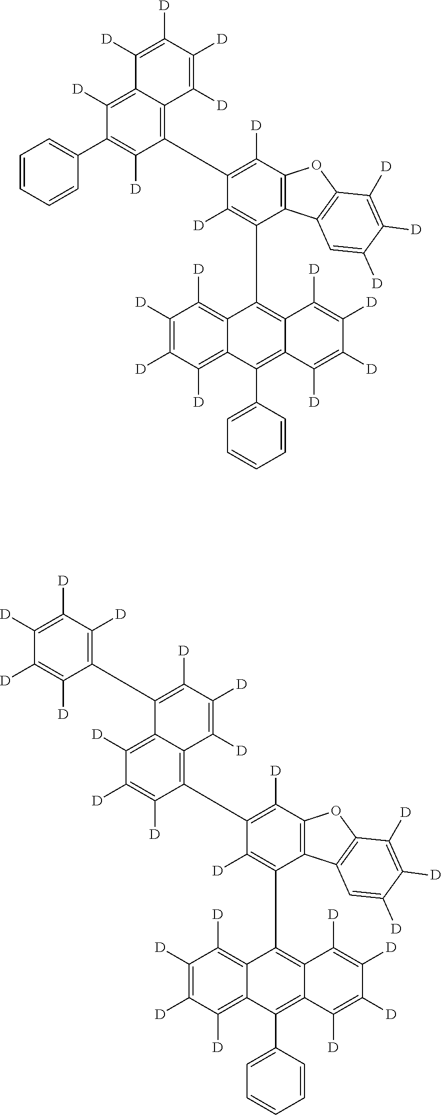 [SMILES: [2H]c1cc2c(oc3c([2H])c(-c4c([2H])c(-c5ccccc5)c([2H])c5c([2H])c([2H])c([2H])c([2H])c45)c([2H])c(-c4c5c([2H])c([2H])c([2H])c([2H])c5c(-c5ccccc5)c5c([2H])c([2H])c([2H])c([2H])c45)c32)c([2H])c1[2H] ; [2H]c1cc2c(oc3c([2H])c(-c4c([2H])c([2H])c([2H])c5c(-c6c([2H])c([2H])c([2H])c([2H])c6[2H])c([2H])c([2H])c([2H])c45)c([2H])c(-c4c5c([2H])c([2H])c([2H])c([2H])c5c(-c5ccccc5)c5c([2H])c([2H])c([2H])c([2H])c45)c32)c([2H])c1[2H]]

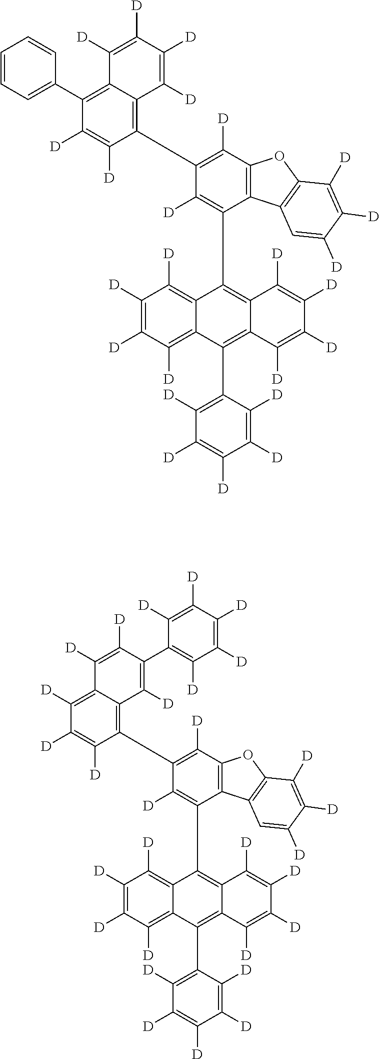 [SMILES: [2H]c1cc2c(oc3c([2H])c(-c4c([2H])c([2H])c(-c5ccccc5)c5c([2H])c([2H])c([2H])c([2H])c45)c([2H])c(-c4c5c([2H])c([2H])c([2H])c([2H])c5c(-c5c([2H])c([2H])c([2H])c([2H])c5[2H])c5c([2H])c([2H])c([2H])c([2H])c45)c32)c([2H])c1[2H] ; [2H]c1cc2c(oc3c([2H])c(-c4c([2H])c([2H])c([2H])c5c([2H])c([2H])c(-c6c([2H])c([2H])c([2H])c([2H])c6[2H])c([2H])c45)c([2H])c(-c4c5c([2H])c([2H])c([2H])c([2H])c5c(-c5c([2H])c([2H])c([2H])c([2H])c5[2H])c5c([2H])c([2H])c([2H])c([2H])c45)c32)c([2H])c1[2H]]

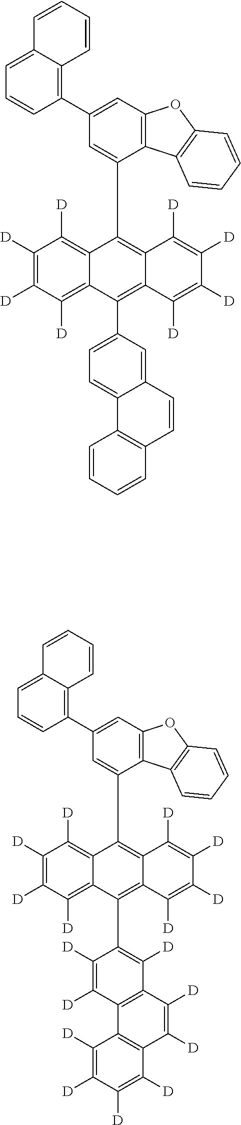 [SMILES: [2H]c1c([2H])c([2H])c2c(-c3cc(-c4cccc5ccccc45)cc4oc5ccccc5c34)c3c([2H])c([2H])c([2H])c([2H])c3c(-c3ccc4c(ccc5ccccc54)c3)c2c1[2H] ; [2H]c1c([2H])c([2H])c2c(-c3cc(-c4cccc5ccccc45)cc4oc5ccccc5c34)c3c([2H])c([2H])c([2H])c([2H])c3c(-c3c([2H])c([2H])c4c(c3[2H])c([2H])c([2H])c3c([2H])c([2H])c([2H])c([2H])c34)c2c1[2H]]

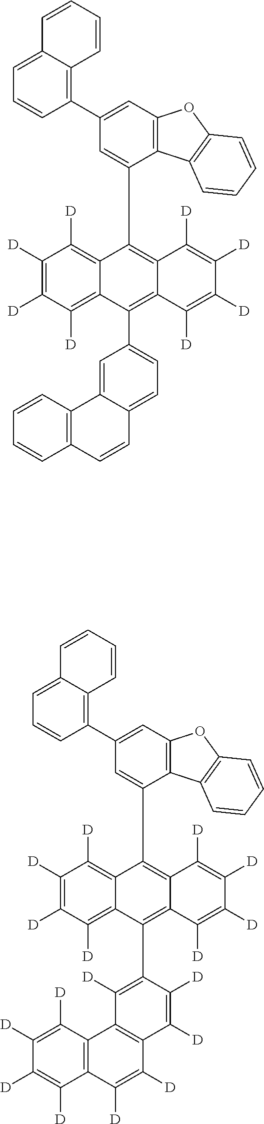 [SMILES: [2H]c1c([2H])c([2H])c2c(-c3cc(-c4cccc5ccccc45)cc4oc5ccccc5c34)c3c([2H])c([2H])c([2H])c([2H])c3c(-c3ccc4ccc5ccccc5c4c3)c2c1[2H] ; [2H]c1c([2H])c([2H])c2c(-c3cc(-c4cccc5ccccc45)cc4oc5ccccc5c34)c3c([2H])c([2H])c([2H])c([2H])c3c(-c3c([2H])c([2H])c4c([2H])c([2H])c5c([2H])c([2H])c([2H])c([2H])c5c4c3[2H])c2c1[2H]]

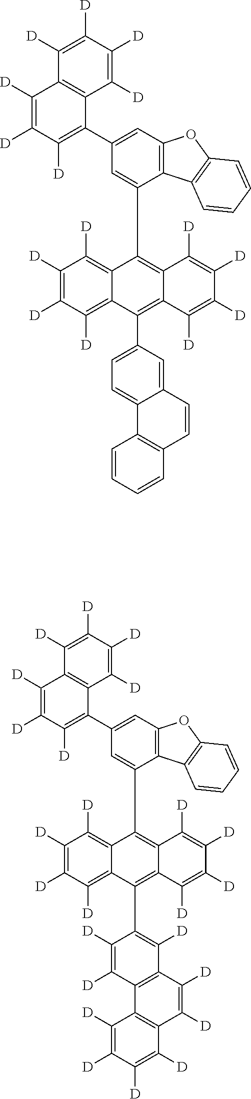 [SMILES: [2H]c1c([2H])c([2H])c2c(-c3cc(-c4c5c([2H])c([2H])c([2H])c([2H])c5c(-c5ccc6c(ccc7ccccc76)c5)c5c([2H])c([2H])c([2H])c([2H])c45)c4c(c3)oc3ccccc34)c([2H])c([2H])c([2H])c2c1[2H] ; [2H]c1c([2H])c([2H])c2c(-c3cc(-c4c5c([2H])c([2H])c([2H])c([2H])c5c(-c5c([2H])c([2H])c6c(c5[2H])c([2H])c([2H])c5c([2H])c([2H])c([2H])c([2H])c56)c5c([2H])c([2H])c([2H])c([2H])c45)c4c(c3)oc3ccccc34)c([2H])c([2H])c([2H])c2c1[2H]]

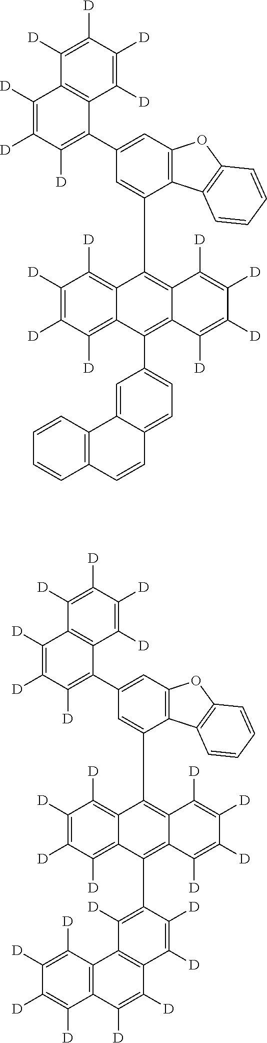 [SMILES: [2H]c1c([2H])c([2H])c2c(-c3cc(-c4c5c([2H])c([2H])c([2H])c([2H])c5c(-c5ccc6ccc7ccccc7c6c5)c5c([2H])c([2H])c([2H])c([2H])c45)c4c(c3)oc3ccccc34)c([2H])c([2H])c([2H])c2c1[2H] ; [2H]c1c([2H])c([2H])c2c(-c3cc(-c4c5c([2H])c([2H])c([2H])c([2H])c5c(-c5c([2H])c([2H])c6c([2H])c([2H])c7c([2H])c([2H])c([2H])c([2H])c7c6c5[2H])c5c([2H])c([2H])c([2H])c([2H])c45)c4c(c3)oc3ccccc34)c([2H])c([2H])c([2H])c2c1[2H]]

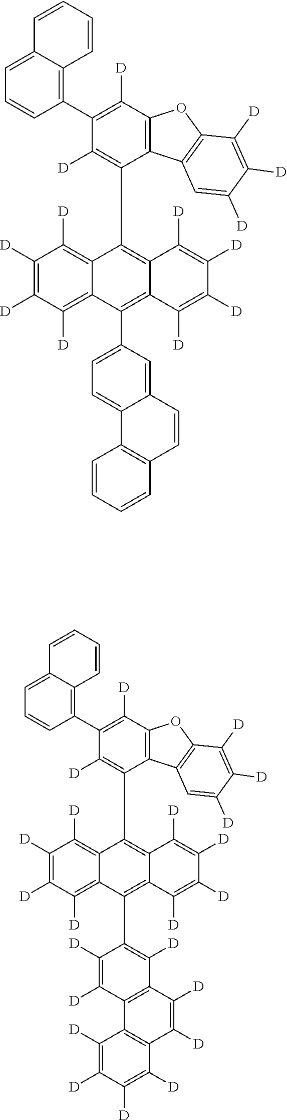 [SMILES: [2H]c1cc2c(oc3c([2H])c(-c4cccc5ccccc45)c([2H])c(-c4c5c([2H])c([2H])c([2H])c([2H])c5c(-c5ccc6c(ccc7ccccc76)c5)c5c([2H])c([2H])c([2H])c([2H])c45)c32)c([2H])c1[2H] ; [2H]c1cc2c(oc3c([2H])c(-c4cccc5ccccc45)c([2H])c(-c4c5c([2H])c([2H])c([2H])c([2H])c5c(-c5c([2H])c([2H])c6c(c5[2H])c([2H])c([2H])c5c([2H])c([2H])c([2H])c([2H])c56)c5c([2H])c([2H])c([2H])c([2H])c45)c32)c([2H])c1[2H]]

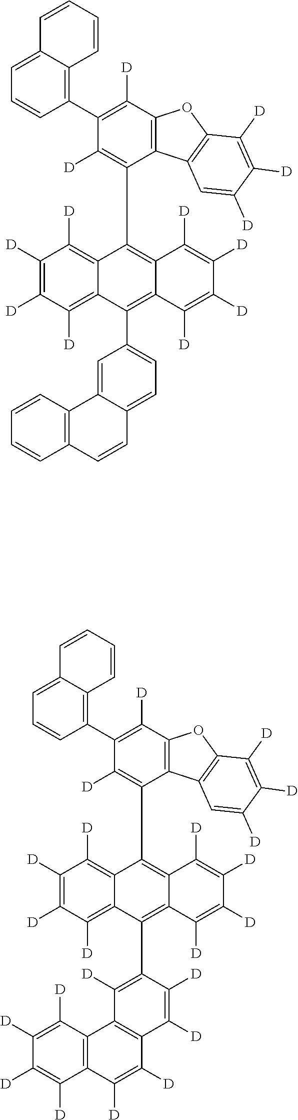 [SMILES: [2H]c1cc2c(oc3c([2H])c(-c4cccc5ccccc45)c([2H])c(-c4c5c([2H])c([2H])c([2H])c([2H])c5c(-c5ccc6ccc7ccccc7c6c5)c5c([2H])c([2H])c([2H])c([2H])c45)c32)c([2H])c1[2H] ; [2H]c1cc2c(oc3c([2H])c(-c4cccc5ccccc45)c([2H])c(-c4c5c([2H])c([2H])c([2H])c([2H])c5c(-c5c([2H])c([2H])c6c([2H])c([2H])c7c([2H])c([2H])c([2H])c([2H])c7c6c5[2H])c5c([2H])c([2H])c([2H])c([2H])c45)c32)c([2H])c1[2H]]

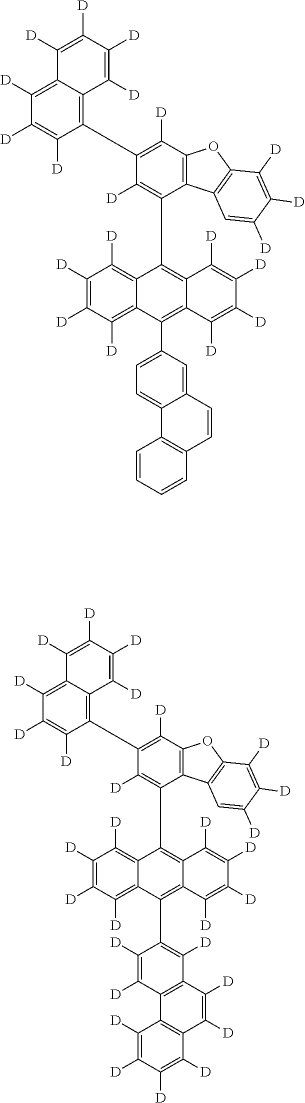 [SMILES: [2H]c1cc2c(oc3c([2H])c(-c4c([2H])c([2H])c([2H])c5c([2H])c([2H])c([2H])c([2H])c45)c([2H])c(-c4c5c([2H])c([2H])c([2H])c([2H])c5c(-c5ccc6c(ccc7ccccc76)c5)c5c([2H])c([2H])c([2H])c([2H])c45)c32)c([2H])c1[2H] ; [2H]c1cc2c(oc3c([2H])c(-c4c([2H])c([2H])c([2H])c5c([2H])c([2H])c([2H])c([2H])c45)c([2H])c(-c4c5c([2H])c([2H])c([2H])c([2H])c5c(-c5c([2H])c([2H])c6c(c5[2H])c([2H])c([2H])c5c([2H])c([2H])c([2H])c([2H])c56)c5c([2H])c([2H])c([2H])c([2H])c45)c32)c([2H])c1[2H]]

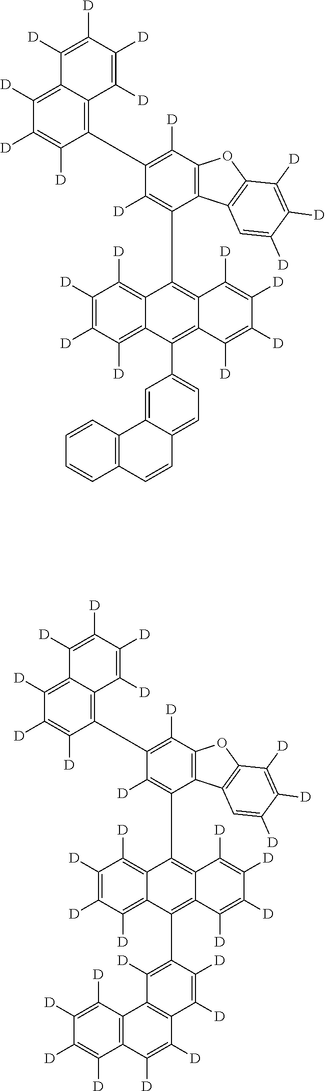 [SMILES: [2H]c1cc2c(oc3c([2H])c(-c4c([2H])c([2H])c([2H])c5c([2H])c([2H])c([2H])c([2H])c45)c([2H])c(-c4c5c([2H])c([2H])c([2H])c([2H])c5c(-c5ccc6ccc7ccccc7c6c5)c5c([2H])c([2H])c([2H])c([2H])c45)c32)c([2H])c1[2H] ; [2H]c1cc2c(oc3c([2H])c(-c4c([2H])c([2H])c([2H])c5c([2H])c([2H])c([2H])c([2H])c45)c([2H])c(-c4c5c([2H])c([2H])c([2H])c([2H])c5c(-c5c([2H])c([2H])c6c([2H])c([2H])c7c([2H])c([2H])c([2H])c([2H])c7c6c5[2H])c5c([2H])c([2H])c([2H])c([2H])c45)c32)c([2H])c1[2H]]

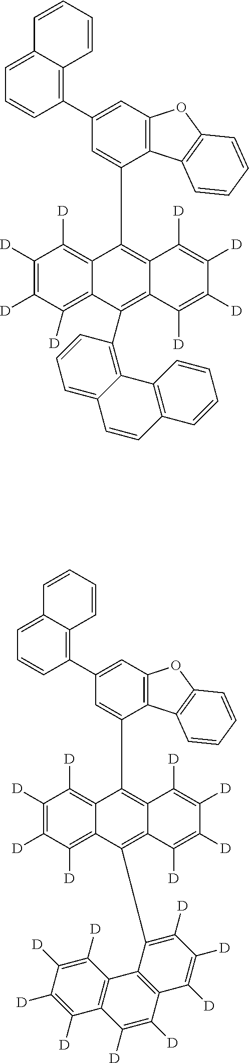 [SMILES: [2H]c1c([2H])c([2H])c2c(-c3cc(-c4cccc5ccccc45)cc4oc5ccccc5c34)c3c([2H])c([2H])c([2H])c([2H])c3c(-c3cccc4ccc5ccccc5c34)c2c1[2H] ; [2H]c1c([2H])c([2H])c2c(-c3c([2H])c([2H])c([2H])c4c([2H])c([2H])c5c([2H])c([2H])c([2H])c([2H])c5c34)c3c([2H])c([2H])c([2H])c([2H])c3c(-c3cc(-c4cccc5ccccc45)cc4oc5ccccc5c34)c2c1[2H]]

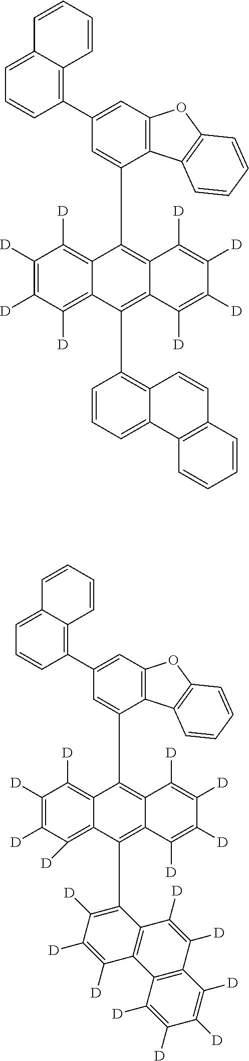 [SMILES: [2H]c1c([2H])c([2H])c2c(-c3cc(-c4cccc5ccccc45)cc4oc5ccccc5c34)c3c([2H])c([2H])c([2H])c([2H])c3c(-c3cccc4c3ccc3ccccc34)c2c1[2H] ; [2H]c1c([2H])c([2H])c2c(-c3cc(-c4cccc5ccccc45)cc4oc5ccccc5c34)c3c([2H])c([2H])c([2H])c([2H])c3c(-c3c([2H])c([2H])c([2H])c4c3c([2H])c([2H])c3c([2H])c([2H])c([2H])c([2H])c34)c2c1[2H]]

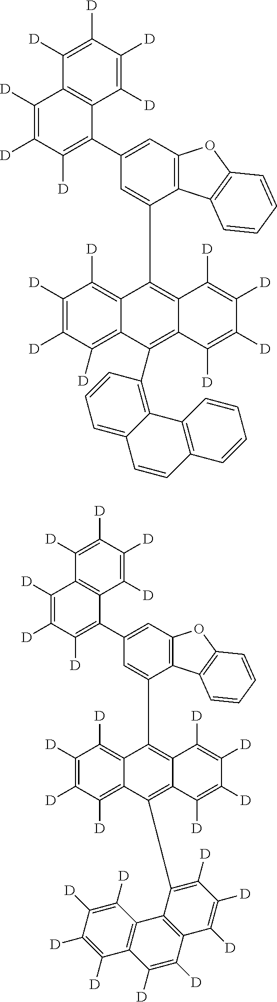 [SMILES: [2H]c1c([2H])c([2H])c2c(-c3cc(-c4c5c([2H])c([2H])c([2H])c([2H])c5c(-c5cccc6ccc7ccccc7c56)c5c([2H])c([2H])c([2H])c([2H])c45)c4c(c3)oc3ccccc34)c([2H])c([2H])c([2H])c2c1[2H] ; [2H]c1c([2H])c([2H])c2c(-c3cc(-c4c5c([2H])c([2H])c([2H])c([2H])c5c(-c5c([2H])c([2H])c([2H])c6c([2H])c([2H])c7c([2H])c([2H])c([2H])c([2H])c7c56)c5c([2H])c([2H])c([2H])c([2H])c45)c4c(c3)oc3ccccc34)c([2H])c([2H])c([2H])c2c1[2H]]

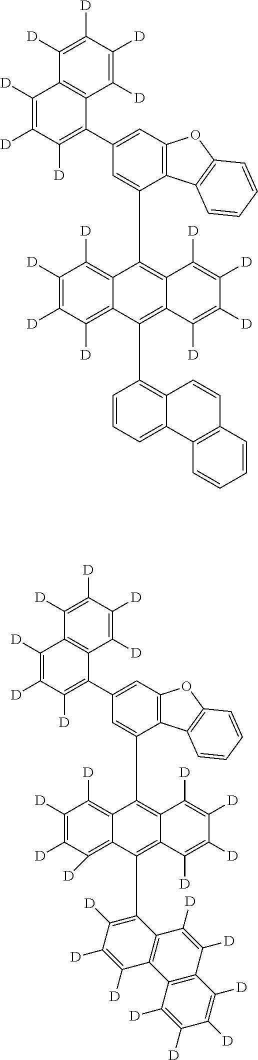 [SMILES: [2H]c1c([2H])c([2H])c2c(-c3cc(-c4c5c([2H])c([2H])c([2H])c([2H])c5c(-c5cccc6c5ccc5ccccc56)c5c([2H])c([2H])c([2H])c([2H])c45)c4c(c3)oc3ccccc34)c([2H])c([2H])c([2H])c2c1[2H] ; [2H]c1c([2H])c([2H])c2c(-c3cc(-c4c5c([2H])c([2H])c([2H])c([2H])c5c(-c5c([2H])c([2H])c([2H])c6c5c([2H])c([2H])c5c([2H])c([2H])c([2H])c([2H])c56)c5c([2H])c([2H])c([2H])c([2H])c45)c4c(c3)oc3ccccc34)c([2H])c([2H])c([2H])c2c1[2H]]

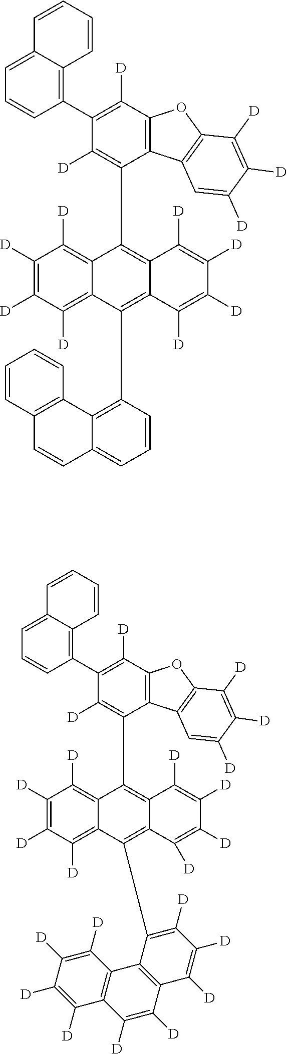 [SMILES: [2H]c1cc2c(oc3c([2H])c(-c4cccc5ccccc45)c([2H])c(-c4c5c([2H])c([2H])c([2H])c([2H])c5c(-c5cccc6ccc7ccccc7c56)c5c([2H])c([2H])c([2H])c([2H])c45)c32)c([2H])c1[2H] ; [2H]c1cc2c(oc3c([2H])c(-c4cccc5ccccc45)c([2H])c(-c4c5c([2H])c([2H])c([2H])c([2H])c5c(-c5c([2H])c([2H])c([2H])c6c([2H])c([2H])c7c([2H])c([2H])c([2H])c([2H])c7c56)c5c([2H])c([2H])c([2H])c([2H])c45)c32)c([2H])c1[2H]]

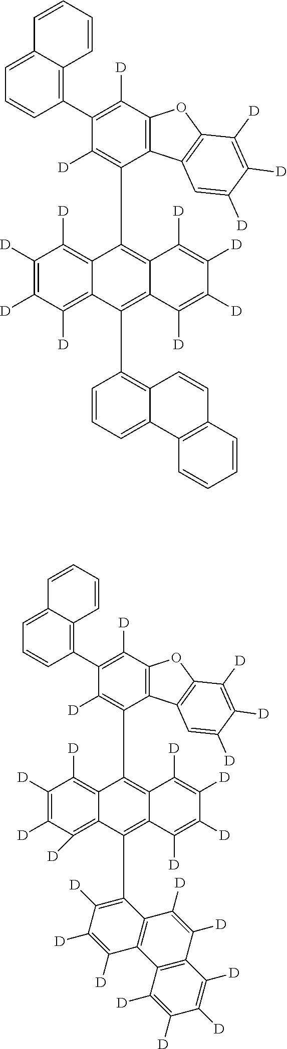 [SMILES: [2H]c1cc2c(oc3c([2H])c(-c4cccc5ccccc45)c([2H])c(-c4c5c([2H])c([2H])c([2H])c([2H])c5c(-c5cccc6c5ccc5ccccc56)c5c([2H])c([2H])c([2H])c([2H])c45)c32)c([2H])c1[2H] ; [2H]c1cc2c(oc3c([2H])c(-c4cccc5ccccc45)c([2H])c(-c4c5c([2H])c([2H])c([2H])c([2H])c5c(-c5c([2H])c([2H])c([2H])c6c5c([2H])c([2H])c5c([2H])c([2H])c([2H])c([2H])c56)c5c([2H])c([2H])c([2H])c([2H])c45)c32)c([2H])c1[2H]]

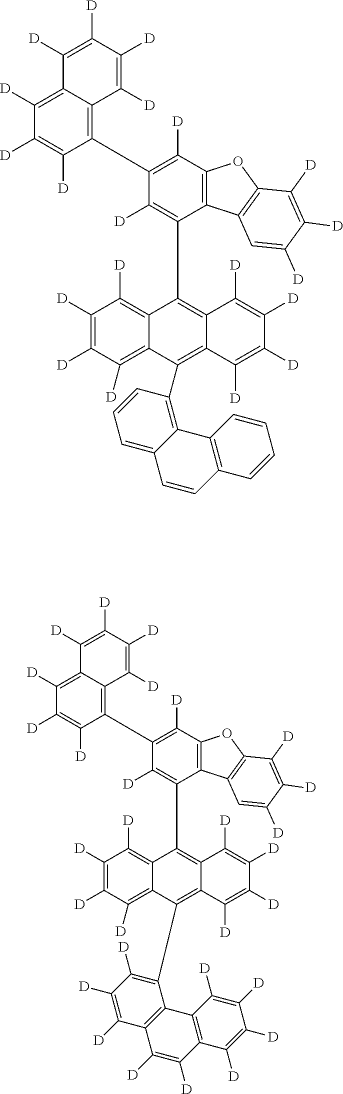 [SMILES: [2H]c1cc2c(oc3c([2H])c(-c4c([2H])c([2H])c([2H])c5c([2H])c([2H])c([2H])c([2H])c45)c([2H])c(-c4c5c([2H])c([2H])c([2H])c([2H])c5c(-c5cccc6ccc7ccccc7c56)c5c([2H])c([2H])c([2H])c([2H])c45)c32)c([2H])c1[2H] ; [2H]c1cc2c(oc3c([2H])c(-c4c([2H])c([2H])c([2H])c5c([2H])c([2H])c([2H])c([2H])c45)c([2H])c(-c4c5c([2H])c([2H])c([2H])c([2H])c5c(-c5c([2H])c([2H])c([2H])c6c([2H])c([2H])c7c([2H])c([2H])c([2H])c([2H])c7c56)c5c([2H])c([2H])c([2H])c([2H])c45)c32)c([2H])c1[2H]]

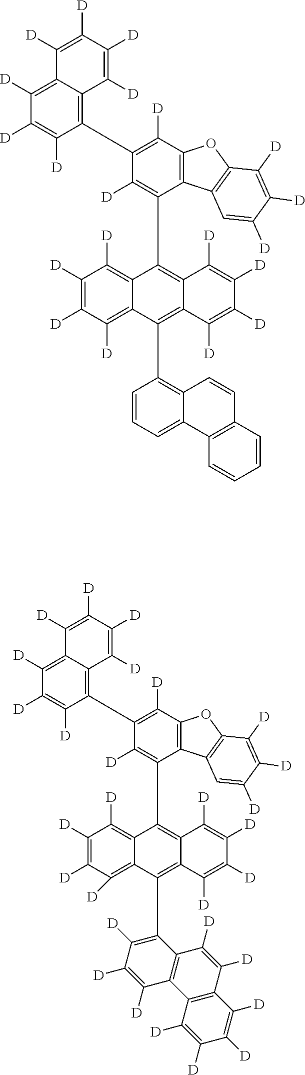 [SMILES: [2H]c1cc2c(oc3c([2H])c(-c4c([2H])c([2H])c([2H])c5c([2H])c([2H])c([2H])c([2H])c45)c([2H])c(-c4c5c([2H])c([2H])c([2H])c([2H])c5c(-c5cccc6c5ccc5ccccc56)c5c([2H])c([2H])c([2H])c([2H])c45)c32)c([2H])c1[2H] ; [2H]c1cc2c(oc3c([2H])c(-c4c([2H])c([2H])c([2H])c5c([2H])c([2H])c([2H])c([2H])c45)c([2H])c(-c4c5c([2H])c([2H])c([2H])c([2H])c5c(-c5c([2H])c([2H])c([2H])c6c5c([2H])c([2H])c5c([2H])c([2H])c([2H])c([2H])c56)c5c([2H])c([2H])c([2H])c([2H])c45)c32)c([2H])c1[2H]]

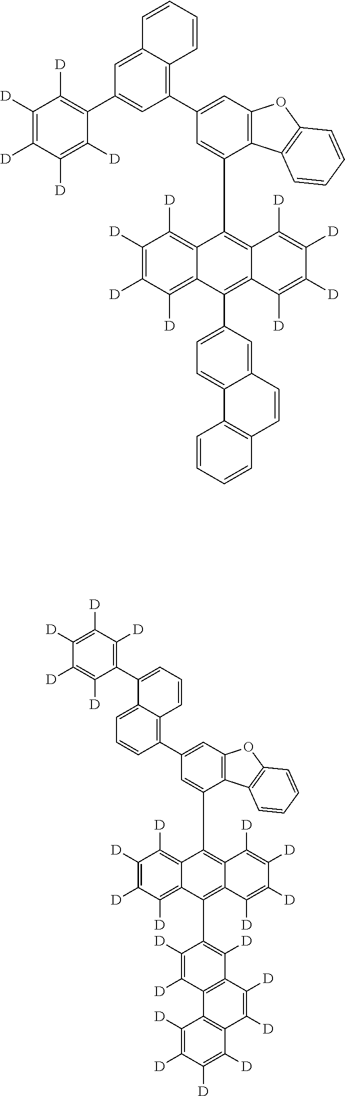 [SMILES: [2H]c1c([2H])c([2H])c(-c2cc(-c3cc(-c4c5c([2H])c([2H])c([2H])c([2H])c5c(-c5ccc6c(ccc7ccccc76)c5)c5c([2H])c([2H])c([2H])c([2H])c45)c4c(c3)oc3ccccc34)c3ccccc3c2)c([2H])c1[2H] ; [2H]c1c([2H])c([2H])c(-c2cccc3c(-c4cc(-c5c6c([2H])c([2H])c([2H])c([2H])c6c(-c6c([2H])c([2H])c7c(c6[2H])c([2H])c([2H])c6c([2H])c([2H])c([2H])c([2H])c67)c6c([2H])c([2H])c([2H])c([2H])c56)c5c(c4)oc4ccccc45)cccc23)c([2H])c1[2H]]

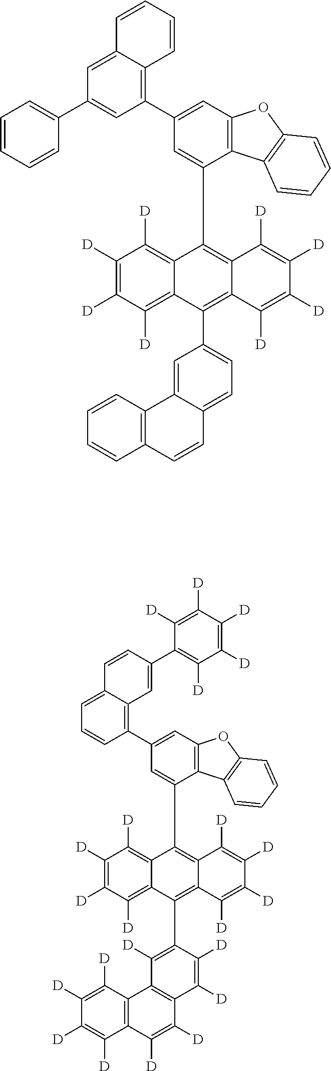 [SMILES: [2H]c1c([2H])c([2H])c2c(-c3cc(-c4cc(-c5ccccc5)cc5ccccc45)cc4oc5ccccc5c34)c3c([2H])c([2H])c([2H])c([2H])c3c(-c3ccc4ccc5ccccc5c4c3)c2c1[2H] ; [2H]c1c([2H])c([2H])c(-c2ccc3cccc(-c4cc(-c5c6c([2H])c([2H])c([2H])c([2H])c6c(-c6c([2H])c([2H])c7c([2H])c([2H])c8c([2H])c([2H])c([2H])c([2H])c8c7c6[2H])c6c([2H])c([2H])c([2H])c([2H])c56)c5c(c4)oc4ccccc45)c3c2)c([2H])c1[2H]]

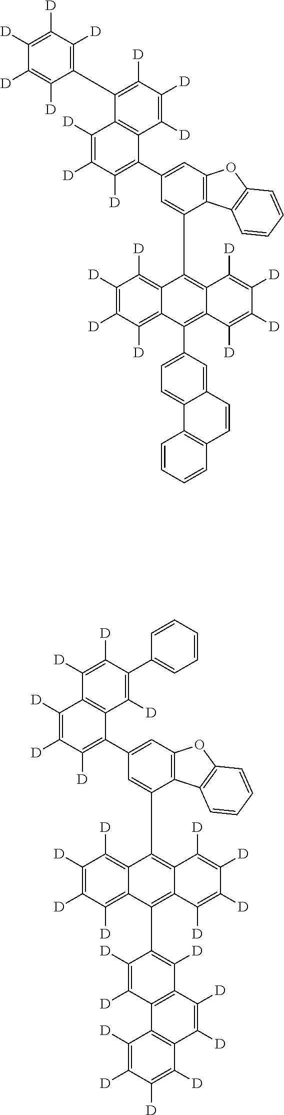 [SMILES: [2H]c1c([2H])c([2H])c(-c2c([2H])c([2H])c([2H])c3c(-c4cc(-c5c6c([2H])c([2H])c([2H])c([2H])c6c(-c6ccc7c(ccc8ccccc87)c6)c6c([2H])c([2H])c([2H])c([2H])c56)c5c(c4)oc4ccccc45)c([2H])c([2H])c([2H])c23)c([2H])c1[2H] ; [2H]c1c([2H])c(-c2cc(-c3c4c([2H])c([2H])c([2H])c([2H])c4c(-c4c([2H])c([2H])c5c(c4[2H])c([2H])c([2H])c4c([2H])c([2H])c([2H])c([2H])c45)c4c([2H])c([2H])c([2H])c([2H])c34)c3c(c2)oc2ccccc23)c2c([2H])c(-c3ccccc3)c([2H])c([2H])c2c1[2H]]

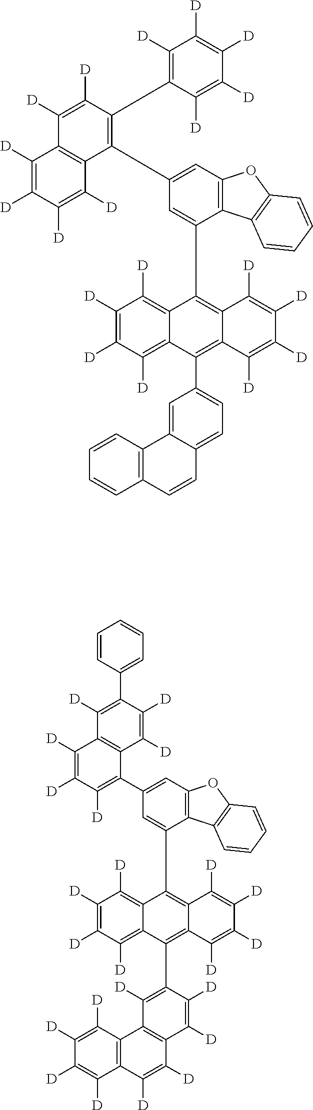 [SMILES: [2H]c1c([2H])c([2H])c(-c2c([2H])c([2H])c3c([2H])c([2H])c([2H])c([2H])c3c2-c2cc(-c3c4c([2H])c([2H])c([2H])c([2H])c4c(-c4ccc5ccc6ccccc6c5c4)c4c([2H])c([2H])c([2H])c([2H])c34)c3c(c2)oc2ccccc23)c([2H])c1[2H] ; [2H]c1c([2H])c(-c2cc(-c3c4c([2H])c([2H])c([2H])c([2H])c4c(-c4c([2H])c([2H])c5c([2H])c([2H])c6c([2H])c([2H])c([2H])c([2H])c6c5c4[2H])c4c([2H])c([2H])c([2H])c([2H])c34)c3c(c2)oc2ccccc23)c2c([2H])c([2H])c(-c3ccccc3)c([2H])c2c1[2H]]

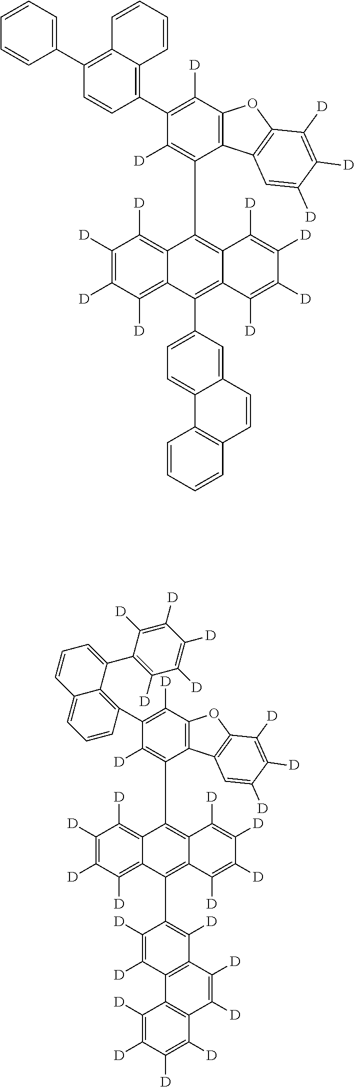 [SMILES: [2H]c1cc2c(oc3c([2H])c(-c4ccc(-c5ccccc5)c5ccccc45)c([2H])c(-c4c5c([2H])c([2H])c([2H])c([2H])c5c(-c5ccc6c(ccc7ccccc76)c5)c5c([2H])c([2H])c([2H])c([2H])c45)c32)c([2H])c1[2H] ; [2H]c1cc2c(oc3c([2H])c(-c4cccc5cccc(-c6c([2H])c([2H])c([2H])c([2H])c6[2H])c45)c([2H])c(-c4c5c([2H])c([2H])c([2H])c([2H])c5c(-c5c([2H])c([2H])c6c(c5[2H])c([2H])c([2H])c5c([2H])c([2H])c([2H])c([2H])c56)c5c([2H])c([2H])c([2H])c([2H])c45)c32)c([2H])c1[2H]]

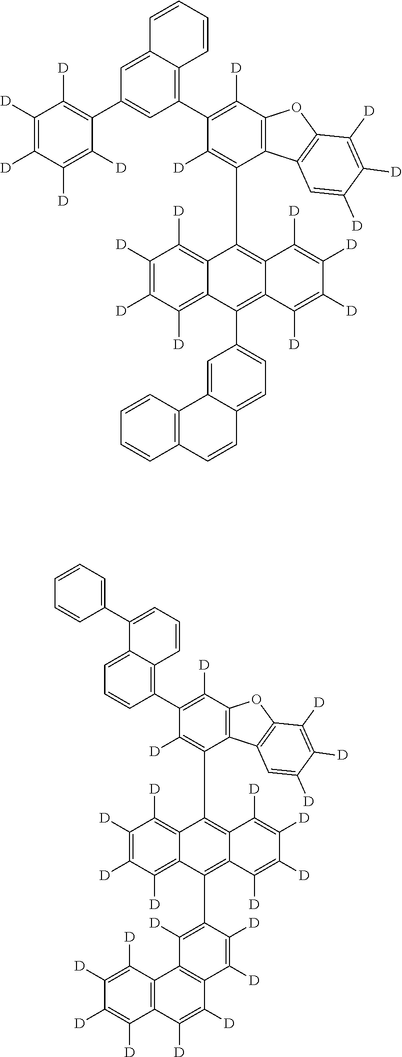 [SMILES: [2H]c1cc2c(oc3c([2H])c(-c4cc(-c5c([2H])c([2H])c([2H])c([2H])c5[2H])cc5ccccc45)c([2H])c(-c4c5c([2H])c([2H])c([2H])c([2H])c5c(-c5ccc6ccc7ccccc7c6c5)c5c([2H])c([2H])c([2H])c([2H])c45)c32)c([2H])c1[2H] ; [2H]c1cc2c(oc3c([2H])c(-c4cccc5c(-c6ccccc6)cccc45)c([2H])c(-c4c5c([2H])c([2H])c([2H])c([2H])c5c(-c5c([2H])c([2H])c6c([2H])c([2H])c7c([2H])c([2H])c([2H])c([2H])c7c6c5[2H])c5c([2H])c([2H])c([2H])c([2H])c45)c32)c([2H])c1[2H]]

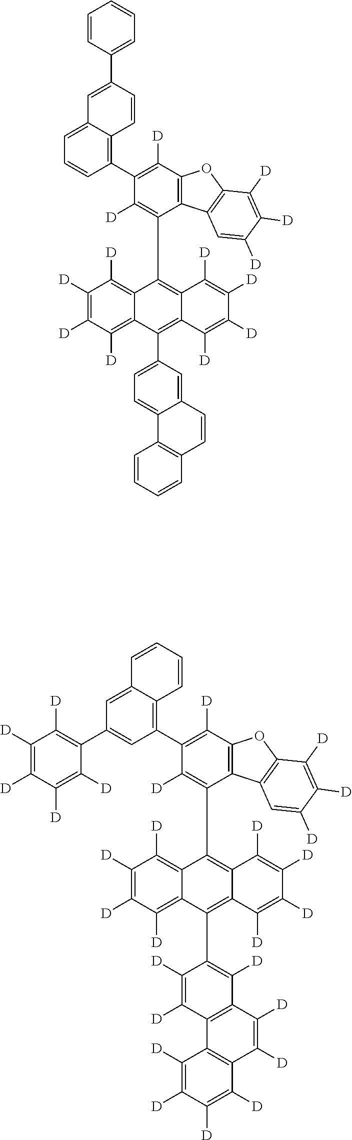 [SMILES: [2H]c1cc2c(oc3c([2H])c(-c4cccc5cc(-c6ccccc6)ccc45)c([2H])c(-c4c5c([2H])c([2H])c([2H])c([2H])c5c(-c5ccc6c(ccc7ccccc76)c5)c5c([2H])c([2H])c([2H])c([2H])c45)c32)c([2H])c1[2H] ; [2H]c1cc2c(oc3c([2H])c(-c4cc(-c5c([2H])c([2H])c([2H])c([2H])c5[2H])cc5ccccc45)c([2H])c(-c4c5c([2H])c([2H])c([2H])c([2H])c5c(-c5c([2H])c([2H])c6c(c5[2H])c([2H])c([2H])c5c([2H])c([2H])c([2H])c([2H])c56)c5c([2H])c([2H])c([2H])c([2H])c45)c32)c([2H])c1[2H]]

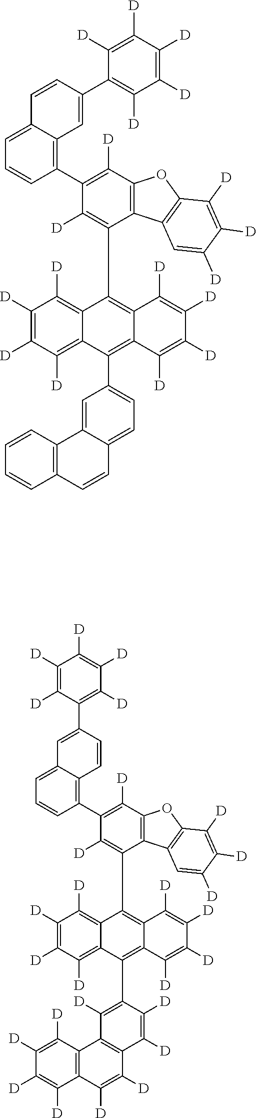 [SMILES: [2H]c1cc2c(oc3c([2H])c(-c4cccc5ccc(-c6c([2H])c([2H])c([2H])c([2H])c6[2H])cc45)c([2H])c(-c4c5c([2H])c([2H])c([2H])c([2H])c5c(-c5ccc6ccc7ccccc7c6c5)c5c([2H])c([2H])c([2H])c([2H])c45)c32)c([2H])c1[2H] ; [2H]c1cc2c(oc3c([2H])c(-c4cccc5cc(-c6c([2H])c([2H])c([2H])c([2H])c6[2H])ccc45)c([2H])c(-c4c5c([2H])c([2H])c([2H])c([2H])c5c(-c5c([2H])c([2H])c6c([2H])c([2H])c7c([2H])c([2H])c([2H])c([2H])c7c6c5[2H])c5c([2H])c([2H])c([2H])c([2H])c45)c32)c([2H])c1[2H]]

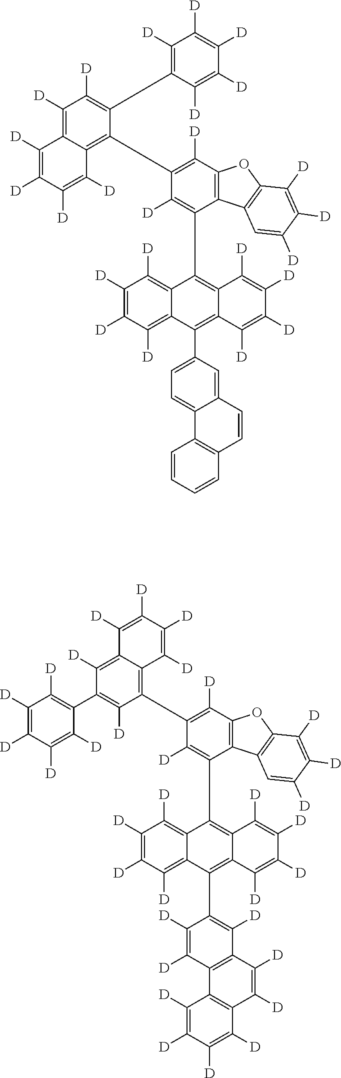

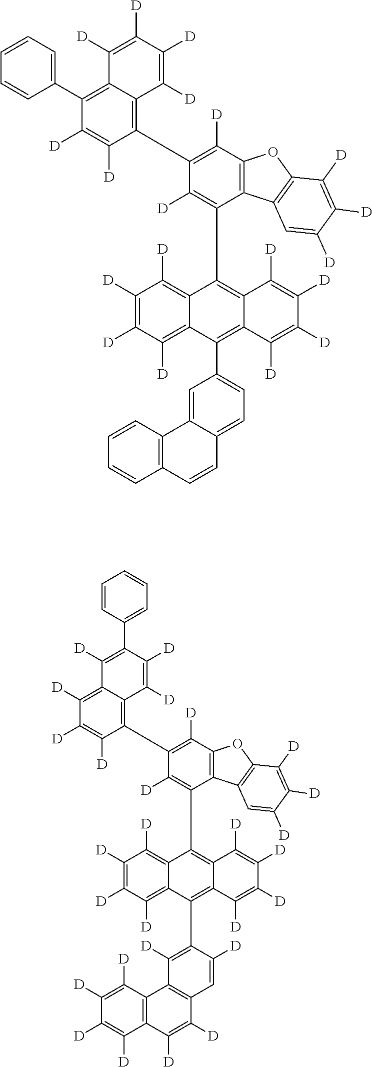 [SMILES: [2H]c1cc2c(oc3c([2H])c(-c4c([2H])c([2H])c(-c5ccccc5)c5c([2H])c([2H])c([2H])c([2H])c45)c([2H])c(-c4c5c([2H])c([2H])c([2H])c([2H])c5c(-c5ccc6ccc7ccccc7c6c5)c5c([2H])c([2H])c([2H])c([2H])c45)c32)c([2H])c1[2H] ; [2H]c1cc2c(oc3c([2H])c(-c4c([2H])c([2H])c([2H])c5c([2H])c(-c6ccccc6)c([2H])c([2H])c45)c([2H])c(-c4c5c([2H])c([2H])c([2H])c([2H])c5c(-c5c([2H])cc6c([2H])c([2H])c7c([2H])c([2H])c([2H])c([2H])c7c6c5[2H])c5c([2H])c([2H])c([2H])c([2H])c45)c32)c([2H])c1[2H]]

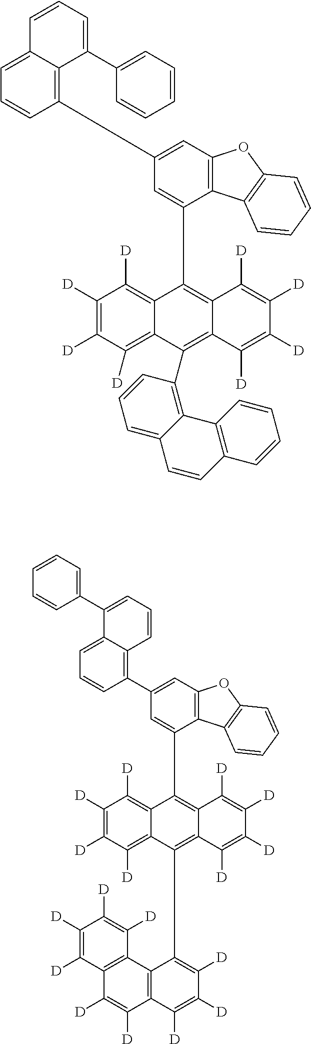 [SMILES: [2H]c1c([2H])c([2H])c2c(-c3cc(-c4cccc5cccc(-c6ccccc6)c45)cc4oc5ccccc5c34)c3c([2H])c([2H])c([2H])c([2H])c3c(-c3cccc4ccc5ccccc5c34)c2c1[2H] ; [2H]c1c([2H])c([2H])c2c(-c3c([2H])c([2H])c([2H])c4c([2H])c([2H])c5c([2H])c([2H])c([2H])c([2H])c5c34)c3c([2H])c([2H])c([2H])c([2H])c3c(-c3cc(-c4cccc5c(-c6ccccc6)cccc45)cc4oc5ccccc5c34)c2c1[2H]]

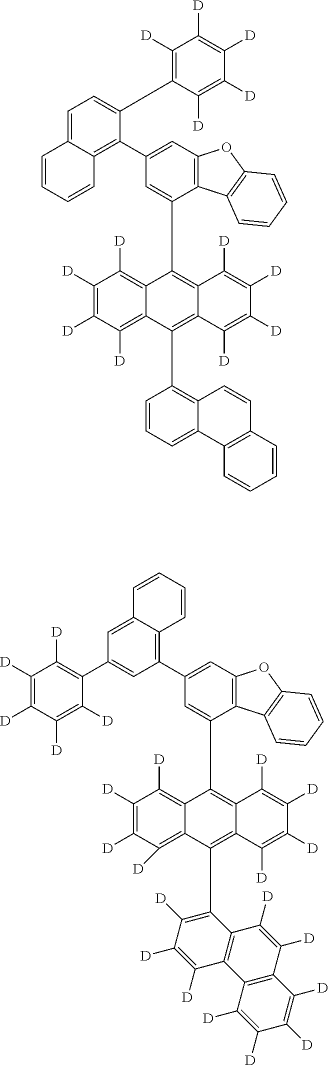 [SMILES: [2H]c1c([2H])c([2H])c(-c2ccc3ccccc3c2-c2cc(-c3c4c([2H])c([2H])c([2H])c([2H])c4c(-c4cccc5c4ccc4ccccc45)c4c([2H])c([2H])c([2H])c([2H])c34)c3c(c2)oc2ccccc23)c([2H])c1[2H] ; [2H]c1c([2H])c([2H])c(-c2cc(-c3cc(-c4c5c([2H])c([2H])c([2H])c([2H])c5c(-c5c([2H])c([2H])c([2H])c6c5c([2H])c([2H])c5c([2H])c([2H])c([2H])c([2H])c56)c5c([2H])c([2H])c([2H])c([2H])c45)c4c(c3)oc3ccccc34)c3ccccc3c2)c([2H])c1[2H]]

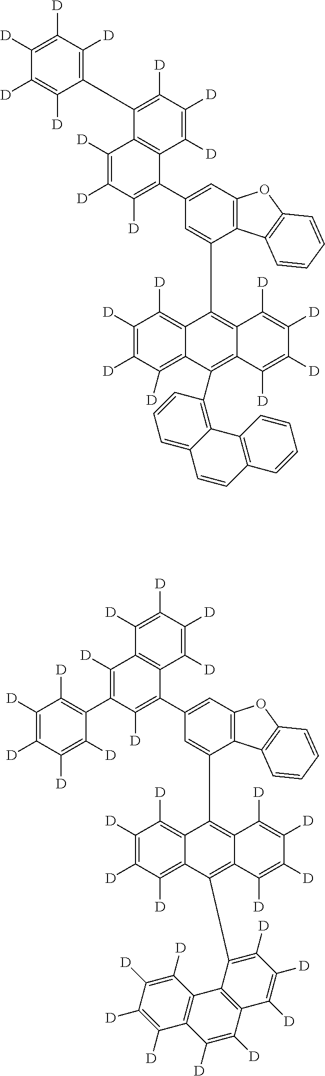

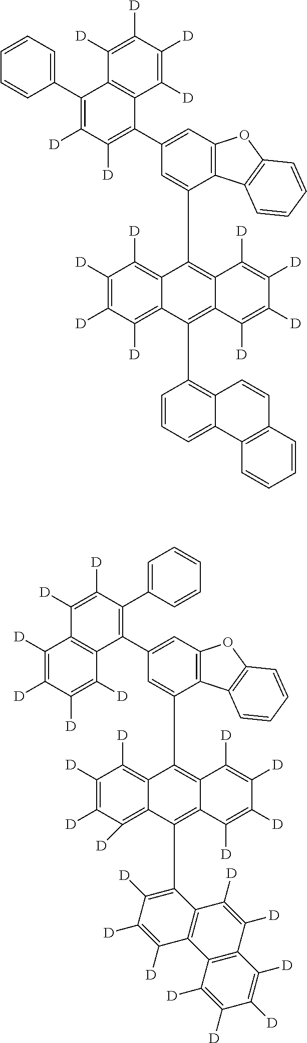 [SMILES: [2H]c1c([2H])c([2H])c2c(-c3cc(-c4c5c([2H])c([2H])c([2H])c([2H])c5c(-c5cccc6c5ccc5ccccc56)c5c([2H])c([2H])c([2H])c([2H])c45)c4c(c3)oc3ccccc34)c([2H])c([2H])c(-c3ccccc3)c2c1[2H] ; [2H]c1c([2H])c([2H])c2c(-c3cc(-c4c5c([2H])c([2H])c([2H])c([2H])c5c(-c5c([2H])c([2H])c([2H])c6c5c([2H])c([2H])c5c([2H])c([2H])c([2H])c([2H])c56)c5c([2H])c([2H])c([2H])c([2H])c45)c4c(c3)oc3ccccc34)c(-c3ccccc3)c([2H])c([2H])c2c1[2H]]

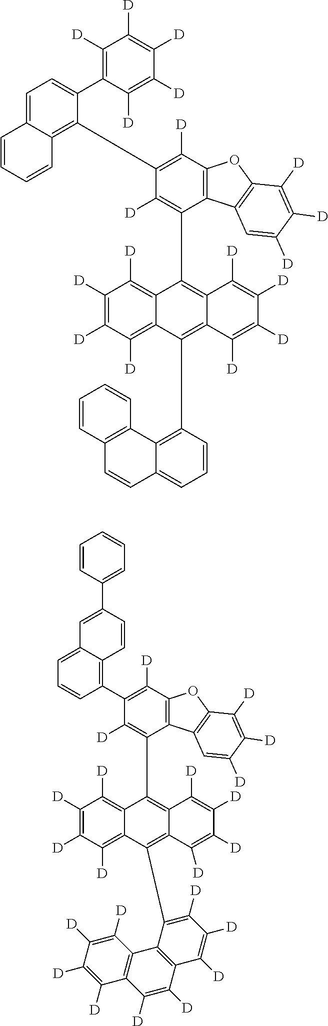 [SMILES: [2H]c1cc2c(oc3c([2H])c(-c4c(-c5c([2H])c([2H])c([2H])c([2H])c5[2H])ccc5ccccc45)c([2H])c(-c4c5c([2H])c([2H])c([2H])c([2H])c5c(-c5cccc6ccc7ccccc7c56)c5c([2H])c([2H])c([2H])c([2H])c45)c32)c([2H])c1[2H] ; [2H]c1cc2c(oc3c([2H])c(-c4cccc5cc(-c6ccccc6)ccc45)c([2H])c(-c4c5c([2H])c([2H])c([2H])c([2H])c5c(-c5c([2H])c([2H])c([2H])c6c([2H])c([2H])c7c([2H])c([2H])c([2H])c([2H])c7c56)c5c([2H])c([2H])c([2H])c([2H])c45)c32)c([2H])c1[2H]]

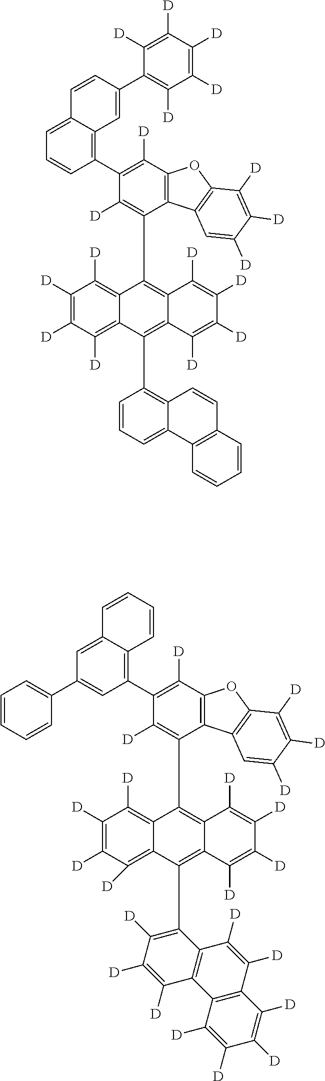 [SMILES: [2H]c1cc2c(oc3c([2H])c(-c4cccc5ccc(-c6c([2H])c([2H])c([2H])c([2H])c6[2H])cc45)c([2H])c(-c4c5c([2H])c([2H])c([2H])c([2H])c5c(-c5cccc6c5ccc5ccccc56)c5c([2H])c([2H])c([2H])c([2H])c45)c32)c([2H])c1[2H] ; [2H]c1cc2c(oc3c([2H])c(-c4cc(-c5ccccc5)cc5ccccc45)c([2H])c(-c4c5c([2H])c([2H])c([2H])c([2H])c5c(-c5c([2H])c([2H])c([2H])c6c5c([2H])c([2H])c5c([2H])c([2H])c([2H])c([2H])c56)c5c([2H])c([2H])c([2H])c([2H])c45)c32)c([2H])c1[2H]]

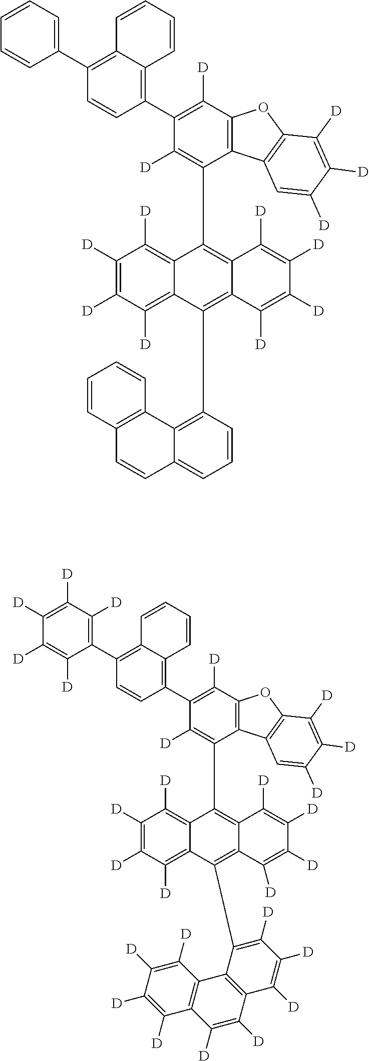 [SMILES: [2H]c1cc2c(oc3c([2H])c(-c4ccc(-c5ccccc5)c5ccccc45)c([2H])c(-c4c5c([2H])c([2H])c([2H])c([2H])c5c(-c5cccc6ccc7ccccc7c56)c5c([2H])c([2H])c([2H])c([2H])c45)c32)c([2H])c1[2H] ; [2H]c1cc2c(oc3c([2H])c(-c4ccc(-c5c([2H])c([2H])c([2H])c([2H])c5[2H])c5ccccc45)c([2H])c(-c4c5c([2H])c([2H])c([2H])c([2H])c5c(-c5c([2H])c([2H])c([2H])c6c([2H])c([2H])c7c([2H])c([2H])c([2H])c([2H])c7c56)c5c([2H])c([2H])c([2H])c([2H])c45)c32)c([2H])c1[2H]]

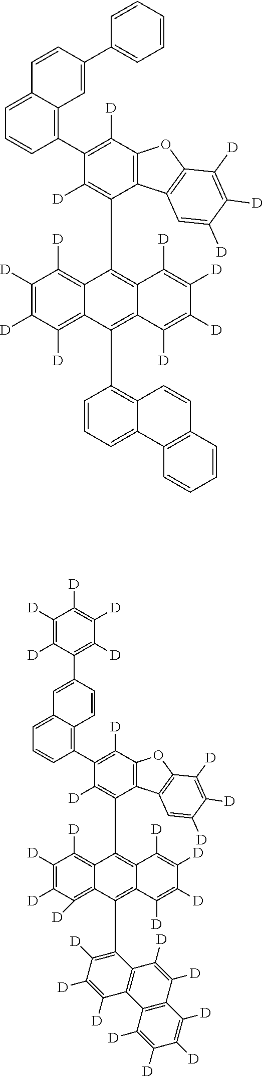 [SMILES: [2H]c1cc2c(oc3c([2H])c(-c4cccc5ccc(-c6ccccc6)cc45)c([2H])c(-c4c5c([2H])c([2H])c([2H])c([2H])c5c(-c5cccc6c5ccc5ccccc56)c5c([2H])c([2H])c([2H])c([2H])c45)c32)c([2H])c1[2H] ; [2H]c1cc2c(oc3c([2H])c(-c4cccc5cc(-c6c([2H])c([2H])c([2H])c([2H])c6[2H])ccc45)c([2H])c(-c4c5c([2H])c([2H])c([2H])c([2H])c5c(-c5c([2H])c([2H])c([2H])c6c5c([2H])c([2H])c5c([2H])c([2H])c([2H])c([2H])c56)c5c([2H])c([2H])c([2H])c([2H])c45)c32)c([2H])c1[2H]]

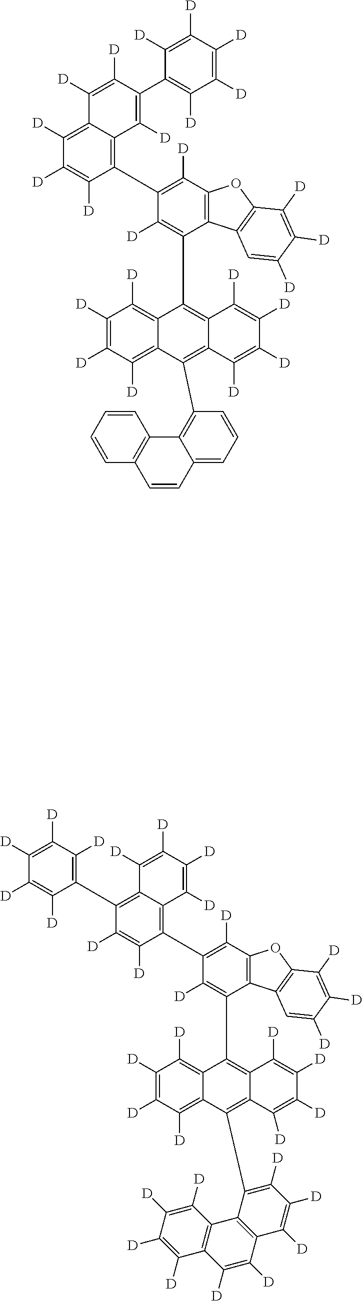

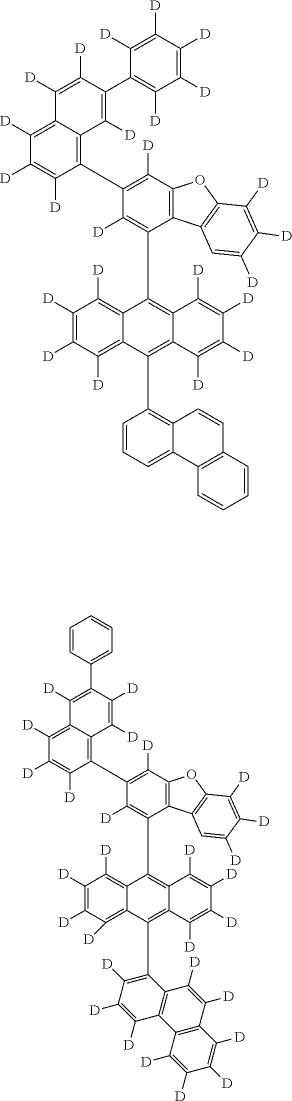 [SMILES: [2H]c1cc2c(oc3c([2H])c(-c4c([2H])c([2H])c([2H])c5c([2H])c([2H])c(-c6c([2H])c([2H])c([2H])c([2H])c6[2H])c([2H])c45)c([2H])c(-c4c5c([2H])c([2H])c([2H])c([2H])c5c(-c5cccc6c5ccc5ccccc56)c5c([2H])c([2H])c([2H])c([2H])c45)c32)c([2H])c1[2H] ; [2H]c1cc2c(oc3c([2H])c(-c4c([2H])c([2H])c([2H])c5c([2H])c(-c6ccccc6)c([2H])c([2H])c45)c([2H])c(-c4c5c([2H])c([2H])c([2H])c([2H])c5c(-c5c([2H])c([2H])c([2H])c6c5c([2H])c([2H])c5c([2H])c([2H])c([2H])c([2H])c56)c5c([2H])c([2H])c([2H])c([2H])c45)c32)c([2H])c1[2H]]

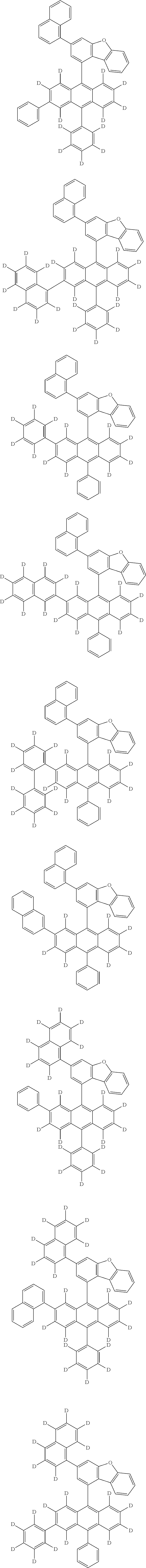

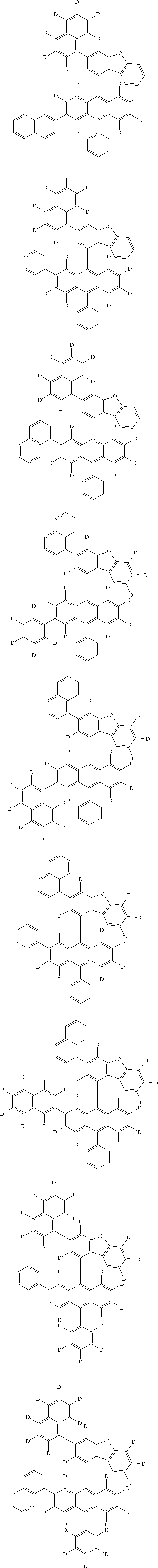

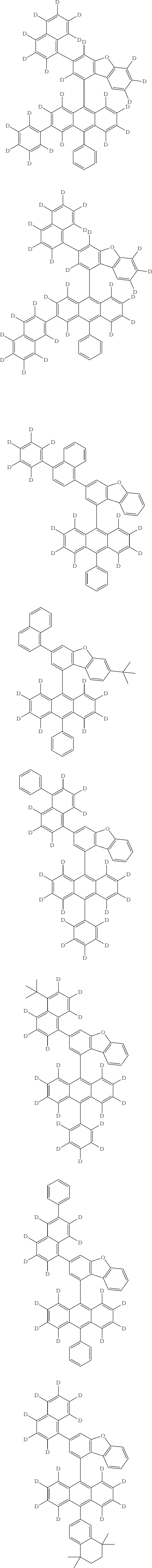

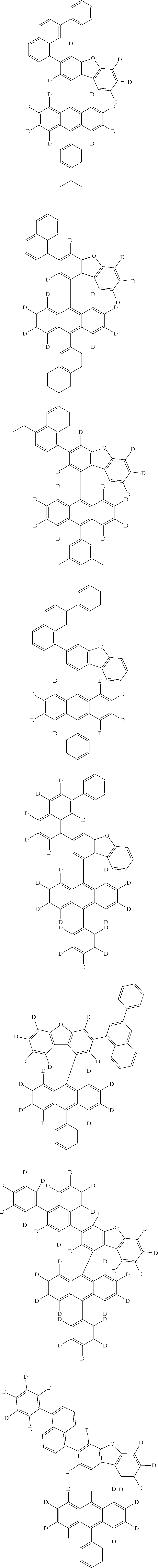

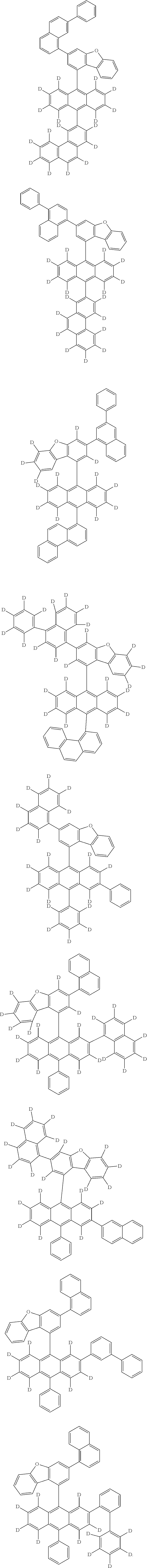

14. An organic light-emitting device comprising: a first electrode; a second electrode provided opposite to the first electrode; and one or more organic layers provided between the first electrode and the second electrode, wherein one or more of the one or more organic layers comprises the compound of Chemical Formula 1-1 or Chemical Formula 1-2 according to claim 1.

15. The organic light-emitting device according to claim 14, wherein the organic layer comprises a light-emitting layer, and the light-emitting layer comprises the compound of Chemical Formula 1-1 or Chemical Formula 1-2.

16. The organic light-emitting device according to claim 14, wherein the organic layer comprises a light-emitting layer, and the light-emitting layer comprises the compound Chemical Formula 1-1 or Chemical Formula 1-2 as a host of the light-emitting layer.

17. The organic light-emitting device according to claim 16, wherein the light-emitting layer further comprises a dopant.
